(19) United States
(12) Patent Application Publication
(10) Pub. No.: US 2023/0165140 A1
(43) Pub. Date: May 25, 2023

(54) COMPOUND AND ORGANIC LIGHT-EMITTING DEVICE COMPRISING SAME

(71) Applicant: LG CHEM, LTD., Seoul (KR)

(72) Inventors: Miyeon Han, Daejeon (KR); Sung Kil Hong, Daejeon (KR); Dong Uk Heo, Daejeon (KR); Jung Min Yoon, Daejeon (KR); Heekyung Yun, Daejeon (KR)

(21) Appl. No.: 17/919,193

(22) PCT Filed: May 27, 2021
(86) PCT No.: PCT/KR2021/006581

§ 371 Date: Oct. 14, 2022

(30) Foreign Application Priority Data
Jun. 3, 2020 (KR) 10-2020-0066777

(51) Int. Cl. H10K 85/60; C07D 251/24; C09K 11/06; C07D 239/26; C07D 405/04

(52) U.S. Cl. CPC H10K 85/654; C07D 251/24; C09K 11/06; H10K 85/622; H10K 85/626; C07D 239/26; H10K 85/623; C07D 405/04; H10K 85/6574; H10K 50/18

(57) ABSTRACT

A compound of Chemical Formula 1, and an organic light emitting device including the same are provided. The compound is used as a material of an organic material layer of an organic light emitting device, and provides enhanced efficiency, low driving voltage and/or increased lifetime properties of the organic light emitting device.

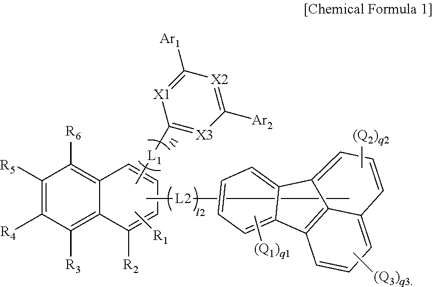

### CROSS-REFERENCE TO RELATED APPLICATION

This application is a National Stage Application of International Application No. PCT/KR2021/006581 filed on May 27, 2021, which claims priority to and the benefits of Korean Patent Application No. 10-2020-0066777 filed on Jun. 3, 2020, the disclosures of which are incorporated herein by reference in their entireties.

### FIELD OF DISCLOSURE

The present specification relates to a compound, and an organic light emitting device including the same.

### BACKGROUND

An organic light emission phenomenon generally refers to a phenomenon converting electrical energy to light energy using an organic material. An organic light emitting device using an organic light emission phenomenon normally has a structure including an anode, a cathode, and an organic material layer therebetween. Herein, the organic material layer is often formed in a multilayer structure formed with different materials in order to increase efficiency and stability of the organic light emitting device, and for example, may be formed with a hole injection layer, a hole transfer layer, a light emitting layer, an electron transfer layer, an electron injection layer and the like. When a voltage is applied between the two electrodes in such an organic light emitting device structure, holes and electrons are injected to the organic material layer from the anode and the cathode, respectively, and when the injected holes and electrons meet, excitons are formed, and light emits when these excitons fall back to the ground state.

Development of new materials for such an organic light emitting device has been continuously required.

### SUMMARY

The present specification is directed to providing a compound, and an organic light emitting device including the same.

One embodiment of the present specification provides a compound represented by the following Chemical Formula 1.

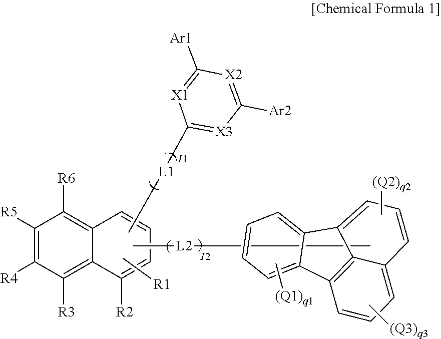

In Chemical Formula 1,

at least two of X1 to X3 are N, and the rest is CR,

R is hydrogen or deuterium,

L1 and L2 are the same as or different from each other, and each independently a direct bond; a substituted or unsubstituted alkylene group; a substituted or unsubstituted alkenylene group; a substituted or unsubstituted arylene group; or a substituted or unsubstituted heteroarylene group,

Ar1, Ar2, R1 to R6 and Q1 to Q3 are the same as or different from each other, and each independently hydrogen; deuterium; a halogen group; a cyano group; a nitro group; a hydroxyl group; a substituted or unsubstituted alkyl group; a substituted or unsubstituted cycloalkyl group; a substituted or unsubstituted alkoxy group; a substituted or unsubstituted aryloxy group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted amine group; a substituted or unsubstituted arylphosphine group; a substituted or unsubstituted phosphine oxide group; a substituted or unsubstituted aryl group; or a substituted or unsubstituted heteroaryl group, or adjacent groups among Ar1, Ar2, R1 to R6 and Q1 to Q3 bond to each other to form a substituted or unsubstituted ring,

l1 is an integer of 1 to 5,

l2 is an integer of 1 to 5,

q1 is an integer of 1 to 4,

q2 is an integer of 1 to 3,

q3 is an integer of 1 to 3,

when l1 is 2 or greater, the two or more L1s are the same as or different from each other,

when l2 is 2 or greater, the two or more L2s are the same as or different from each other,

when q1 is 2 or greater, the two or more Q1s are the same as or different from each other,

when q2 is 2 or greater, the two or more Q2s are the same as or different from each other,

when q3 is 2 or greater, the two or more Q3s are the same as or different from each other, and

2≤q1+q2≤6.

Another embodiment of the present specification provides an organic light emitting device including a first electrode; a second electrode; and one or more organic material layers provided between the first electrode and the second electrode, wherein one or more layers of the organic material layers include the compound.

A compound according to one embodiment of the present specification can be used as a material of an organic material layer of an organic light emitting device, and by using the same, efficiency can be enhanced, low driving voltage can be obtained and/or lifetime properties can be enhanced in the organic light emitting device.

### BRIEF DESCRIPTION OF DRAWINGS

FIG. 1 and FIG. 2 illustrate embodiments of organic light emitting devices according to the present specification.

### DESCRIPTION OF REFERENCE NUMERALS

101: Substrate
102: First Electrode
111: Organic Material Layer
110: Second Electrode
103: Hole Injection Layer
104: First Hole Transfer Layer
105: Second Hole Transfer Layer
106: Light Emitting Layer
107: Electron Injection and Transfer Layer

### DETAILED DESCRIPTION

Hereinafter, the present specification will be described in more detail.

One embodiment of the present specification provides a compound represented by Chemical Formula 1.

Chemical Formula 1 according to one embodiment of the present specification is a compound in which an N-containing heterocyclic group and a fluoranthene derivative bond at positions of 1,2, positions of 1,3 or positions of 2,3 of naphthalene, and, by being included in an organic material layer of an organic light emitting device, is capable of enhancing efficiency, obtaining a low driving voltage, enhancing lifetime properties, and the like. Specifically, the N-containing heterocyclic group of Chemical Formula 1 is capable of increasing molecular polarity (dipole moment) by having a substituent with an electron-deficient structure, and therefore, electron mobility may be smoothly controlled when manufacturing an organic light emitting device including the compound represented by Chemical Formula 1, and efficiency and lifetime of the organic light emitting device including the compound represented by Chemical Formula 1 may be enhanced.

In addition, steric hindrance obtained by the structure of the fluoranthene derivative of Chemical Formula 1 prevents crystallization occurring when forming a film, and has an effect of being very stable even at a high deposition temperature by maintaining high thermal stability. Accordingly, an organic light emitting device including the compound according to one embodiment of the present specification is capable of enhancing efficiency, obtaining a low driving voltage, enhancing lifetime properties, and the like.

Examples of substituents in the present specification are described below, however, the substituents are not limited thereto.

In the present specification, [symbol] means a linking site.

The term “substitution” means a hydrogen atom bonding to a carbon atom of a compound being changed to another substituent, and the position of substitution is not limited as long as it is a position at which the hydrogen atom is substituted, that is, a position at which a substituent can substitute, and when two or more substituents substitute, the two or more substituents may be the same as or different from each other.

In the present specification, a term “substituted or unsubstituted” means being substituted with one or more substituents selected from the group consisting of deuterium; a halogen group; a cyano group; an alkyl group; a cycloalkyl group; an alkoxy group; an alkenyl group; a haloalkyl group; a silyl group; a boron group; an amine group; an aryl group; and a heteroaryl group, or being substituted with a substituent linking two or more substituents among the substituents illustrated above, or having no substituents.

In the present specification, linking two or more substituents refers to linking hydrogen of any one substituent to another substituent. For example, linking two or more substituents may include a phenyl group and a naphthyl group being linked to become a substituent of

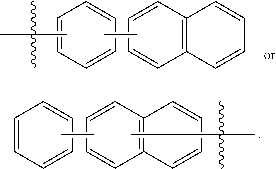

In addition, linking three substituents includes not only continuously linking (substituent 1)-(substituent 2)-(substituent 3), but also linking (substituent 2) and (substituent 3) to (substituent 1). For example, a phenyl group, a naphthyl group and an isopropyl group may be linked to become a substituent of

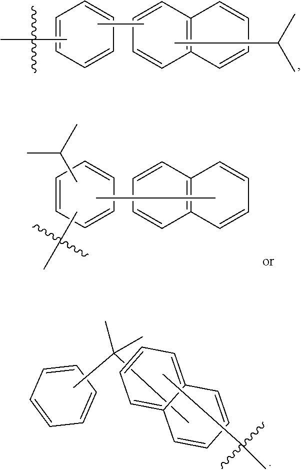

The same definition described above also applies when linking four or more substituents.

In the present specification, examples of the halogen group may include fluorine, chlorine, bromine or iodine.

In the present specification, the alkyl group may be linear or branched, and although not particularly limited thereto, the number of carbon atoms is preferably from 1 to 30. Specific examples thereof may include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl and the like, but are not limited thereto.

In the present specification, the cycloalkyl group is not particularly limited, but preferably has 3 to 30 carbon atoms. Specific examples thereof may include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, an adamantyl group and the like, but are not limited thereto.

In the present specification, the alkoxy group may be linear, branched or cyclic. The number of carbon atoms of the alkoxy group is not particularly limited, but is preferably from 1 to 30. Specific examples thereof may include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy and the like, but are not limited thereto.

In the present specification, the alkenyl group may be linear or branched, and although not particularly limited thereto, the number of carbon atoms is preferably from 2 to 30. Specific examples thereof may include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl yl)vinyl-1-yl, a stilbenyl group, a styrenyl group and the like, but are not limited thereto.

In the present specification, the haloalkyl group means, in the definition of the alkyl group, hydrogen of the alkyl group being substituted with at least one halogen group.

In the present specification, the aryl group is not particularly limited, but preferably has 6 to 30 carbon atoms, and the aryl group may be monocyclic or polycyclic.

When the aryl group is a monocyclic aryl group, the number of carbon atoms is not particularly limited, but is preferably from 6 to 30. Specific examples of the monocyclic aryl group may include a phenyl group, a biphenyl group, a terphenyl group and the like, but are not limited thereto.

When the aryl group is a polycyclic aryl group, the number of carbon atoms is not particularly limited, but is preferably from 10 to 30. Specific examples of the polycyclic aryl group may include a naphthyl group, an anthracene group, a phenanthrene group, a triphenylene group, a pyrene group, a phenalene group, a perylene group, a chrysene group, a fluorene group and the like, but are not limited thereto.

In the present specification, the fluorene group may be substituted, and adjacent groups may bond to each other to form a ring.

When the fluorene group is substituted,

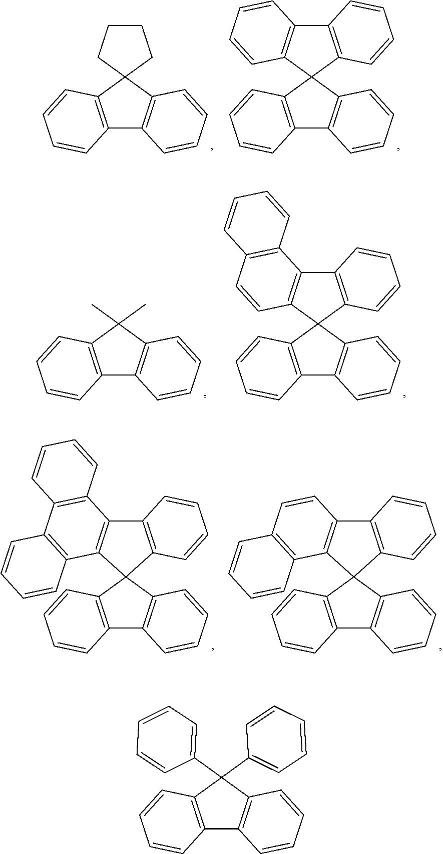

and the like may be included, however, the structure is not limited thereto.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring may be interpreted as groups “adjacent” to each other.

In the present specification, the heteroaryl group is a group including one or more atoms that are not carbon, that is, heteroatoms, and specifically, the heteroatom may include one or more atoms selected from the group consisting of O, N, Se, S and the like. The number of carbon atoms is not particularly limited, but is preferably from 2 to 30, and the heteroaryl group may be monocyclic or polycyclic. Examples of the heterocyclic group may include a thiophene group, a furan group, a pyrrole group, an imidazole group, a thiazole group, an oxazole group, an oxadiazole group, a pyridine group, a bipyridine group, a pyrimidine group, a triazine group, a triazole group, an acridine group, a pyridazine group, a pyrazine group, a quinoline group, a quinazoline group, a quinoxaline group, a phthalazine group, a pyridopyrimidine group, a pyridopyrazine group, a pyrazinopyrazine group, an isoquinoline group, an indole group, a carbazole group, a benzoxazole group, a benzimidazole group, a benzothiazole group, a benzocarbazole group, a benzothiophene group, a dibenzothiophene group, a benzofuran group, a phenanthridine group, a phenanthroline group, an isoxazole group, a thiadiazole group, a dibenzofuran group, dibenzosilole group, a phenoxanthine group, a phenoxazine group, a phenothiazine group, a dihydroindenocarbazole group, a spirofluorenexanthene group, a spirofluorenethioxanthene group and the like, but are not limited thereto.

In the present specification, the silyl group may be an alkylsilyl group, an arylsilyl group, a heteroarylsilyl group or the like. As the alkyl group in the alkylsilyl group, the examples of the alkyl group described above may be applied, and as the aryl group in the arylsilyl group, the examples of the aryl group described above may be applied, and as the heteroaryl group in the heteroarylsilyl group, the examples of the heteroaryl group may be applied.

In the present specification, the boron group may be —BR₁₀₀R₁₀₁. R₁₀₀ and R₁₀₁ are the same as or different from each other, and may be each independently selected from the group consisting of hydrogen; deuterium; halogen; a nitrile group; a substituted or unsubstituted monocyclic or polycyclic cycloalkyl group having 3 to 30 carbon atoms; a substituted or unsubstituted linear or branched alkyl group having 1 to 30 carbon atoms; a substituted or unsubstituted monocyclic or polycyclic aryl group having 6 to 30 carbon atoms; and a substituted or unsubstituted monocyclic or polycyclic heteroaryl group having 2 to 30 carbon atoms. Specific examples of the boron group may include a trimethylboron group, a triethylboron group, a t-butyldimethylboron group, a triphenylboron group, a phenylboron group and the like, but are not limited thereto.

In the present specification, the amine group may be selected from the group consisting of —NH₂, an alkylamine group, an N-alkylarylamine group, an arylamine group, an N-arylheteroarylamine group, an N-alkylheteroarylamine group and a heteroarylamine group, and although not particularly limited thereto, the number of carbon atoms is preferably from 1 to 30. Specific examples of the amine group may include a methylamine group, a dimethylamine group, an ethylamine group, a diethylamine group, a phenylamine group, a naphthylamine group, a biphenylamine group, an anthracenylamine group, a 9-methyl-anthracenylamine group, a diphenylamine group, a ditolylamine group, an N-phenyltolylamine group, a triphenylamine group, an N-phenylbiphenylamine group, an N-phenylnaphthylamine group, an N-biphenylnaphthylamine group, an N-naphthylfluorenylamine group, an N-phenylphenanthrenylamine group, an N-biphenylphenanthrenylamine group, an N-phenylfluorenylamine group, an N-phenylterphenylamine group, an N-phenanthrenylfluorenylamine group, an N-biphenylfluorenylamine group and the like, but are not limited thereto.

In the present specification, the N-alkylarylamine group means an amine group in which N of the amine group is substituted with an alkyl group and an aryl group. The alkyl group and the aryl group in the N-alkylarylamine group are the same as the examples of the alkyl group and the aryl group described above.

In the present specification, the N-arylheteroarylamine group means an amine group in which N of the amine group is substituted with an aryl group and a heteroaryl group. The aryl group and the heteroaryl group in the N-arylheteroarylamine group are the same as the examples of the aryl group and the heteroaryl group described above.

In the present specification, the N-alkylheteroarylamine group means an amine group in which N of the amine group is substituted with an alkyl group and a heteroaryl group. The alkyl group and the heteroaryl group in the N-alkylheteroarylamine group are the same as the examples of the alkyl group and the heteroaryl group described above.

In the present specification, examples of the arylamine group include a substituted or unsubstituted monoarylamine group, or a substituted or unsubstituted diarylamine group. The arylamine group including two or more aryl groups may include monocyclic aryl groups, polycyclic aryl groups, or both monocyclic aryl groups and polycyclic aryl groups. For example, the aryl group in the arylamine group may be selected from among the examples of the aryl group described above.

In the present specification, examples of the heteroarylamine group include a substituted or unsubstituted monoheteroarylamine group, or a substituted or unsubstituted diheteroarylamine group. The heteroarylamine group including two or more heteroaryl groups may include monocyclic heteroaryl groups, polycyclic heteroaryl groups, or both monocyclic heteroaryl groups and polycyclic heteroaryl groups. For example, the heteroaryl group in the heteroarylamine group may be selected from among the examples of the heteroaryl group described above.

In the present specification, the alkyl group in the alkylthioxy group and the alkylsulfoxy group is the same as the examples of the alkyl group described above. Specific examples of the alkylthioxy group may include a methylthioxy group, an ethylthioxy group, a tert-butylthioxy group, a hexylthioxy group, an octylthioxy group and the like, and specific examples of the alkylsulfoxy group may include a methylsulfoxy group, an ethylsulfoxy group, a propylsulfoxy group, a butylsulfoxy group and the like, however, the alkylthioxy group and the alkylsulfoxy group are not limited thereto.

In the present specification, the phosphine oxide group may specifically be an alkylphosphine oxide group, an arylphosphine oxide group or the like, and more specifically be a diphenylphosphine oxide group, a dinaphthylphosphine oxide group or the like, however, the phosphine oxide group is not limited thereto.

In the present specification, the aryl group in the aryloxy group, the arylthioxy group, the arylsulfoxy group and the arylphosphine group is the same as the examples of the aryl group described above. Specific examples of the aryloxy group may include a phenoxy group, a p-tolyloxy group, an m-tolyloxy group, a 3,5-dimethyl-phenoxy group, a 2,4,6-trimethylphenoxy group, a p-tert-butylphenoxy group, a 3-biphenyloxy group, a 4-biphenyloxy group, a 1-naphthyloxy group, a 2-naphthyloxy group, a 4-methyl-1-naphthyloxy group, a 5-methyl-2-naphthyloxy group, a 1-anthryloxy group, a 2-anthryloxy group, a 9-anthryloxy group, a 1-phenanthryloxy group, a 3-phenanthryloxy group, a 9-phenanthryloxy group and the like, examples of the arylthioxy group may include a phenylthioxy group, a 2-methylphenylthioxy group, a 4-tert-butylphenylthioxy group and the like, and examples of the arylsulfoxy group may include a benzenesulfoxy group, a p-toluenesulfoxy group and the like, however, the aryloxy group, the arylthioxy group and the arylsulfoxy group are not limited thereto.

In the present specification, the meaning of “adjacent groups among substituents bonding to each other to form a ring” means bonding to adjacent groups to form a substituted or unsubstituted hydrocarbon ring; or a substituted or unsubstituted heteroring.

In the present specification, the “ring” in the substituted or unsubstituted ring formed by bonding to each other means a substituted or unsubstituted hydrocarbon ring; or a substituted or unsubstituted heteroring.

In the present specification, the hydrocarbon ring may be an aromatic hydrocarbon ring, an aliphatic hydrocarbon ring, or a fused ring of aromatic hydrocarbon and aliphatic hydrocarbon, and may be selected from among the examples of the cycloalkyl group or the aryl group except for those that are not monovalent.

In the present specification, the heteroring means a ring including one or more atoms that are that carbon, that is, heteroatoms, and specifically, the heteroatom may include one or more atoms selected form the group consisting of O, N, Se, S and the like. The heteroring may be monocyclic or polycyclic, and may be aromatic, aliphatic, or a fused ring of aromatic and aliphatic. The aromatic heteroring may be selected from among the examples of the heteroaryl group except for those that are not monovalent.

In the present specification, the aliphatic heteroring means an aliphatic ring including one or more of heteroatoms. Examples of the aliphatic heteroring may include oxirane, tetrahydrofuran, 1,4-dioxane, pyrrolidine, piperidine, morpholine, oxepane, azokane, thiokane and the like, but are not limited thereto.

In the present specification, the alkylene group means the alkyl group having two bonding sites, that is, a divalent group. The descriptions on the alkyl group provided above may be applied thereto except that these are each a divalent group.

In the present specification, the alkenylene group means the alkenyl group having two bonding sites, that is, a divalent group. The descriptions on the alkenyl group provided above may be applied thereto except that these are each a divalent group.

In the present specification, the arylene group means the aryl group having two bonding sites, that is, a divalent group. The descriptions on the aryl group provided above may be applied thereto except that these are each a divalent group.

In the present specification, the heteroarylene group means the heteroaryl group having two bonding sites, that is, a divalent group. The descriptions on the heteroaryl group provided above may be applied thereto except that these are each a divalent group.

Hereinafter, the compound represented by Chemical Formula 1 will be described in detail.

According to one embodiment of the present specification, the compound of Chemical Formula 1 is represented by any one of the following Chemical Formulae 1-1 to 1-6.

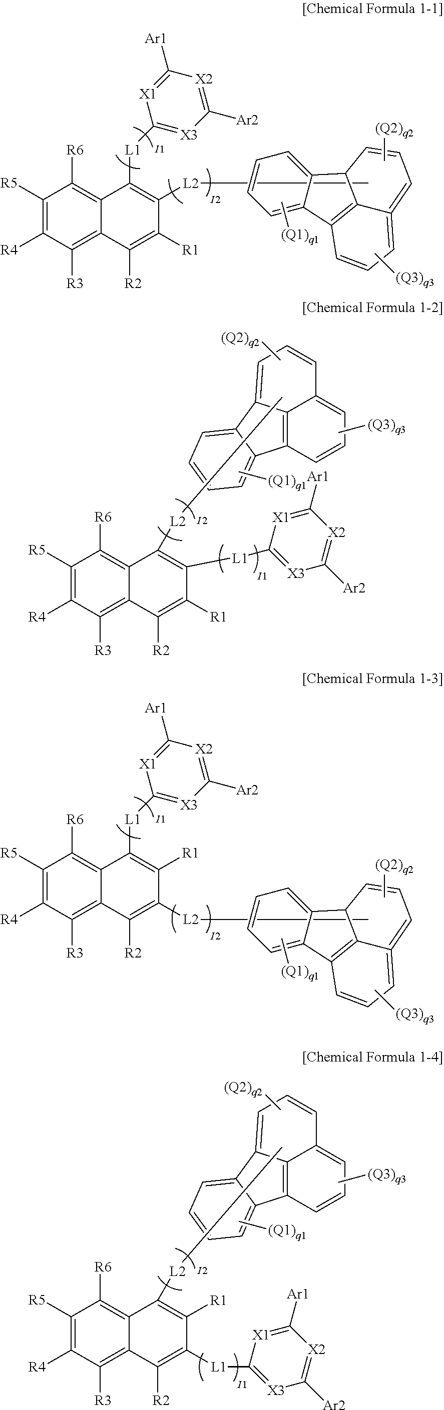

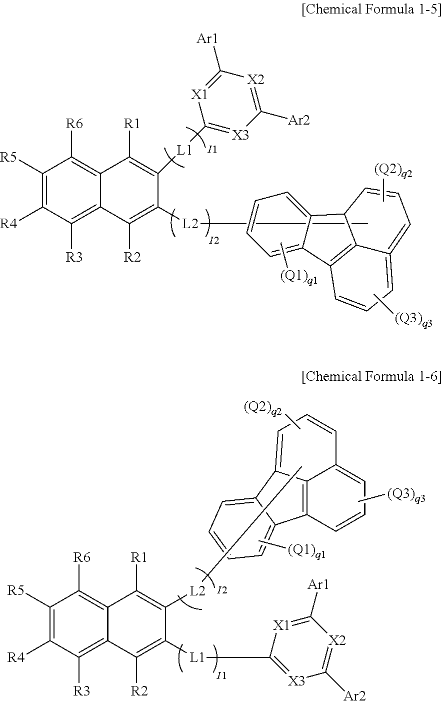

In Chemical Formulae 1-1 to 1-6,

X1 to X3, L1, L2, l1, l2, R1 to R6, Ar1, Ar2, Q1 to Q3 and q1 to q3 have the same definitions as in Chemical Formula 1.

According to one embodiment of the present specification, X1 to X3 are N.

According to one embodiment of the present specification, X1 and X2 are N, and X3 is CR.

According to one embodiment of the present specification, X1 and X3 are N, and X2 is CR.

According to one embodiment of the present specification, X3 and X2 are N, and X1 is CR.

According to one embodiment of the present specification, the compound of Chemical Formula 1 is represented by any one of the following Chemical Formulae 1-7 to 1-9.

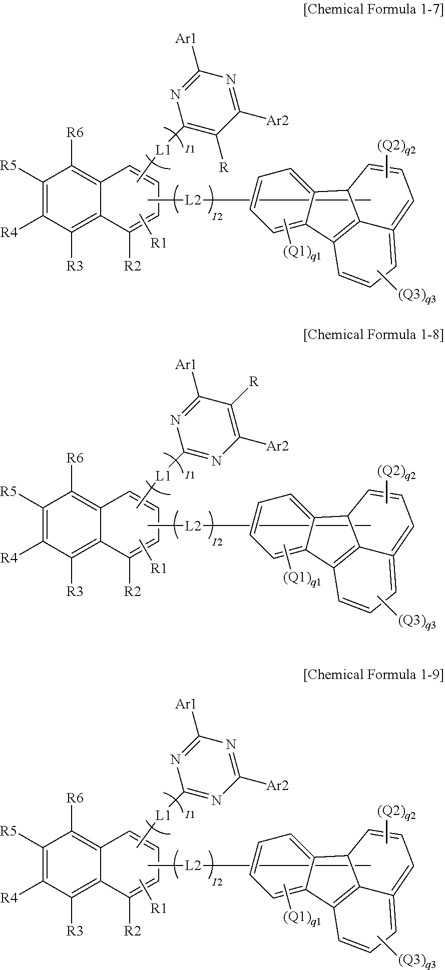

In Chemical Formulae 1-7 to 1-9,

L1, L2, l1, l2, R1 to R6, Ar1, Ar2, Q1 to Q3 and q1 to q3 have the same definitions as in Chemical Formula 1, and

R is hydrogen; or deuterium.

According to one embodiment of the present specification, R is hydrogen; or deuterium.

According to one embodiment of the present specification, R is hydrogen.

According to one embodiment of the present specification, R is deuterium.

According to one embodiment of the present specification, R1 to R6 are hydrogen.

According to one embodiment of the present specification, l1 is 1.

According to one embodiment of the present specification, l2 is 1.

According to one embodiment of the present specification, L1 and L2 are the same as or different from each other, and each independently a direct bond; or a substituted or unsubstituted arylene group.

According to one embodiment of the present specification, L1 and L2 are the same as or different from each other, and each independently a direct bond; or a substituted or unsubstituted monocyclic or polycyclic arylene group having 6 to 30 carbon atoms.

According to one embodiment of the present specification, L1 and L2 are the same as or different from each other, and each independently a direct bond; or a substituted or unsubstituted monocyclic or polycyclic arylene group having 6 to 20 carbon atoms.

According to one embodiment of the present specification, L1 and L2 are the same as or different from each other, and each independently a direct bond; or a substituted or unsubstituted monocyclic or polycyclic arylene group having 6 to 10 carbon atoms.

According to one embodiment of the present specification, L1 and L2 are the same as or different from each other, and each independently a direct bond; or an arylene group.

According to one embodiment of the present specification,

L1 and L2 are the same as or different from each other, and each independently a direct bond; or a monocyclic or polycyclic arylene group having 6 to 30 carbon atoms.

According to one embodiment of the present specification, L1 and L2 are the same as or different from each other, and each independently a direct bond; or a monocyclic or polycyclic arylene group having 6 to 20 carbon atoms.

According to one embodiment of the present specification, L1 and L2 are the same as or different from each other, and each independently a direct bond; or a monocyclic or polycyclic arylene group having 6 to 10 carbon atoms.

According to one embodiment of the present specification, L1 and L2 are the same as or different from each other, and each independently a direct bond; a phenylene group; or a naphthylene group.

According to one embodiment of the present specification, Ar1 and Ar2 are the same as or different from each other, and each independently a substituted or unsubstituted alkyl group; a substituted or unsubstituted aryl group; or a substituted or unsubstituted heteroaryl group.

According to one embodiment of the present specification, Ar1 and Ar2 are the same as or different from each other, and each independently a substituted or unsubstituted linear or branched alkyl group having 1 to 30 carbon atoms; a substituted or unsubstituted monocyclic or polycyclic aryl group having 6 to 30 carbon atoms; or a substituted or unsubstituted monocyclic or polycyclic heteroaryl group having 2 to 30 carbon atoms.

According to one embodiment of the present specification, Ar1 and Ar2 are the same as or different from each other, and each independently a substituted or unsubstituted linear or branched alkyl group having 1 to 20 carbon atoms; a substituted or unsubstituted monocyclic or polycyclic aryl group having 6 to 20 carbon atoms; or a substituted or unsubstituted monocyclic or polycyclic heteroaryl group having 2 to 20 carbon atoms.

According to one embodiment of the present specification, Ar1 and Ar2 are the same as or different from each other, and each independently an alkyl group; an aryl group unsubstituted or substituted with an alkyl group; or a heteroaryl group.

According to one embodiment of the present specification, Ar1 and Ar2 are the same as or different from each other, and each independently a linear or branched alkyl group having 1 to 30 carbon atoms; a monocyclic or polycyclic aryl group having 6 to 30 carbon atoms unsubstituted or substituted with a linear or branched alkyl group having 1 to 30 carbon atoms; or a monocyclic or polycyclic heteroaryl group having 2 to 30 carbon atoms.

According to one embodiment of the present specification, Ar1 and Ar2 are the same as or different from each other, and each independently a linear or branched alkyl group having 1 to 20 carbon atoms; a monocyclic or polycyclic aryl group having 6 to 20 carbon atoms unsubstituted or substituted with a linear or branched alkyl group having 1 to 20 carbon atoms; or a monocyclic or polycyclic heteroaryl group having 2 to 20 carbon atoms.

According to one embodiment of the present specification, Ar1 and Ar2 are the same as or different from each other, and each independently a methyl group; a phenyl group unsubstituted or substituted with a tert-butyl group; a biphenyl group; a naphthyl group; a phenanthrene group; or a dibenzofuran group.

According to one embodiment of the present specification, Q1 to Q3 are the same as or different from each other, and each independently hydrogen; a cyano group; or a substituted or unsubstituted aryl group, or adjacent groups among Q1 to Q3 bond to each other to form a substituted or unsubstituted ring.

According to one embodiment of the present specification, Q1 to Q3 are the same as or different from each other, and each independently hydrogen; a cyano group; or a substituted or unsubstituted monocyclic or polycyclic aryl group having 6 to 30 carbon atoms, or adjacent groups among Q1 to Q3 bond to each other to form a substituted or unsubstituted monocyclic or polycyclic aromatic hydrocarbon ring having 6 to 30 carbon atoms.

According to one embodiment of the present specification, Q1 to Q3 are the same as or different from each other, and each independently hydrogen; a cyano group; or a substituted or unsubstituted monocyclic or polycyclic aryl group having 6 to 20 carbon atoms, or adjacent groups among Q1 to Q3 bond to each other to form a substituted or unsubstituted monocyclic or polycyclic aromatic hydrocarbon ring having 6 to 20 carbon atoms.

According to one embodiment of the present specification, Q1 to Q3 are the same as or different from each other, and each independently hydrogen; a cyano group; or an aryl group, or adjacent groups among Q1 to Q3 bond to each other to form a ring.

According to one embodiment of the present specification, Q1 to Q3 are the same as or different from each other, and each independently hydrogen; a cyano group; or a monocyclic or polycyclic aryl group having 6 to 30 carbon atoms, or adjacent groups among Q1 to Q3 bond to each other to form a monocyclic or polycyclic aromatic hydrocarbon ring having 6 to 30 carbon atoms.

According to one embodiment of the present specification, Q1 to Q3 are the same as or different from each other, and each independently hydrogen; a cyano group; or a monocyclic or polycyclic aryl group having 6 to 20 carbon atoms, or adjacent groups among Q1 to Q3 bond to each other to form a monocyclic or polycyclic aromatic hydrocarbon ring having 6 to 20 carbon atoms.

According to one embodiment of the present specification, Q1 to Q3 are the same as or different from each other, and each independently hydrogen; a cyano group; or a phenyl group, or adjacent groups among Q1 to Q3 bond to each other to form a benzene ring.

According to one embodiment of the present specification, R1 to R6 and R are hydrogen, L1 and L2 are the same as or different from each other and each independently a direct bond; or a monocyclic or polycyclic arylene group having 6 to 30 carbon atoms, Ar1 and Ar2 are the same as or different from each other and each independently a linear or branched alkyl group having 1 to 30 carbon atoms; a monocyclic or polycyclic aryl group having 6 to 30 carbon atoms unsubstituted or substituted with a linear or branched alkyl group having 1 to 30 carbon atoms; or a monocyclic or polycyclic heteroaryl group having 2 to 30 carbon atoms, and Q1 to Q3 are the same as or different from each other and each independently hydrogen; a cyano group; or a monocyclic or polycyclic aryl group having 6 to 30 carbon atoms, or adjacent groups among Q1 to Q3 bond to each other to foil a monocyclic or polycyclic aromatic hydrocarbon ring having 6 to 30 carbon atoms.

According to one embodiment of the present specification, Chemical Formula 1 is any one selected from among the following compounds.

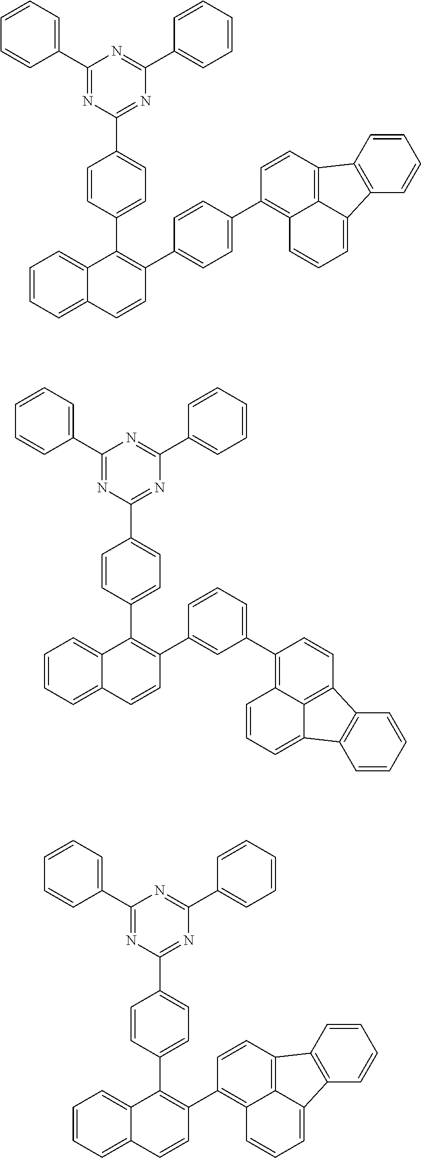 [SMILES: c1ccc(-c2nc(-c3ccccc3)nc(-c3ccc(-c4c(-c5ccc(-c6ccc7c8c(cccc68)-c6ccccc6-7)cc5)ccc5ccccc45)cc3)n2)cc1 ; c1ccc(-c2nc(-c3ccccc3)nc(-c3ccc(-c4c(-c5cccc(-c6ccc7c8c(cccc68)-c6ccccc6-7)c5)ccc5ccccc45)cc3)n2)cc1 ; c1ccc(-c2nc(-c3ccccc3)nc(-c3ccc(-c4c(-c5ccc6c7c(cccc57)-c5ccccc5-6)ccc5ccccc45)cc3)n2)cc1]

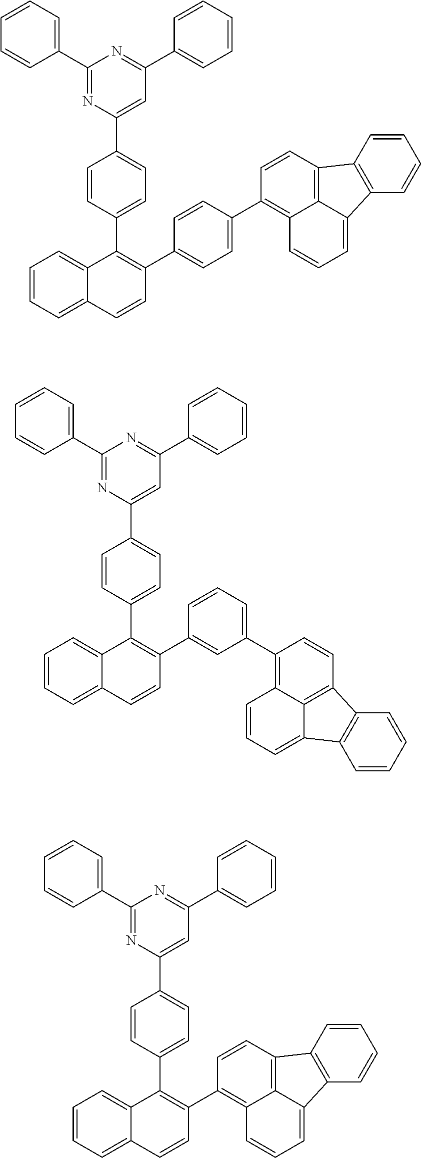 [SMILES: c1ccc(-c2cc(-c3ccc(-c4c(-c5ccc(-c6ccc7c8c(cccc68)-c6ccccc6-7)cc5)ccc5ccccc45)cc3)nc(-c3ccccc3)n2)cc1 ; c1ccc(-c2cc(-c3ccc(-c4c(-c5cccc(-c6ccc7c8c(cccc68)-c6ccccc6-7)c5)ccc5ccccc45)cc3)nc(-c3ccccc3)n2)cc1 ; c1ccc(-c2cc(-c3ccc(-c4c(-c5ccc6c7c(cccc57)-c5ccccc5-6)ccc5ccccc45)cc3)nc(-c3ccccc3)n2)cc1]

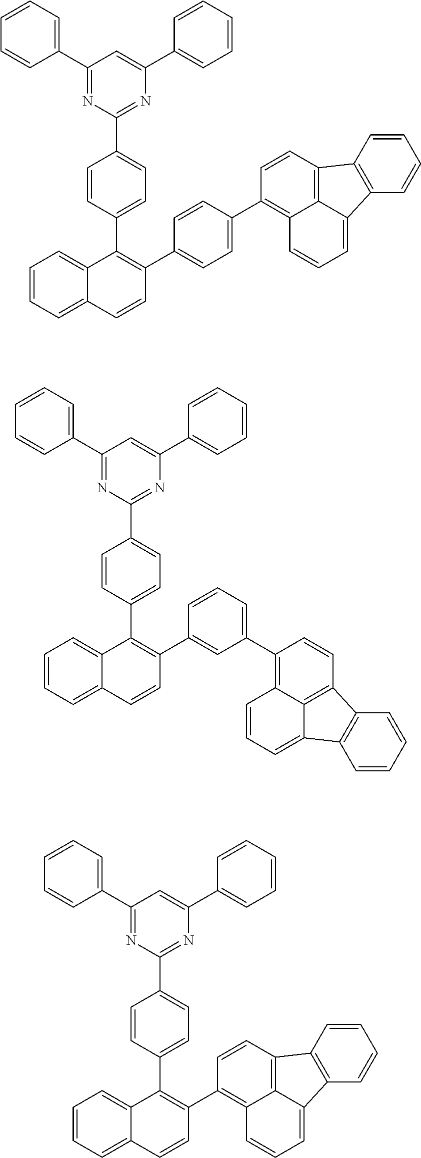 [SMILES: c1ccc(-c2cc(-c3ccccc3)nc(-c3ccc(-c4c(-c5ccc(-c6ccc7c8c(cccc68)-c6ccccc6-7)cc5)ccc5ccccc45)cc3)n2)cc1 ; c1ccc(-c2cc(-c3ccccc3)nc(-c3ccc(-c4c(-c5cccc(-c6ccc7c8c(cccc68)-c6ccccc6-7)c5)ccc5ccccc45)cc3)n2)cc1 ; c1ccc(-c2cc(-c3ccccc3)nc(-c3ccc(-c4c(-c5ccc6c7c(cccc57)-c5ccccc5-6)ccc5ccccc45)cc3)n2)cc1]

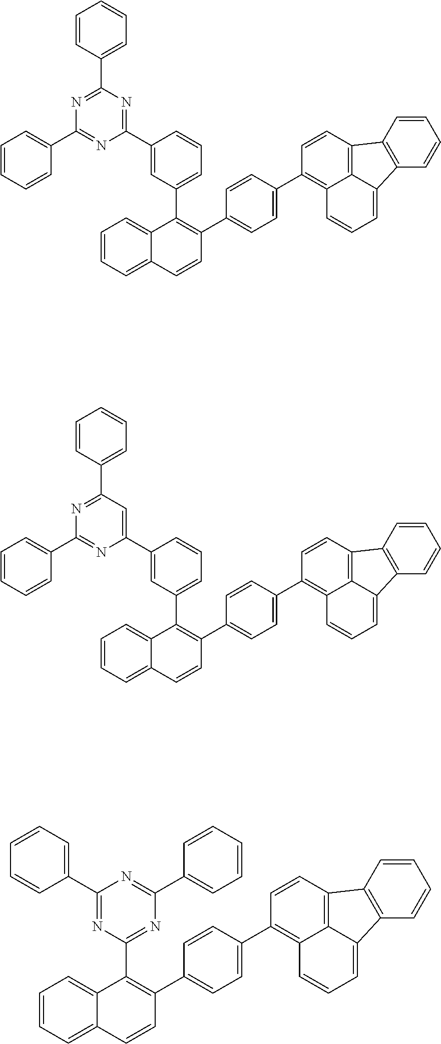 [SMILES: c1ccc(-c2nc(-c3ccccc3)nc(-c3cccc(-c4c(-c5ccc(-c6ccc7c8c(cccc68)-c6ccccc6-7)cc5)ccc5ccccc45)c3)n2)cc1 ; c1ccc(-c2cc(-c3cccc(-c4c(-c5ccc(-c6ccc7c8c(cccc68)-c6ccccc6-7)cc5)ccc5ccccc45)c3)nc(-c3ccccc3)n2)cc1 ; c1ccc(-c2nc(-c3ccccc3)nc(-c3c(-c4ccc(-c5ccc6c7c(cccc57)-c5ccccc5-6)cc4)ccc4ccccc34)n2)cc1]

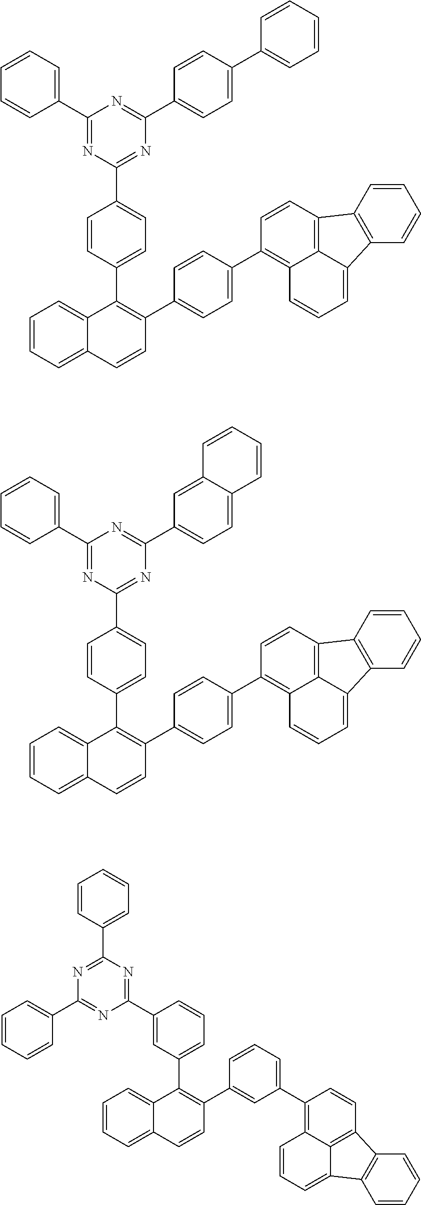 [SMILES: c1ccc(-c2ccc(-c3nc(-c4ccccc4)nc(-c4ccc(-c5c(-c6ccc(-c7ccc8c9c(cccc79)-c7ccccc7-8)cc6)ccc6ccccc56)cc4)n3)cc2)cc1 ; c1ccc(-c2nc(-c3ccc(-c4c(-c5ccc(-c6ccc7c8c(cccc68)-c6ccccc6-7)cc5)ccc5ccccc45)cc3)nc(-c3ccc4ccccc4c3)n2)cc1 ; c1ccc(-c2nc(-c3ccccc3)nc(-c3cccc(-c4c(-c5cccc(-c6ccc7c8c(cccc68)-c6ccccc6-7)c5)ccc5ccccc45)c3)n2)cc1]

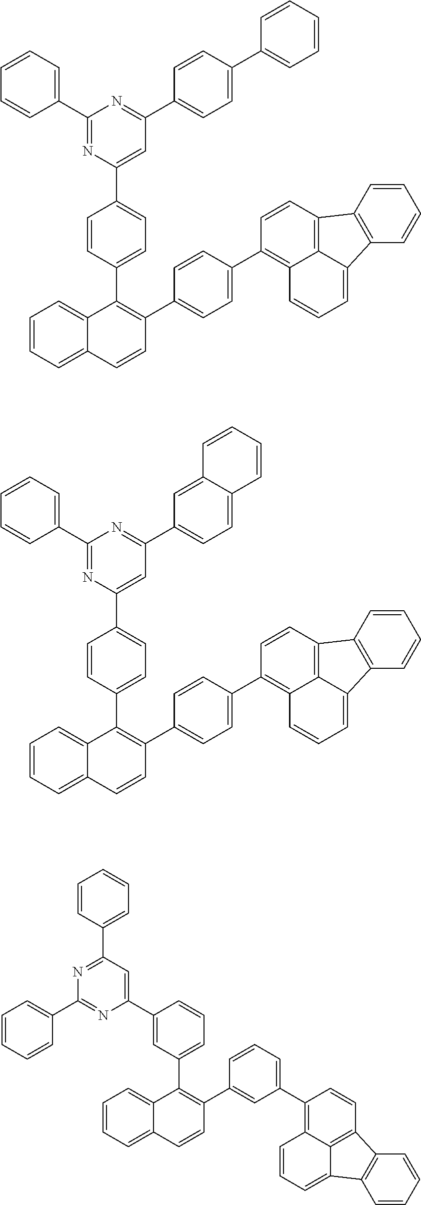 [SMILES: c1ccc(-c2ccc(-c3cc(-c4ccc(-c5c(-c6ccc(-c7ccc8c9c(cccc79)-c7ccccc7-8)cc6)ccc6ccccc56)cc4)nc(-c4ccccc4)n3)cc2)cc1 ; c1ccc(-c2nc(-c3ccc(-c4c(-c5ccc(-c6ccc7c8c(cccc68)-c6ccccc6-7)cc5)ccc5ccccc45)cc3)cc(-c3ccc4ccccc4c3)n2)cc1 ; c1ccc(-c2cc(-c3cccc(-c4c(-c5cccc(-c6ccc7c8c(cccc68)-c6ccccc6-7)c5)ccc5ccccc45)c3)nc(-c3ccccc3)n2)cc1]

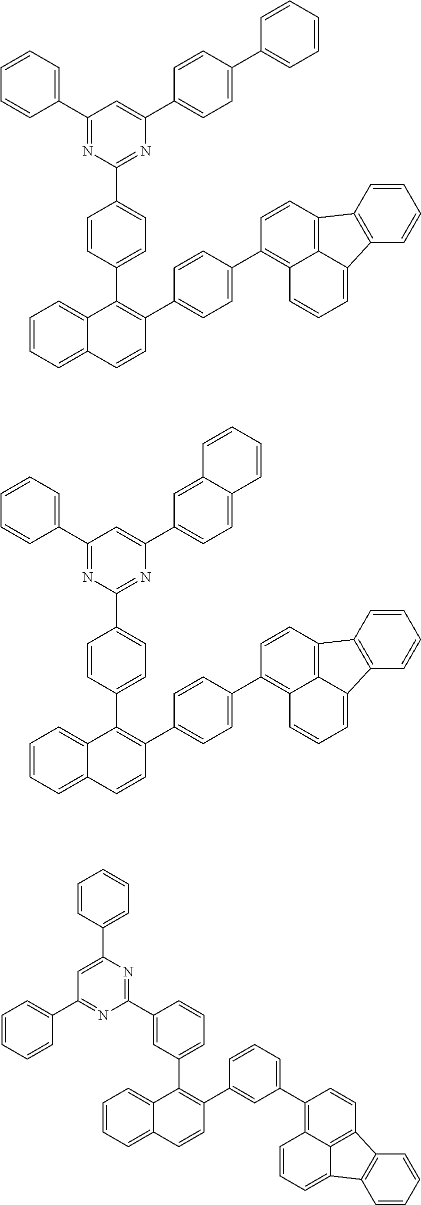 [SMILES: c1ccc(-c2ccc(-c3cc(-c4ccccc4)nc(-c4ccc(-c5c(-c6ccc(-c7ccc8c9c(cccc79)-c7ccccc7-8)cc6)ccc6ccccc56)cc4)n3)cc2)cc1 ; c1ccc(-c2cc(-c3ccc4ccccc4c3)nc(-c3ccc(-c4c(-c5ccc(-c6ccc7c8c(cccc68)-c6ccccc6-7)cc5)ccc5ccccc45)cc3)n2)cc1 ; c1ccc(-c2cc(-c3ccccc3)nc(-c3cccc(-c4c(-c5cccc(-c6ccc7c8c(cccc68)-c6ccccc6-7)c5)ccc5ccccc45)c3)n2)cc1]

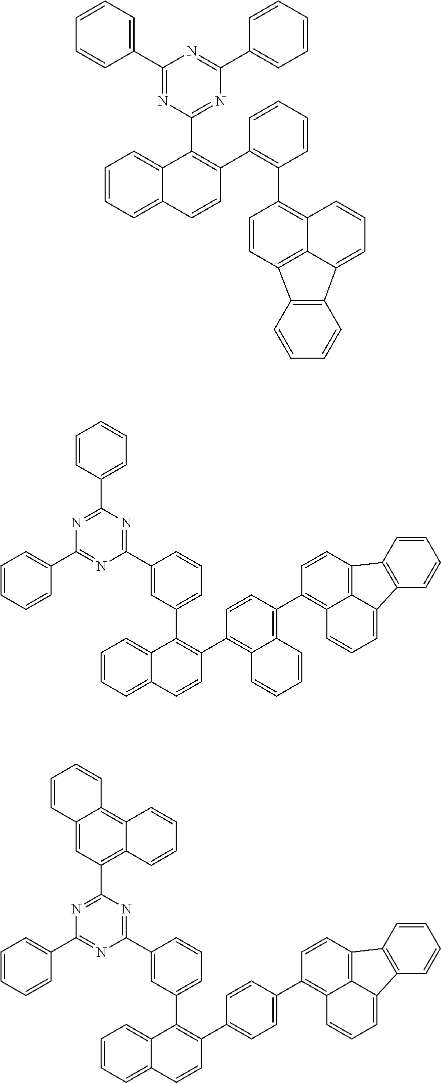 [SMILES: c1ccc(-c2nc(-c3ccccc3)nc(-c3c(-c4ccccc4-c4ccc5c6c(cccc46)-c4ccccc4-5)ccc4ccccc34)n2)cc1 ; c1ccc(-c2nc(-c3ccccc3)nc(-c3cccc(-c4c(-c5ccc(-c6ccc7c8c(cccc68)-c6ccccc6-7)c6ccccc56)ccc5ccccc45)c3)n2)cc1 ; c1ccc(-c2nc(-c3cccc(-c4c(-c5ccc(-c6ccc7c8c(cccc68)-c6ccccc6-7)cc5)ccc5ccccc45)c3)nc(-c3cc4ccccc4c4ccccc34)n2)cc1]

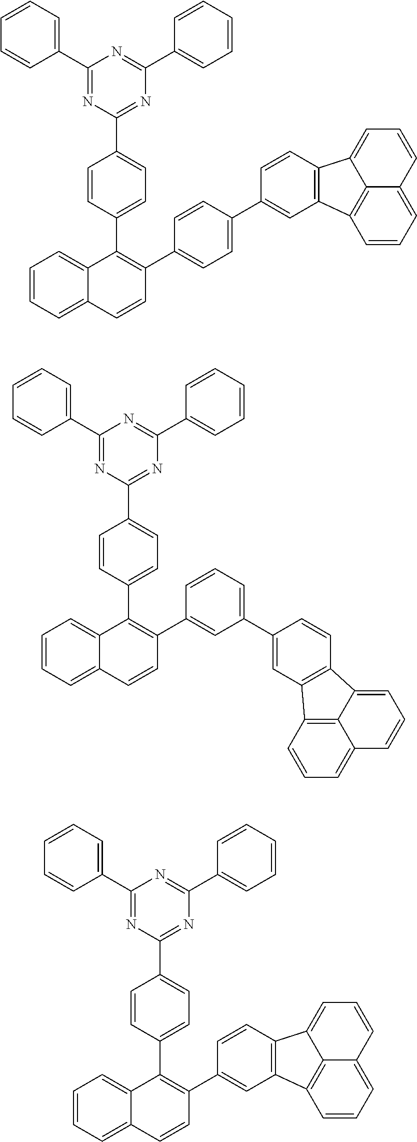 [SMILES: c1ccc(-c2nc(-c3ccccc3)nc(-c3ccc(-c4c(-c5ccc(-c6ccc7c(c6)-c6cccc8cccc-7c68)cc5)ccc5ccccc45)cc3)n2)cc1 ; c1ccc(-c2nc(-c3ccccc3)nc(-c3ccc(-c4c(-c5cccc(-c6ccc7c(c6)-c6cccc8cccc-7c68)c5)ccc5ccccc45)cc3)n2)cc1 ; c1ccc(-c2nc(-c3ccccc3)nc(-c3ccc(-c4c(-c5ccc6c(c5)-c5cccc7cccc-6c57)ccc5ccccc45)cc3)n2)cc1]

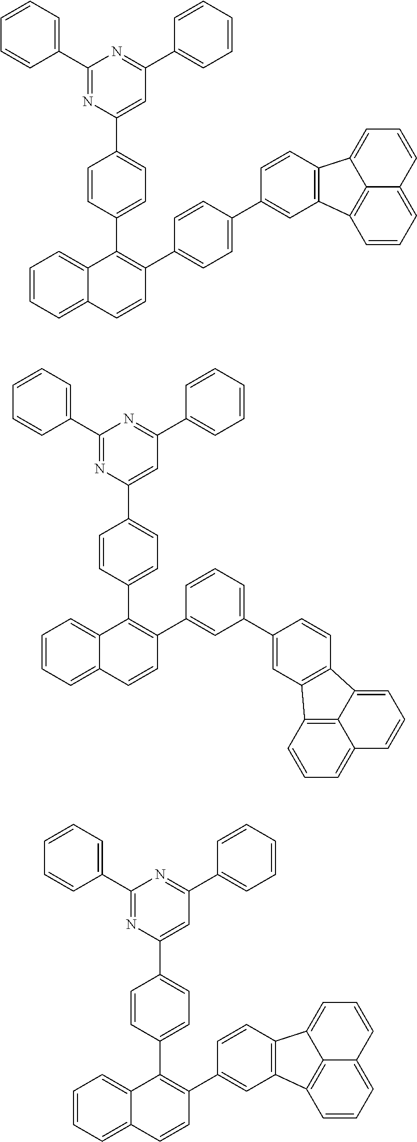 [SMILES: c1ccc(-c2cc(-c3ccc(-c4c(-c5ccc(-c6ccc7c(c6)-c6cccc8cccc-7c68)cc5)ccc5ccccc45)cc3)nc(-c3ccccc3)n2)cc1 ; c1ccc(-c2cc(-c3ccc(-c4c(-c5cccc(-c6ccc7c(c6)-c6cccc8cccc-7c68)c5)ccc5ccccc45)cc3)nc(-c3ccccc3)n2)cc1 ; c1ccc(-c2cc(-c3ccc(-c4c(-c5ccc6c(c5)-c5cccc7cccc-6c57)ccc5ccccc45)cc3)nc(-c3ccccc3)n2)cc1]

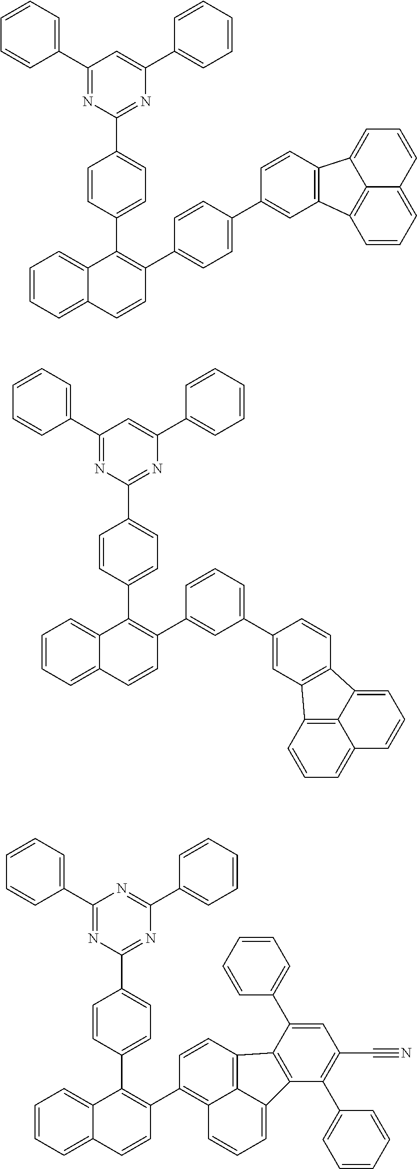 [SMILES: c1ccc(-c2cc(-c3ccccc3)nc(-c3ccc(-c4c(-c5ccc(-c6ccc7c(c6)-c6cccc8cccc-7c68)cc5)ccc5ccccc45)cc3)n2)cc1 ; c1ccc(-c2cc(-c3ccccc3)nc(-c3ccc(-c4c(-c5cccc(-c6ccc7c(c6)-c6cccc8cccc-7c68)c5)ccc5ccccc45)cc3)n2)cc1 ; N#Cc1cc(-c2ccccc2)c2c(c1-c1ccccc1)-c1cccc3c(-c4ccc5ccccc5c4-c4ccc(-c5nc(-c6ccccc6)nc(-c6ccccc6)n5)cc4)ccc-2c13]

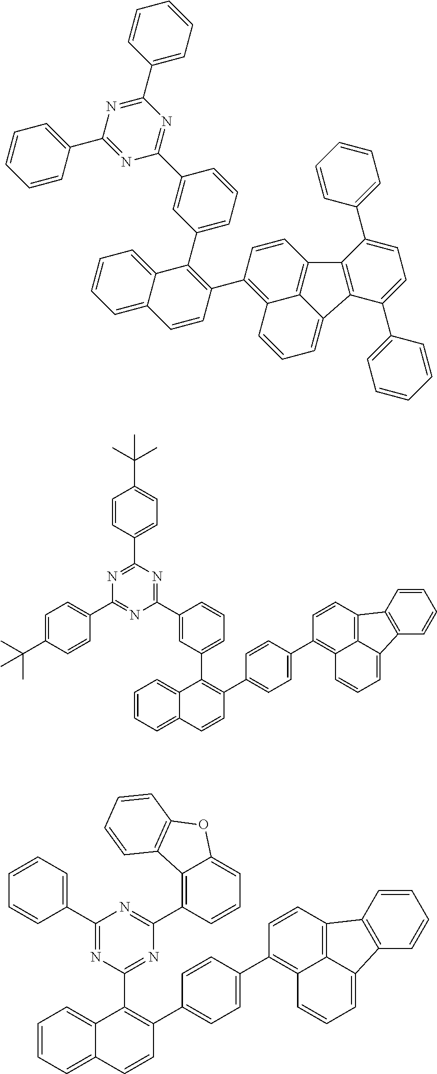 [SMILES: c1ccc(-c2nc(-c3ccccc3)nc(-c3cccc(-c4c(-c5ccc6c7c(cccc57)-c5c(-c7ccccc7)ccc(-c7ccccc7)c5-6)ccc5ccccc45)c3)n2)cc1 ; CC(C)(C)c1ccc(-c2nc(-c3ccc(C(C)(C)C)cc3)nc(-c3cccc(-c4c(-c5ccc(-c6ccc7c8c(cccc68)-c6ccccc6-7)cc5)ccc5ccccc45)c3)n2)cc1 ; c1ccc(-c2nc(-c3c(-c4ccc(-c5ccc6c7c(cccc57)-c5ccccc5-6)cc4)ccc4ccccc34)nc(-c3cccc4oc5ccccc5c34)n2)cc1]

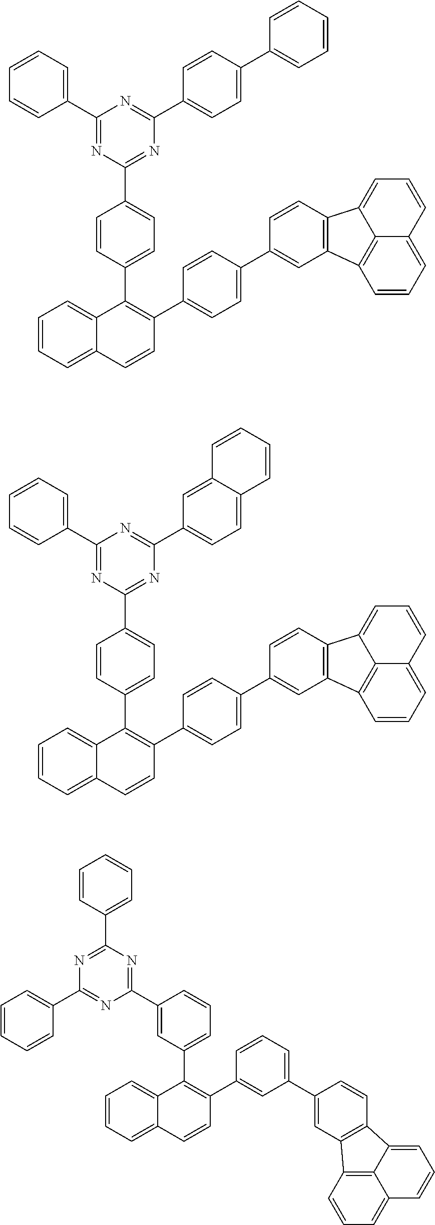 [SMILES: c1ccc(-c2ccc(-c3nc(-c4ccccc4)nc(-c4ccc(-c5c(-c6ccc(-c7ccc8c(c7)-c7cccc9cccc-8c79)cc6)ccc6ccccc56)cc4)n3)cc2)cc1 ; c1ccc(-c2nc(-c3ccc(-c4c(-c5ccc(-c6ccc7c(c6)-c6cccc8cccc-7c68)cc5)ccc5ccccc45)cc3)nc(-c3ccc4ccccc4c3)n2)cc1 ; c1ccc(-c2nc(-c3ccccc3)nc(-c3cccc(-c4c(-c5cccc(-c6ccc7c(c6)-c6cccc8cccc-7c68)c5)ccc5ccccc45)c3)n2)cc1]

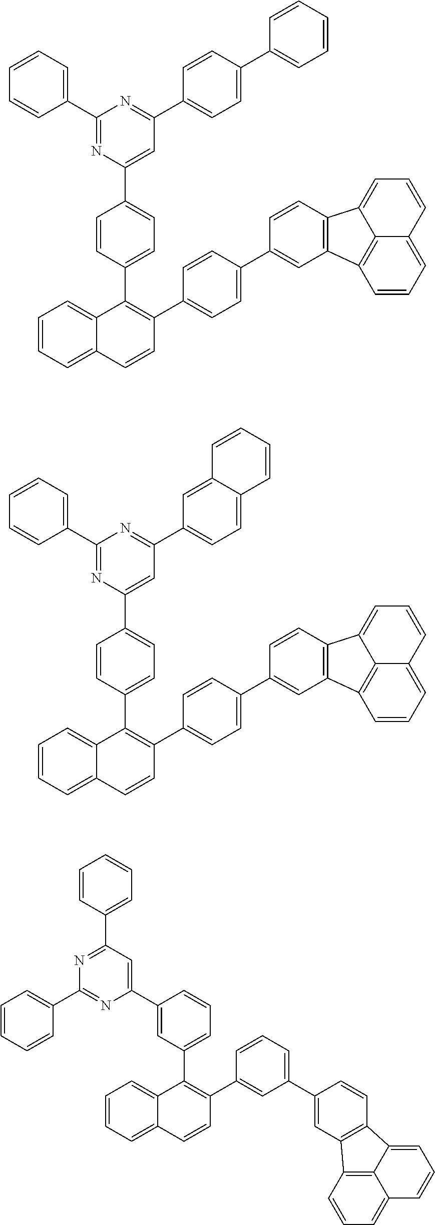 [SMILES: c1ccc(-c2ccc(-c3cc(-c4ccc(-c5c(-c6ccc(-c7ccc8c(c7)-c7cccc9cccc-8c79)cc6)ccc6ccccc56)cc4)nc(-c4ccccc4)n3)cc2)cc1 ; c1ccc(-c2nc(-c3ccc(-c4c(-c5ccc(-c6ccc7c(c6)-c6cccc8cccc-7c68)cc5)ccc5ccccc45)cc3)cc(-c3ccc4ccccc4c3)n2)cc1 ; c1ccc(-c2cc(-c3cccc(-c4c(-c5cccc(-c6ccc7c(c6)-c6cccc8cccc-7c68)c5)ccc5ccccc45)c3)nc(-c3ccccc3)n2)cc1]

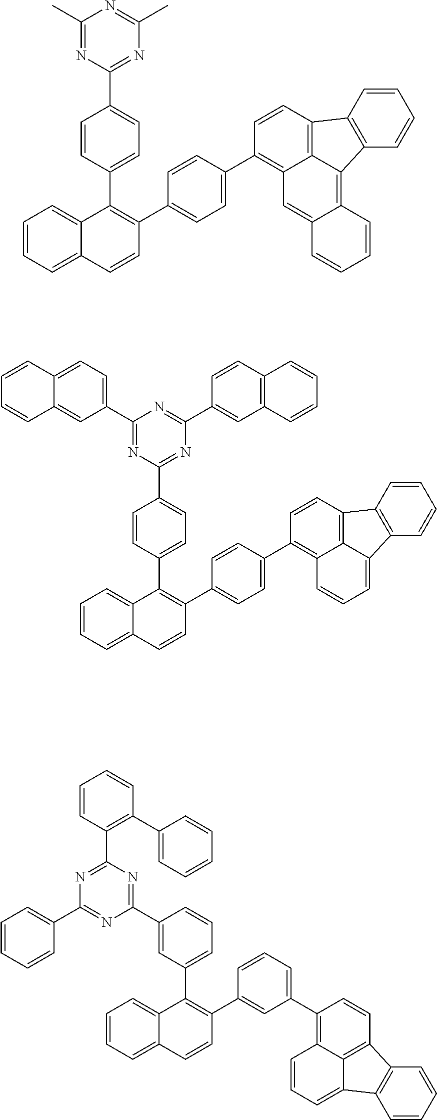 [SMILES: Cc1nc(C)nc(-c2ccc(-c3c(-c4ccc(-c5ccc6c7c(c8ccccc8cc57)-c5ccccc5-6)cc4)ccc4ccccc34)cc2)n1 ; c1ccc2c(c1)-c1cccc3c(-c4ccc(-c5ccc6ccccc6c5-c5ccc(-c6nc(-c7ccc8ccccc8c7)nc(-c7ccc8ccccc8c7)n6)cc5)cc4)ccc-2c13 ; c1ccc(-c2nc(-c3cccc(-c4c(-c5cccc(-c6ccc7c8c(cccc68)-c6ccccc6-7)c5)ccc5ccccc45)c3)nc(-c3ccccc3-c3ccccc3)n2)cc1]

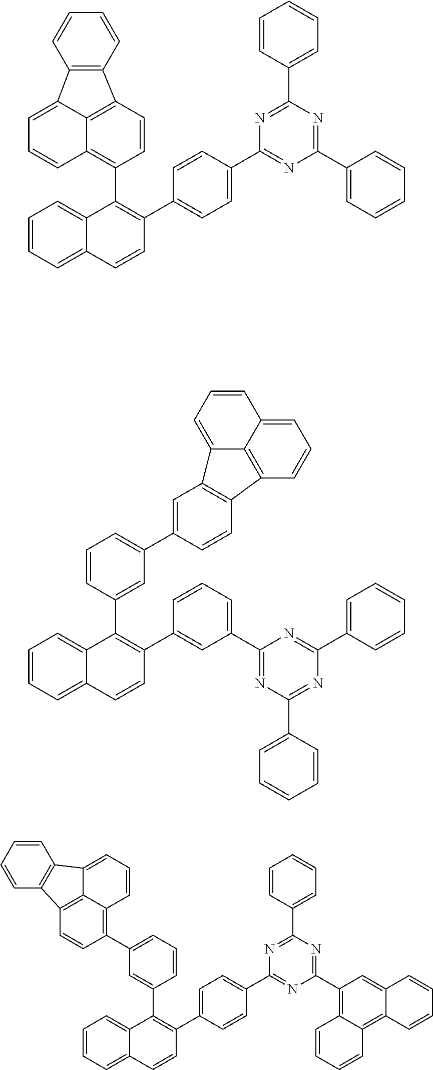 [SMILES: c1ccc(-c2nc(-c3ccccc3)nc(-c3ccc(-c4ccc5ccccc5c4-c4ccc5c6c(cccc46)-c4ccccc4-5)cc3)n2)cc1 ; c1ccc(-c2nc(-c3ccccc3)nc(-c3cccc(-c4ccc5ccccc5c4-c4cccc(-c5ccc6c(c5)-c5cccc7cccc-6c57)c4)c3)n2)cc1 ; c1ccc(-c2nc(-c3ccc(-c4ccc5ccccc5c4-c4cccc(-c5ccc6c7c(cccc57)-c5ccccc5-6)c4)cc3)nc(-c3cc4ccccc4c4ccccc34)n2)cc1]

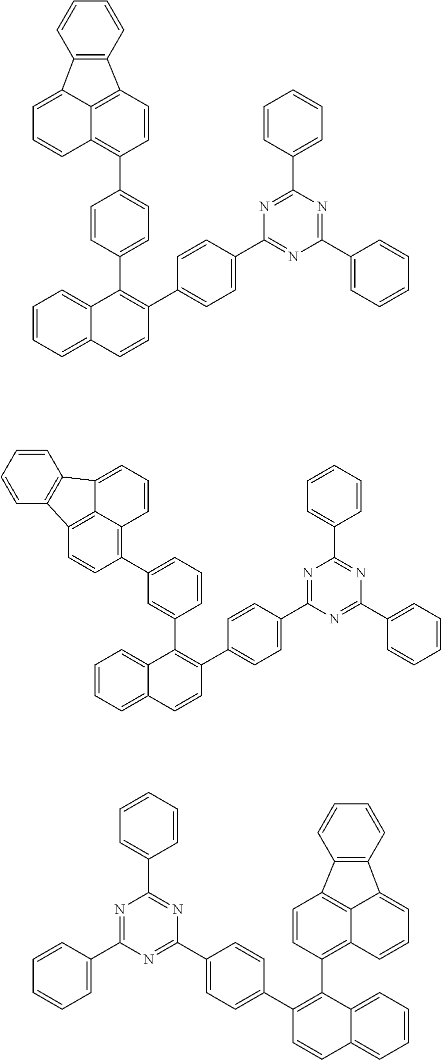 [SMILES: c1ccc(-c2nc(-c3ccccc3)nc(-c3ccc(-c4ccc5ccccc5c4-c4ccc(-c5ccc6c7c(cccc57)-c5ccccc5-6)cc4)cc3)n2)cc1 ; c1ccc(-c2nc(-c3ccccc3)nc(-c3ccc(-c4ccc5ccccc5c4-c4cccc(-c5ccc6c7c(cccc57)-c5ccccc5-6)c4)cc3)n2)cc1 ; c1ccc(-c2nc(-c3ccccc3)nc(-c3ccc(-c4ccc5ccccc5c4-c4ccc5c6c(cccc46)-c4ccccc4-5)cc3)n2)cc1]

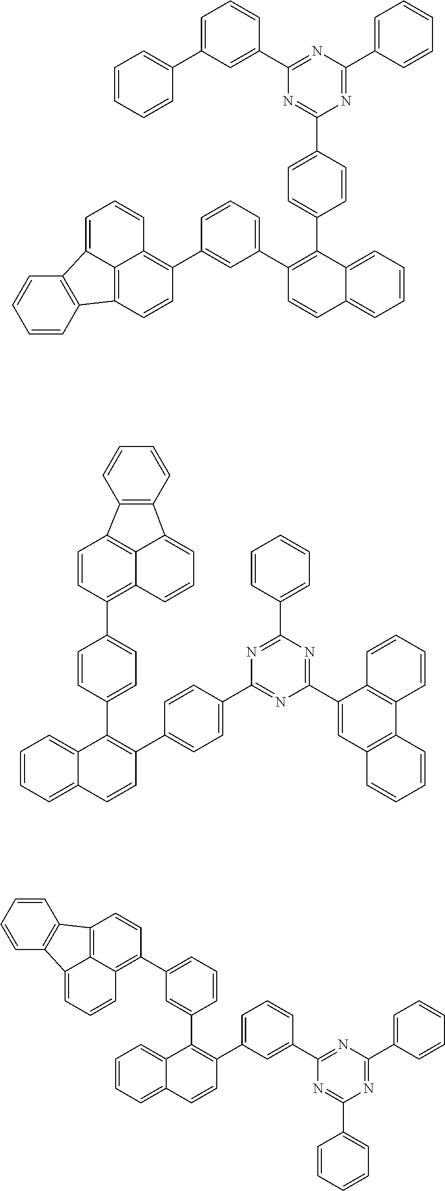 [SMILES: c1ccc(-c2cccc(-c3nc(-c4ccccc4)nc(-c4ccc(-c5c(-c6cccc(-c7ccc8c9c(cccc79)-c7ccccc7-8)c6)ccc6ccccc56)cc4)n3)c2)cc1 ; c1ccc(-c2nc(-c3ccc(-c4ccc5ccccc5c4-c4ccc(-c5ccc6c7c(cccc57)-c5ccccc5-6)cc4)cc3)nc(-c3cc4ccccc4c4ccccc34)n2)cc1 ; c1ccc(-c2nc(-c3ccccc3)nc(-c3cccc(-c4ccc5ccccc5c4-c4cccc(-c5ccc6c7c(cccc57)-c5ccccc5-6)c4)c3)n2)cc1]

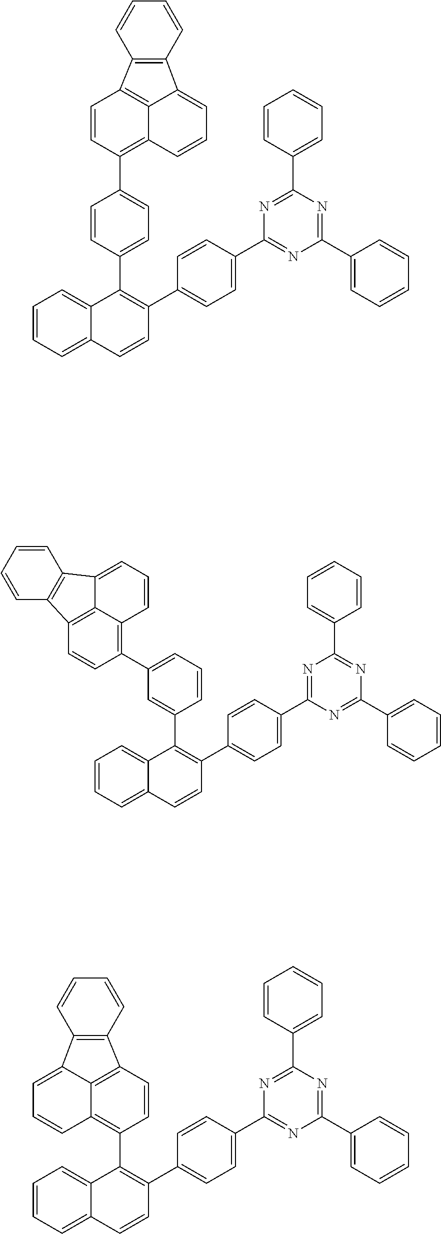 [SMILES: c1ccc(-c2nc(-c3ccccc3)nc(-c3ccc(-c4ccc5ccccc5c4-c4ccc(-c5ccc6c7c(cccc57)-c5ccccc5-6)cc4)cc3)n2)cc1 ; c1ccc(-c2nc(-c3ccccc3)nc(-c3ccc(-c4ccc5ccccc5c4-c4cccc(-c5ccc6c7c(cccc57)-c5ccccc5-6)c4)cc3)n2)cc1 ; c1ccc(-c2nc(-c3ccccc3)nc(-c3ccc(-c4ccc5ccccc5c4-c4ccc5c6c(cccc46)-c4ccccc4-5)cc3)n2)cc1]

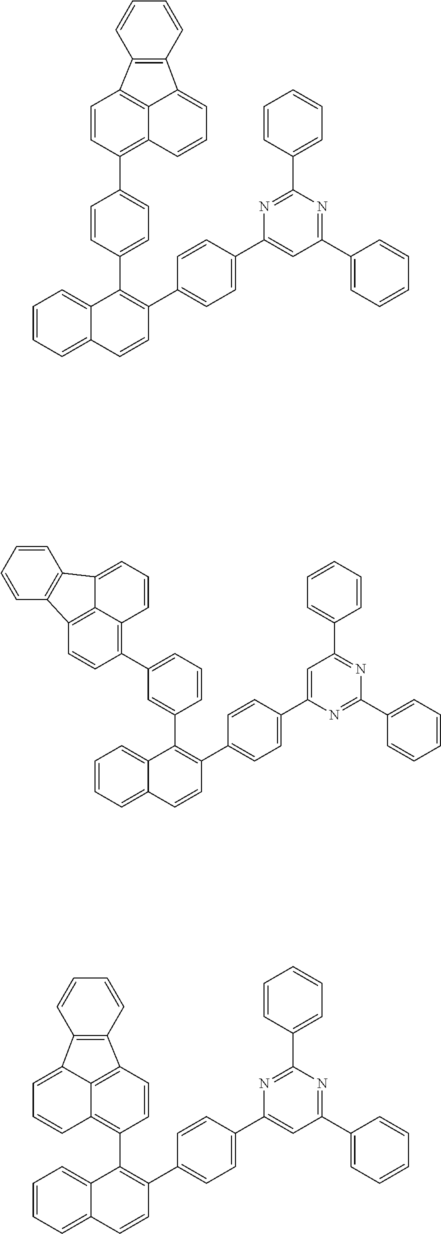 [SMILES: c1ccc(-c2cc(-c3ccc(-c4ccc5ccccc5c4-c4ccc(-c5ccc6c7c(cccc57)-c5ccccc5-6)cc4)cc3)nc(-c3ccccc3)n2)cc1 ; c1ccc(-c2cc(-c3ccc(-c4ccc5ccccc5c4-c4cccc(-c5ccc6c7c(cccc57)-c5ccccc5-6)c4)cc3)nc(-c3ccccc3)n2)cc1 ; c1ccc(-c2cc(-c3ccc(-c4ccc5ccccc5c4-c4ccc5c6c(cccc46)-c4ccccc4-5)cc3)nc(-c3ccccc3)n2)cc1]

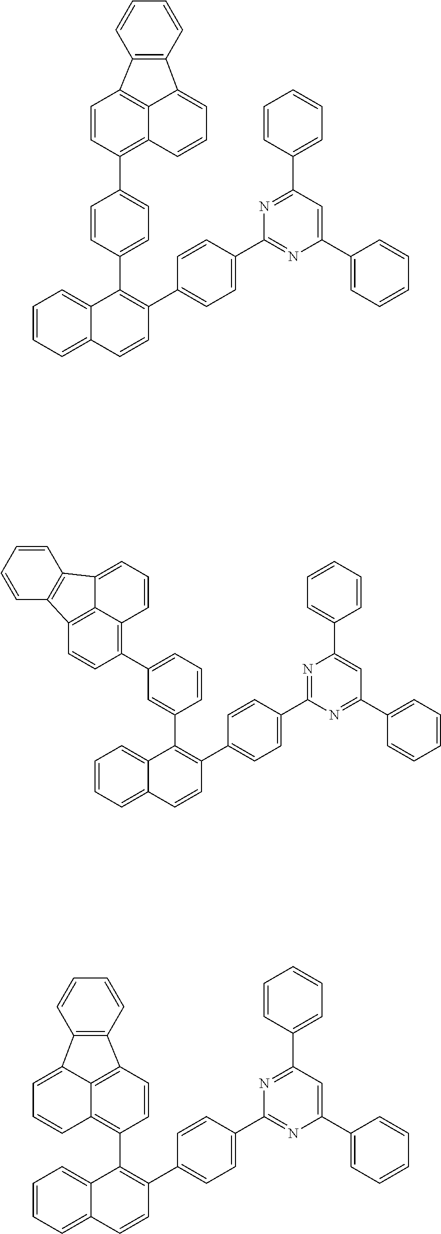 [SMILES: c1ccc(-c2cc(-c3ccccc3)nc(-c3ccc(-c4ccc5ccccc5c4-c4ccc(-c5ccc6c7c(cccc57)-c5ccccc5-6)cc4)cc3)n2)cc1 ; c1ccc(-c2cc(-c3ccccc3)nc(-c3ccc(-c4ccc5ccccc5c4-c4cccc(-c5ccc6c7c(cccc57)-c5ccccc5-6)c4)cc3)n2)cc1 ; c1ccc(-c2cc(-c3ccccc3)nc(-c3ccc(-c4ccc5ccccc5c4-c4ccc5c6c(cccc46)-c4ccccc4-5)cc3)n2)cc1]

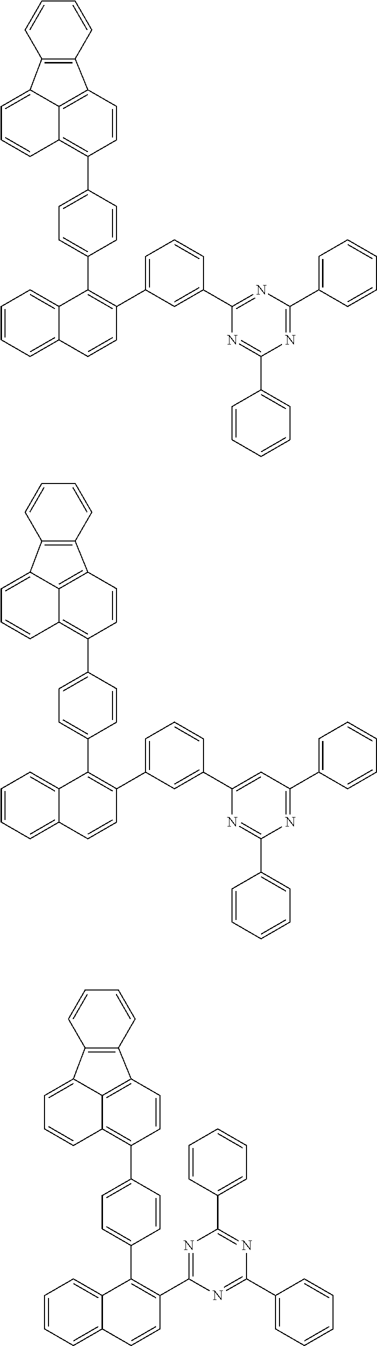 [SMILES: c1ccc(-c2nc(-c3ccccc3)nc(-c3cccc(-c4ccc5ccccc5c4-c4ccc(-c5ccc6c7c(cccc57)-c5ccccc5-6)cc4)c3)n2)cc1 ; c1ccc(-c2cc(-c3cccc(-c4ccc5ccccc5c4-c4ccc(-c5ccc6c7c(cccc57)-c5ccccc5-6)cc4)c3)nc(-c3ccccc3)n2)cc1 ; c1ccc(-c2nc(-c3ccccc3)nc(-c3ccc4ccccc4c3-c3ccc(-c4ccc5c6c(cccc46)-c4ccccc4-5)cc3)n2)cc1]

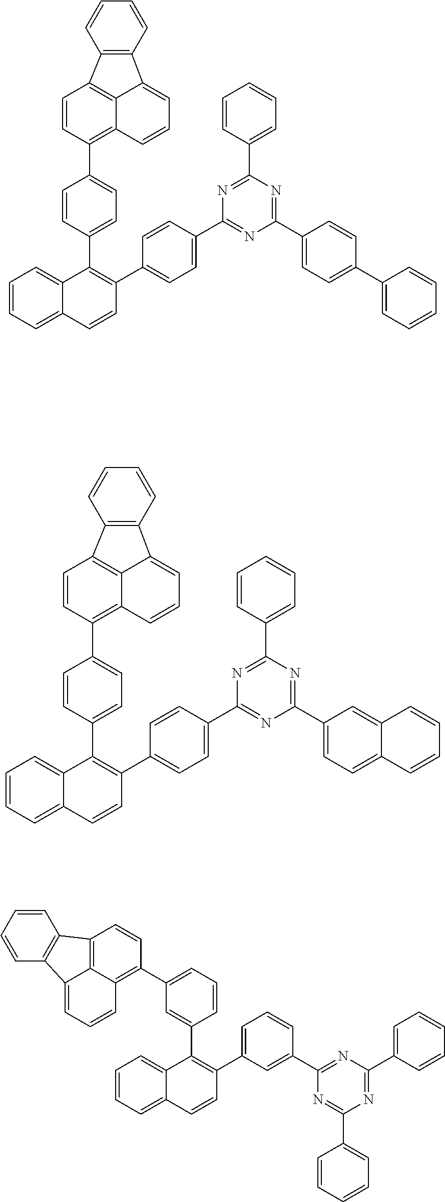 [SMILES: c1ccc(-c2ccc(-c3nc(-c4ccccc4)nc(-c4ccc(-c5ccc6ccccc6c5-c5ccc(-c6ccc7c8c(cccc68)-c6ccccc6-7)cc5)cc4)n3)cc2)cc1 ; c1ccc(-c2nc(-c3ccc(-c4ccc5ccccc5c4-c4ccc(-c5ccc6c7c(cccc57)-c5ccccc5-6)cc4)cc3)nc(-c3ccc4ccccc4c3)n2)cc1 ; c1ccc(-c2nc(-c3ccccc3)nc(-c3cccc(-c4ccc5ccccc5c4-c4cccc(-c5ccc6c7c(cccc57)-c5ccccc5-6)c4)c3)n2)cc1]

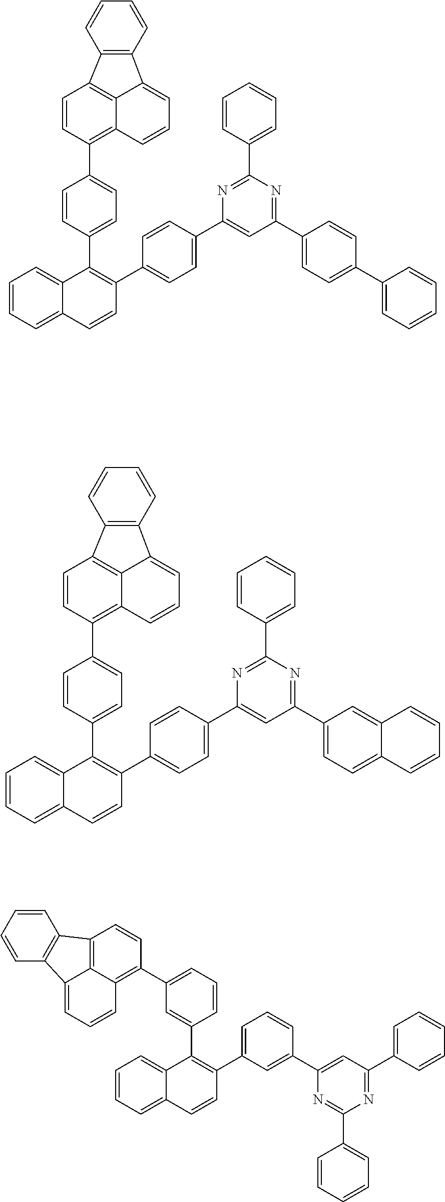 [SMILES: c1ccc(-c2ccc(-c3cc(-c4ccc(-c5ccc6ccccc6c5-c5ccc(-c6ccc7c8c(cccc68)-c6ccccc6-7)cc5)cc4)nc(-c4ccccc4)n3)cc2)cc1 ; c1ccc(-c2nc(-c3ccc(-c4ccc5ccccc5c4-c4ccc(-c5ccc6c7c(cccc57)-c5ccccc5-6)cc4)cc3)cc(-c3ccc4ccccc4c3)n2)cc1 ; c1ccc(-c2cc(-c3cccc(-c4ccc5ccccc5c4-c4cccc(-c5ccc6c7c(cccc57)-c5ccccc5-6)c4)c3)nc(-c3ccccc3)n2)cc1]

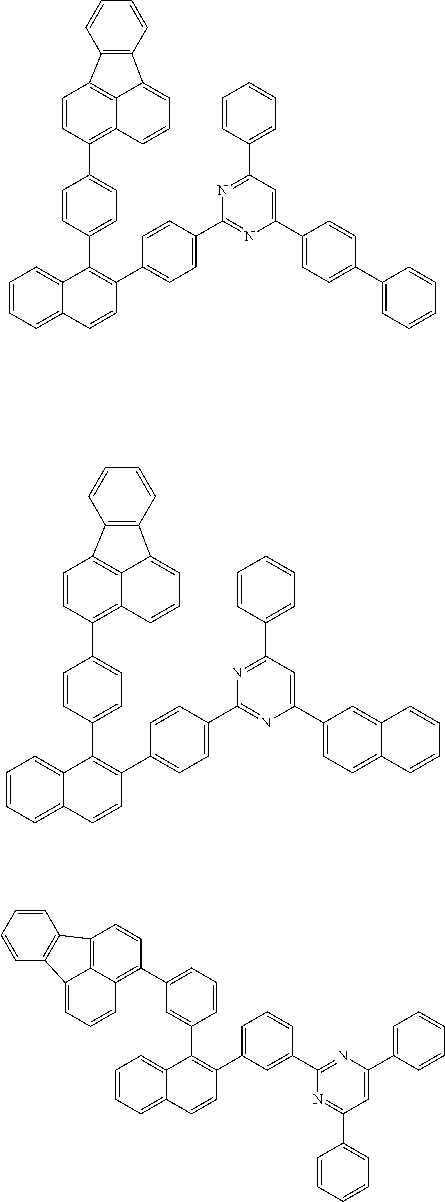 [SMILES: c1ccc(-c2ccc(-c3cc(-c4ccccc4)nc(-c4ccc(-c5ccc6ccccc6c5-c5ccc(-c6ccc7c8c(cccc68)-c6ccccc6-7)cc5)cc4)n3)cc2)cc1 ; c1ccc(-c2cc(-c3ccc4ccccc4c3)nc(-c3ccc(-c4ccc5ccccc5c4-c4ccc(-c5ccc6c7c(cccc57)-c5ccccc5-6)cc4)cc3)n2)cc1 ; c1ccc(-c2cc(-c3ccccc3)nc(-c3cccc(-c4ccc5ccccc5c4-c4cccc(-c5ccc6c7c(cccc57)-c5ccccc5-6)c4)c3)n2)cc1]

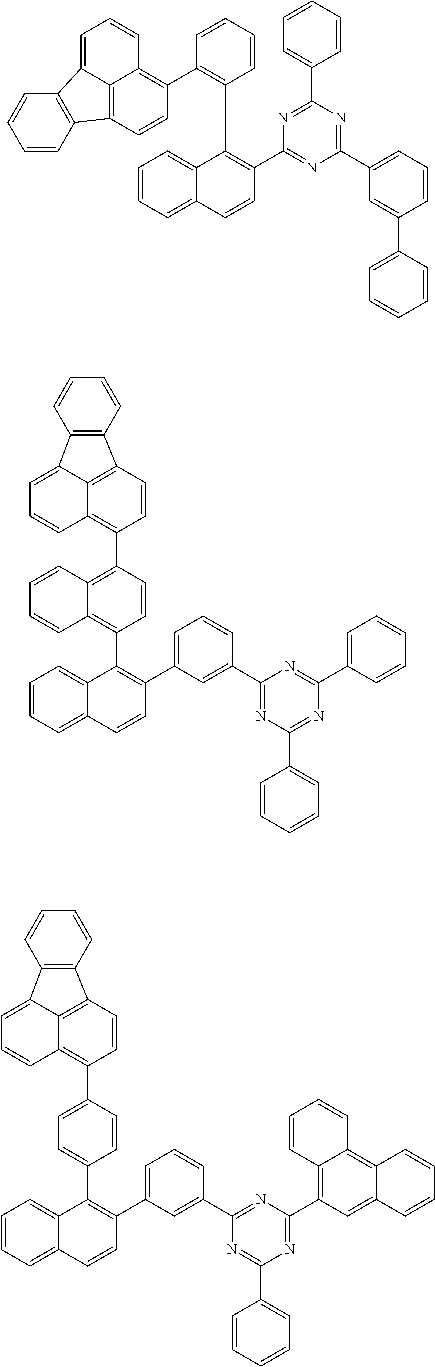 [SMILES: c1ccc(-c2cccc(-c3nc(-c4ccccc4)nc(-c4ccc5ccccc5c4-c4ccccc4-c4ccc5c6c(cccc46)-c4ccccc4-5)n3)c2)cc1 ; c1ccc(-c2nc(-c3ccccc3)nc(-c3cccc(-c4ccc5ccccc5c4-c4ccc(-c5ccc6c7c(cccc57)-c5ccccc5-6)c5ccccc45)c3)n2)cc1 ; c1ccc(-c2nc(-c3cccc(-c4ccc5ccccc5c4-c4ccc(-c5ccc6c7c(cccc57)-c5ccccc5-6)cc4)c3)nc(-c3cc4ccccc4c4ccccc34)n2)cc1]

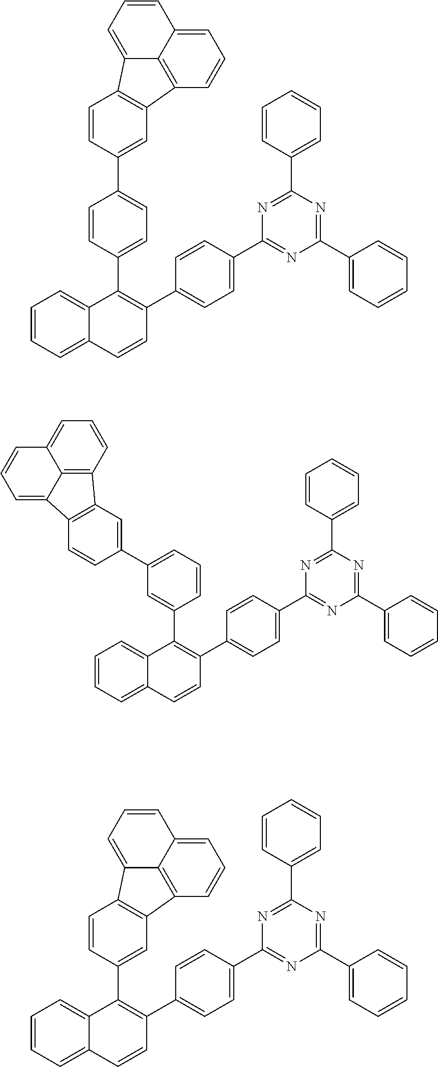 [SMILES: c1ccc(-c2nc(-c3ccccc3)nc(-c3ccc(-c4ccc5ccccc5c4-c4ccc(-c5ccc6c(c5)-c5cccc7cccc-6c57)cc4)cc3)n2)cc1 ; c1ccc(-c2nc(-c3ccccc3)nc(-c3ccc(-c4ccc5ccccc5c4-c4cccc(-c5ccc6c(c5)-c5cccc7cccc-6c57)c4)cc3)n2)cc1 ; c1ccc(-c2nc(-c3ccccc3)nc(-c3ccc(-c4ccc5ccccc5c4-c4ccc5c(c4)-c4cccc6cccc-5c46)cc3)n2)cc1]

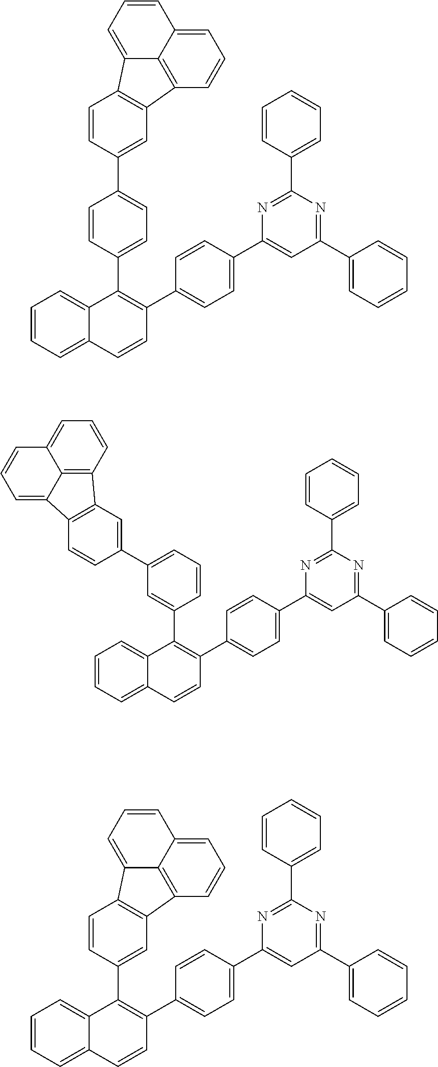 [SMILES: c1ccc(-c2cc(-c3ccc(-c4ccc5ccccc5c4-c4ccc(-c5ccc6c(c5)-c5cccc7cccc-6c57)cc4)cc3)nc(-c3ccccc3)n2)cc1 ; c1ccc(-c2cc(-c3ccc(-c4ccc5ccccc5c4-c4cccc(-c5ccc6c(c5)-c5cccc7cccc-6c57)c4)cc3)nc(-c3ccccc3)n2)cc1 ; c1ccc(-c2cc(-c3ccc(-c4ccc5ccccc5c4-c4ccc5c(c4)-c4cccc6cccc-5c46)cc3)nc(-c3ccccc3)n2)cc1]

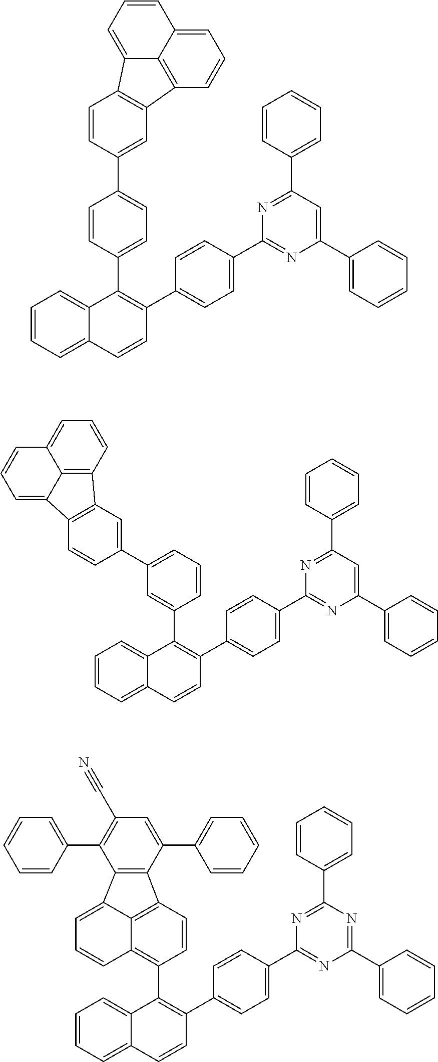 [SMILES: c1ccc(-c2cc(-c3ccccc3)nc(-c3ccc(-c4ccc5ccccc5c4-c4ccc(-c5ccc6c(c5)-c5cccc7cccc-6c57)cc4)cc3)n2)cc1 ; c1ccc(-c2cc(-c3ccccc3)nc(-c3ccc(-c4ccc5ccccc5c4-c4cccc(-c5ccc6c(c5)-c5cccc7cccc-6c57)c4)cc3)n2)cc1 ; N#Cc1cc(-c2ccccc2)c2c(c1-c1ccccc1)-c1cccc3c(-c4c(-c5ccc(-c6nc(-c7ccccc7)nc(-c7ccccc7)n6)cc5)ccc5ccccc45)ccc-2c13]

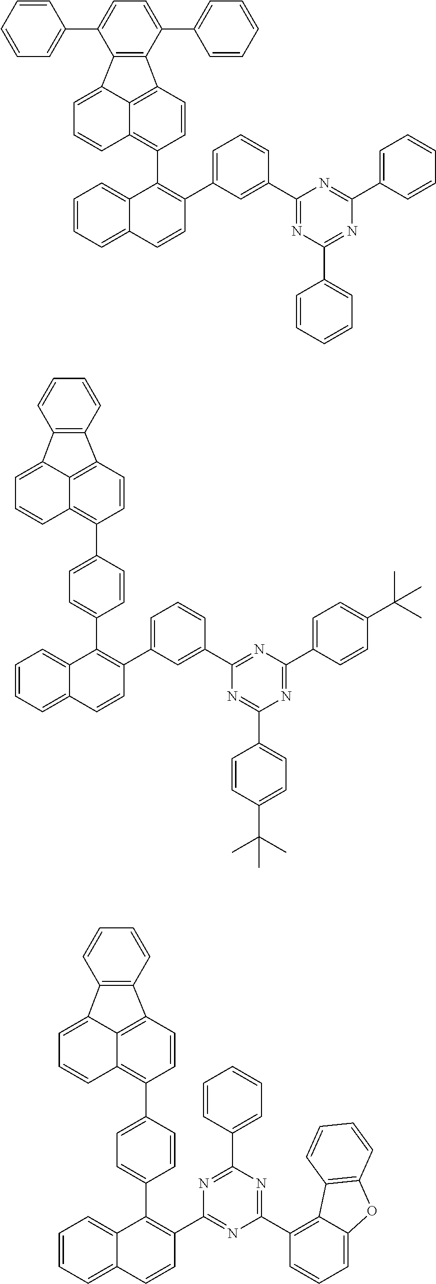 [SMILES: c1ccc(-c2nc(-c3ccccc3)nc(-c3cccc(-c4ccc5ccccc5c4-c4ccc5c6c(cccc46)-c4c(-c6ccccc6)ccc(-c6ccccc6)c4-5)c3)n2)cc1 ; CC(C)(C)c1ccc(-c2nc(-c3ccc(C(C)(C)C)cc3)nc(-c3cccc(-c4ccc5ccccc5c4-c4ccc(-c5ccc6c7c(cccc57)-c5ccccc5-6)cc4)c3)n2)cc1 ; c1ccc(-c2nc(-c3ccc4ccccc4c3-c3ccc(-c4ccc5c6c(cccc46)-c4ccccc4-5)cc3)nc(-c3cccc4oc5ccccc5c34)n2)cc1]

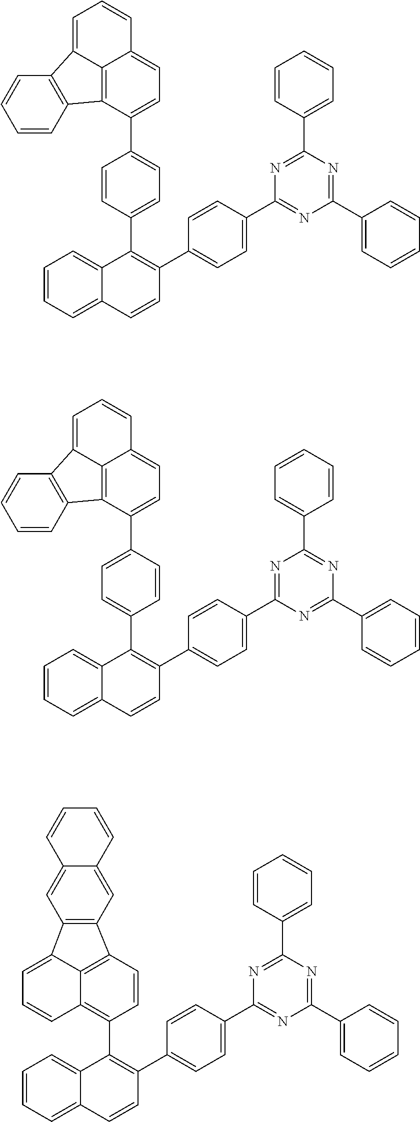 [SMILES: c1ccc(-c2nc(-c3ccccc3)nc(-c3ccc(-c4ccc5ccccc5c4-c4ccc(-c5ccc6cccc7c6c5-c5ccccc5-7)cc4)cc3)n2)cc1 ; c1ccc(-c2nc(-c3ccccc3)nc(-c3ccc(-c4ccc5ccccc5c4-c4ccc(-c5ccc6cccc7c6c5-c5ccccc5-7)cc4)cc3)n2)cc1 ; c1ccc(-c2nc(-c3ccccc3)nc(-c3ccc(-c4ccc5ccccc5c4-c4ccc5c6c(cccc46)-c4cc6ccccc6cc4-5)cc3)n2)cc1]

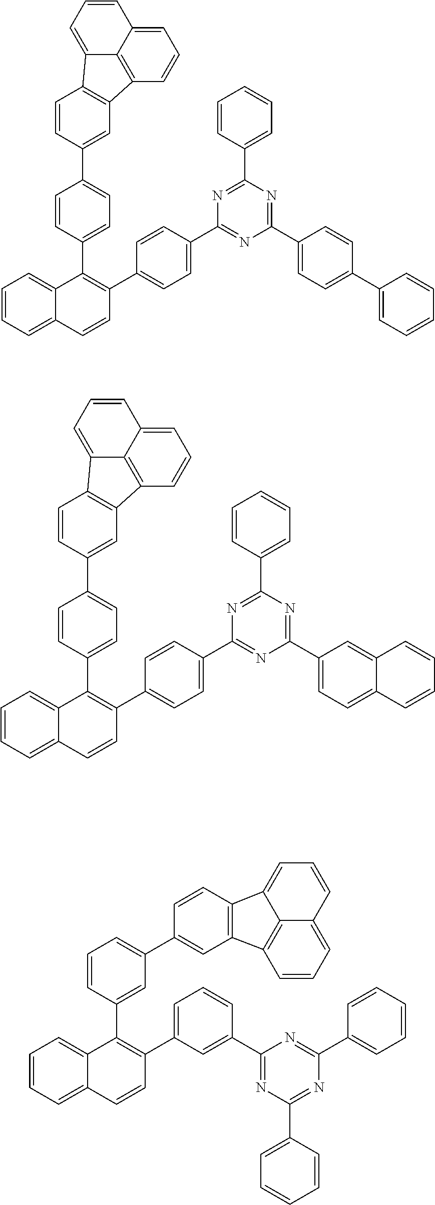 [SMILES: c1ccc(-c2ccc(-c3nc(-c4ccccc4)nc(-c4ccc(-c5ccc6ccccc6c5-c5ccc(-c6ccc7c(c6)-c6cccc8cccc-7c68)cc5)cc4)n3)cc2)cc1 ; c1ccc(-c2nc(-c3ccc(-c4ccc5ccccc5c4-c4ccc(-c5ccc6c(c5)-c5cccc7cccc-6c57)cc4)cc3)nc(-c3ccc4ccccc4c3)n2)cc1 ; c1ccc(-c2nc(-c3ccccc3)nc(-c3cccc(-c4ccc5ccccc5c4-c4cccc(-c5ccc6c(c5)-c5cccc7cccc-6c57)c4)c3)n2)cc1]

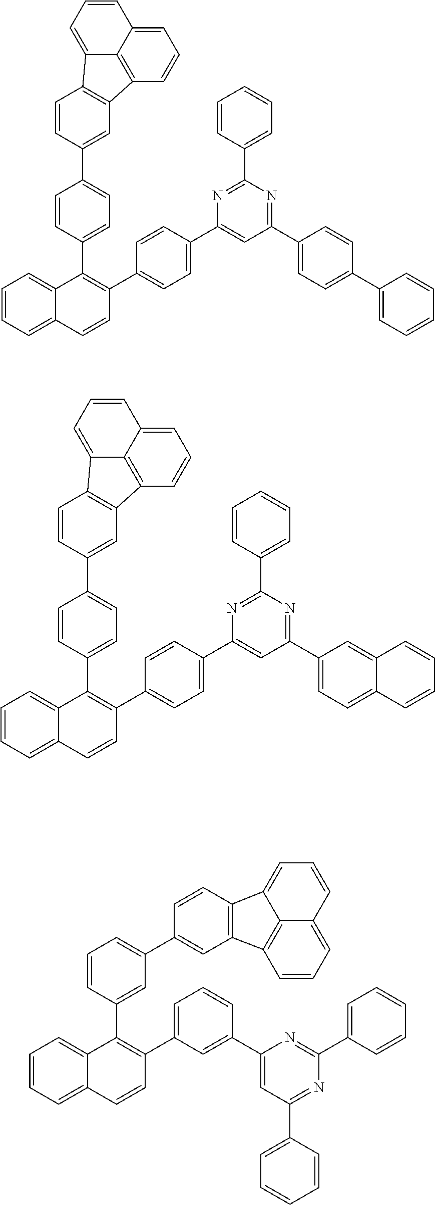 [SMILES: c1ccc(-c2ccc(-c3cc(-c4ccc(-c5ccc6ccccc6c5-c5ccc(-c6ccc7c(c6)-c6cccc8cccc-7c68)cc5)cc4)nc(-c4ccccc4)n3)cc2)cc1 ; c1ccc(-c2nc(-c3ccc(-c4ccc5ccccc5c4-c4ccc(-c5ccc6c(c5)-c5cccc7cccc-6c57)cc4)cc3)cc(-c3ccc4ccccc4c3)n2)cc1 ; c1ccc(-c2cc(-c3cccc(-c4ccc5ccccc5c4-c4cccc(-c5ccc6c(c5)-c5cccc7cccc-6c57)c4)c3)nc(-c3ccccc3)n2)cc1]

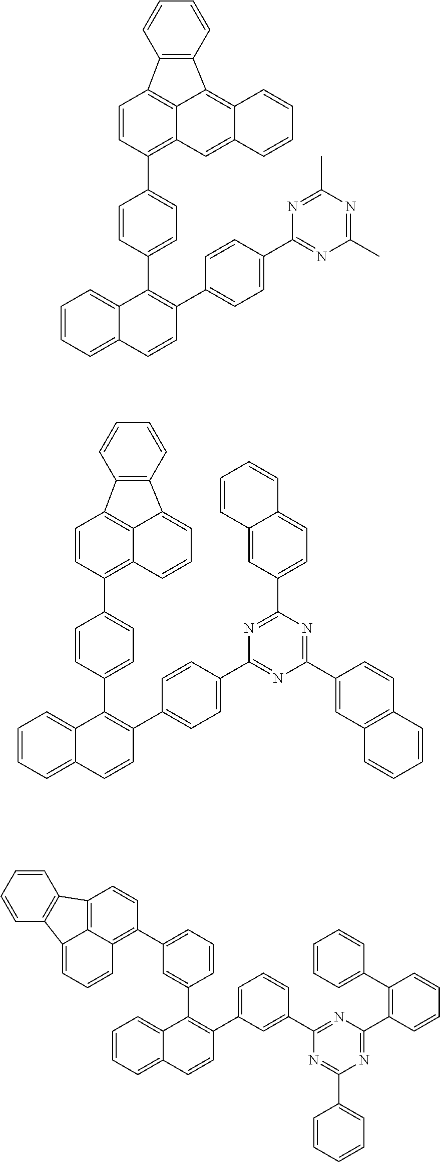 [SMILES: Cc1nc(C)nc(-c2ccc(-c3ccc4ccccc4c3-c3ccc(-c4ccc5c6c(c7ccccc7cc46)-c4ccccc4-5)cc3)cc2)n1 ; c1ccc2c(c1)-c1cccc3c(-c4ccc(-c5c(-c6ccc(-c7nc(-c8ccc9ccccc9c8)nc(-c8ccc9ccccc9c8)n7)cc6)ccc6ccccc56)cc4)ccc-2c13 ; c1ccc(-c2nc(-c3cccc(-c4ccc5ccccc5c4-c4cccc(-c5ccc6c7c(cccc57)-c5ccccc5-6)c4)c3)nc(-c3ccccc3-c3ccccc3)n2)cc1]

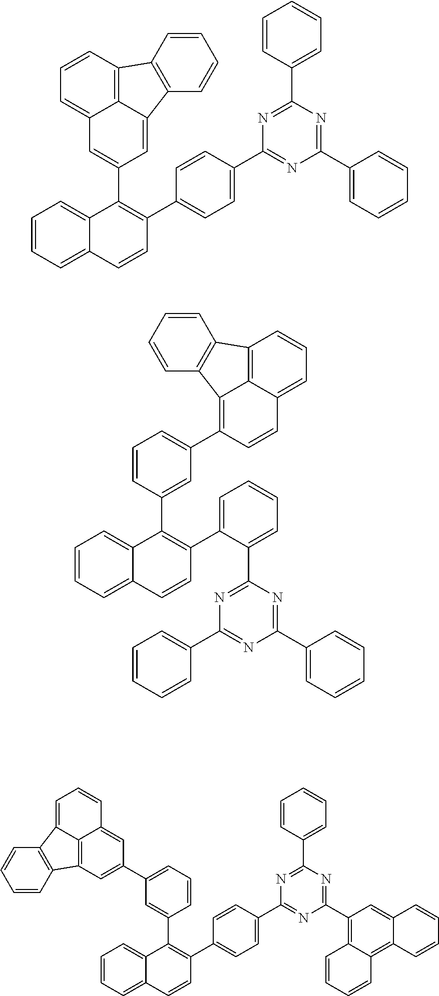 [SMILES: c1ccc(-c2nc(-c3ccccc3)nc(-c3ccc(-c4ccc5ccccc5c4-c4cc5c6c(cccc6c4)-c4ccccc4-5)cc3)n2)cc1 ; c1ccc(-c2nc(-c3ccccc3)nc(-c3ccccc3-c3ccc4ccccc4c3-c3cccc(-c4ccc5cccc6c5c4-c4ccccc4-6)c3)n2)cc1 ; c1ccc(-c2nc(-c3ccc(-c4ccc5ccccc5c4-c4cccc(-c5cc6c7c(cccc7c5)-c5ccccc5-6)c4)cc3)nc(-c3cc4ccccc4c4ccccc34)n2)cc1]

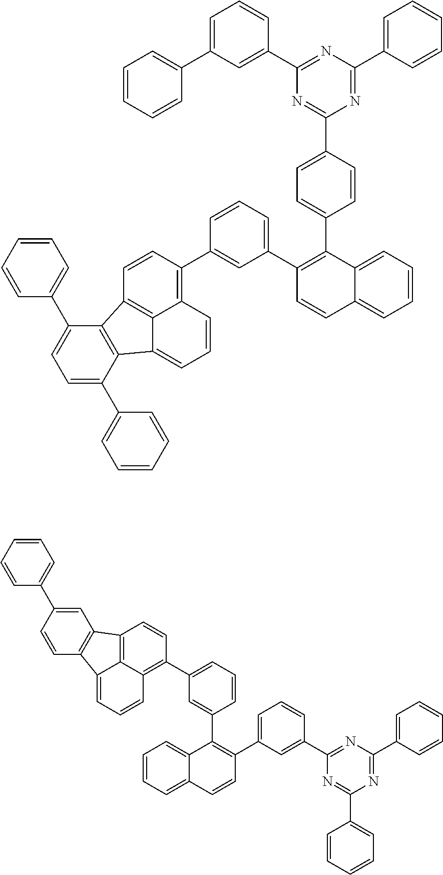 [SMILES: c1ccc(-c2cccc(-c3nc(-c4ccccc4)nc(-c4ccc(-c5c(-c6cccc(-c7ccc8c9c(cccc79)-c7c(-c9ccccc9)ccc(-c9ccccc9)c7-8)c6)ccc6ccccc56)cc4)n3)c2)cc1 ; c1ccc(-c2ccc3c(c2)-c2ccc(-c4cccc(-c5c(-c6cccc(-c7nc(-c8ccccc8)nc(-c8ccccc8)n7)c6)ccc6ccccc56)c4)c4cccc-3c24)cc1]

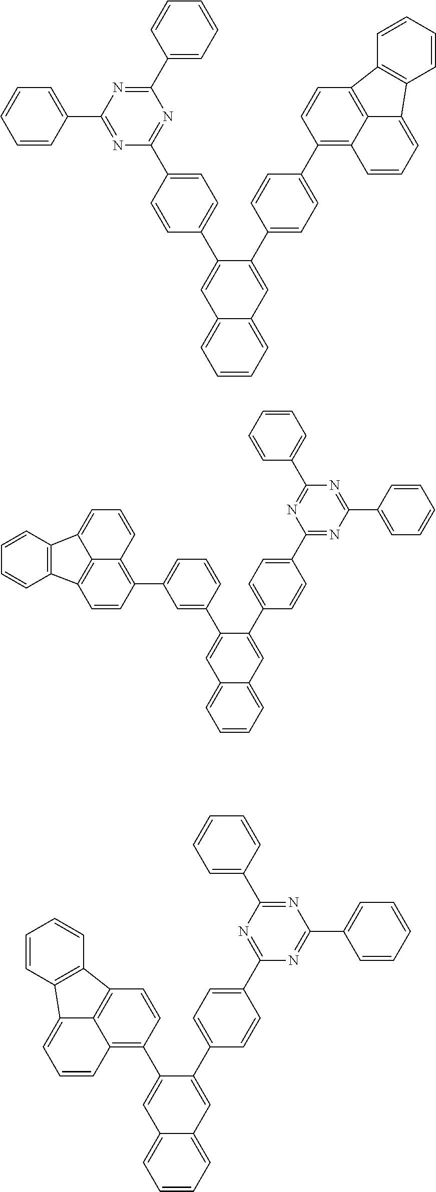

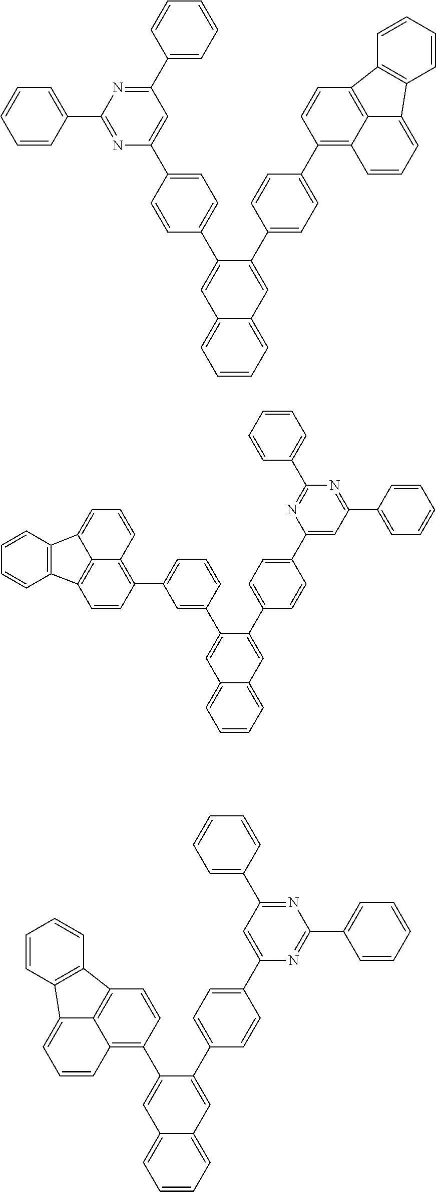

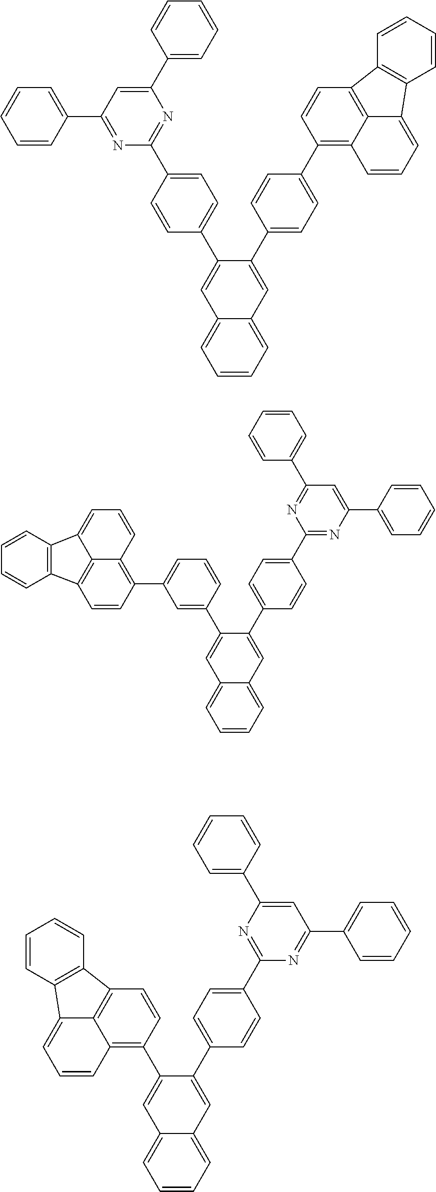

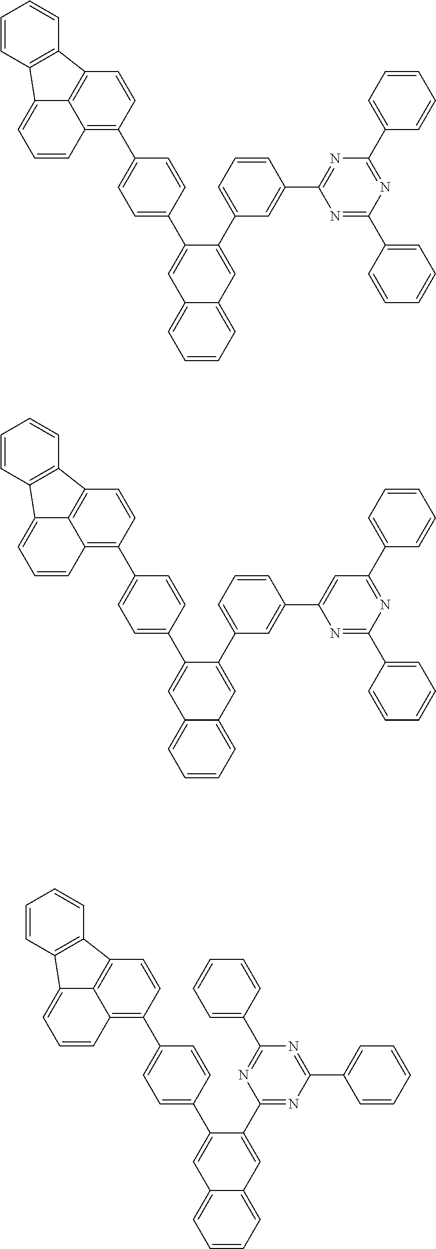 [SMILES: c1ccc(-c2nc(-c3ccccc3)nc(-c3cccc(-c4cc5ccccc5cc4-c4ccc(-c5ccc6c7c(cccc57)-c5ccccc5-6)cc4)c3)n2)cc1 ; c1ccc(-c2cc(-c3cccc(-c4cc5ccccc5cc4-c4ccc(-c5ccc6c7c(cccc57)-c5ccccc5-6)cc4)c3)nc(-c3ccccc3)n2)cc1 ; c1ccc(-c2nc(-c3ccccc3)nc(-c3cc4ccccc4cc3-c3ccc(-c4ccc5c6c(cccc46)-c4ccccc4-5)cc3)n2)cc1]

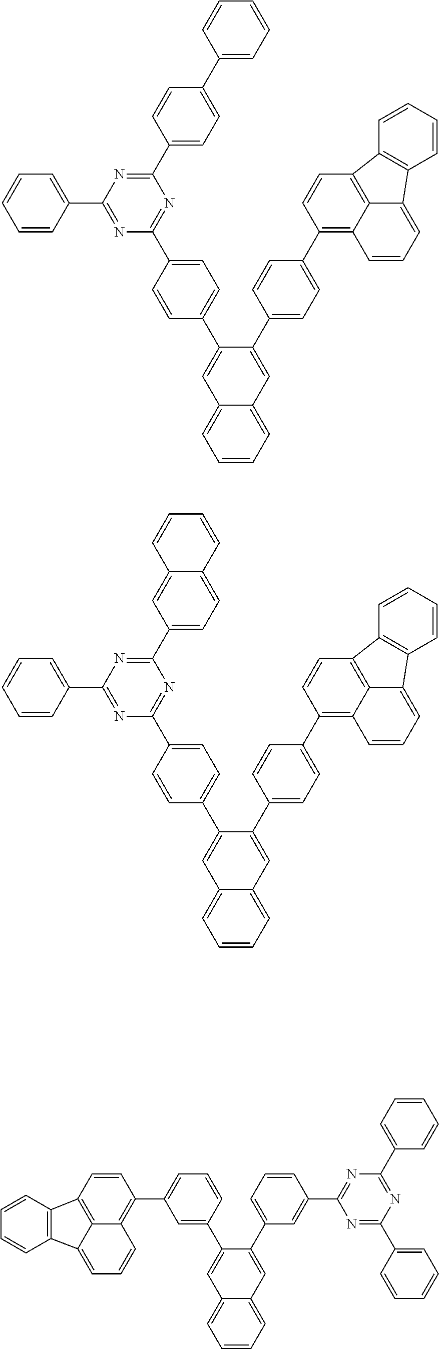 [SMILES: c1ccc(-c2ccc(-c3nc(-c4ccccc4)nc(-c4ccc(-c5cc6ccccc6cc5-c5ccc(-c6ccc7c8c(cccc68)-c6ccccc6-7)cc5)cc4)n3)cc2)cc1 ; c1ccc(-c2nc(-c3ccc(-c4cc5ccccc5cc4-c4ccc(-c5ccc6c7c(cccc57)-c5ccccc5-6)cc4)cc3)nc(-c3ccc4ccccc4c3)n2)cc1 ; c1ccc(-c2nc(-c3ccccc3)nc(-c3cccc(-c4cc5ccccc5cc4-c4cccc(-c5ccc6c7c(cccc57)-c5ccccc5-6)c4)c3)n2)cc1]

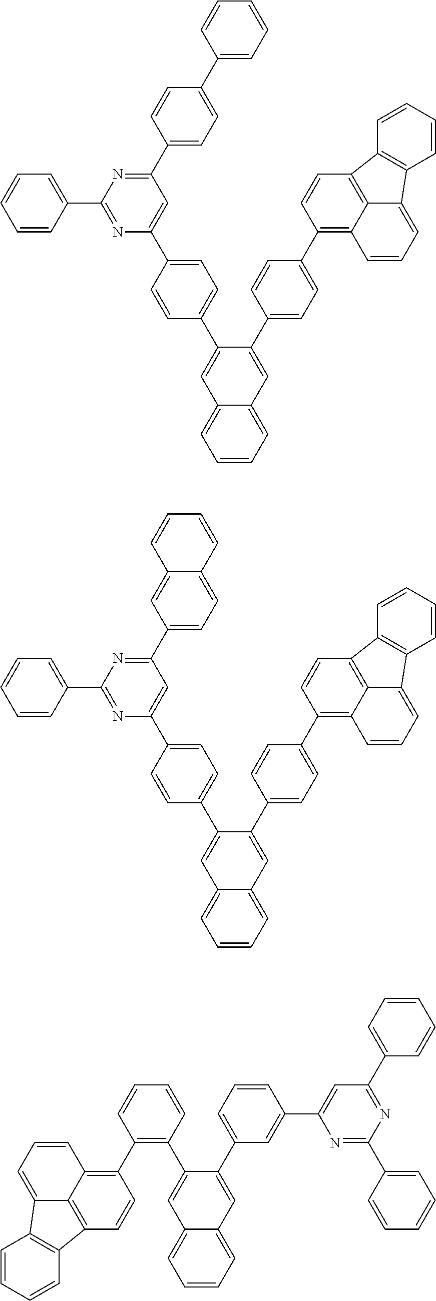 [SMILES: c1ccc(-c2ccc(-c3cc(-c4ccc(-c5cc6ccccc6cc5-c5ccc(-c6ccc7c8c(cccc68)-c6ccccc6-7)cc5)cc4)nc(-c4ccccc4)n3)cc2)cc1 ; c1ccc(-c2nc(-c3ccc(-c4cc5ccccc5cc4-c4ccc(-c5ccc6c7c(cccc57)-c5ccccc5-6)cc4)cc3)cc(-c3ccc4ccccc4c3)n2)cc1 ; c1ccc(-c2cc(-c3cccc(-c4cc5ccccc5cc4-c4ccccc4-c4ccc5c6c(cccc46)-c4ccccc4-5)c3)nc(-c3ccccc3)n2)cc1]

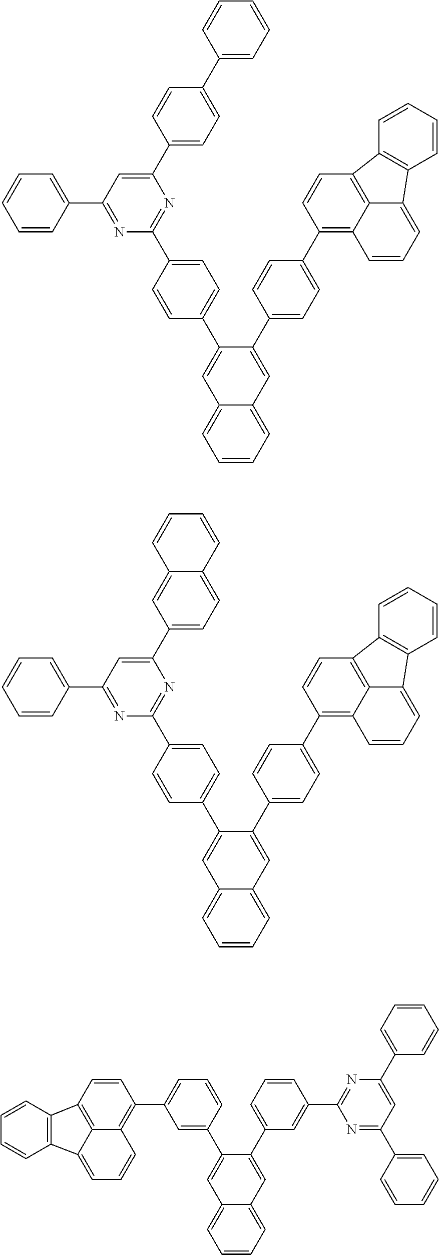 [SMILES: c1ccc(-c2ccc(-c3cc(-c4ccccc4)nc(-c4ccc(-c5cc6ccccc6cc5-c5ccc(-c6ccc7c8c(cccc68)-c6ccccc6-7)cc5)cc4)n3)cc2)cc1 ; c1ccc(-c2cc(-c3ccc4ccccc4c3)nc(-c3ccc(-c4cc5ccccc5cc4-c4ccc(-c5ccc6c7c(cccc57)-c5ccccc5-6)cc4)cc3)n2)cc1 ; c1ccc(-c2cc(-c3ccccc3)nc(-c3cccc(-c4cc5ccccc5cc4-c4cccc(-c5ccc6c7c(cccc57)-c5ccccc5-6)c4)c3)n2)cc1]

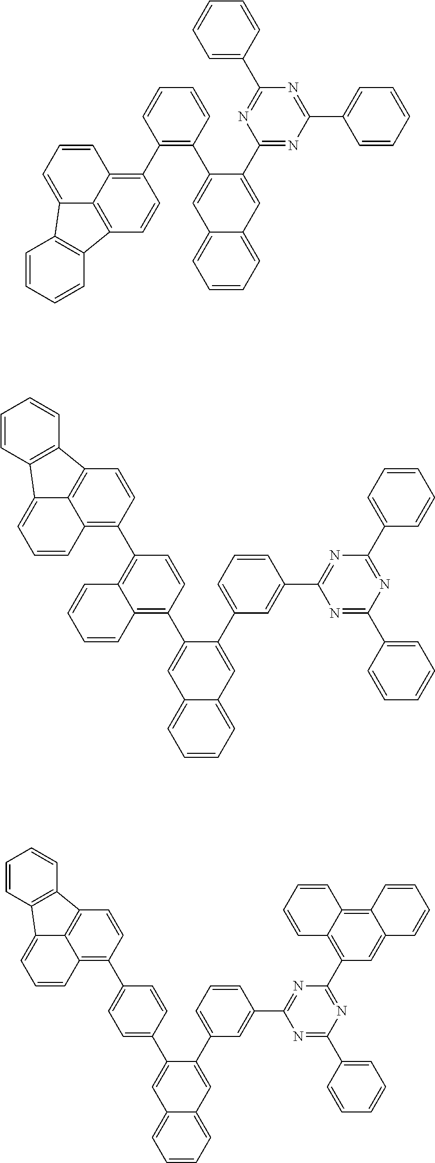 [SMILES: c1ccc(-c2nc(-c3ccccc3)nc(-c3cc4ccccc4cc3-c3ccccc3-c3ccc4c5c(cccc35)-c3ccccc3-4)n2)cc1 ; c1ccc(-c2nc(-c3ccccc3)nc(-c3cccc(-c4cc5ccccc5cc4-c4ccc(-c5ccc6c7c(cccc57)-c5ccccc5-6)c5ccccc45)c3)n2)cc1 ; c1ccc(-c2nc(-c3cccc(-c4cc5ccccc5cc4-c4ccc(-c5ccc6c7c(cccc57)-c5ccccc5-6)cc4)c3)nc(-c3cc4ccccc4c4ccccc34)n2)cc1]

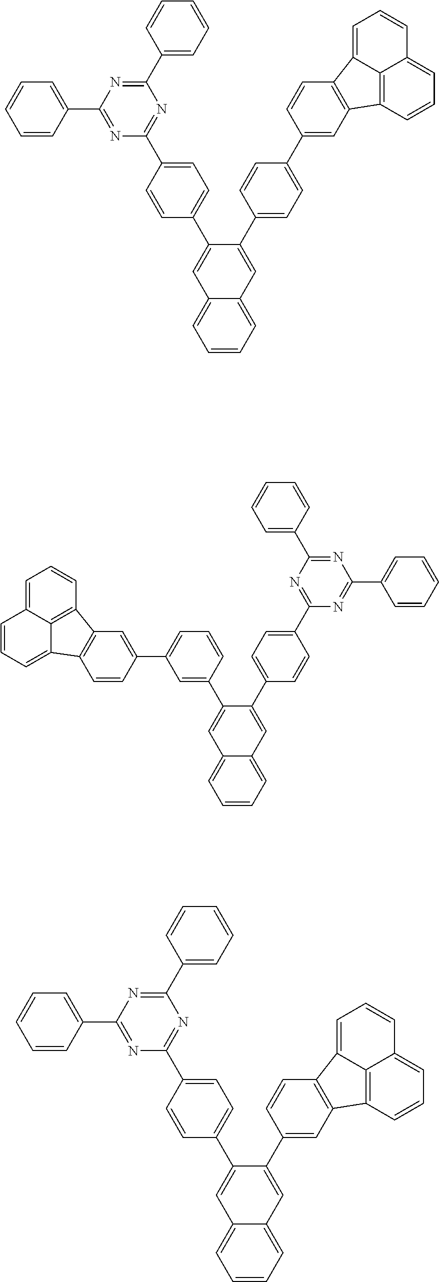 [SMILES: c1ccc(-c2nc(-c3ccccc3)nc(-c3ccc(-c4cc5ccccc5cc4-c4ccc(-c5ccc6c(c5)-c5cccc7cccc-6c57)cc4)cc3)n2)cc1 ; c1ccc(-c2nc(-c3ccccc3)nc(-c3ccc(-c4cc5ccccc5cc4-c4cccc(-c5ccc6c(c5)-c5cccc7cccc-6c57)c4)cc3)n2)cc1 ; c1ccc(-c2nc(-c3ccccc3)nc(-c3ccc(-c4cc5ccccc5cc4-c4ccc5c(c4)-c4cccc6cccc-5c46)cc3)n2)cc1]

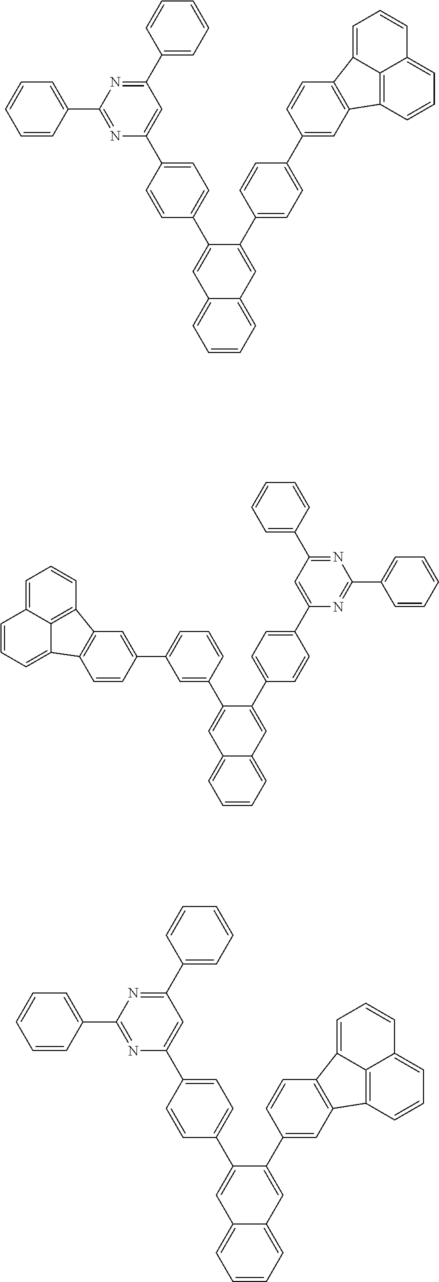 [SMILES: c1ccc(-c2cc(-c3ccc(-c4cc5ccccc5cc4-c4ccc(-c5ccc6c(c5)-c5cccc7cccc-6c57)cc4)cc3)nc(-c3ccccc3)n2)cc1 ; c1ccc(-c2cc(-c3ccc(-c4cc5ccccc5cc4-c4cccc(-c5ccc6c(c5)-c5cccc7cccc-6c57)c4)cc3)nc(-c3ccccc3)n2)cc1 ; c1ccc(-c2cc(-c3ccc(-c4cc5ccccc5cc4-c4ccc5c(c4)-c4cccc6cccc-5c46)cc3)nc(-c3ccccc3)n2)cc1]

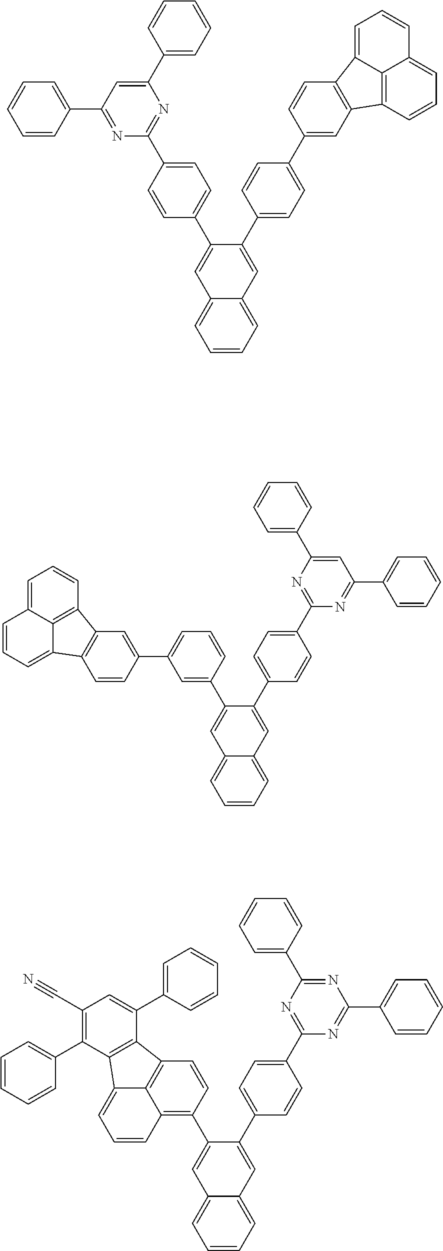 [SMILES: c1ccc(-c2cc(-c3ccccc3)nc(-c3ccc(-c4cc5ccccc5cc4-c4ccc(-c5ccc6c(c5)-c5cccc7cccc-6c57)cc4)cc3)n2)cc1 ; c1ccc(-c2cc(-c3ccccc3)nc(-c3ccc(-c4cc5ccccc5cc4-c4cccc(-c5ccc6c(c5)-c5cccc7cccc-6c57)c4)cc3)n2)cc1 ; N#Cc1cc(-c2ccccc2)c2c(c1-c1ccccc1)-c1cccc3c(-c4cc5ccccc5cc4-c4ccc(-c5nc(-c6ccccc6)nc(-c6ccccc6)n5)cc4)ccc-2c13]

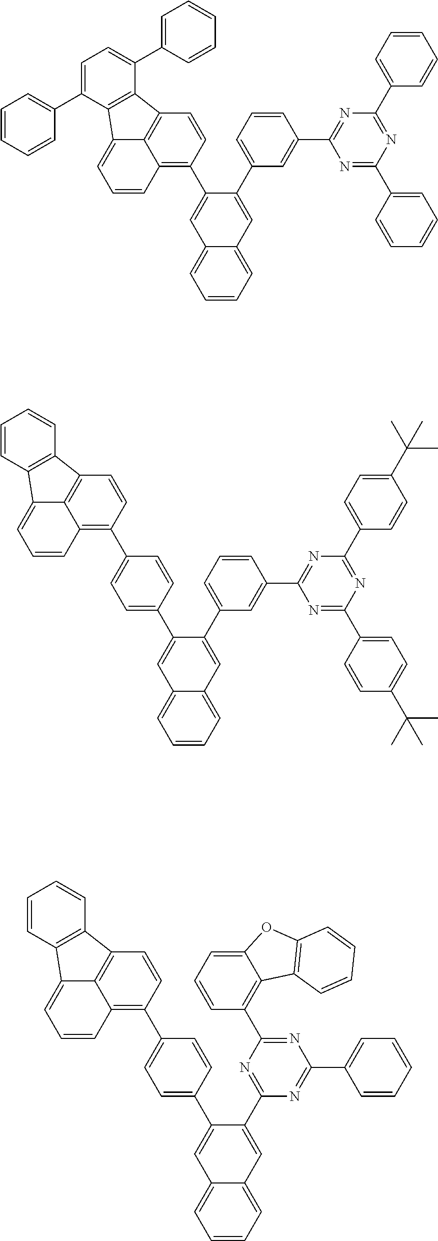 [SMILES: c1ccc(-c2nc(-c3ccccc3)nc(-c3cccc(-c4cc5ccccc5cc4-c4ccc5c6c(cccc46)-c4c(-c6ccccc6)ccc(-c6ccccc6)c4-5)c3)n2)cc1 ; CC(C)(C)c1ccc(-c2nc(-c3ccc(C(C)(C)C)cc3)nc(-c3cccc(-c4cc5ccccc5cc4-c4ccc(-c5ccc6c7c(cccc57)-c5ccccc5-6)cc4)c3)n2)cc1 ; c1ccc(-c2nc(-c3cc4ccccc4cc3-c3ccc(-c4ccc5c6c(cccc46)-c4ccccc4-5)cc3)nc(-c3cccc4oc5ccccc5c34)n2)cc1]

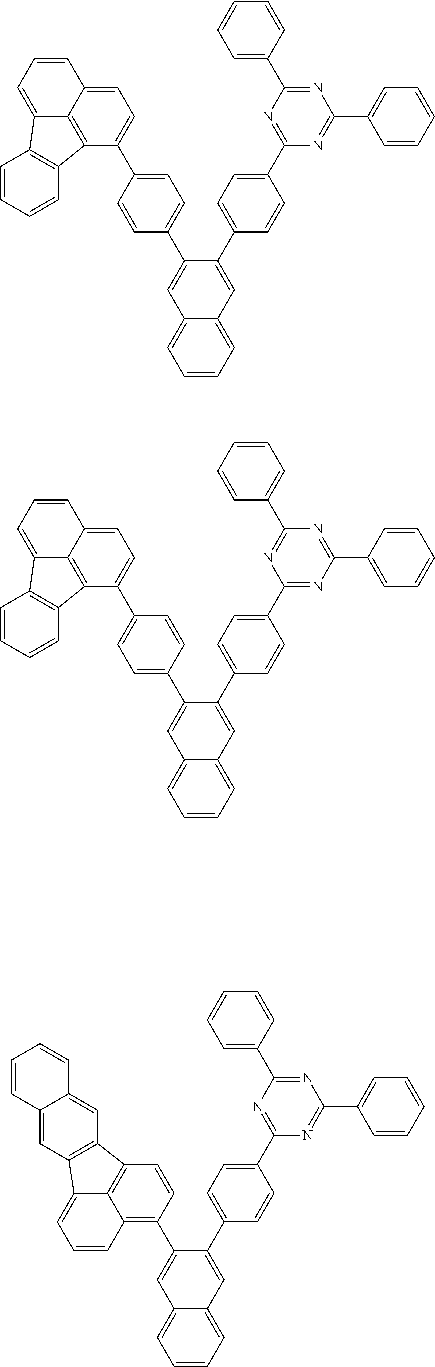 [SMILES: c1ccc(-c2nc(-c3ccccc3)nc(-c3ccc(-c4cc5ccccc5cc4-c4ccc(-c5ccc6cccc7c6c5-c5ccccc5-7)cc4)cc3)n2)cc1 ; c1ccc(-c2nc(-c3ccccc3)nc(-c3ccc(-c4cc5ccccc5cc4-c4ccc(-c5ccc6cccc7c6c5-c5ccccc5-7)cc4)cc3)n2)cc1 ; c1ccc(-c2nc(-c3ccccc3)nc(-c3ccc(-c4cc5ccccc5cc4-c4ccc5c6c(cccc46)-c4cc6ccccc6cc4-5)cc3)n2)cc1]

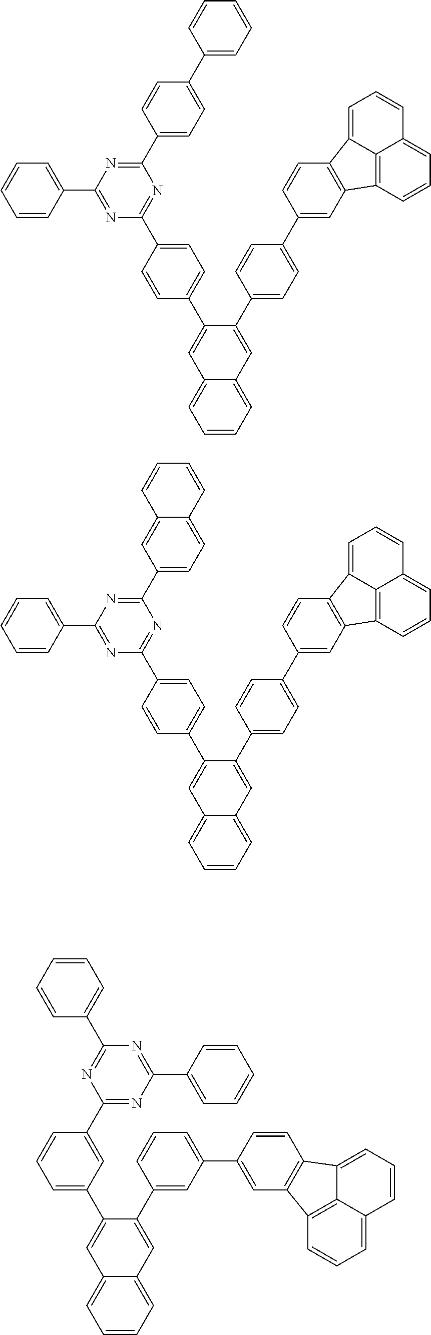

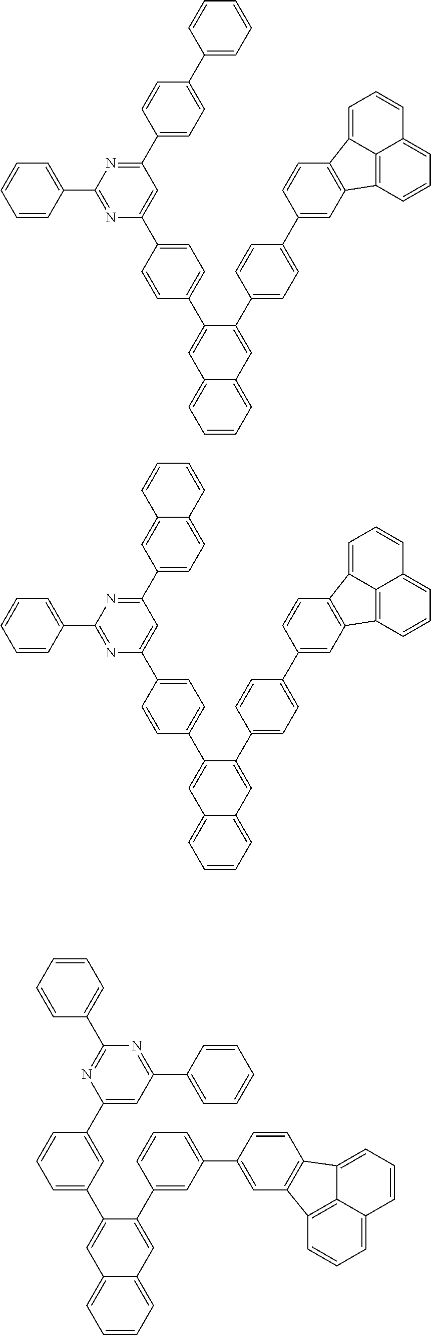

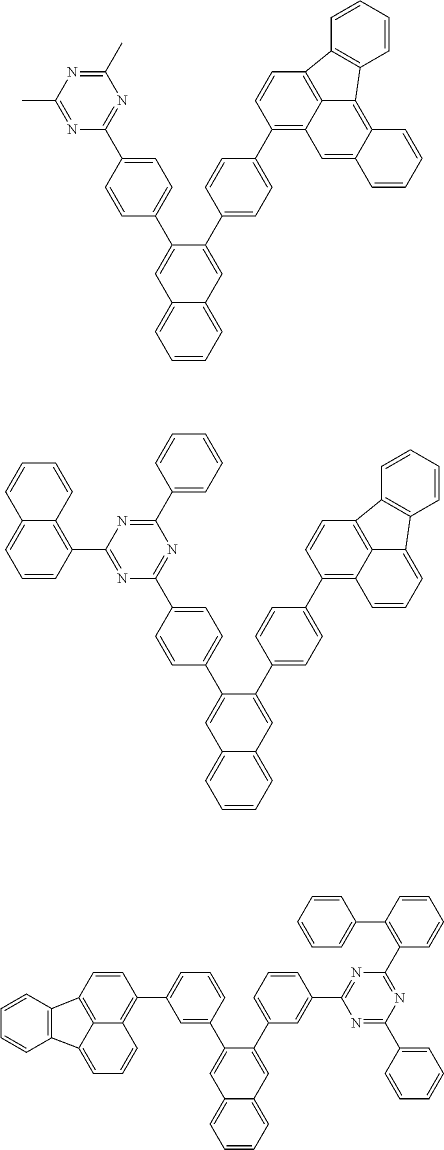 [SMILES: Cc1nc(C)nc(-c2ccc(-c3cc4ccccc4cc3-c3ccc(-c4ccc5c6c(c7ccccc7cc46)-c4ccccc4-5)cc3)cc2)n1 ; c1ccc(-c2nc(-c3ccc(-c4cc5ccccc5cc4-c4ccc(-c5ccc6c7c(cccc57)-c5ccccc5-6)cc4)cc3)nc(-c3cccc4ccccc34)n2)cc1 ; c1ccc(-c2nc(-c3cccc(-c4cc5ccccc5cc4-c4cccc(-c5ccc6c7c(cccc57)-c5ccccc5-6)c4)c3)nc(-c3ccccc3-c3ccccc3)n2)cc1]

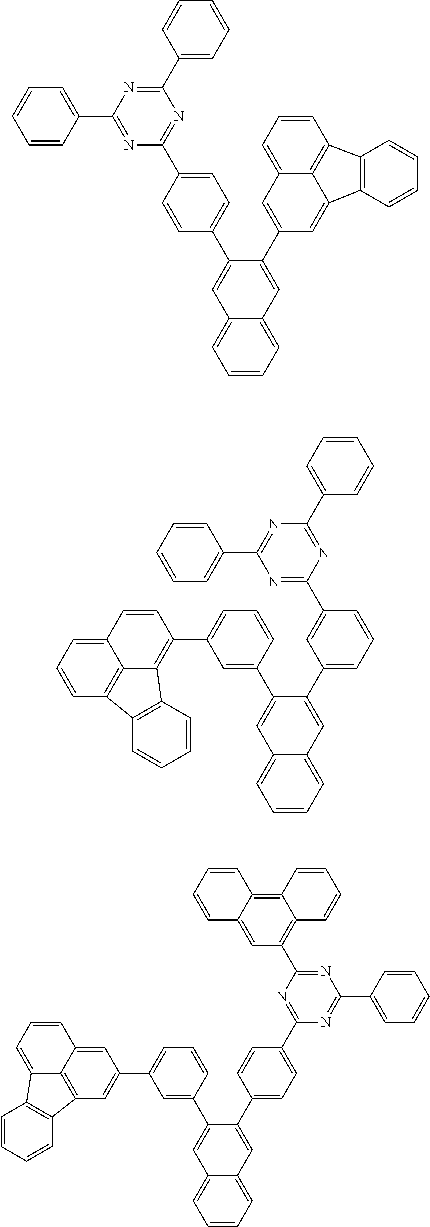 [SMILES: c1ccc(-c2nc(-c3ccccc3)nc(-c3ccc(-c4cc5ccccc5cc4-c4cc5c6c(cccc6c4)-c4ccccc4-5)cc3)n2)cc1 ; c1ccc(-c2nc(-c3ccccc3)nc(-c3cccc(-c4cc5ccccc5cc4-c4cccc(-c5ccc6cccc7c6c5-c5ccccc5-7)c4)c3)n2)cc1 ; c1ccc(-c2nc(-c3ccc(-c4cc5ccccc5cc4-c4cccc(-c5cc6c7c(cccc7c5)-c5ccccc5-6)c4)cc3)nc(-c3cc4ccccc4c4ccccc34)n2)cc1]

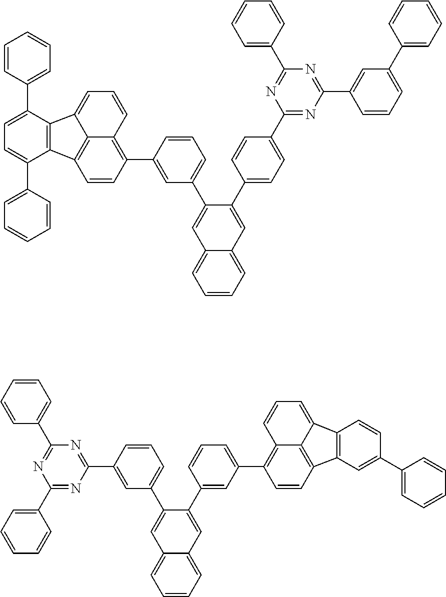 [SMILES: c1ccc(-c2cccc(-c3nc(-c4ccccc4)nc(-c4ccc(-c5cc6ccccc6cc5-c5cccc(-c6ccc7c8c(cccc68)-c6c(-c8ccccc8)ccc(-c8ccccc8)c6-7)c5)cc4)n3)c2)cc1 ; c1ccc(-c2ccc3c(c2)-c2ccc(-c4cccc(-c5cc6ccccc6cc5-c5cccc(-c6nc(-c7ccccc7)nc(-c7ccccc7)n6)c5)c4)c4cccc-3c24)cc1]

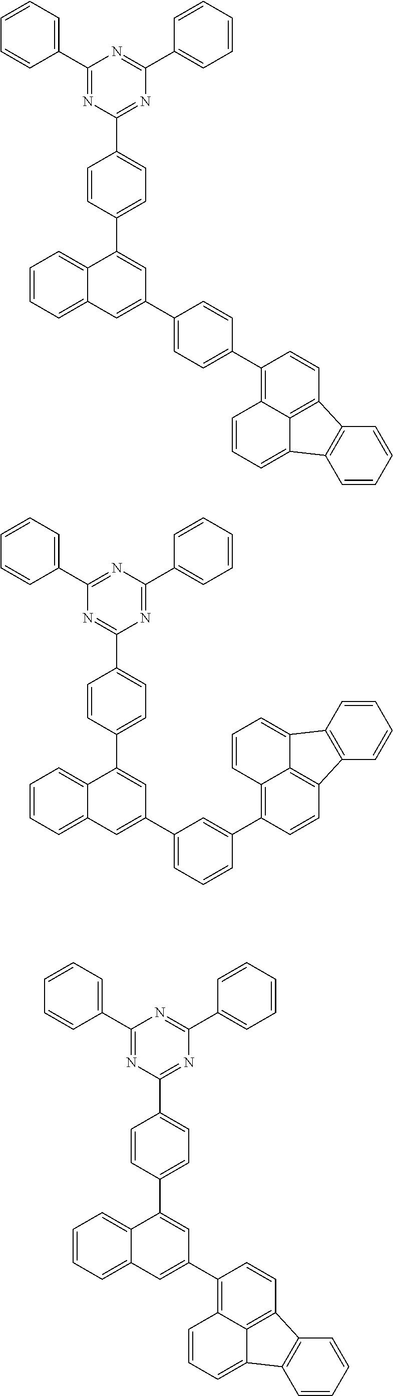 [SMILES: c1ccc(-c2nc(-c3ccccc3)nc(-c3ccc(-c4cc(-c5ccc(-c6ccc7c8c(cccc68)-c6ccccc6-7)cc5)cc5ccccc45)cc3)n2)cc1 ; c1ccc(-c2nc(-c3ccccc3)nc(-c3ccc(-c4cc(-c5cccc(-c6ccc7c8c(cccc68)-c6ccccc6-7)c5)cc5ccccc45)cc3)n2)cc1 ; c1ccc(-c2nc(-c3ccccc3)nc(-c3ccc(-c4cc(-c5ccc6c7c(cccc57)-c5ccccc5-6)cc5ccccc45)cc3)n2)cc1]

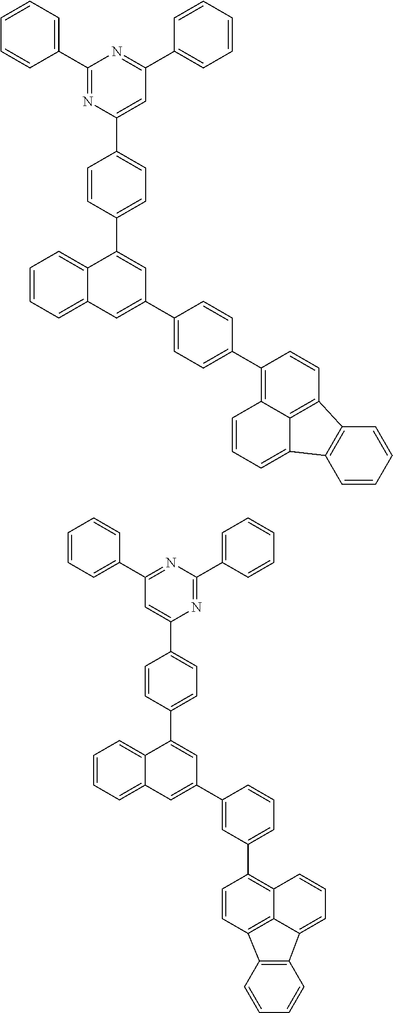 [SMILES: c1ccc(-c2cc(-c3ccc(-c4cc(-c5ccc(-c6ccc7c8c(cccc68)-c6ccccc6-7)cc5)cc5ccccc45)cc3)nc(-c3ccccc3)n2)cc1 ; c1ccc(-c2cc(-c3ccc(-c4cc(-c5cccc(-c6ccc7c8c(cccc68)-c6ccccc6-7)c5)cc5ccccc45)cc3)nc(-c3ccccc3)n2)cc1]

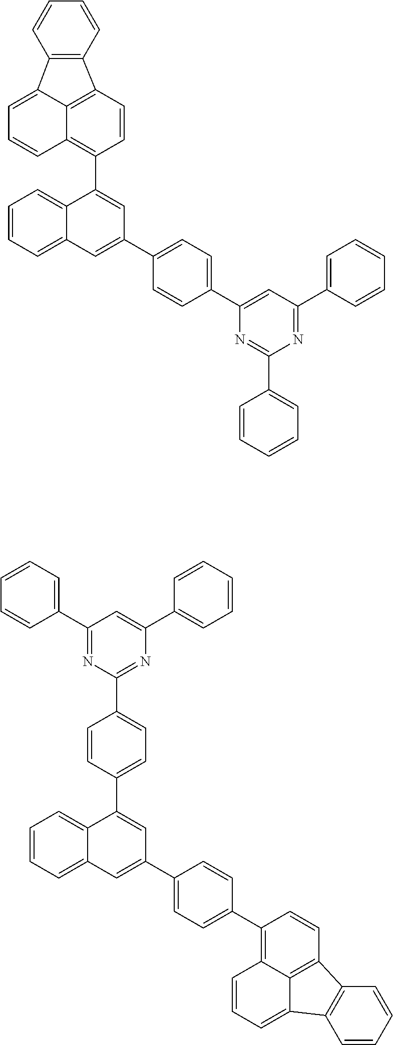 [SMILES: c1ccc(-c2cc(-c3ccc(-c4cc(-c5ccc6c7c(cccc57)-c5ccccc5-6)c5ccccc5c4)cc3)nc(-c3ccccc3)n2)cc1 ; c1ccc(-c2cc(-c3ccccc3)nc(-c3ccc(-c4cc(-c5ccc(-c6ccc7c8c(cccc68)-c6ccccc6-7)cc5)cc5ccccc45)cc3)n2)cc1]

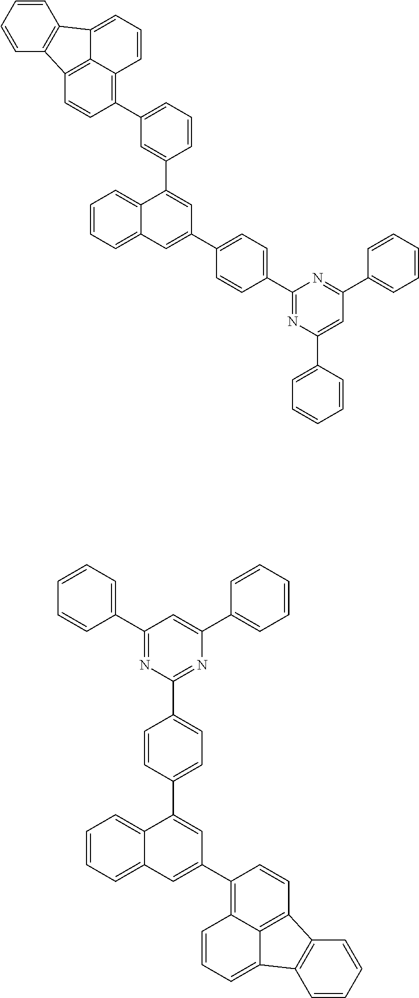 [SMILES: c1ccc(-c2cc(-c3ccccc3)nc(-c3ccc(-c4cc(-c5cccc(-c6ccc7c8c(cccc68)-c6ccccc6-7)c5)c5ccccc5c4)cc3)n2)cc1 ; c1ccc(-c2cc(-c3ccccc3)nc(-c3ccc(-c4cc(-c5ccc6c7c(cccc57)-c5ccccc5-6)cc5ccccc45)cc3)n2)cc1]

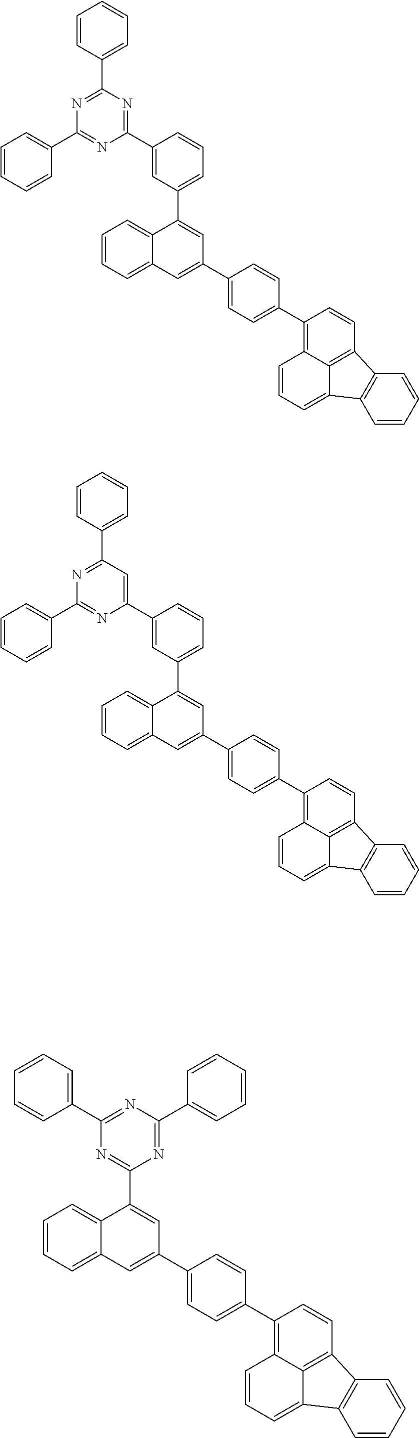 [SMILES: c1ccc(-c2nc(-c3ccccc3)nc(-c3cccc(-c4cc(-c5ccc(-c6ccc7c8c(cccc68)-c6ccccc6-7)cc5)cc5ccccc45)c3)n2)cc1 ; c1ccc(-c2cc(-c3cccc(-c4cc(-c5ccc(-c6ccc7c8c(cccc68)-c6ccccc6-7)cc5)cc5ccccc45)c3)nc(-c3ccccc3)n2)cc1 ; c1ccc(-c2nc(-c3ccccc3)nc(-c3cc(-c4ccc(-c5ccc6c7c(cccc57)-c5ccccc5-6)cc4)cc4ccccc34)n2)cc1]

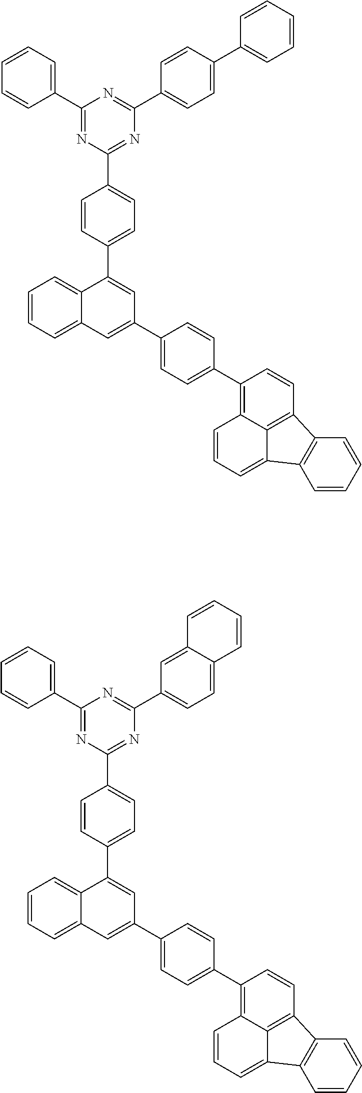 [SMILES: c1ccc(-c2ccc(-c3nc(-c4ccccc4)nc(-c4ccc(-c5cc(-c6ccc(-c7ccc8c9c(cccc79)-c7ccccc7-8)cc6)cc6ccccc56)cc4)n3)cc2)cc1 ; c1ccc(-c2nc(-c3ccc(-c4cc(-c5ccc(-c6ccc7c8c(cccc68)-c6ccccc6-7)cc5)cc5ccccc45)cc3)nc(-c3ccc4ccccc4c3)n2)cc1]

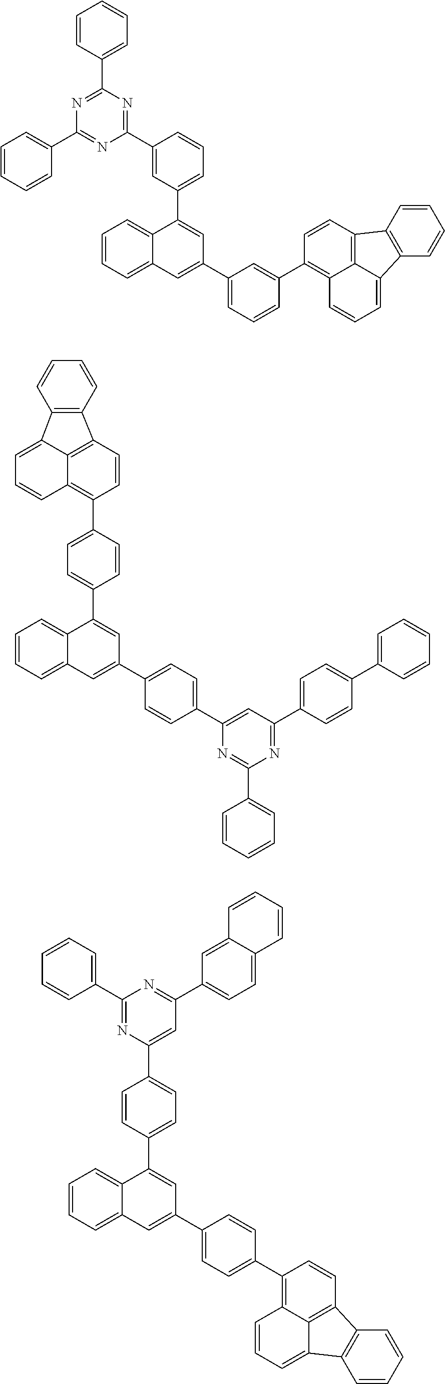

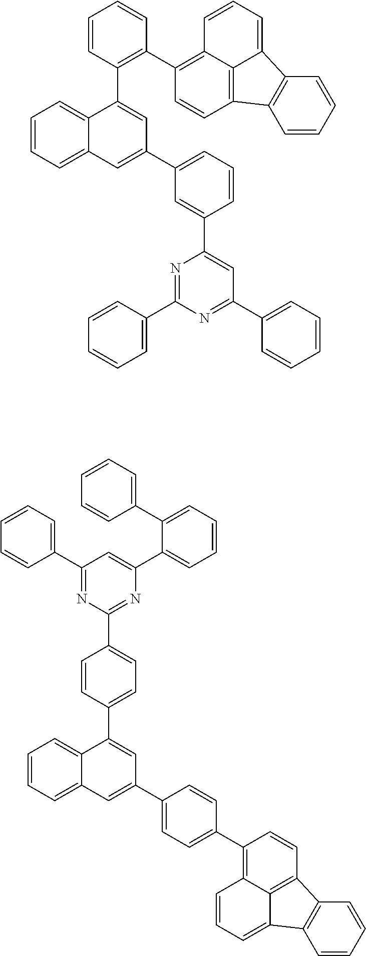 [SMILES: c1ccc(-c2cc(-c3cccc(-c4cc(-c5ccccc5-c5ccc6c7c(cccc57)-c5ccccc5-6)c5ccccc5c4)c3)nc(-c3ccccc3)n2)cc1 ; c1ccc(-c2cc(-c3ccccc3-c3ccccc3)nc(-c3ccc(-c4cc(-c5ccc(-c6ccc7c8c(cccc68)-c6ccccc6-7)cc5)cc5ccccc45)cc3)n2)cc1]

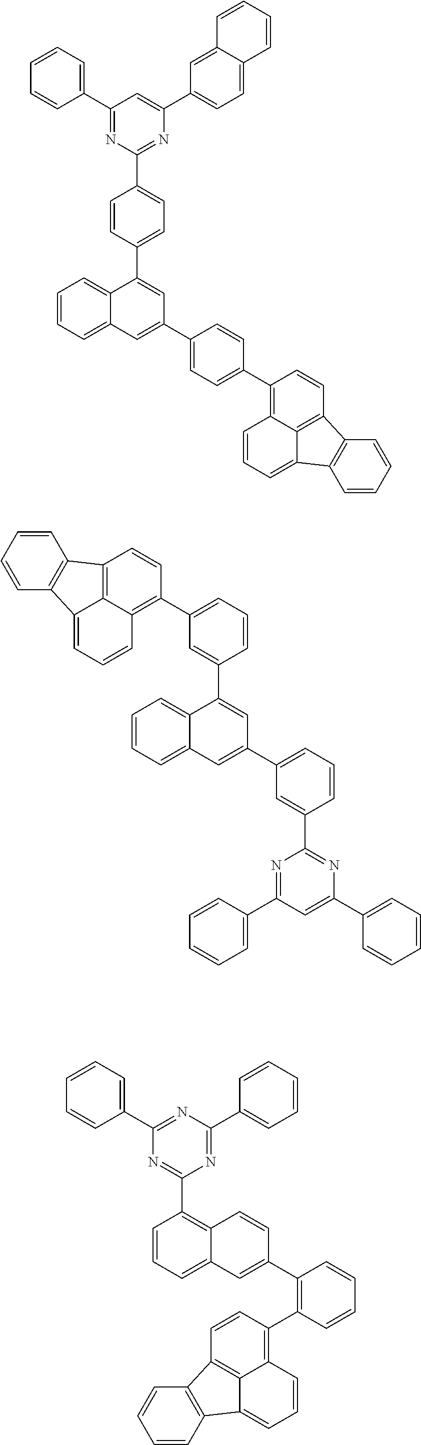 [SMILES: c1ccc(-c2cc(-c3ccc4ccccc4c3)nc(-c3ccc(-c4cc(-c5ccc(-c6ccc7c8c(cccc68)-c6ccccc6-7)cc5)cc5ccccc45)cc3)n2)cc1 ; c1ccc(-c2cc(-c3ccccc3)nc(-c3cccc(-c4cc(-c5cccc(-c6ccc7c8c(cccc68)-c6ccccc6-7)c5)c5ccccc5c4)c3)n2)cc1 ; c1ccc(-c2nc(-c3ccccc3)nc(-c3cccc4cc(-c5ccccc5-c5ccc6c7c(cccc57)-c5ccccc5-6)ccc34)n2)cc1]

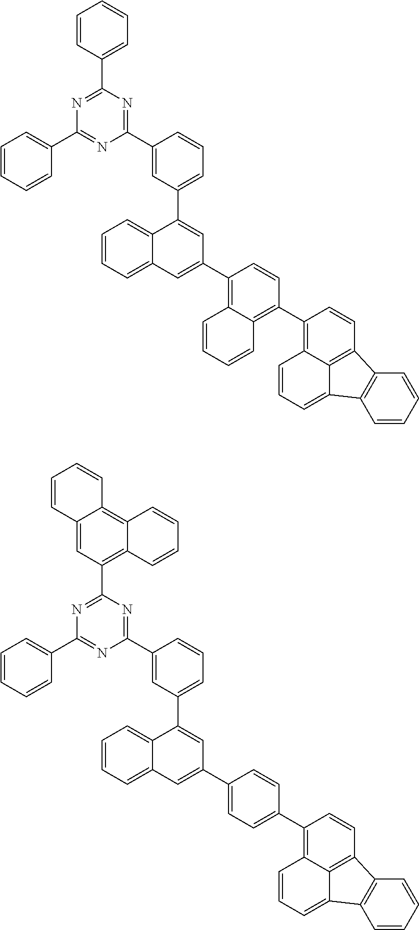 [SMILES: c1ccc(-c2nc(-c3ccccc3)nc(-c3cccc(-c4cc(-c5ccc(-c6ccc7c8c(cccc68)-c6ccccc6-7)c6ccccc56)cc5ccccc45)c3)n2)cc1 ; c1ccc(-c2nc(-c3cccc(-c4cc(-c5ccc(-c6ccc7c8c(cccc68)-c6ccccc6-7)cc5)cc5ccccc45)c3)nc(-c3cc4ccccc4c4ccccc34)n2)cc1]

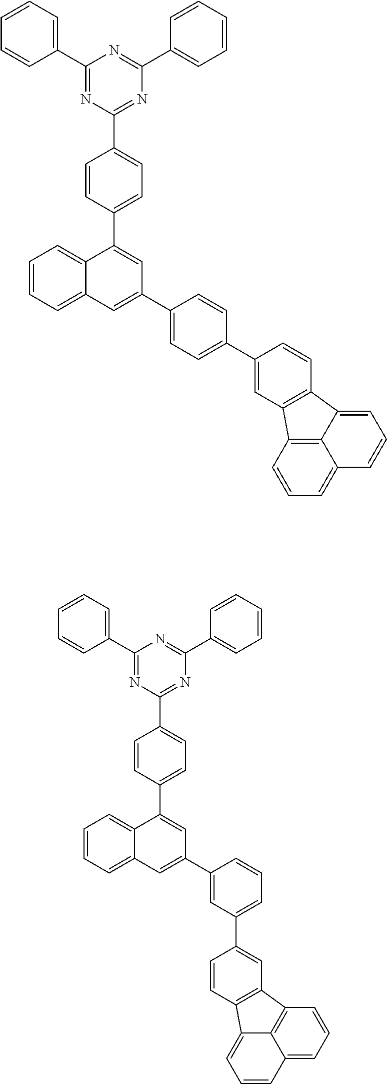 [SMILES: c1ccc(-c2nc(-c3ccccc3)nc(-c3ccc(-c4cc(-c5ccc(-c6ccc7c(c6)-c6cccc8cccc-7c68)cc5)cc5ccccc45)cc3)n2)cc1 ; c1ccc(-c2nc(-c3ccccc3)nc(-c3ccc(-c4cc(-c5cccc(-c6ccc7c(c6)-c6cccc8cccc-7c68)c5)cc5ccccc45)cc3)n2)cc1]

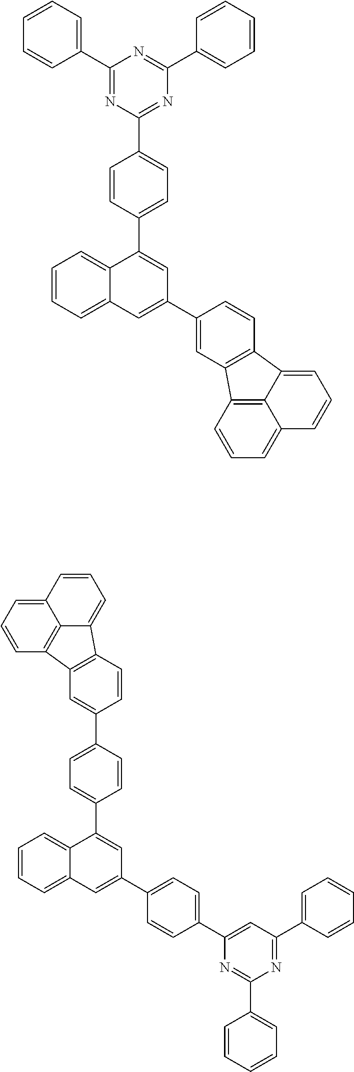 [SMILES: c1ccc(-c2nc(-c3ccccc3)nc(-c3ccc(-c4cc(-c5ccc6c(c5)-c5cccc7cccc-6c57)cc5ccccc45)cc3)n2)cc1 ; c1ccc(-c2cc(-c3ccc(-c4cc(-c5ccc(-c6ccc7c(c6)-c6cccc8cccc-7c68)cc5)c5ccccc5c4)cc3)nc(-c3ccccc3)n2)cc1]

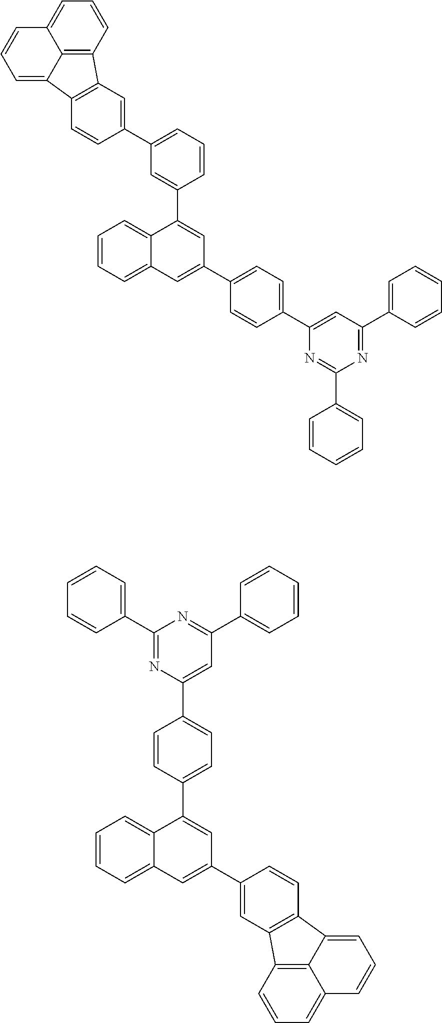 [SMILES: c1ccc(-c2cc(-c3ccc(-c4cc(-c5cccc(-c6ccc7c(c6)-c6cccc8cccc-7c68)c5)c5ccccc5c4)cc3)nc(-c3ccccc3)n2)cc1 ; c1ccc(-c2cc(-c3ccc(-c4cc(-c5ccc6c(c5)-c5cccc7cccc-6c57)cc5ccccc45)cc3)nc(-c3ccccc3)n2)cc1]

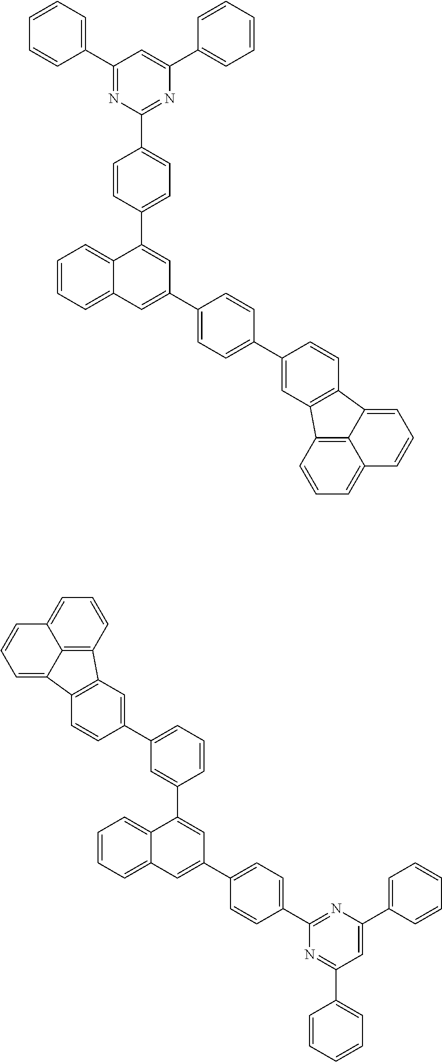 [SMILES: c1ccc(-c2cc(-c3ccccc3)nc(-c3ccc(-c4cc(-c5ccc(-c6ccc7c(c6)-c6cccc8cccc-7c68)cc5)cc5ccccc45)cc3)n2)cc1 ; c1ccc(-c2cc(-c3ccccc3)nc(-c3ccc(-c4cc(-c5cccc(-c6ccc7c(c6)-c6cccc8cccc-7c68)c5)c5ccccc5c4)cc3)n2)cc1]

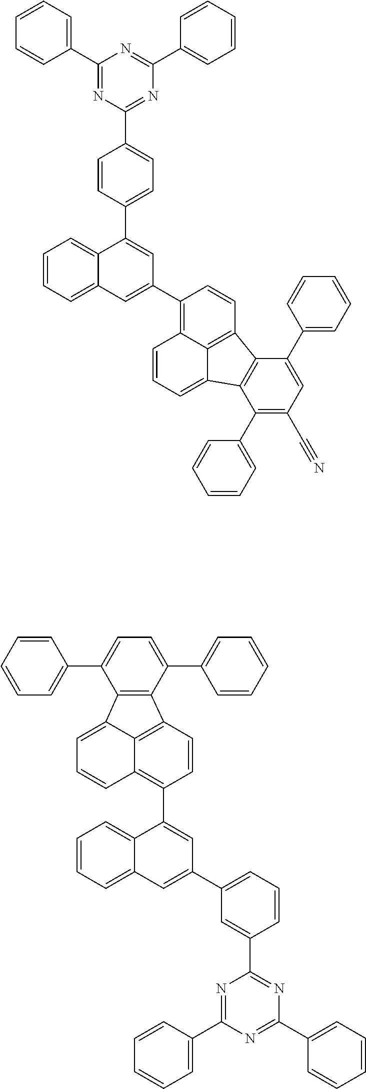 [SMILES: N#Cc1cc(-c2ccccc2)c2c(c1-c1ccccc1)-c1cccc3c(-c4cc(-c5ccc(-c6nc(-c7ccccc7)nc(-c7ccccc7)n6)cc5)c5ccccc5c4)ccc-2c13 ; c1ccc(-c2nc(-c3ccccc3)nc(-c3cccc(-c4cc(-c5ccc6c7c(cccc57)-c5c(-c7ccccc7)ccc(-c7ccccc7)c5-6)c5ccccc5c4)c3)n2)cc1]

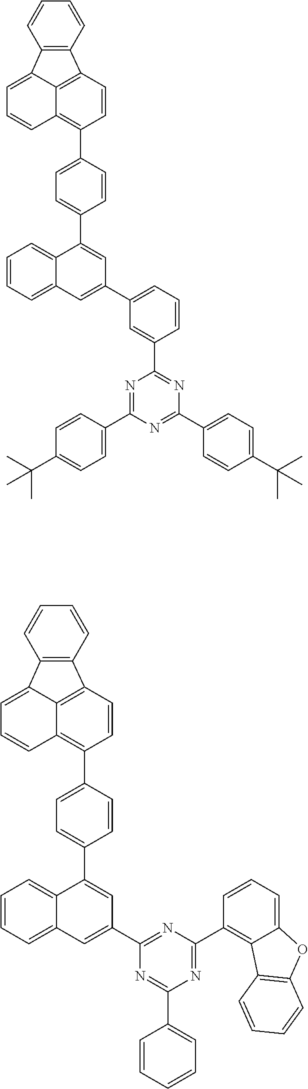 [SMILES: CC(C)(C)c1ccc(-c2nc(-c3ccc(C(C)(C)C)cc3)nc(-c3cccc(-c4cc(-c5ccc(-c6ccc7c8c(cccc68)-c6ccccc6-7)cc5)c5ccccc5c4)c3)n2)cc1 ; c1ccc(-c2nc(-c3cc(-c4ccc(-c5ccc6c7c(cccc57)-c5ccccc5-6)cc4)c4ccccc4c3)nc(-c3cccc4oc5ccccc5c34)n2)cc1]

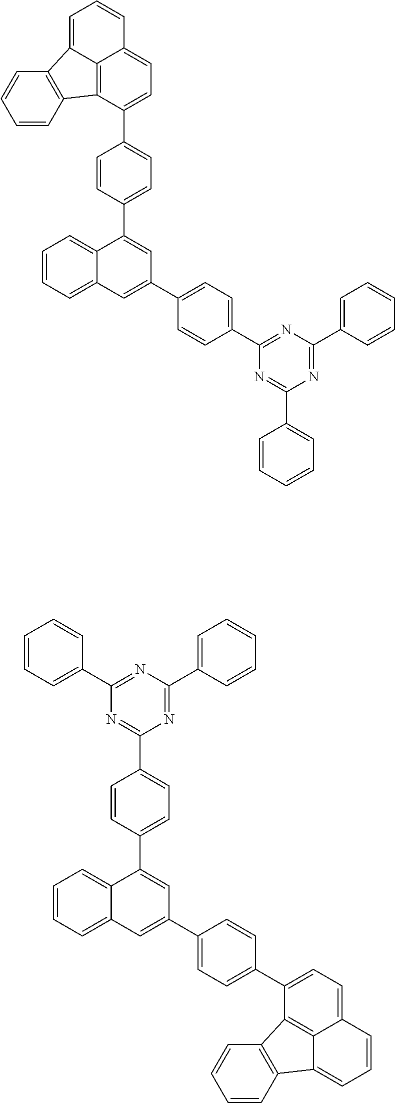 [SMILES: c1ccc(-c2nc(-c3ccccc3)nc(-c3ccc(-c4cc(-c5ccc(-c6ccc7cccc8c7c6-c6ccccc6-8)cc5)c5ccccc5c4)cc3)n2)cc1 ; c1ccc(-c2nc(-c3ccccc3)nc(-c3ccc(-c4cc(-c5ccc(-c6ccc7cccc8c7c6-c6ccccc6-8)cc5)cc5ccccc45)cc3)n2)cc1]

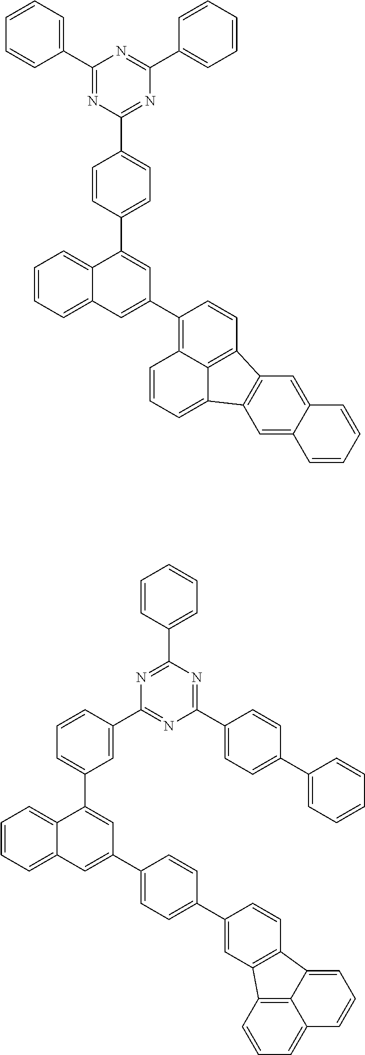 [SMILES: c1ccc(-c2nc(-c3ccccc3)nc(-c3ccc(-c4cc(-c5ccc6c7c(cccc57)-c5cc7ccccc7cc5-6)cc5ccccc45)cc3)n2)cc1 ; c1ccc(-c2ccc(-c3nc(-c4ccccc4)nc(-c4cccc(-c5cc(-c6ccc(-c7ccc8c(c7)-c7cccc9cccc-8c79)cc6)cc6ccccc56)c4)n3)cc2)cc1]

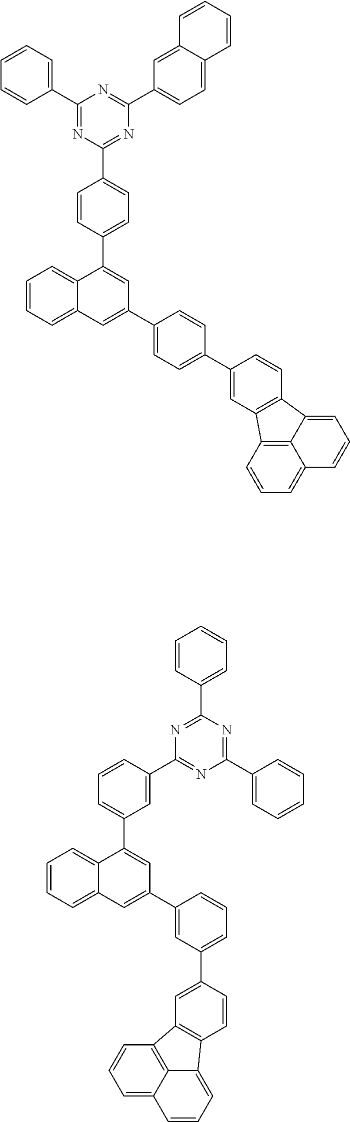 [SMILES: c1ccc(-c2nc(-c3ccc(-c4cc(-c5ccc(-c6ccc7c(c6)-c6cccc8cccc-7c68)cc5)cc5ccccc45)cc3)nc(-c3ccc4ccccc4c3)n2)cc1 ; c1ccc(-c2nc(-c3ccccc3)nc(-c3cccc(-c4cc(-c5cccc(-c6ccc7c(c6)-c6cccc8cccc-7c68)c5)cc5ccccc45)c3)n2)cc1]

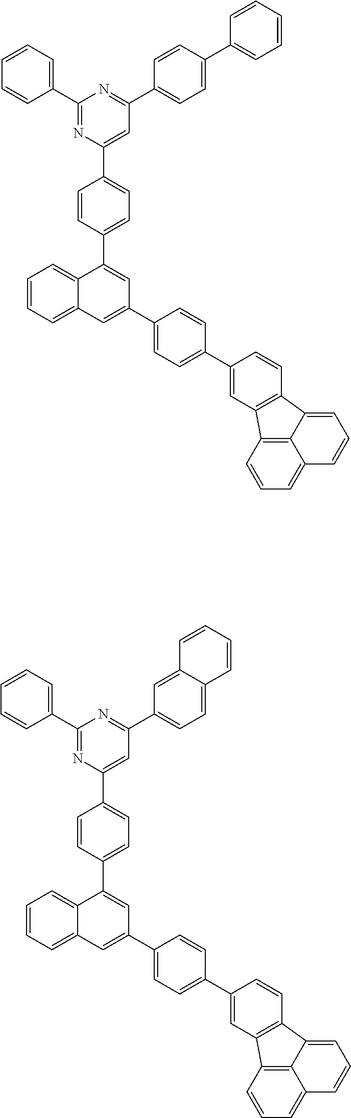 [SMILES: c1ccc(-c2ccc(-c3cc(-c4ccc(-c5cc(-c6ccc(-c7ccc8c(c7)-c7cccc9cccc-8c79)cc6)cc6ccccc56)cc4)nc(-c4ccccc4)n3)cc2)cc1 ; c1ccc(-c2nc(-c3ccc(-c4cc(-c5ccc(-c6ccc7c(c6)-c6cccc8cccc-7c68)cc5)cc5ccccc45)cc3)cc(-c3ccc4ccccc4c3)n2)cc1]

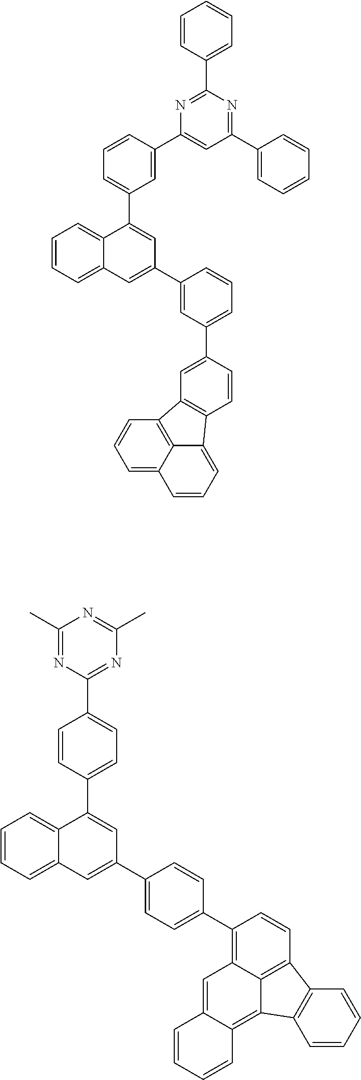 [SMILES: c1ccc(-c2cc(-c3cccc(-c4cc(-c5cccc(-c6ccc7c(c6)-c6cccc8cccc-7c68)c5)cc5ccccc45)c3)nc(-c3ccccc3)n2)cc1 ; Cc1nc(C)nc(-c2ccc(-c3cc(-c4ccc(-c5ccc6c7c(c8ccccc8cc57)-c5ccccc5-6)cc4)cc4ccccc34)cc2)n1]

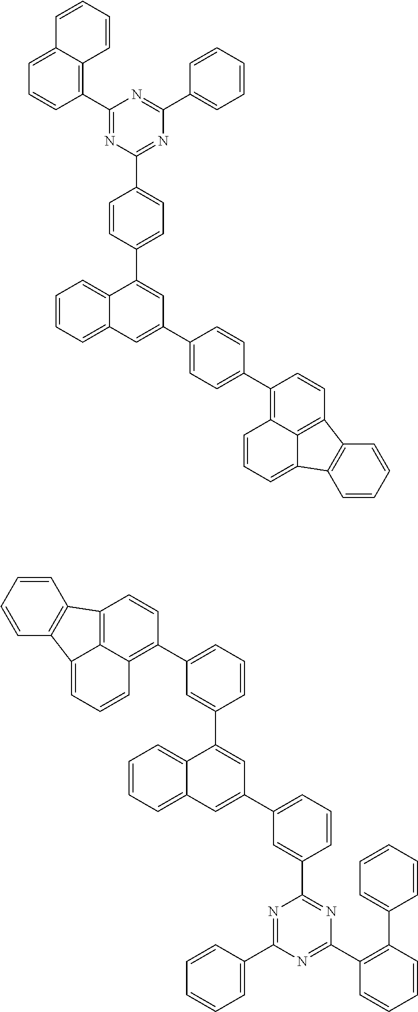 [SMILES: c1ccc(-c2nc(-c3ccc(-c4cc(-c5ccc(-c6ccc7c8c(cccc68)-c6ccccc6-7)cc5)cc5ccccc45)cc3)nc(-c3cccc4ccccc34)n2)cc1 ; c1ccc(-c2nc(-c3cccc(-c4cc(-c5cccc(-c6ccc7c8c(cccc68)-c6ccccc6-7)c5)c5ccccc5c4)c3)nc(-c3ccccc3-c3ccccc3)n2)cc1]

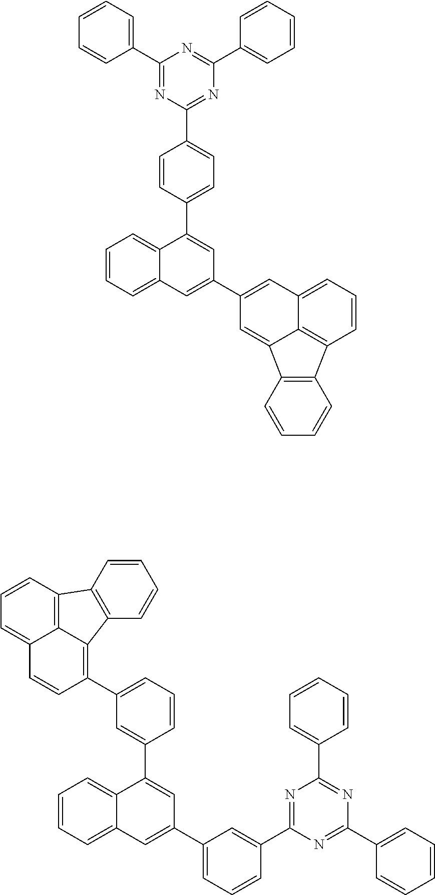 [SMILES: c1ccc(-c2nc(-c3ccccc3)nc(-c3ccc(-c4cc(-c5cc6c7c(cccc7c5)-c5ccccc5-6)cc5ccccc45)cc3)n2)cc1 ; c1ccc(-c2nc(-c3ccccc3)nc(-c3cccc(-c4cc(-c5cccc(-c6ccc7cccc8c7c6-c6ccccc6-8)c5)c5ccccc5c4)c3)n2)cc1]

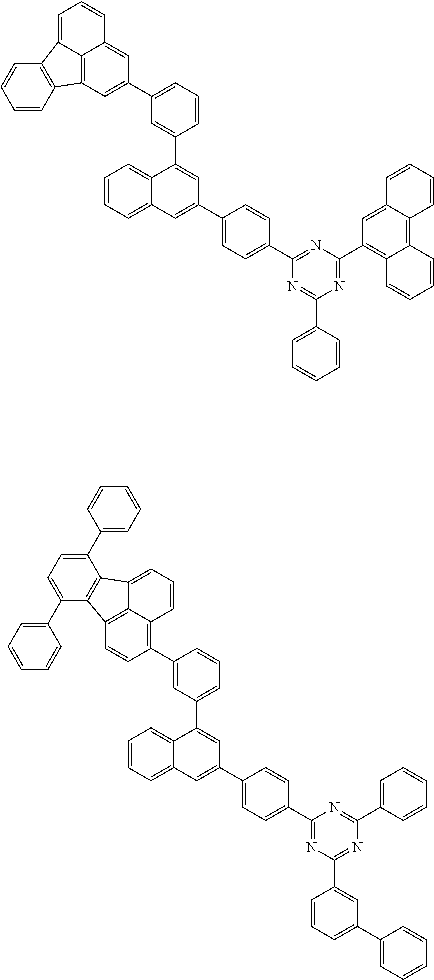 [SMILES: c1ccc(-c2nc(-c3ccc(-c4cc(-c5cccc(-c6cc7c8c(cccc8c6)-c6ccccc6-7)c5)c5ccccc5c4)cc3)nc(-c3cc4ccccc4c4ccccc34)n2)cc1 ; c1ccc(-c2cccc(-c3nc(-c4ccccc4)nc(-c4ccc(-c5cc(-c6cccc(-c7ccc8c9c(cccc79)-c7c(-c9ccccc9)ccc(-c9ccccc9)c7-8)c6)c6ccccc6c5)cc4)n3)c2)cc1]

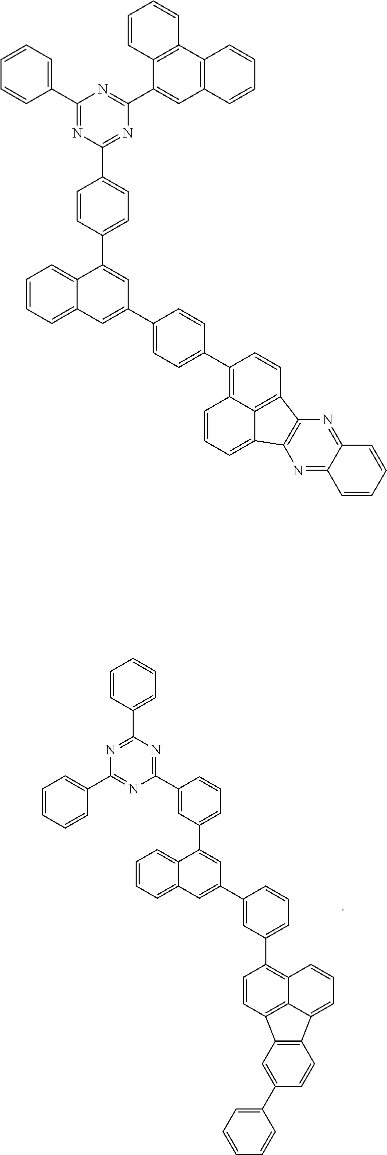 [SMILES: c1ccc(-c2nc(-c3ccc(-c4cc(-c5ccc(-c6ccc7c8c(cccc68)-c6nc8ccccc8nc6-7)cc5)cc5ccccc45)cc3)nc(-c3cc4ccccc4c4ccccc34)n2)cc1 ; c1ccc(-c2ccc3c(c2)-c2ccc(-c4cccc(-c5cc(-c6cccc(-c7nc(-c8ccccc8)nc(-c8ccccc8)n7)c6)c6ccccc6c5)c4)c4cccc-3c24)cc1]

One embodiment of the present specification provides an organic light emitting device including the compound represented by Chemical Formula 1.

In the present specification, a description of a certain member being placed “on” another member includes not only a case of the one member being in contact with the another member but a case of still another member being present between the two members.

In the present specification, a description of a certain part “including” certain constituents means capable of further including other constituents, and does not exclude other constituents unless particularly stated on the contrary.

In the present specification, the “layer” has a meaning compatible with a ‘film’ mainly used in the art, and means coating covering a target area. The size of the “layer” is not limited, and each “layer” may have the same or a different size. According to one embodiment, the size of the “layer” may be the same as the whole device, may correspond to the size of a specific functional area, or may be as small as a single sub-pixel.

In the present specification, a meaning of a specific A material being included in a B layer includes both i) one or more types of A materials being included in one B layer, and ii) a B layer being formed in one or more layers, and an A material being included in one or more of the B layers that is a multilayer.

In the present specification, a meaning of a specific A material being included in a C layer or a D layer includes both i) being included in one or more layers of one or more C layers, ii) being included in one or more layers of one or more D layers, or iii) being included in each of one or more C layers and one or more D layers.

One embodiment of the present specification provides an organic light emitting device including a first electrode; a second electrode; and one or more organic material layers provided between the first electrode and the second electrode, wherein one or more layers of the organic material layers include the compound represented by Chemical Formula 1.

The organic material layer of the organic light emitting device of the present specification may be formed in a single layer structure, but may also be formed in a multilayer structure in which two or more organic material layers are laminated. For example, the organic light emitting device of the present specification may have a structure including a hole injection layer, a hole transfer layer, a light emitting layer, an electron transfer layer, an electron injection layer, an electron blocking layer, a hole blocking layer and the like. However, the structure of the organic light emitting device is not limited thereto, and may include a smaller number of organic layers.

According to one embodiment of the present specification, the organic material layer includes an electron injection layer, an electron transfer layer, or an electron injection and transfer layer, and the electron injection layer, the electron transfer layer, or the electron injection and transfer layer includes the compound.

According to one embodiment of the present specification, the organic material layer includes a hole blocking layer, and the hole blocking layer includes the compound.

According to one embodiment of the present specification, the organic material layer includes a light emitting layer.

According to one embodiment of the present specification, the organic material layer includes a hole injection layer, a hole transfer layer, or a hole injection and transfer layer.

According to one embodiment of the present specification, the organic material layer includes an electron blocking layer.

According to one embodiment of the present specification, the organic material layer includes a hole blocking layer.

According to one embodiment of the present specification, the organic light emitting device further includes one, two or more layers selected from the group consisting of a hole injection layer, a hole transfer layer, a hole injection and transfer layer, a light emitting layer, an electron transfer layer, an electron injection layer, an electron injection and transfer layer, a hole blocking layer and an electron blocking layer.

According to one embodiment of the present specification, the organic light emitting device includes a first electrode; a second electrode provided opposite to the first electrode; a light emitting layer provided between the first electrode and the second electrode; and two or more organic material layers provided either between the light emitting layer and the first electrode or between the light emitting layer and the second electrode.

According to one embodiment of the present specification, as the two or more organic material layers, two or more may be selected from the group consisting of a hole injection layer, a hole transfer layer, a hole injection and transfer layer, a light emitting layer, an electron transfer layer, an electron injection layer, an electron injection and transfer layer, a hole blocking layer and an electron blocking layer.

According to one embodiment of the present specification, two or more hole transfer layers are included between the light emitting layer and the first electrode. The two or more hole transfer layers may include materials the same as or different from each other.

According to one embodiment of the present specification, the first electrode is an anode or a cathode.

According to one embodiment of the present specification, the second electrode is a cathode or an anode.

According to one embodiment of the present specification, the organic light emitting device may be an organic light emitting device having a structure in which an anode, one or more organic material layers and a cathode are consecutively laminated on a substrate (normal type).

According to one embodiment of the present specification, the organic light emitting device may be an organic light emitting device having a structure in a reverse direction in which a cathode, one or more organic material layers and an anode are consecutively laminated on a substrate (inverted type).

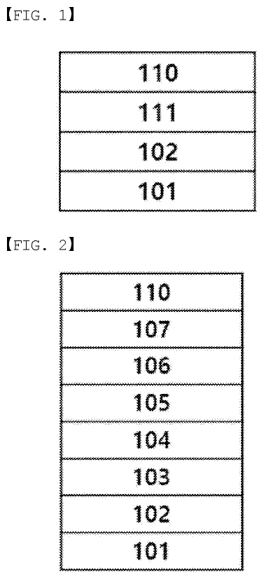

For example, structures of the organic light emitting device according to one embodiment of the present specification are illustrated in FIG. 1 and FIG. 2. FIG. 1 and FIG. 2 only illustrate the organic light emitting device, and the organic light emitting device is not limited thereto.

FIG. 1 illustrates a structure of the organic light emitting device in which a first electrode (102), an organic material layer (111) and a second electrode (110) are consecutively laminated on a substrate (101). The compound represented by Chemical Formula 1 is included in the organic material layer.

FIG. 2 illustrates a structure of the organic light emitting device in which a first electrode (102), a hole injection layer (103), a first hole transfer layer (104), a second hole transfer layer (105), a light emitting layer (106), an electron injection and transfer layer (107) and a second electrode (110) are consecutively laminated on a substrate (101). The compound represented by Chemical Formula 1 is included in the electron injection and transfer layer.

The organic light emitting device of the present specification may be manufactured using materials and methods known in the art, except that the electron injection layer, the electron transfer layer, the electron injection and transfer layer, or the hole blocking layer includes the compound, that is, the compound represented by Chemical Formula 1.

When the organic light emitting device includes a plurality of organic material layers, the organic material layers may be formed with the same materials or different materials.

For example, the organic light emitting device of the present specification may be manufactured by consecutively laminating a first electrode, an organic material layer and a second electrode on a substrate. Herein, the organic light emitting device may be manufactured by forming an anode on a substrate by depositing a metal, a metal oxide having conductivity, or an alloy thereof using a physical vapor deposition (PVD) method such as sputtering or e-beam evaporation, and forming an organic material layer including a hole injection layer, a hole transfer layer, a light emitting layer and an electron transfer layer thereon, and then depositing a material usable as a cathode thereon. In addition to such a method, the organic light emitting device may also be manufactured by consecutively depositing a cathode material, an organic material layer and an anode material on a substrate.

In addition, the compound represented by Chemical Formula 1 may be formed into an organic material layer using a solution coating method as well as a vacuum deposition method when manufacturing the organic light emitting device. Herein, the solution coating method means spin coating, dip coating, doctor blading, inkjet printing, screen printing, a spray method, roll coating and the like, but is not limited thereto.

In addition to such a method, the organic light emitting device may also be manufactured by consecutively laminating a cathode material, an organic material layer and an anode material on a substrate. However, the manufacturing method is not limited thereto.

As the anode material, materials having large work function are normally preferred so that hole injection to an organic material layer is smooth. Examples thereof include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, but are not limited thereto.

As the cathode material, materials having small work function are normally preferred so that electron injection to an organic material layer is smooth. Examples thereof include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

The light emitting layer may include a host material and a dopant material. The host material includes fused aromatic ring derivatives, heteroring-containing compounds or the like. Specifically, the fused aromatic ring derivative includes anthracene derivatives, pyrene derivatives, naphthalene derivatives, pentacene derivatives, phenanthrene compounds, fluoranthene compounds and the like, and the heteroring-containing compound includes dibenzofuran derivatives, ladder-type furan compounds, pyrimidine derivatives and the like, however, the material is not limited thereto.

According to one embodiment of the present specification, the host includes a compound represented by the following Chemical Formula H-1, but is not limited thereto.

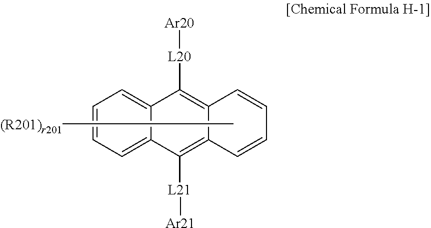

In Chemical Formula H-1,

L20 and L21 are the same as or different from each other, and each independently a direct bond; a substituted or unsubstituted arylene group; or a substituted or unsubstituted heterocyclic group,

Ar20 and Ar21 are the same as or different from each other, and each independently hydrogen; deuterium; a substituted or unsubstituted aryl group; or a substituted or unsubstituted heterocyclic group,

R201 is hydrogen; deuterium; a halogen group; a substituted or unsubstituted alkyl group; a substituted or unsubstituted cycloalkyl group; a substituted or unsubstituted aryl group; or a substituted or unsubstituted heterocyclic group, and

r201 is an integer of 1 to 8, and when r201 is 2 or greater, the two or more R201s are the same as or different from each other.

In one embodiment of the present specification, L20 and L21 are the same as or different from each other, and each independently a direct bond; a monocyclic or polycyclic arylene group having 6 to 30 carbon atoms; or a monocyclic or polycyclic heteroarylene group having 2 to 30 carbon atoms.

In one embodiment of the present specification, L20 and L21 are the same as or different from each other, and each independently a direct bond; a phenylene group unsubstituted or substituted with deuterium; a biphenylylene group unsubstituted or substituted with deuterium; a naphthylene group unsubstituted or substituted with deuterium; a divalent dibenzofuran group; or a divalent dibenzothiophene group.

In one embodiment of the present specification, Ar20 and Ar21 are the same as or different from each other, and each independently a substituted or unsubstituted monocyclic or polycyclic aryl group having 6 to 30 carbon atoms; or a substituted or unsubstituted monocyclic or polycyclic heterocyclic group having 2 to 30 carbon atoms.

In one embodiment of the present specification, Ar20 and Ar21 are the same as or different from each other, and each independently a substituted or unsubstituted monocyclic to tetracyclic aryl group having 6 to 20 carbon atoms; or a substituted or unsubstituted monocyclic to tetracyclic heterocyclic group having 6 to 20 carbon atoms.

In one embodiment of the present specification, Ar20 and Ar21 are the same as or different from each other, and each independently a phenyl group unsubstituted or substituted with deuterium or a monocyclic or polycyclic aryl group having 6 to 20 carbon atoms; a biphenyl group unsubstituted or substituted with deuterium or a monocyclic or polycyclic aryl group having 6 to 20 carbon atoms; a naphthyl group unsubstituted or substituted with a monocyclic or polycyclic aryl group having 6 to 20 carbon atoms; a dibenzofuran group unsubstituted or substituted with a monocyclic or polycyclic aryl group having 6 to 20 carbon atoms; a naphthobenzofuran group unsubstituted or substituted with a monocyclic or polycyclic aryl group having 6 to 20 carbon atoms; a dibenzothiophene group unsubstituted or substituted with a monocyclic or polycyclic aryl group having 6 to 20 carbon atoms; or a naphthobenzothiophene group unsubstituted or substituted with a monocyclic or polycyclic aryl group having 6 to 20 carbon atoms.

In one embodiment of the present specification, Ar20 and Ar21 are the same as or different from each other, and each independently a phenyl group unsubstituted or substituted with deuterium; a biphenyl group unsubstituted or substituted with deuterium; a terphenyl group; a naphthyl group unsubstituted or substituted with deuterium; a phenanthrene group; a dibenzofuran group; a naphthobenzofuran group; a dibenzothiophene group; or a naphthobenzothiophene group.

In one embodiment of the present specification, Ar20 is a substituted or unsubstituted heterocyclic group, and Ar21 is a substituted or unsubstituted aryl group.

According to one embodiment of the present specification, R201 is hydrogen.

According to one embodiment of the present specification, the compound of Chemical Formula H-1 is represented by the following compound.

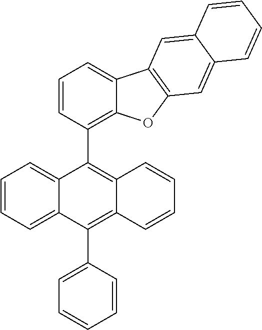 [SMILES: c1ccc(-c2c3ccccc3c(-c3cccc4c3oc3cc5ccccc5cc34)c3ccccc23)cc1]

The dopant material includes aromatic amine derivatives, styrylamine compounds, boron complexes, fluoranthene compounds, metal complexes and the like. Specifically, the aromatic amine derivative is a fused aromatic ring derivative having a substituted or unsubstituted arylamine group and includes arylamine group-including pyrene, anthracene, chrysene, peryflanthene and the like. In addition, the styrylamine compound is a compound in which substituted or unsubstituted arylamine is substituted with at least one arylvinyl group, and one, two or more substituents selected from the group consisting of an aryl group, a silyl group, an alkyl group, a cycloalkyl group and an arylamine group are substituted or unsubstituted. Specifically, styrylamine, styryldiamine, styryltriamine, styryltetramine or the like is included, however, the styrylamine compound is not limited thereto. In addition, the metal complex includes iridium complexes, platinum complexes or the like, but is not limited thereto.

According to one embodiment of the present specification, the dopant includes a compound represented by the following Chemical Formula D-1, but is not limited thereto.

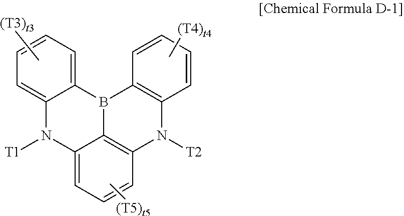

In Chemical Formula D-1,

T1 to T5 are the same as or different from each other, and each independently hydrogen; a substituted or unsubstituted alkyl group; a substituted or unsubstituted amine group; or a substituted or unsubstituted aryl group,

t3 and t4 are each an integer of 1 to 4,

t5 is an integer of 1 to 3,

when t3 is 2 or greater, the two or more T3s are the same as or different from each other,

when t4 is 2 or greater, the two or more T4s are the same as or different from each other, and

when t5 is 2 or greater, the two or more T5s are the same as or different from each other.

According to one embodiment of the present specification, T1 to T5 are the same as or different from each other, and each independently hydrogen; a substituted or unsubstituted linear or branched alkyl group having 1 to 30 carbon atoms; a substituted or unsubstituted monocyclic or polycyclic arylamine group having 6 to 30 carbon atoms; or a substituted or unsubstituted monocyclic or polycyclic aryl group having 6 to 30 carbon atoms.

According to one embodiment of the present specification, T1 to T5 are the same as or different from each other, and each independently hydrogen; a linear or branched alkyl group having 1 to 30 carbon atoms; a monocyclic or polycyclic arylamine group having 6 to 30 carbon atoms; or a monocyclic or polycyclic aryl group having 6 to 30 carbon atoms unsubstituted or substituted with a linear or branched alkyl group having 1 to 30 carbon atoms.

According to one embodiment of the present specification, T1 to T5 are the same as or different from each other, and each independently hydrogen; a methyl group; a tert-butyl group; or a phenyl group unsubstituted or substituted with a tert-butyl group.

According to one embodiment of the present specification, the compound of Chemical Formula D-1 is represented by the following compound.

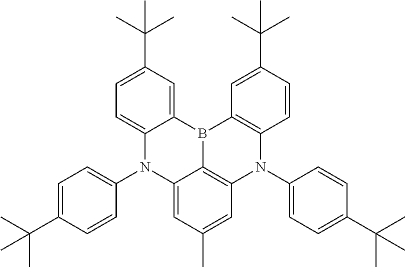 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

The hole injection layer is a layer receiving holes from an electrode. The hole injection material preferably has, by having an ability to transfer holes, a hole receiving effect from an anode and an excellent hole injection effect for a light emitting layer or a light emitting material. In addition, the hole injection material is preferably a material having an excellent ability to prevent excitons generated in the light emitting layer from moving to an electron injection layer or an electron injection material. In addition, a material having an excellent thin film forming ability is preferred. In addition, the highest occupied molecular orbital (HOMO) of the hole injection material is preferably in between the work function of an anode material and the HOMO of surrounding organic material layers. Specific examples of the hole injection material include metal porphyrins, oligothiophene, arylamine-based organic materials; hexanitrile hexaazatriphenylene-based organic materials; quinacridone-based organic materials; perylene-based organic materials; polythiophene-based conductive polymers such as anthraquinone or polyaniline, and the like, but are not limited thereto.

According to one embodiment of the present specification, the hole injection layer includes a compound represented by the following Chemical Formula HI-1, but is not limited thereto.

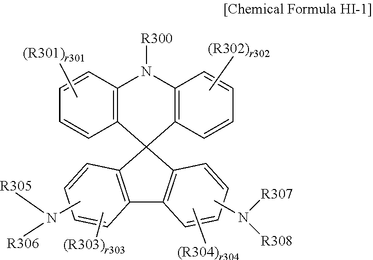

In Chemical Formula HI-1,

R300 to R308 are the same as or different from each other, and each independently hydrogen; deuterium; a cyano group; a substituted or unsubstituted alkyl group; a substituted or unsubstituted amine group; a substituted or unsubstituted aryl group; or a substituted or unsubstituted heteroaryl group, or bond to adjacent groups to form a substituted or unsubstituted ring,

r301 and r302 are each an integer of 1 to 4,

r303 and r304 are each an integer of 1 to 3,

when r301 is 2 or greater, R301s are the same as or different from each other,

when r302 is 2 or greater, R302s are the same as or different from each other,

when r303 is 2 or greater, R303s are the same as or different from each other, and

when r304 is 2 or greater, R304s are the same as or different from each other.

According to one embodiment of the present specification, R301 to R304 are hydrogen.

According to one embodiment of the present specification, R300 is a substituted or unsubstituted aryl group.

According to one embodiment of the present specification, R300 is a monocyclic or polycyclic aryl group having 6 to 30 carbon atoms.

According to one embodiment of the present specification, R300 is a phenyl group.

According to one embodiment of the present specification, R305 to R308 are the same as or different from each other, and each independently a substituted or unsubstituted aryl group; or a substituted or unsubstituted heteroaryl group.

According to one embodiment of the present specification, R305 to R308 are the same as or different from each other, and each independently a monocyclic or polycyclic aryl group having 6 to 30 carbon atoms; or a monocyclic or polycyclic heteroaryl group having 2 to 30 carbon atoms unsubstituted or substituted with a monocyclic or polycyclic aryl group having 6 to 30 carbon atoms.

According to one embodiment of the present specification, R305 to R308 are the same as or different from each other, and each independently a phenyl group; or a carbazole group unsubstituted or substituted with a phenyl group.

According to one embodiment of the present specification, the compound of Chemical Formula HI-1 is represented by the following compound.

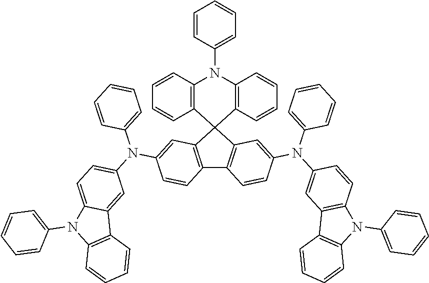 [SMILES: c1ccc(N(c2ccc3c(c2)C2(c4cc(N(c5ccccc5)c5ccc6c(c5)c5ccccc5n6-c5ccccc5)ccc4-3)c3ccccc3N(c3ccccc3)c3ccccc32)c2ccc3c(c2)c2ccccc2n3-c2ccccc2)cc1]

The hole transfer layer is a layer receiving holes from a hole injection layer and transferring the holes to a light emitting layer. As the hole transfer material, materials having, as a material capable of receiving holes from an anode or a hole injection layer and moving the holes to a light emitting layer, high mobility for the holes are preferred. Specific examples thereof include arylamine-based organic materials, conductive polymers, block copolymers having conjugated parts and non-conjugated parts together, and the like, but are not limited thereto.

According to one embodiment of the present specification, the hole transfer layer includes a compound represented by the following Chemical Formula HT-1, but is not limited thereto.

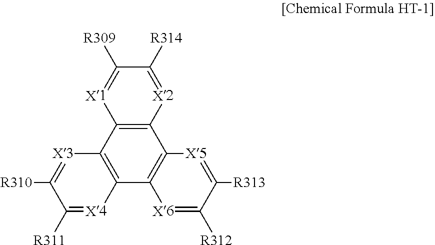

In Chemical Formula HT-1,

at least one of X′1 to X′6 is N, and the rest are CH, and

R309 to R314 are the same as or different from each other, and each independently hydrogen; deuterium; a cyano group; a substituted or unsubstituted alkyl group; a substituted or unsubstituted amine group; a substituted or unsubstituted aryl group; or a substituted or unsubstituted heteroaryl group, or bond to adjacent groups to form a substituted or unsubstituted ring.

According to one embodiment of the present specification, X′1 to X′6 are N.

According to one embodiment of the present specification, R309 to R314 are a cyano group.

According to one embodiment of the present specification, the compound of Chemical Formula HT-1 is represented by the following compound.

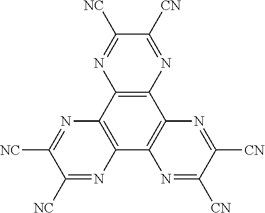 [SMILES: N#Cc1nc2c3nc(C#N)c(C#N)nc3c3nc(C#N)c(C#N)nc3c2nc1C#N]

According to one embodiment of the present specification, the hole transfer layer includes a compound represented by the following Chemical Formula HT-2, but is not limited thereto.

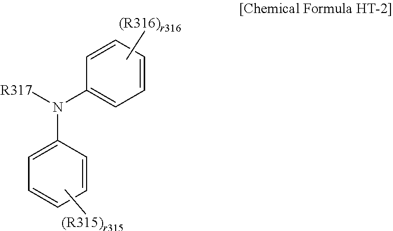

In Chemical Formula HT-2,

R315 to R317 are the same as or different from each other, and each independently any one selected from the group consisting of hydrogen; deuterium; a substituted or unsubstituted alkyl group; a substituted or unsubstituted aryl group; a substituted or unsubstituted heteroaryl group; and combinations thereof, or bond to adjacent groups to form a substituted or unsubstituted ring,

r315 is an integer of 1 to 5, and when r315 is 2 or greater, the two or more R315s are the same as or different from each other, and

r316 is an integer of 1 to 5, and when r316 is 2 or greater, the two or more R316s are the same as or different from each other.

According to one embodiment of the present specification, R317 is any one selected from the group consisting of a substituted or unsubstituted aryl group; a substituted or unsubstituted heteroaryl group; and combinations thereof.

According to one embodiment of the present specification, R317 is any one selected from the group consisting of a carbazole group; a phenyl group; a biphenyl group; and combinations thereof.

According to one embodiment of the present specification, R315 and R316 are the same as or different from each other, and each independently a substituted or unsubstituted aryl group.

According to one embodiment of the present specification, R315 and R316 are a phenyl group.

According to one embodiment of the present specification, the compound of Chemical Formula HT-2 is represented by the following compound.

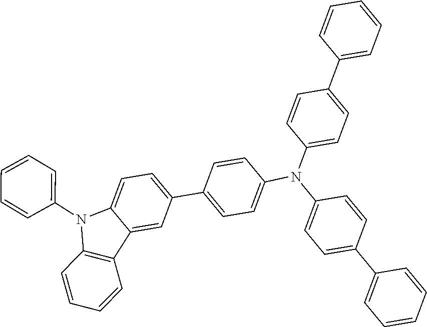 [SMILES: c1ccc(-c2ccc(N(c3ccc(-c4ccccc4)cc3)c3ccc(-c4ccc5c(c4)c4ccccc4n5-c4ccccc4)cc3)cc2)cc1]

The electron transfer layer is a layer receiving electrons from an electron injection layer and transferring the electrons to a light emitting layer. When the organic light emitting device according to one embodiment of the present specification includes an additional electron transfer layer other than the electron transfer layer including Chemical Formula 1, materials capable of favorably receiving electrons from a cathode, moving the electrons to a light emitting layer, and having high mobility for the electrons are preferred as the electron transfer material. Specific examples thereof include Al complexes of 8-hydroxyquinoline; complexes including Alq₃; organic radical compounds; hydroxyflavon-metal complexes and the like, but are not limited thereto. The electron transfer layer may be used together with any desired cathode material as used in the art. Particularly, the suitable cathode material is a common material having low work function and having an aluminum layer or a silver layer following. Specifically, cesium, barium, calcium, ytterbium, samarium and the like are included, and in each case, an aluminum layer or a silver layer follows.

The electron injection layer is a layer receiving electrons from an electrode. When the organic light emitting device according to one embodiment of the present specification includes an additional electron injection other than the electron injection layer including Chemical Formula 1, materials having an excellent electron transferring ability, having an electron receiving effect from a second electrode, and having an excellent electron injection effect for a light emitting layer or light emitting material are preferred as the electron injection material. In addition, materials preventing excitons generated in the light emitting layer from moving to a hole injection layer, and having an excellent thin film forming ability are preferred. Specific examples thereof include fluorenone, anthraquinodimethane, diphenoquinone, thiopyran dioxide, oxazole, oxadiazole, triazole, imidazole, perylene tetracarboxylic acid, fluorenylidene methane, anthrone or the like, and derivatives thereof, metal complex compounds, nitrogen-containing 5-membered ring derivatives, and the like, but are not limited thereto.

The metal complex compound includes 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato) (o-cresolato)gallium, bis(2-methyl-8-quinolinato) (1-naphtholato)aluminum, bis(2-methyl-8-quinolinato) (2-naphtholato)gallium and the like, but is not limited thereto.

The electron blocking layer is a layer capable of enhancing lifetime and efficiency of a device by preventing electrons injected from an electron injection layer from passing through a light emitting layer and entering a hole injection layer. Known material may be used without limit, and the electron blocking layer may be formed between the light emitting layer and the hole injection layer, or between the light emitting layer and a layer carrying out hole injection and hole transfer at the same time.

The hole blocking layer is a layer blocking holes from reaching a cathode, and may be generally formed under the same condition as the electron injection layer. When the organic light emitting device according to one embodiment of the present specification includes an additional hole blocking layer other than the hole blocking layer including Chemical Formula 1, specific examples thereof may include oxadiazole derivatives, triazole derivatives, phenanthroline derivatives, aluminum complexes and the like, but are not limited thereto.

The organic light emitting device according to the present specification may be a top-emission type, a bottom-emission type or a dual-emission type depending on the materials used.

Hereinafter, the present specification will be described in detail with reference to examples, comparative examples and the like. However, the examples and the comparative examples according to the present specification may be modified to various other forms, and the scope of the present specification is not to be construed as being limited to the examples and the comparative examples described below. Examples and comparative examples of the present specification are provided in order to more fully describe the present specification to those having average knowledge in the art.

### Synthesis Example

<Synthesis of Compound 1-1>

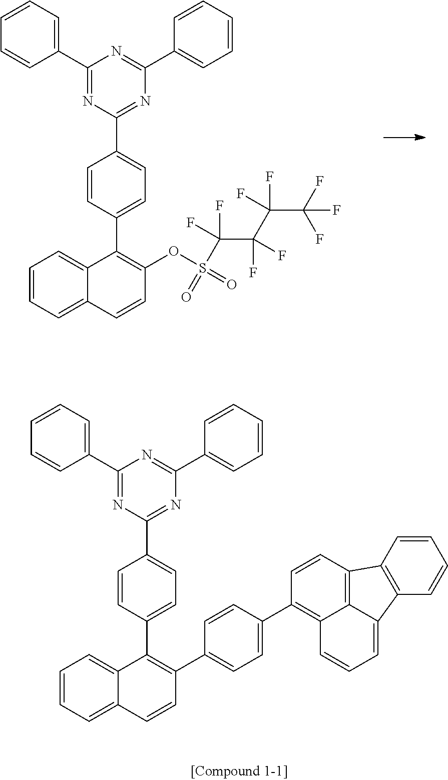 [SMILES: O=S(=O)(Oc1ccc2ccccc2c1-c1ccc(-c2nc(-c3ccccc3)nc(-c3ccccc3)n2)cc1)C(F)(F)C(F)(F)C(F)(F)C(F)(F)F ; c1ccc(-c2nc(-c3ccccc3)nc(-c3ccc(-c4c(-c5ccc(-c6ccc7c8c(cccc68)-c6ccccc6-7)cc5)ccc5ccccc45)cc3)n2)cc1]

1-(4-(4,6-Diphenyl-1,3,5-triazin-2-yl)phenyl)naphthalen-2-yl 1,1,2,2,3,3,4,4,4-nonafluorobutane-1-sulfonate (20 g, 27.26 mmol) and 2-(4-(fluoranthen-3-yl)phenyl)-4,4,5,5-tetramethyl-1,3,2-dioxaborolane (11.58 g, 28.64 mmol) were dissolved in tetrahydrofuran (300 mL) and stirred. An aqueous potassium carbonate (7.53 g, 54.52 mmol) solution was introduced thereto, and the temperature was raised. When it started to reflux, tetrakis(triphenylphosphine)palladium(0) (0.95 g, 0.82 mmol) was introduced thereto, and the result was further stirred for 2 hours. After terminating the reaction, the result was cooled, and ethanol slurry purified to prepare [Compound 1-1] (18 g, yield 93%).

[M+H]⁺=712

<Synthesis of Compound 1-2>

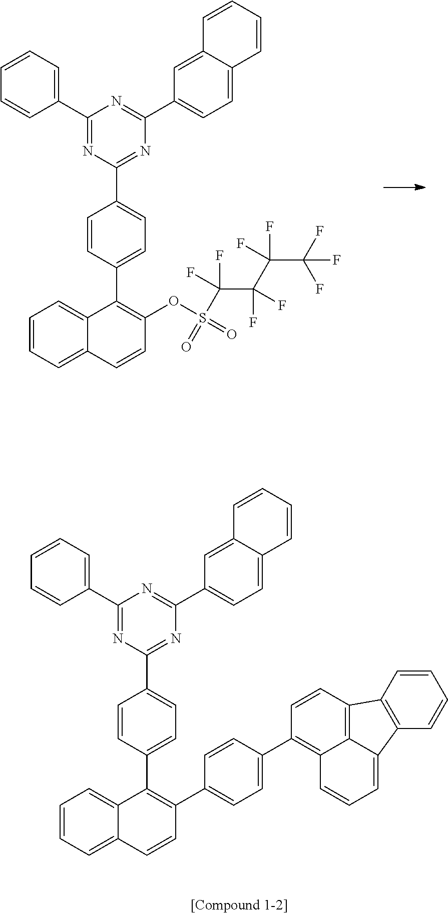 [SMILES: O=S(=O)(Oc1ccc2ccccc2c1-c1ccc(-c2nc(-c3ccccc3)nc(-c3ccc4ccccc4c3)n2)cc1)C(F)(F)C(F)(F)C(F)(F)C(F)(F)F ; c1ccc(-c2nc(-c3ccc(-c4c(-c5ccc(-c6ccc7c8c(cccc68)-c6ccccc6-7)cc5)ccc5ccccc45)cc3)nc(-c3ccc4ccccc4c3)n2)cc1]

[Compound 1-2] was prepared in the same manner as in Synthesis of [Compound 1-1] except that 1-(4-(4-(naphthalen-2-yl)-6-phenyl-1,3,5-triazin-2-yl)phenyl)naphthalen-2-yl 1,1,2,2,3,3,4,4,4-nonafluorobutane-1-sulfonate was used instead of 1-(4-(4,6-diphenyl-1,3,5-triazin-2-yl)phenyl)naphthalen-2-yl 1,1,2,2,3,3,4,4,4-nonafluorobutane-1-sulfonate.

[M+H]⁺=762

<Synthesis of Compound 1-3>

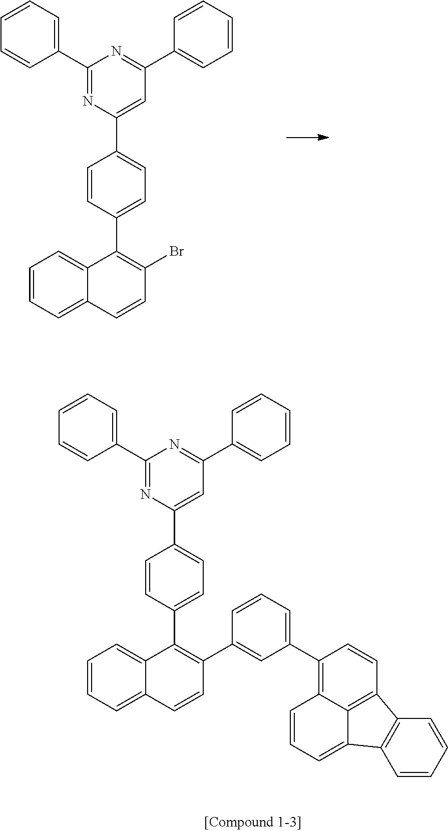 [SMILES: Brc1ccc2ccccc2c1-c1ccc(-c2cc(-c3ccccc3)nc(-c3ccccc3)n2)cc1 ; c1ccc(-c2cc(-c3ccc(-c4c(-c5cccc(-c6ccc7c8c(cccc68)-c6ccccc6-7)c5)ccc5ccccc45)cc3)nc(-c3ccccc3)n2)cc1]

[Compound 1-3] was prepared in the same manner as in Synthesis of [Compound 1-1] except that 4-(4-(2-bromonaphthalen-1-yl)phenyl)-2,6-diphenylpyrimidine was used instead of 1-(4-(4,6-diphenyl-1,3,5-triazin-2-yl)phenyl)naphthalen-2-yl 1,1,2,2,3,3,4,4,4-nonafluorobutane-1-sulfonate, and 2-(3-(fluoranthen-3-yl)phenyl)-4,4,5,5-tetramethyl-1,3,2-dioxaborolane was used instead of 2-(4-(fluoranthen-3-yl)phenyl)-4,4,5,5-tetramethyl-1,3,2-dioxaborolane.

[M+H]⁺=711

<Synthesis of Compound 1-4>

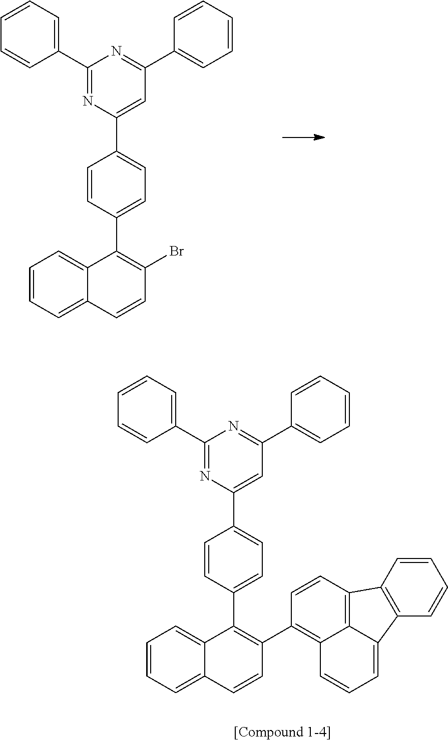 [SMILES: Brc1ccc2ccccc2c1-c1ccc(-c2cc(-c3ccccc3)nc(-c3ccccc3)n2)cc1 ; c1ccc(-c2cc(-c3ccc(-c4c(-c5ccc6c7c(cccc57)-c5ccccc5-6)ccc5ccccc45)cc3)nc(-c3ccccc3)n2)cc1]

[Compound 1-4] was prepared in the same manner as in Synthesis of [Compound 1-3] except that fluoranthen-3-ylboronic acid was used instead of 2-(3-(fluoranthen-3-yl)phenyl)-4,4,5,5-tetramethyl-1,3,2-dioxaborolane.

[M+H]⁺=635

<Synthesis of Compound 1-5>

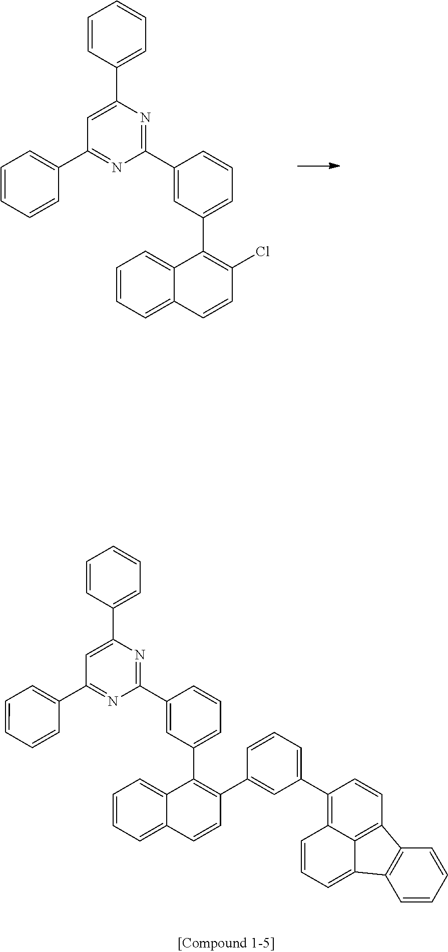 [SMILES: Clc1ccc2ccccc2c1-c1cccc(-c2nc(-c3ccccc3)cc(-c3ccccc3)n2)c1 ; c1ccc(-c2cc(-c3ccccc3)nc(-c3cccc(-c4c(-c5cccc(-c6ccc7c8c(cccc68)-c6ccccc6-7)c5)ccc5ccccc45)c3)n2)cc1]

[Compound 1-5] was prepared in the same manner as in Synthesis of [Compound 1-3] except that 2-(3-(2-chloronaphthalen-1-yl)phenyl)-4,6-diphenylpyrimidine was used instead of 4-(4-(2-bromonaphthalen-1-yl)phenyl)-2,6-diphenylpyrimidine.

[M+H]⁺=711

<Synthesis of Compound 1-6>

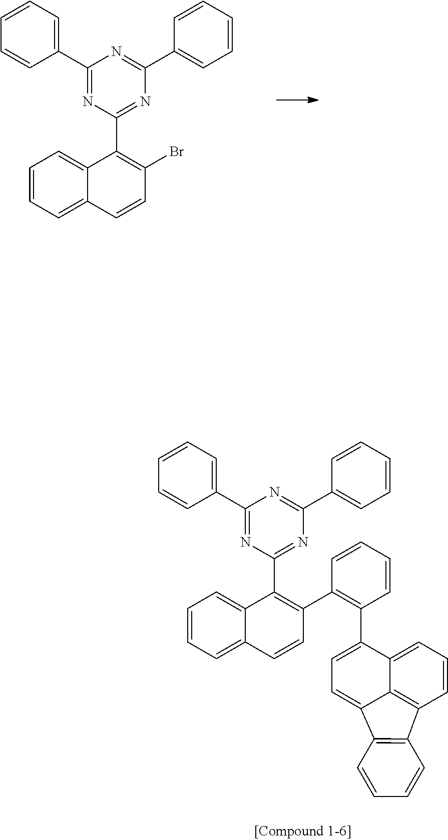 [SMILES: Brc1ccc2ccccc2c1-c1nc(-c2ccccc2)nc(-c2ccccc2)n1 ; c1ccc(-c2nc(-c3ccccc3)nc(-c3c(-c4ccccc4-c4ccc5c6c(cccc46)-c4ccccc4-5)ccc4ccccc34)n2)cc1]

[Compound 1-6] was prepared in the same manner as in Synthesis of [Compound 1-1] except that 2-(2-bromonaphthalen-1-yl)-4,6-diphenyl-1,3,5-triazine was used instead of 1-(4-(4,6-diphenyl-1,3,5-triazin-2-yl)phenyl)naphthalen-2-yl 1,1,2,2,3,3,4,4,4-nonafluorobutane-1-sulfonate, and 2-(2-(fluoranthen-3-yl)phenyl)-4,4,5,5-tetramethyl-1,3,2-dioxaborolane was used instead of 2-(4-(fluoranthen-3-yl)phenyl)-4,4,5,5-tetramethyl-1,3,2-dioxaborolane.

[M+H]⁺=636

<Synthesis of Compound 1-7>

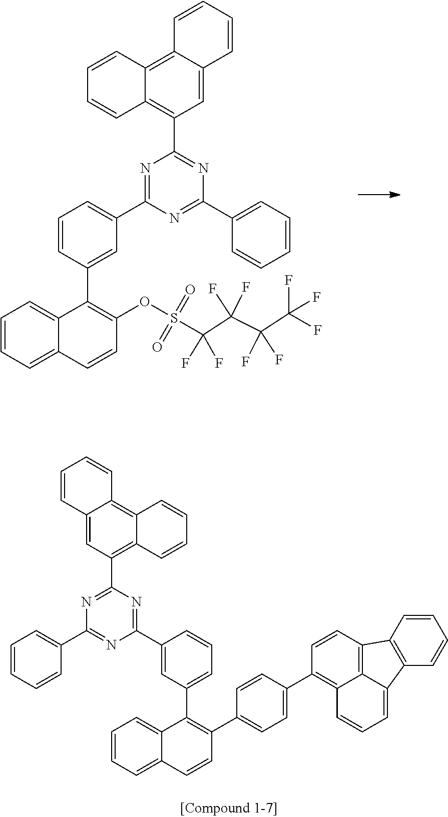 [SMILES: O=S(=O)(Oc1ccc2ccccc2c1-c1cccc(-c2nc(-c3ccccc3)nc(-c3cc4ccccc4c4ccccc34)n2)c1)C(F)(F)C(F)(F)C(F)(F)C(F)(F)F ; c1ccc(-c2nc(-c3cccc(-c4c(-c5ccc(-c6ccc7c8c(cccc68)-c6ccccc6-7)cc5)ccc5ccccc45)c3)nc(-c3cc4ccccc4c4ccccc34)n2)cc1]

[Compound 1-7] was prepared in the same manner as in Synthesis of [Compound 1-1] except that 1-(3-(4-(phenanthren yl)-6-phenyl-1,3,5-triazin-2-yl)phenyl)naphthalen-2-yl 1,1,2,2,3,3,4,4,4-nonafluorobutane-1-sulfonate was used instead of 1-(4-(4,6-diphenyl-1,3,5-triazin-2-yl)phenyl)naphthalen-2-yl 1,1,2,2,3,3,4,4,4-nonafluorobutane-1-sulfonate.

[M+H]⁺=812

<Synthesis of Compound 1-8>

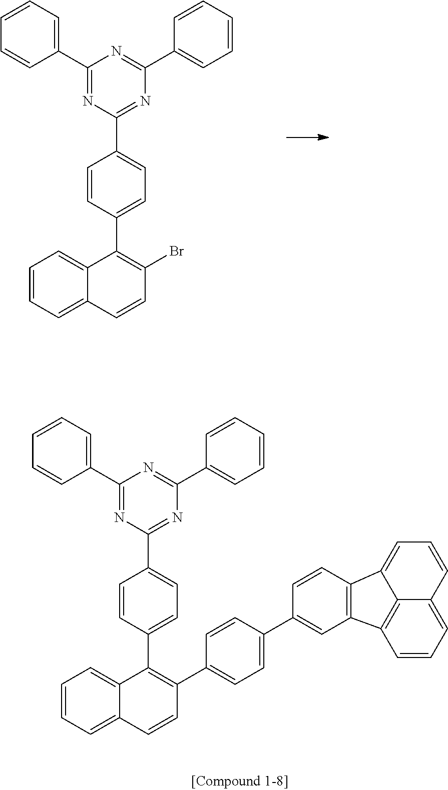 [SMILES: Brc1ccc2ccccc2c1-c1ccc(-c2nc(-c3ccccc3)nc(-c3ccccc3)n2)cc1 ; c1ccc(-c2nc(-c3ccccc3)nc(-c3ccc(-c4c(-c5ccc(-c6ccc7c(c6)-c6cccc8cccc-7c68)cc5)ccc5ccccc45)cc3)n2)cc1]

[Compound 1-8] was prepared in the same manner as in Synthesis of [Compound 1-1] except that 2-(4-(2-bromonaphthalen-1-yl)phenyl)-4,6-diphenyl-1,3,5-triazine was used instead of 1-(4-(4,6-diphenyl-1,3,5-triazin-2-yl)phenyl)naphthalen-2-yl 1,1,2,2,3,3,4,4,4-nonafluorobutane-1-sulfonate, and 2-(4-(fluoranthen-8-yl)phenyl)-4,4,5,5-tetramethyl-1,3,2-dioxaborolane was used instead of 2-(4-(fluoranthen-3-yl)phenyl)-4,4,5,5-tetramethyl-1,3,2-dioxaborolane.

[M+H]⁺=712

<Synthesis of Compound 1-9>

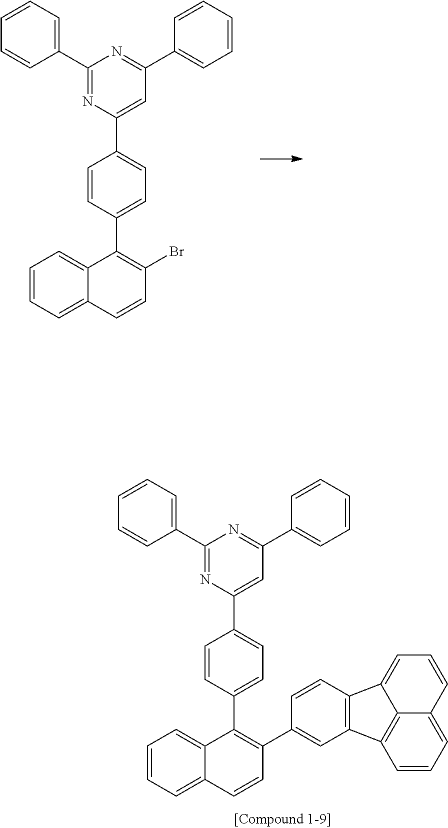 [SMILES: Brc1ccc2ccccc2c1-c1ccc(-c2cc(-c3ccccc3)nc(-c3ccccc3)n2)cc1 ; c1ccc(-c2cc(-c3ccc(-c4c(-c5ccc6c(c5)-c5cccc7cccc-6c57)ccc5ccccc45)cc3)nc(-c3ccccc3)n2)cc1]

[Compound 1-9] was prepared in the same manner as in Synthesis of [Compound 1-1] except that 4-(4-(2-bromonaphthalen-1-yl)phenyl)-2,6-diphenylpyrimidine was used instead of 1-(4-(4,6-diphenyl-1,3,5-triazin-2-yl)phenyl)naphthalen-2-yl 1,1,2,2,3,3,4,4,4-nonafluorobutane-1-sulfonate, and fluoranthen-8-ylboronic acid was used instead of 2-(4-(fluoranthen-3-yl)phenyl)-4,4,5,5-tetramethyl-1,3,2-dioxaborolane.

[M+H]⁺=635

<Synthesis of Compound 1-10>

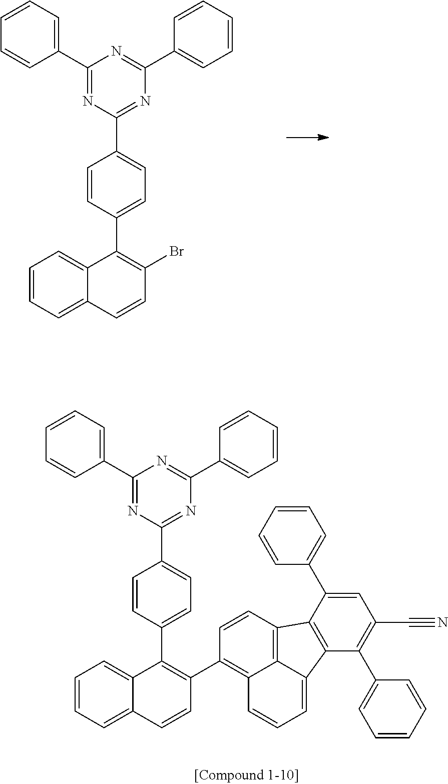 [SMILES: Brc1ccc2ccccc2c1-c1ccc(-c2nc(-c3ccccc3)nc(-c3ccccc3)n2)cc1 ; N#Cc1cc(-c2ccccc2)c2c(c1-c1ccccc1)-c1cccc3c(-c4ccc5ccccc5c4-c4ccc(-c5nc(-c6ccccc6)nc(-c6ccccc6)n5)cc4)ccc-2c13]

[Compound 1-10] was prepared in the same manner as in Synthesis of [Compound 1-8] except that (8-cyano-7,10-diphenylfluoranthen-3-yl)boronic acid was used instead of 2-(4-(fluoranthen-3-yl)phenyl)-4,4,5,5-tetramethyl-1,3,2-dioxaborolane.

[M+H]⁺=813

<Synthesis of Compound 1-11>

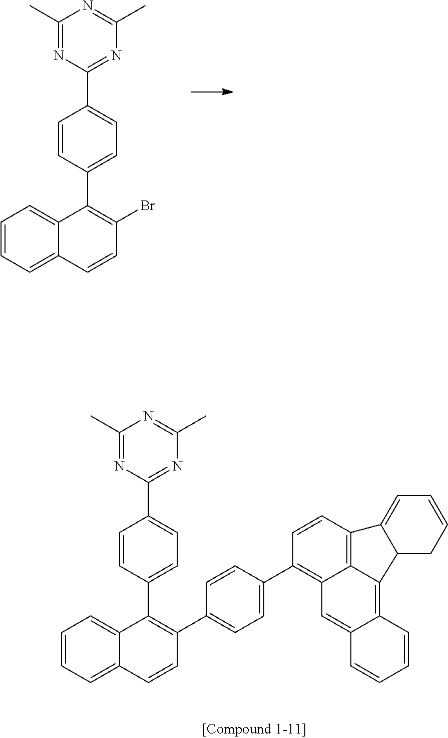 [SMILES: Cc1nc(C)nc(-c2ccc(-c3c(Br)ccc4ccccc34)cc2)n1 ; Cc1nc(C)nc(-c2ccc(-c3c(-c4ccc(-c5ccc6c7c(c8ccccc8cc57)C5CC=CC=C65)cc4)ccc4ccccc34)cc2)n1]

[Compound 1-11] was prepared in the same manner as in Synthesis of [Compound 1-1] except that 2-(4-(2-bromonaphthalen-1-yl)phenyl)-4,6-dimethyl-1,3,5-triazine was used instead of 1-(4-(4,6-diphenyl-1,3,5-triazin-2-yl)phenyl)naphthalen-2-yl 1,1,2,2,3,3,4,4,4-nonafluorobutane-1-sulfonate, and (4-(benzo[a]aceanthrylen-7-yl)phenyl)boronic acid was used instead of 2-(4-(fluoranthen-3-yl)phenyl)-4,4,5,5-tetramethyl-1,3,2-dioxaborolane.

[M+H]⁺=638

<Synthesis of Compound 1-12>

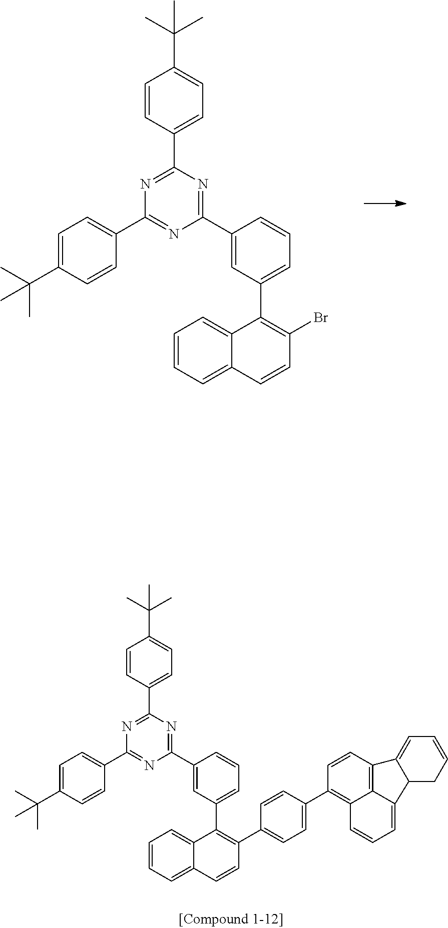 [SMILES: CC(C)(C)c1ccc(-c2nc(-c3ccc(C(C)(C)C)cc3)nc(-c3cccc(-c4c(Br)ccc5ccccc45)c3)n2)cc1 ; CC(C)(C)c1ccc(-c2nc(-c3ccc(C(C)(C)C)cc3)nc(-c3cccc(-c4c(-c5ccc(-c6ccc7c8c(cccc68)C6CC=CC=C76)cc5)ccc5ccccc45)c3)n2)cc1]

[Compound 1-12] was prepared in the same manner as in Synthesis of [Compound 1-1] except that 2-(3-(2-bromonaphthalen-1-yl)phenyl)-4,6-bis(4-(tert-butyl)phenyl)-1,3,5-triazine was used instead of 1-(4-(4,6-diphenyl-1,3,5-triazin yl)phenyl)naphthalen-2-yl 1,1,2,2,3,3,4,4,4-nonafluorobutane sulfonate.

[M+H]⁺=824

<Synthesis of Compound 1-13>

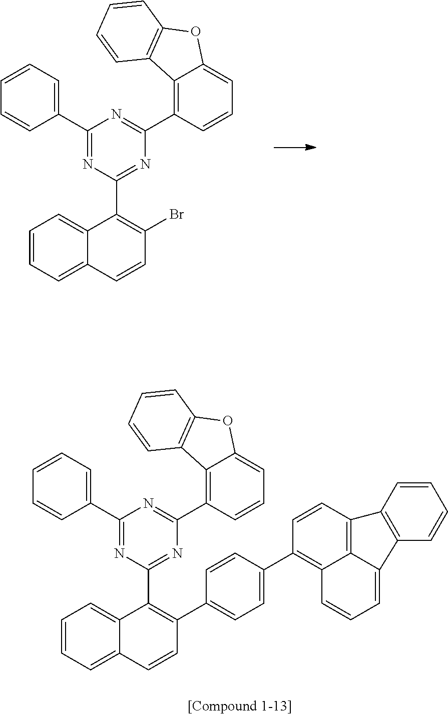 [SMILES: Brc1ccc2ccccc2c1-c1nc(-c2ccccc2)nc(-c2cccc3oc4ccccc4c23)n1 ; c1ccc(-c2nc(-c3c(-c4ccc(-c5ccc6c7c(cccc57)-c5ccccc5-6)cc4)ccc4ccccc34)nc(-c3cccc4oc5ccccc5c34)n2)cc1]

[Compound 1-13] was prepared in the same manner as in Synthesis of [Compound 1-1] except that 2-(2-bromonaphthalen-1-yl)-4-(dibenzo[b,d]furan-1-yl)-6-phenyl-1,3,5-triazine was used instead of 1-(4-(4,6-diphenyl-1,3,5-triazin-2-yl)phenyl)naphthalen-2-yl 1,1,2,2,3,3,4,4,4-nonafluorobutane-1-sulfonate.

[M+H]⁺=726

<Synthesis of Compound 1-14>

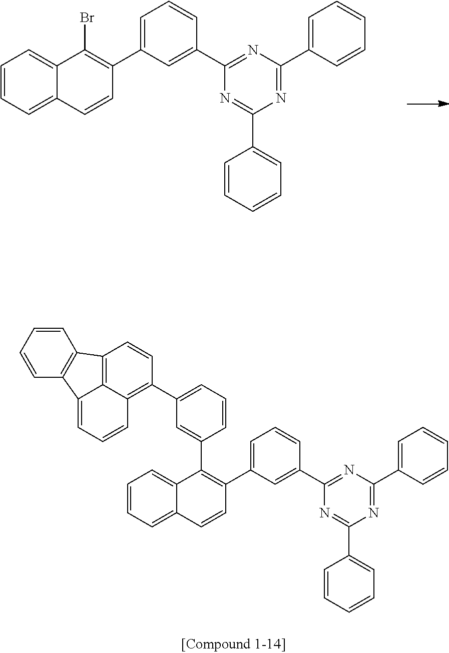 [SMILES: Brc1c(-c2cccc(-c3nc(-c4ccccc4)nc(-c4ccccc4)n3)c2)ccc2ccccc12 ; c1ccc(-c2nc(-c3ccccc3)nc(-c3cccc(-c4ccc5ccccc5c4-c4cccc(-c5ccc6c7c(cccc57)-c5ccccc5-6)c4)c3)n2)cc1]

[Compound 1-14] was prepared in the same manner as in Synthesis of [Compound 1-1] except that 2-(3-(1-bromonaphthalen-2-yl)phenyl)-4,6-diphenyl-1,3,5-triazine was used instead of 1-(4-(4,6-diphenyl-1,3,5-triazin-2-yl)phenyl)naphthalen-2-yl 1,1,2,2,3,3,4,4,4-nonafluorobutane-1-sulfonate, and (3-(fluoranthen-3-yl)phenyl)boronic acid was used instead of 2-(4-(fluoranthen-3-yl)phenyl)-4,4,5,5-tetramethyl-1,3,2-dioxaborolane.

[M+H]⁺=712

<Synthesis of Compound 1-15>

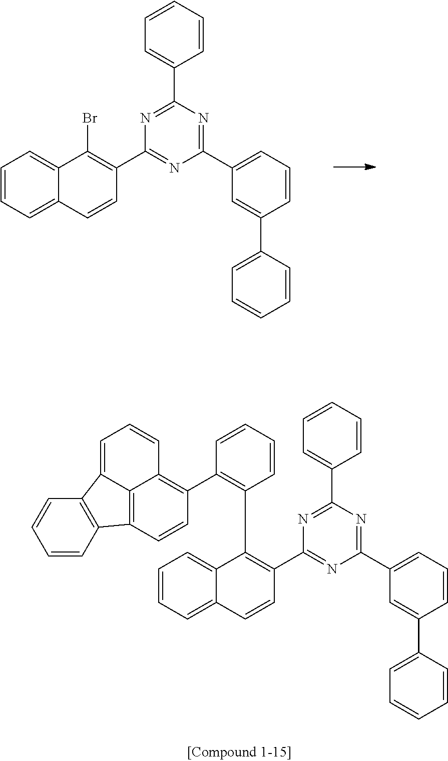 [SMILES: Brc1c(-c2nc(-c3ccccc3)nc(-c3cccc(-c4ccccc4)c3)n2)ccc2ccccc12 ; c1ccc(-c2cccc(-c3nc(-c4ccccc4)nc(-c4ccc5ccccc5c4-c4ccccc4-c4ccc5c6c(cccc46)-c4ccccc4-5)n3)c2)cc1]

[Compound 1-15] was prepared in the same manner as in Synthesis of [Compound 1-1] except that 2-([1,1′-biphenyl]-3-yl)-4-(1-bromonaphthalen-2-yl)-6-phenyl-1,3,5-triazine was used instead of 1-(4-(4,6-diphenyl-1,3,5-triazin-2-yl)phenyl)naphthalen-2-yl 1,1,2,2,3,3,4,4,4-nonafluorobutane-1-sulfonate, and (2-(fluoranthen-3-yl)phenyl)boronic acid was used instead of 2-(4-(fluoranthen-3-yl)phenyl)-4,4,5,5-tetramethyl-1,3,2-dioxaborolane.

[M+H]⁺=712

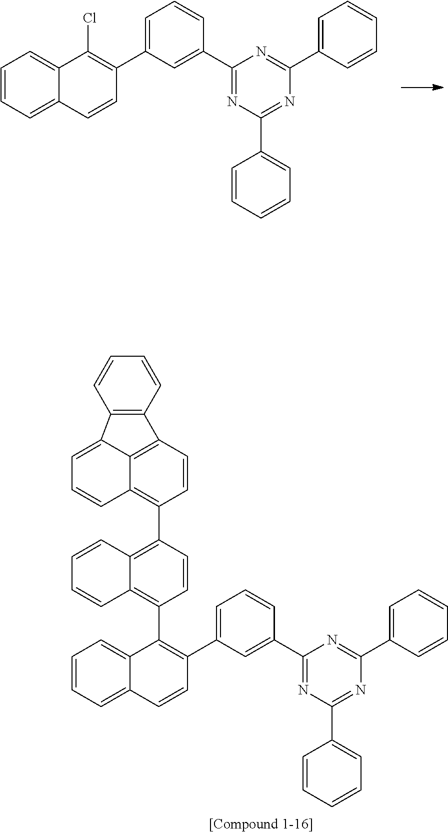 [SMILES: Clc1c(-c2cccc(-c3nc(-c4ccccc4)nc(-c4ccccc4)n3)c2)ccc2ccccc12 ; c1ccc(-c2nc(-c3ccccc3)nc(-c3cccc(-c4ccc5ccccc5c4-c4ccc(-c5ccc6c7c(cccc57)-c5ccccc5-6)c5ccccc45)c3)n2)cc1]

[Compound 1-16] was prepared in the same manner as in Synthesis of [Compound 1-1] except that 2-(3-(1-chloronaphthalen-2-yl)phenyl)-4,6-diphenyl-1,3,5-triazine was used instead of 1-(4-(4,6-diphenyl-1,3,5-triazin-2-yl)phenyl)naphthalen-2-yl 1,1,2,2,3,3,4,4,4-nonafluorobutane-1-sulfonate, and 2-(4-(fluoranthen-3-yl)naphthalen-1-yl)-4,4,5,5-tetramethyl-1,3,2-dioxaborolane was used instead of 2-(4-(fluoranthen-3-yl)phenyl)-4,4,5,5-tetramethyl-1,3,2-dioxaborolane.

[M+H]⁺=762

<Synthesis of Compound 1-17>

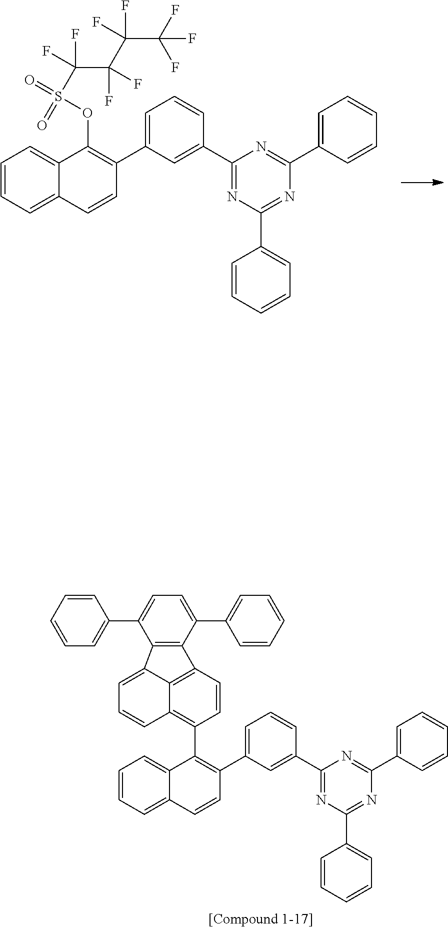 [SMILES: O=S(=O)(Oc1c(-c2cccc(-c3nc(-c4ccccc4)nc(-c4ccccc4)n3)c2)ccc2ccccc12)C(F)(F)C(F)(F)C(F)(F)C(F)(F)F ; c1ccc(-c2nc(-c3ccccc3)nc(-c3cccc(-c4ccc5ccccc5c4-c4ccc5c6c(cccc46)-c4c(-c6ccccc6)ccc(-c6ccccc6)c4-5)c3)n2)cc1]

[Compound 1-17] was prepared in the same manner as in Synthesis of [Compound 1-1] except that 2-(3-(4,6-diphenyl-1,3,5-triazin-2-yl)phenyl)naphthalen-1-yl 1,1,2,2,3,3,4,4,4-nonafluorobutane-1-sulfonate was used instead of 1-(4-(4,6-diphenyl-1,3,5-triazin-2-yl)phenyl)naphthalen-2-yl 1,1,2,2,3,3,4,4,4-nonafluorobutane-1-sulfonate, and (7,10-diphenylfluoranthen-3-yl)boronic acid was used instead of 2-(4-(fluoranthen-3-yl)phenyl)-4,4,5,5-tetramethyl-1,3,2-dioxaborolane.

[M+H]⁺=788

<Synthesis of Compound 1-18>

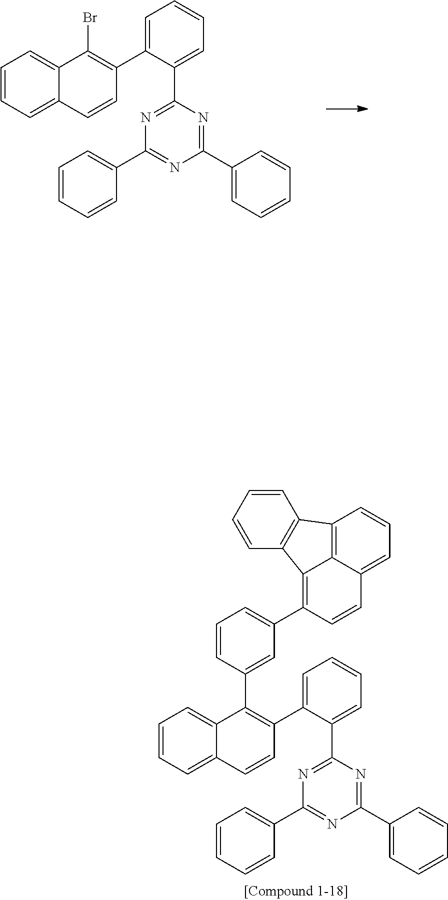 [SMILES: Brc1c(-c2ccccc2-c2nc(-c3ccccc3)nc(-c3ccccc3)n2)ccc2ccccc12 ; c1ccc(-c2nc(-c3ccccc3)nc(-c3ccccc3-c3ccc4ccccc4c3-c3cccc(-c4ccc5cccc6c5c4-c4ccccc4-6)c3)n2)cc1]

[Compound 1-18] was prepared in the same manner as in Synthesis of [Compound 1-1] except that 2-(2-(1-bromonaphthalen-2-yl)phenyl)-4,6-diphenyl-1,3,5-triazine was used instead of 1-(4-(4,6-diphenyl-1,3,5-triazin-2-yl)phenyl)naphthalen-2-yl 1,1,2,2,3,3,4,4,4-nonafluorobutane-1-sulfonate, and 2-(3-(fluoranthen-1-yl)phenyl)-4,4,5,5-tetramethyl-1,3,2-dioxaborolane was used instead of 2-(4-(fluoranthen-3-yl)phenyl)-4,4,5,5-tetramethyl-1,3,2-dioxaborolane.

[M+H]⁺=712

<Synthesis of Compound 1-19>

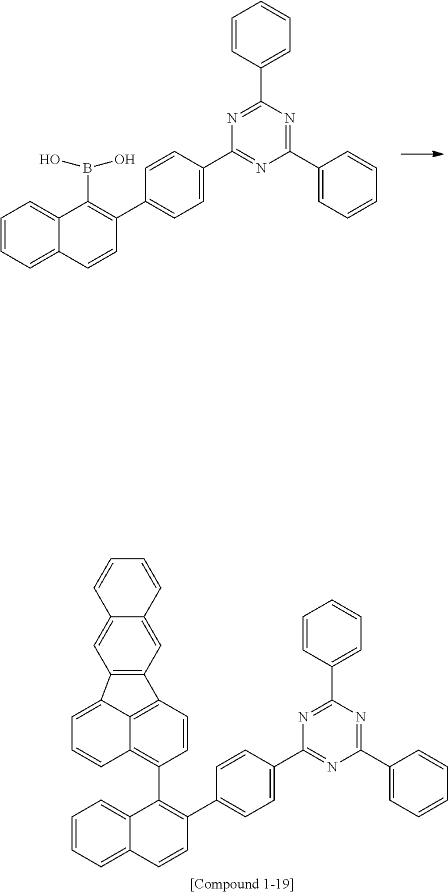 [SMILES: OB(O)c1c(-c2ccc(-c3nc(-c4ccccc4)nc(-c4ccccc4)n3)cc2)ccc2ccccc12 ; c1ccc(-c2nc(-c3ccccc3)nc(-c3ccc(-c4ccc5ccccc5c4-c4ccc5c6c(cccc46)-c4cc6ccccc6cc4-5)cc3)n2)cc1]

[Compound 1-19] was prepared in the same manner as in Synthesis of [Compound 1-1] except that (2-(4-(4,6-diphenyl-1,3,5-triazin-2-yl)phenyl)naphthalen-1-yl)boronic acid was used instead of 1-(4-(4,6-diphenyl-1,3,5-triazin-2-yl)phenyl)naphthalen-2-yl 1,1,2,2,3,3,4,4,4-nonafluorobutane-1-sulfonate, and 3-bromobenzo[k]fluoranthene was used instead of 2-(4-(fluoranthen-3-yl)phenyl)-4,4,5,5-tetramethyl-1,3,2-dioxaborolane.

[M+H]⁺=686

<Synthesis of Compound 1-20>

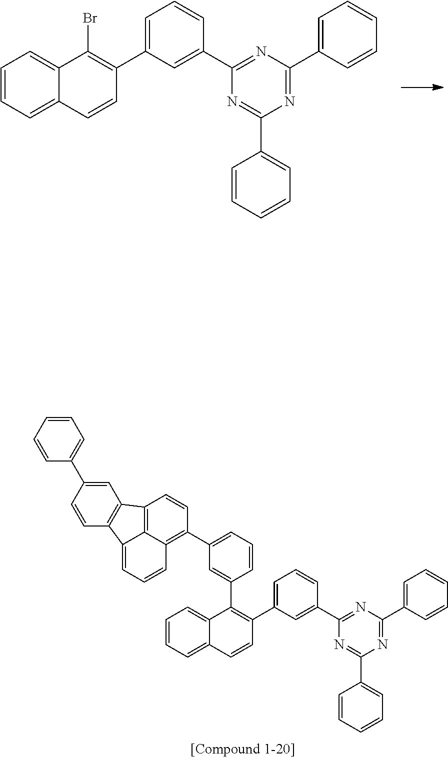 [SMILES: Brc1c(-c2cccc(-c3nc(-c4ccccc4)nc(-c4ccccc4)n3)c2)ccc2ccccc12 ; c1ccc(-c2ccc3c(c2)-c2ccc(-c4cccc(-c5c(-c6cccc(-c7nc(-c8ccccc8)nc(-c8ccccc8)n7)c6)ccc6ccccc56)c4)c4cccc-3c24)cc1]

[Compound 1-20] was prepared in the same manner as in Synthesis of [Compound 1-1] except that 2-(3-(1-bromonaphthalen-2-yl)phenyl)-4,6-diphenyl-1,3,5-triazine was used instead of 1-(4-(4,6-diphenyl-1,3,5-triazin-2-yl)phenyl)naphthalen-2-yl 1,1,2,2,3,3,4,4,4-nonafluorobutane-1-sulfonate, and (3-(9-phenylfluoranthen-3-yl)phenyl)boronic acid was used instead of 2-(4-(fluoranthen-3-yl)phenyl)-4,4,5,5-tetramethyl-1,3,2-dioxaborolane.

[M+H]⁺=788

<Synthesis of Compound 1-21>

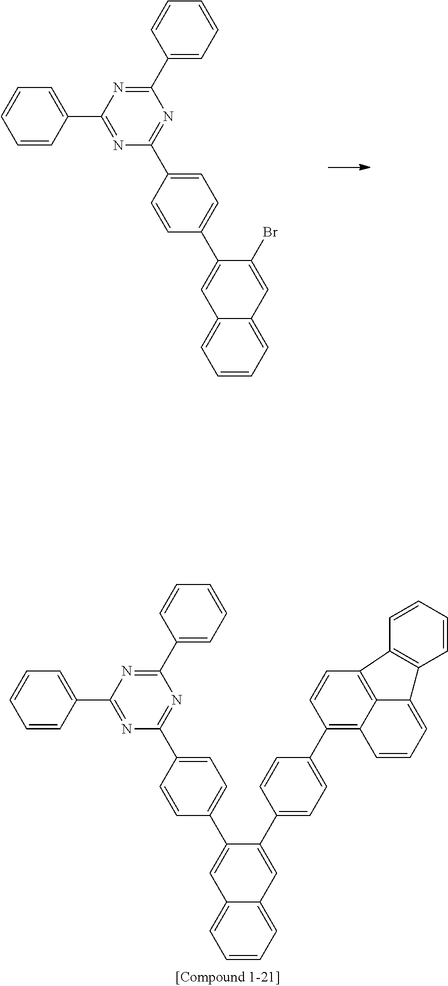 [SMILES: Brc1cc2ccccc2cc1-c1ccc(-c2nc(-c3ccccc3)nc(-c3ccccc3)n2)cc1 ; c1ccc(-c2nc(-c3ccccc3)nc(-c3ccc(-c4cc5ccccc5cc4-c4ccc(-c5ccc6c7c(cccc57)-c5ccccc5-6)cc4)cc3)n2)cc1]

[Compound 1-21] was prepared in the same manner as in Synthesis of [Compound 1-1] except that 2-(4-(3-bromonaphthalen-2-yl)phenyl)-4,6-diphenyl-1,3,5-triazine was used instead of 1-(4-(4,6-diphenyl-1,3,5-triazin-2-yl)phenyl)naphthalen-2-yl 1,1,2,2,3,3,4,4,4-nonafluorobutane-1-sulfonate.

[M+H]⁺=712

<Synthesis of Compound 1-22>

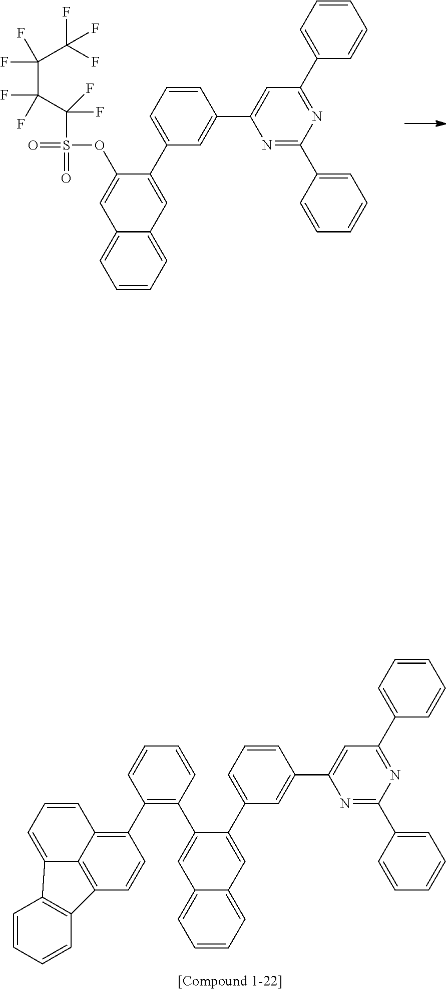 [SMILES: O=S(=O)(Oc1cc2ccccc2cc1-c1cccc(-c2cc(-c3ccccc3)nc(-c3ccccc3)n2)c1)C(F)(F)C(F)(F)C(F)(F)C(F)(F)F ; c1ccc(-c2cc(-c3cccc(-c4cc5ccccc5cc4-c4ccccc4-c4ccc5c6c(cccc46)-c4ccccc4-5)c3)nc(-c3ccccc3)n2)cc1]

[Compound 1-22] was prepared in the same manner as in Synthesis of [Compound 1-1] except that 3-(3-(2,6-diphenylpyrimidin-4-yl)phenyl)naphthalen-2-yl 1,1,2,2,3,3,4,4,4-nonafluorobutane-1-sulfonate was used instead of 1-(4-(4,6-diphenyl-1,3,5-triazin-2-yl)phenyl)naphthalen-2-yl 1,1,2,2,3,3,4,4,4-nonafluorobutane-1-sulfonate, and 2-(2-(fluoranthen-3-yl)phenyl)-4,4,5,5-tetramethyl-1,3,2-dioxaborolane was used instead of 2-(4-(fluoranthen-3-yl)phenyl)-4,4,5,5-tetramethyl-1,3,2-dioxaborolane.

[M+H]⁺=711

<Synthesis of Compound 1-23>

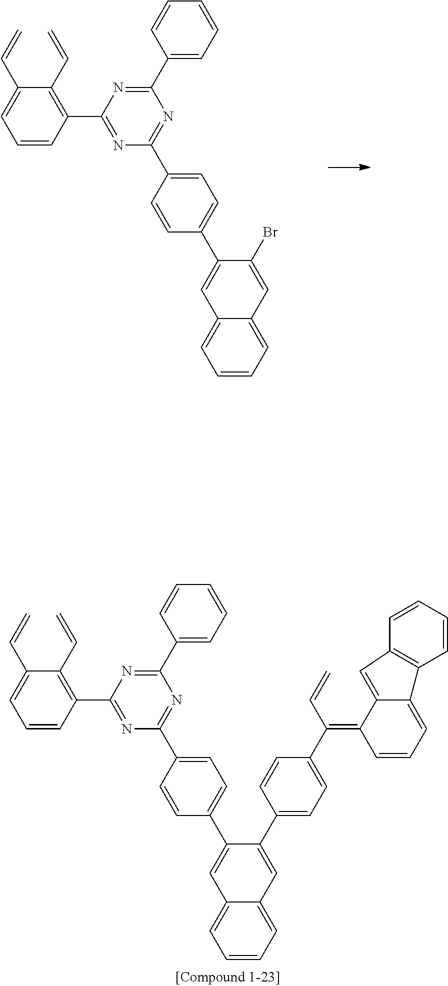 [SMILES: C=Cc1cccc(-c2nc(-c3ccccc3)nc(-c3ccc(-c4cc5ccccc5cc4Br)cc3)n2)c1C=C ; C=C/C(c1ccc(-c2cc3ccccc3cc2-c2ccc(-c3nc(-c4ccccc4)nc(-c4cccc(C=C)c4C=C)n3)cc2)cc1)=c1/cccc2c1=Cc1ccccc1-2]

[Compound 1-23] was prepared in the same manner as in Synthesis of [Compound 1-1] except that 2-(4-(3-bromonaphthalen-2-yl)phenyl)-4-(naphthalen-1-yl)-6-phenyl-1,3,5-triazine was used instead of 1-(4-(4,6-diphenyl-1,3,5-triazin-2-yl)phenyl)naphthalen-2-yl 1,1,2,2,3,3,4,4,4-nonafluorobutane-1-sulfonate.

[M+H]⁺=762

<Synthesis of Compound 1-24>

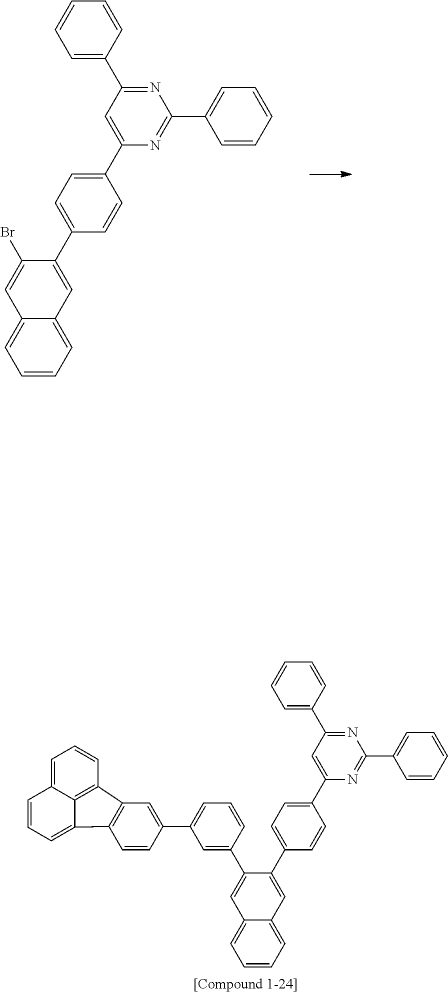 [SMILES: Brc1cc2ccccc2cc1-c1ccc(-c2cc(-c3ccccc3)nc(-c3ccccc3)n2)cc1 ; c1ccc(-c2cc(-c3ccc(-c4cc5ccccc5cc4-c4cccc(-c5ccc6c(c5)-c5cccc7cccc-6c57)c4)cc3)nc(-c3ccccc3)n2)cc1]

[Compound 1-24] was prepared in the same manner as in Synthesis of [Compound 1-1] except that 4-(4-(3-bromonaphthalen-2-yl)phenyl)-2,6-diphenylpyrimidine was used instead of 1-(4-(4,6-diphenyl-1,3,5-triazin-2-yl)phenyl)naphthalen-2-yl 1,1,2,2,3,3,4,4,4-nonafluorobutane-1-sulfonate, and 2-(3-(fluoranthen-8-yl)phenyl)-4,4,5,5-tetramethyl-1,3,2-dioxaborolane was used instead of 2-(4-(fluoranthen-3-yl)phenyl)-4,4,5,5-tetramethyl-1,3,2-dioxaborolane.

[M+H]⁺=711

<Synthesis of Compound 1-25>

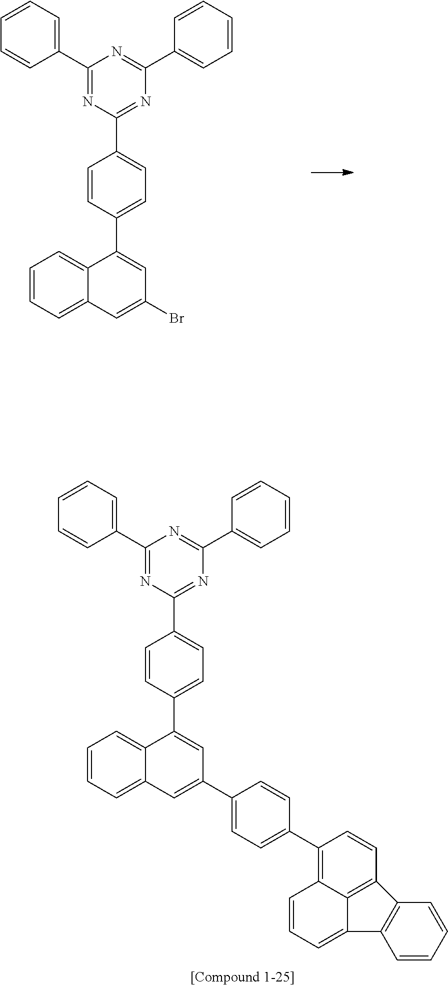 [SMILES: Brc1cc(-c2ccc(-c3nc(-c4ccccc4)nc(-c4ccccc4)n3)cc2)c2ccccc2c1 ; c1ccc(-c2nc(-c3ccccc3)nc(-c3ccc(-c4cc(-c5ccc(-c6ccc7c8c(cccc68)-c6ccccc6-7)cc5)cc5ccccc45)cc3)n2)cc1]

[Compound 1-25] was prepared in the same manner as in Synthesis of [Compound 1-1] except that 2-(4-(3-bromonaphthalen-1-yl)phenyl)-4,6-diphenyl-1,3,5-triazine was used instead of 1-(4-(4,6-diphenyl-1,3,5-triazin-2-yl)phenyl)naphthalen-2-yl 1,1,2,2,3,3,4,4,4-nonafluorobutane-1-sulfonate.

[M+H]⁺=712

<Synthesis of Compound 1-26>

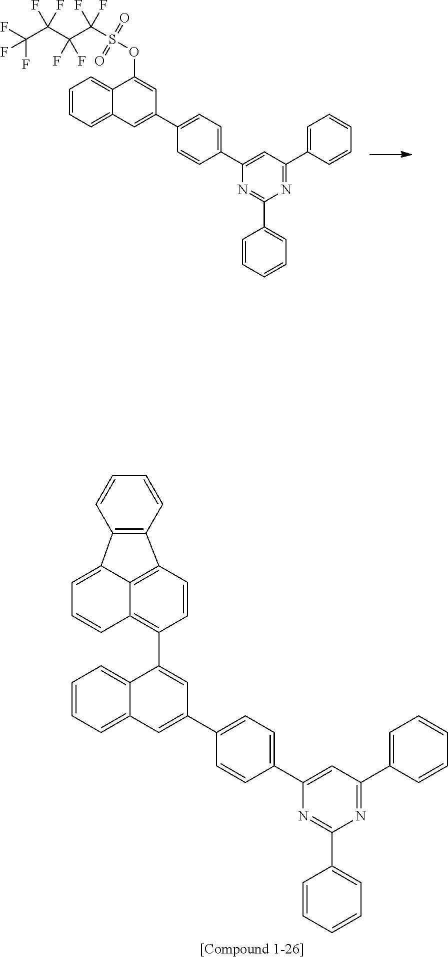 [SMILES: O=S(=O)(Oc1cc(-c2ccc(-c3cc(-c4ccccc4)nc(-c4ccccc4)n3)cc2)cc2ccccc12)C(F)(F)C(F)(F)C(F)(F)C(F)(F)F ; c1ccc(-c2cc(-c3ccc(-c4cc(-c5ccc6c7c(cccc57)-c5ccccc5-6)c5ccccc5c4)cc3)nc(-c3ccccc3)n2)cc1]

[Compound 1-26] was prepared in the same manner as in Synthesis of [Compound 1-1] except that 3-(4-(2,6-diphenylpyrimidin-4-yl)phenyl)naphthalen-1-yl 1,1,2,2,3,3,4,4,4-nonafluorobutane-1-sulfonate was used instead of 1-(4-(4,6-diphenyl-1,3,5-triazin-2-yl)phenyl)naphthalen-2-yl 1,1,2,2,3,3,4,4,4-nonafluorobutane-1-sulfonate, and 2-(fluoranthen-3-yl)-4,4,5,5-tetramethyl-1,3,2-dioxaborolane was used instead of 2-(4-(fluoranthen-3-yl)phenyl)-4,4,5,5-tetramethyl-1,3,2-dioxaborolane.

[M+H]⁺=635

<Synthesis of Compound 1-27>

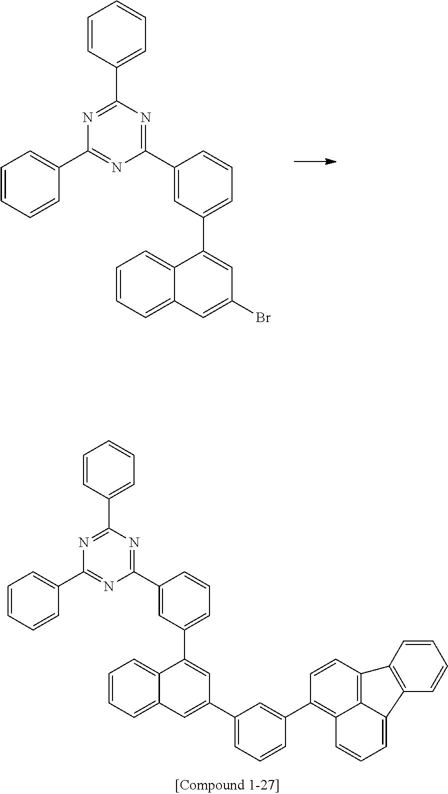 [SMILES: Brc1cc(-c2cccc(-c3nc(-c4ccccc4)nc(-c4ccccc4)n3)c2)c2ccccc2c1 ; c1ccc(-c2nc(-c3ccccc3)nc(-c3cccc(-c4cc(-c5cccc(-c6ccc7c8c(cccc68)-c6ccccc6-7)c5)cc5ccccc45)c3)n2)cc1]

[Compound 1-27] was prepared in the same manner as in Synthesis of [Compound 1-1] except that 2-(3-(3-bromonaphthalen-1-yl)phenyl)-4,6-diphenyl-1,3,5-triazine was used instead of 1-(4-(4,6-diphenyl-1,3,5-triazin-2-yl)phenyl)naphthalen-2-yl 1,1,2,2,3,3,4,4,4-nonafluorobutane-1-sulfonate, and (3-(fluoranthen-3-yl)phenyl)boronic acid was used instead of 2-(4-(fluoranthen-3-yl)phenyl)-4,4,5,5-tetramethyl-1,3,2-dioxaborolane.

[M+H]⁺=712

<Synthesis of Compound 1-28>

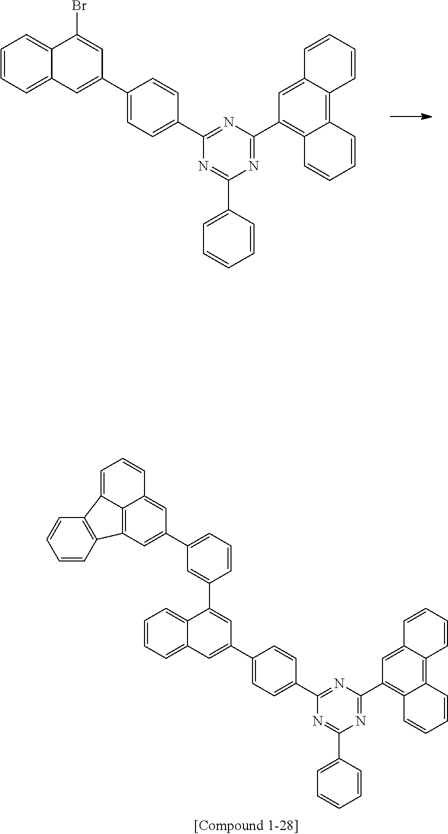 [SMILES: Brc1cc(-c2ccc(-c3nc(-c4ccccc4)nc(-c4cc5ccccc5c5ccccc45)n3)cc2)cc2ccccc12 ; c1ccc(-c2nc(-c3ccc(-c4cc(-c5cccc(-c6cc7c8c(cccc8c6)-c6ccccc6-7)c5)c5ccccc5c4)cc3)nc(-c3cc4ccccc4c4ccccc34)n2)cc1]

[Compound 1-28] was prepared in the same manner as in Synthesis of [Compound 1-1] except that 2-(4-(4-bromonaphthalen-2-yl)phenyl)-4-(phenanthren-9-yl)-6-phenyl-1,3,5-triazine was used instead of 1-(4-(4,6-diphenyl-1,3,5-triazin-2-yl)phenyl)naphthalen-2-yl 1,1,2,2,3,3,4,4,4-nonafluorobutane-1-sulfonate, and 2-(3-(fluoranthen-2-yl)phenyl)-4,4,5,5-tetramethyl-1,3,2-dioxaborolane was used instead of 2-(4-(fluoranthen-3-yl)phenyl)-4,4,5,5-tetramethyl-1,3,2-dioxaborolane.

[M+H]⁺=812

<Synthesis of Compound 1-29>

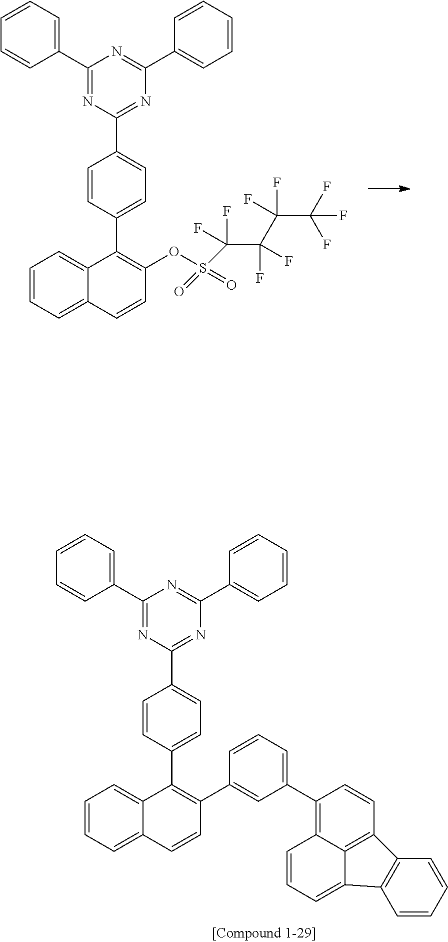 [SMILES: O=S(=O)(Oc1ccc2ccccc2c1-c1ccc(-c2nc(-c3ccccc3)nc(-c3ccccc3)n2)cc1)C(F)(F)C(F)(F)C(F)(F)C(F)(F)F ; c1ccc(-c2nc(-c3ccccc3)nc(-c3ccc(-c4c(-c5cccc(-c6ccc7c8c(cccc68)-c6ccccc6-7)c5)ccc5ccccc45)cc3)n2)cc1]

[Compound 1-29] was prepared in the same manner as in Synthesis of [Compound 1-1] except that 2-(3-(fluoranthen-3-yl)phenyl)-4,4,5,5-tetramethyl-1,3,2-dioxaborolane was used instead of 2-(4-(fluoranthen-3-yl)phenyl)-4,4,5,5-tetramethyl-1,3,2-dioxaborolane.

[M+H]⁺=712

<Synthesis of Compound 1-30>

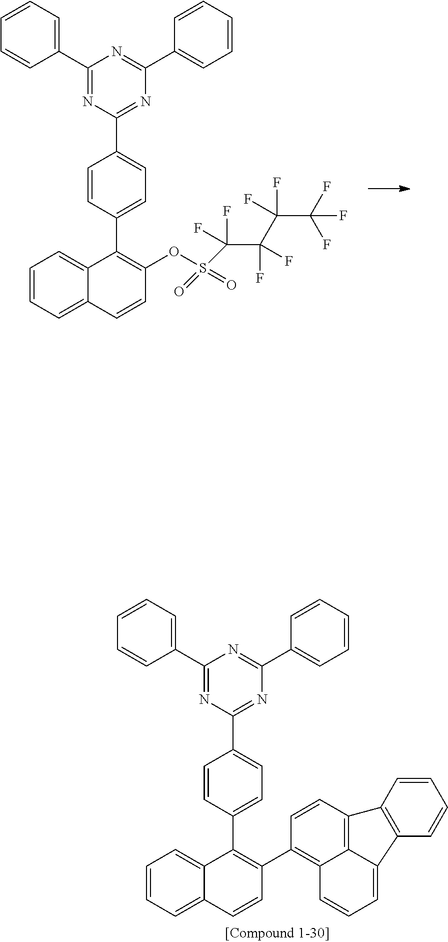 [SMILES: O=S(=O)(Oc1ccc2ccccc2c1-c1ccc(-c2nc(-c3ccccc3)nc(-c3ccccc3)n2)cc1)C(F)(F)C(F)(F)C(F)(F)C(F)(F)F ; c1ccc(-c2nc(-c3ccccc3)nc(-c3ccc(-c4c(-c5ccc6c7c(cccc57)-c5ccccc5-6)ccc5ccccc45)cc3)n2)cc1]

[Compound 1-30] was prepared in the same manner as in Synthesis of [Compound 1-1] except that fluoranthen-3-ylboronic acid was used instead of 2-(4-(fluoranthen-3-yl)phenyl)-4,4,5,5-tetramethyl-1,3,2-dioxaborolane.

[M+H]⁺=636

Manufacture of Organic Light Emitting Device

### Example 1

A glass substrate on which indium tin oxide (ITO) was coated as a thin film to a thickness of 100 nm was placed in detergent-dissolved distilled water and ultrasonic cleaned. Herein, a product of Fischer Co. was used as the detergent, and as the distilled water, distilled water filtered twice with a filter manufactured by Millipore Co. was used. After the ITO was cleaned for 30 minutes, ultrasonic cleaning was repeated twice using distilled water for 10 minutes. After the cleaning with distilled water was finished, the substrate was ultrasonic cleaned with solvents of isopropyl alcohol, acetone and methanol, then dried, and then transferred to a plasma cleaner. In addition, the substrate was cleaned for 5 minutes using oxygen plasma, and then transferred to a vacuum deposition apparatus.

On the transparent ITO electrode prepared as above, the following Compound HI-A was thermal vacuum deposited to a thickness of 60 nm to form a hole injection layer.

On the hole injection layer, the following Compound HAT was vacuum deposited to form a first hole transfer layer having a thickness of 5 nm, and the following Compound HT-A was vacuum deposited on the first hole transfer layer to form a second hole transfer layer having a thickness of 50 nm.

Subsequently, Compound BH and Compound BD were vacuum deposited on the second hole transfer layer in a weight ratio of 25:1 to form a light emitting layer having a thickness of 20 nm.

On the light emitting layer, [Compound 1-1] and the following Compound LiQ were vacuum deposited in a weight ratio of 1:1 to form an electron injection and transfer layer having a thickness of 35 nm.

A cathode was formed by depositing lithium fluoride (LiF) on the electron injection and transfer layer to a thickness of 1 nm and then depositing aluminum to a thickness of 100 nm, and as a result, an organic light emitting device was manufactured.

In the above-described process, the deposition rates of the organic materials were maintained at 0.04 nm/sec to 0.09 nm/sec, the deposition rate of the lithium fluoride was maintained at 0.03 nm/sec, and the deposition rate of the aluminum was maintained at 0.2 nm/sec. The degree of vacuum during the deposition was maintained at 1×10⁻⁷ torr to 5×10⁻⁵ torr.

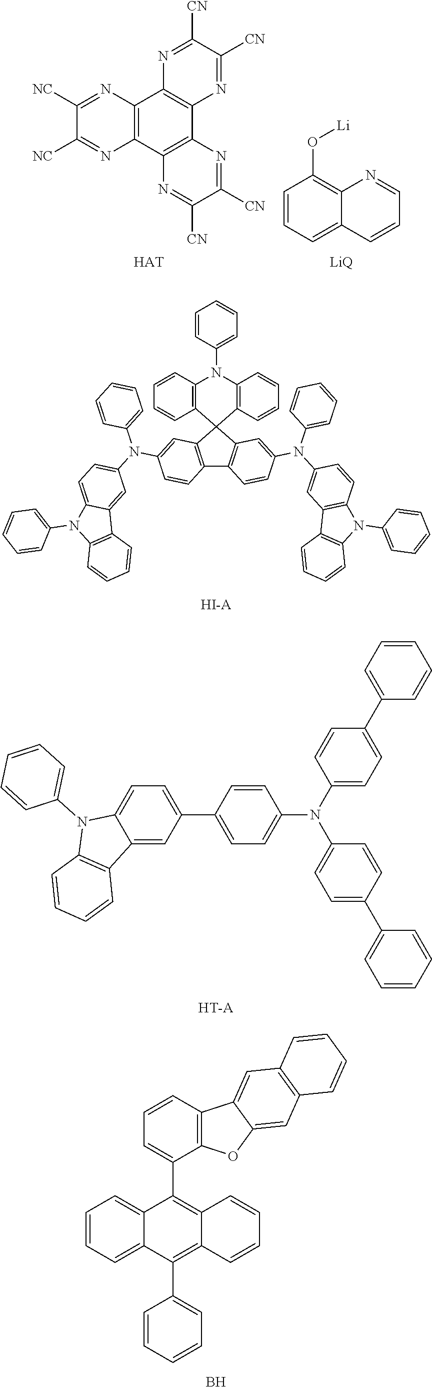

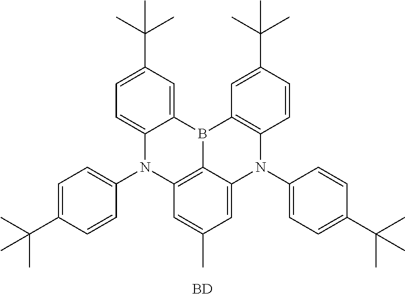

### Examples 2 to 30

Organic light emitting devices were manufactured in the same manner as in Example 1 except that compounds of the following Table 1 were used instead of [Compound 1-1] of Example 1.

### Comparative Examples 1 to 12

Organic light emitting devices were manufactured in the same manner as in Example 1 except that compounds of the following Table 1 were used instead of [Compound 1-1] of Example 1.

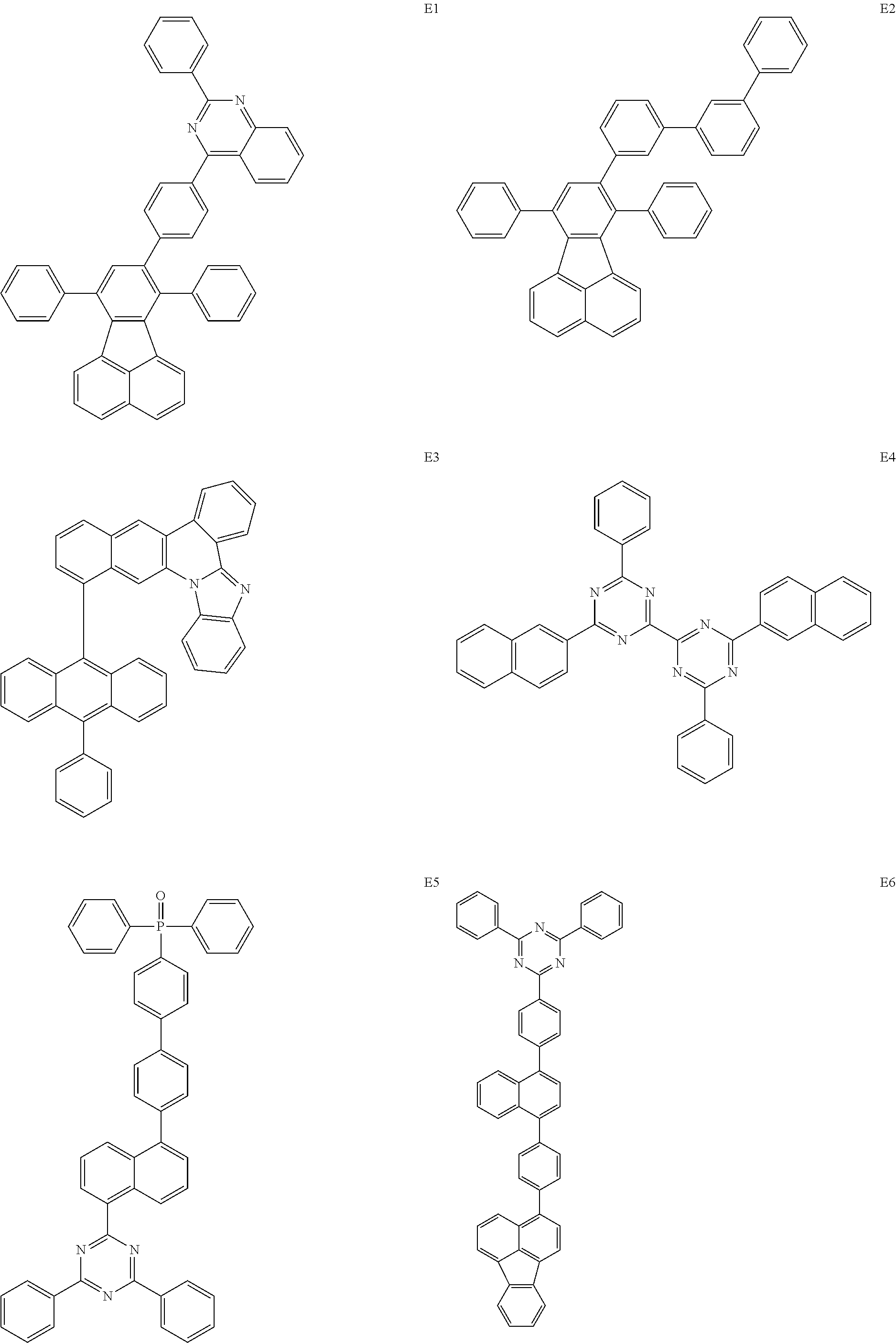

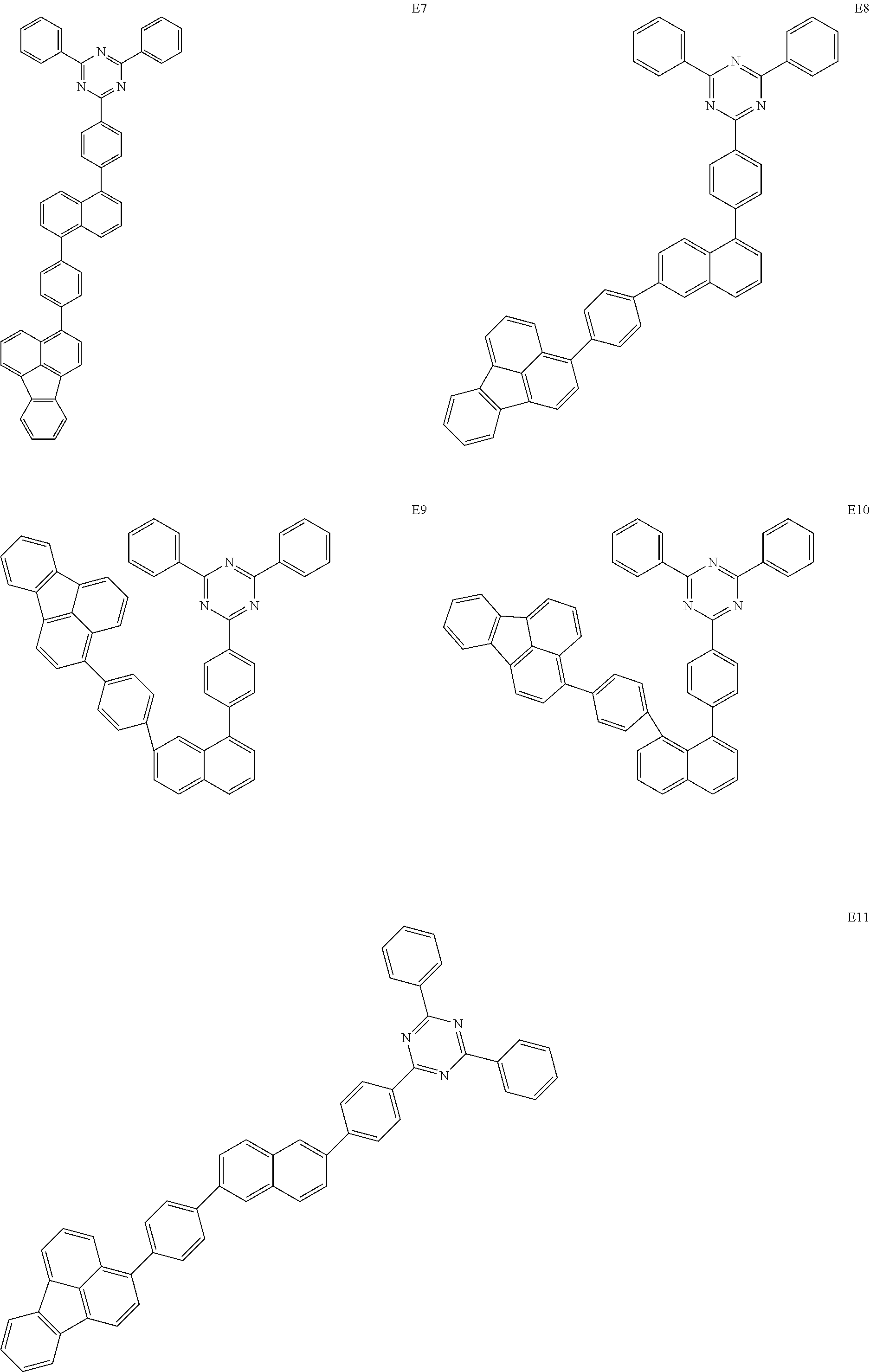

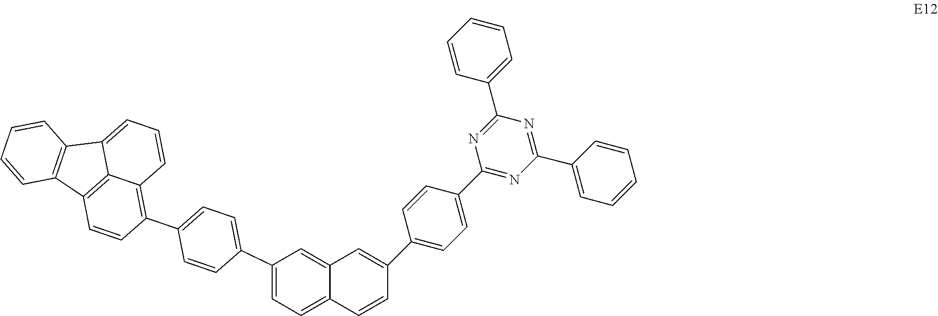 [SMILES: c1ccc(-c2nc(-c3ccccc3)nc(-c3ccc(-c4ccc5ccc(-c6ccc(-c7ccc8c9c(cccc79)-c7ccccc7-8)cc6)cc5c4)cc3)n2)cc1]

For each of the organic light emitting devices of Examples 1 to 30 and Comparative Examples 1 to 12, voltage and efficiency were measured under current density of 10 mA/cm² and lifetime (LT₉₅) was measured under current density of 20 mA/cm², and the results are shown in the following Table 1. Herein, LT₉₅ means time taken for luminance to become 95% with respect to initial luminance. Color coordinate (x, y) means a CIE color coordinate.

TABLE 1

| | Electron Injection and | Driving | Efficiency | Color | LT₉₅ |
|---|---|---|---|---|---|
| | Transfer Layer | Voltage (V) | (cd/A) | Coordinate (x, y) | (h) |
| Example 1 | Compound 1-1 | 3.76 | 5.21 | (0.142, 0.097) | 277 |
| Example 2 | Compound 1-2 | 3.84 | 5.08 | (0.142, 0.097) | 280 |
| Example 3 | Compound 1-3 | 3.74 | 5.30 | (0.142, 0.097) | 268 |
| Example 4 | Compound 1-4 | 3.82 | 5.10 | (0.142, 0.096) | 276 |
| Example 5 | Compound 1-5 | 4.01 | 5.02 | (0.142, 0.097) | 290 |
| Example 6 | Compound 1-6 | 3.97 | 5.00 | (0.142, 0.097) | 285 |
| Example 7 | Compound 1-7 | 3.89 | 5.12 | (0.142, 0.096) | 288 |
| Example 8 | Compound 1-8 | 3.80 | 5.16 | (0.142, 0.096) | 274 |
| Example 9 | Compound 1-9 | 3.86 | 5.05 | (0.142, 0.097) | 273 |
| Example 10 | Compound 1-10 | 4.09 | 4.96 | (0.142, 0.097) | 320 |
| Example 11 | Compound 1-11 | 4.03 | 4.98 | (0.142, 0.097) | 271 |
| Example 12 | Compound 1-12 | 4.04 | 5.05 | (0.142, 0.096) | 290 |
| Example 13 | Compound 1-13 | 3.99 | 5.02 | (0.142, 0.096) | 285 |
| Example 14 | Compound 1-14 | 3.87 | 5.08 | (0.142, 0.096) | 282 |
| Example 15 | Compound 1-15 | 3.80 | 5.11 | (0.142, 0.097) | 273 |
| Example 16 | Compound 1-16 | 4.00 | 4.97 | (0.142, 0.096) | 292 |
| Example 17 | Compound 1-17 | 4.08 | 4.87 | (0.142, 0.097) | 304 |
| Example 18 | Compound 1-18 | 3.85 | 5.16 | (0.142, 0.096) | 270 |
| Example 19 | Compound 1-19 | 4.05 | 5.00 | (0.142, 0.097) | 282 |
| Example 20 | Compound 1-20 | 3.99 | 5.10 | (0.142, 0.097) | 293 |
| Example 21 | Compound 1-21 | 3.79 | 5.19 | (0.142, 0.097) | 275 |
| Example 22 | Compound 1-22 | 3.86 | 5.04 | (0.142, 0.097) | 271 |
| Example 23 | Compound 1-23 | 4.02 | 5.06 | (0.142, 0.097) | 279 |
| Example 24 | Compound 1-24 | 3.99 | 5.13 | (0.142, 0.097) | 284 |
| Example 25 | Compound 1-25 | 4.03 | 5.09 | (0.142, 0.097) | 294 |
| Example 26 | Compound 1-26 | 4.04 | 5.07 | (0.142, 0.097) | 283 |
| Example 27 | Compound 1-27 | 4.00 | 5.10 | (0.142, 0.097) | 286 |
| Example 28 | Compound 1-28 | 4.04 | 5.14 | (0.142, 0.097) | 297 |
| Example 29 | Compound 1-29 | 3.78 | 5.25 | (0.142, 0.096) | 276 |
| Example 30 | Compound 1-30 | 3.81 | 5.18 | (0.142, 0.096) | 286 |
| Comparative | Compound E1 | 4.26 | 4.47 | (0.142, 0.097) | 139 |
| Example 1 | | | | | |
| Comparative | Compound E2 | 4.19 | 4.32 | (0.142, 0.097) | 83 |
| Example 2 | | | | | |
| Comparative | Compound E3 | 4.16 | 4.52 | (0.142, 0.096) | 128 |
| Example 3 | | | | | |
| Comparative | Compound E4 | 4.22 | 4.55 | (0.142, 0.097) | 116 |
| Example 4 | | | | | |
| Comparative | Compound E5 | 4.28 | 4.56 | (0.142, 0.097) | 140 |
| Example 5 | | | | | |
| Comparative | Compound E6 | 4.18 | 4.53 | (0.142, 0.097) | 120 |
| Example 6 | | | | | |
| Comparative | Compound E7 | 4.20 | 4.55 | (0.142, 0.096) | 126 |
| Example 7 | | | | | |
| Comparative | Compound E8 | 4.27 | 4.49 | (0.142, 0.097) | 127 |
| Example 8 | | | | | |
| Comparative | Compound E9 | 4.25 | 4.50 | (0.142, 0.097) | 123 |
| Example 9 | | | | | |
| Comparative | Compound E10 | 4.17 | 4.56 | (0.142, 0.097) | 122 |
| Example 10 | | | | | |
| Comparative | Compound E11 | 4.27 | 4.48 | (0.142, 0.097) | 136 |
| Example 11 | | | | | |
| Comparative | Compound E12 | 4.25 | 4.51 | (0.142, 0.097) | 135 |
| Example 12 | | | | | |

In Table 1, it was identified that Examples 1 to 30 using Chemical Formula 1 according to one embodiment of the present specification in the electron injection and transfer layer of the organic light emitting device had lower driving voltage, higher efficiency and more superior lifetime properties compared to Comparative Examples 1 to 12. Particularly, it was seen that the organic light emitting devices of Examples 1 to 30 including the compound in which an N-containing heterocyclic group and a fluoranthene derivative bond at positions of 1,2, positions of 1,3 or positions of 2,3 of naphthalene as in Chemical Formula 1 according to one embodiment of the present specification had more superior driving voltage, efficiency and lifetime compared to the organic light emitting devices of Comparative Examples 6 to 12 having substituents bonding at positions other than the positions of 1,2, the positions of 1,3 or the positions of 2,3 of naphthalene since the N-containing heterocyclic group smoothly controlled the electron mobility when manufacturing the organic light emitting device by having a substituent with an electron-deficient structure and thereby increasing molecular polarity (dipole moment).

Hereinbefore, preferred embodiments of the present disclosure have been described, however, the present disclosure is not limited thereto, and various modifications may be made within the scope of the claims and the scope of the detailed descriptions, and these also fall within the category of the present disclosure.

1. A compound represented by the following Chemical Formula 1: 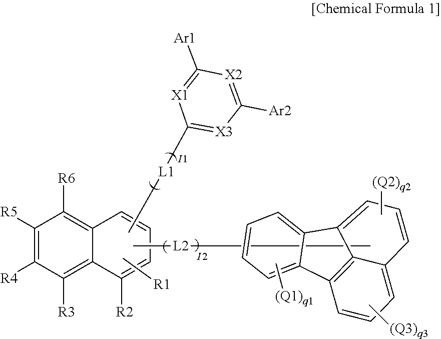
wherein in Chemical Formula 1,
at least two of X1 to X3 are N, and the rest is
R is hydrogen or deuterium,
L1 and L2 are the same as or different from each other, and each independently a direct bond; a substituted or unsubstituted alkylene group; a substituted or unsubstituted alkenylene group; a substituted or unsubstituted arylene group; or a substituted or unsubstituted heteroarylene group,
Ar1, Ar2, R1 to R6 and Q1 to Q3 are the same as or different from each other, and each independently hydrogen; deuterium; a halogen group; a cyano group; a nitro group; a hydroxyl group; a substituted or unsubstituted alkyl group; a substituted or unsubstituted cycloalkyl group; a substituted or unsubstituted alkoxy group; a substituted or unsubstituted aryloxy group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted amine group; a substituted or unsubstituted arylphosphine group; a substituted or unsubstituted phosphine oxide group; a substituted or unsubstituted aryl group; or a substituted or unsubstituted heteroaryl group, or adjacent groups among Ar1, Ar2, R1 to R6 and Q1 to Q3 bond to each other to form a substituted or unsubstituted ring,
l1 is an integer of 1 to 5,
l2 is an integer of 1 to 5,
q1 is an integer of 1 to 4,
q2 is an integer of 1 to 3,
q3 is an integer of 1 to 3,
when l1 is 2 or greater, the two or more L1s are the same as or different from each other,
when l2 is 2 or greater, the two or more L2s are the same as or different from each other,
when q1 is 2 or greater, the two or more Q1s are the same as or different from each other,
when q2 is 2 or greater, the two or more Q2s are the same as or different from each other,
when q3 is 2 or greater, the two or more Q3 s are the same as or different from each other, and
2≤q1+q2≤6.

2. The compound of claim 1, wherein the compound of Chemical Formula 1 is represented by any one of the following Chemical Formulae 1-1 to 1-6: 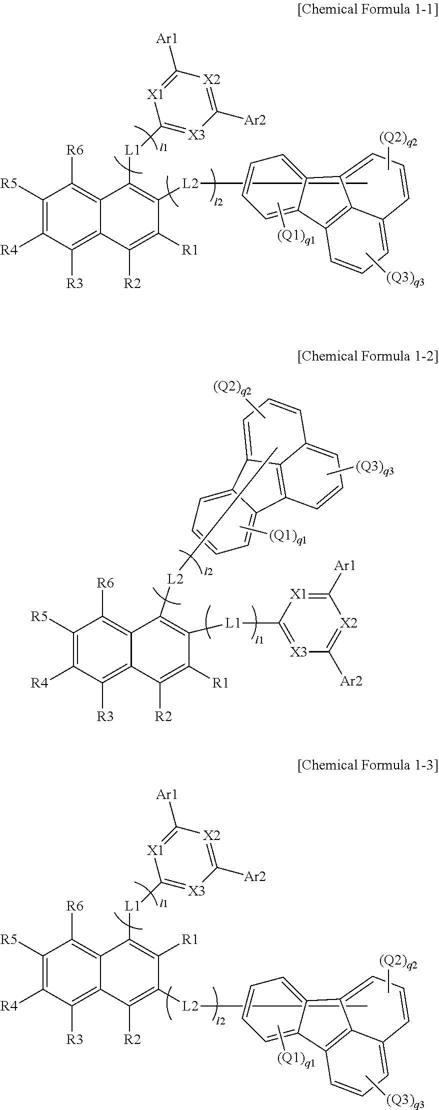 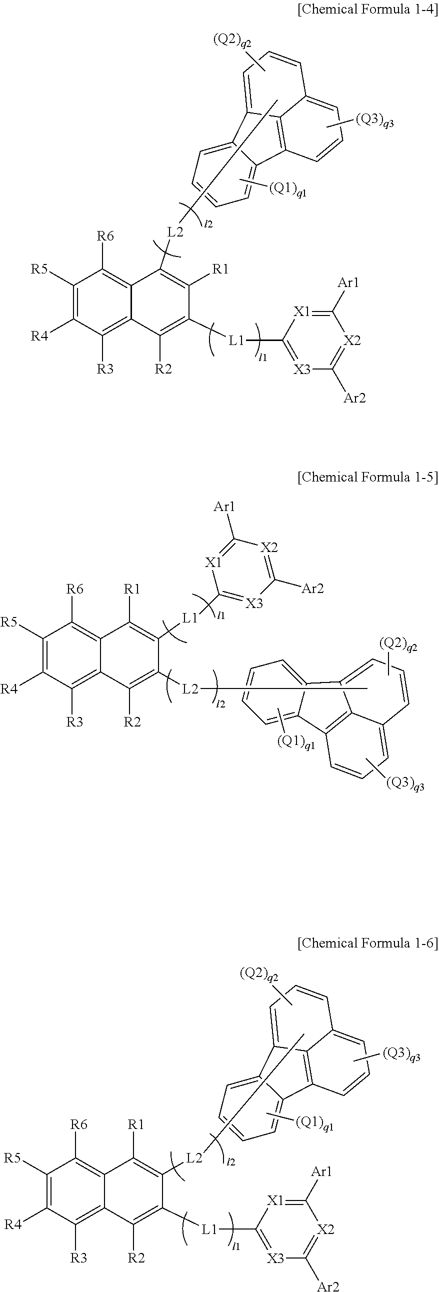
wherein in Chemical Formulae 1-1 to 1-6,
X1 to X3, L1, L2, l1, l2, R1 to R6, Ar1, Ar2, Q1 to Q3 and q1 to q3 have the same definitions as in Chemical Formula 1.

3. The compound of claim 1, wherein the compound of Chemical Formula 1 is represented by any one of the following Chemical Formulae 1-7 to 1-9: 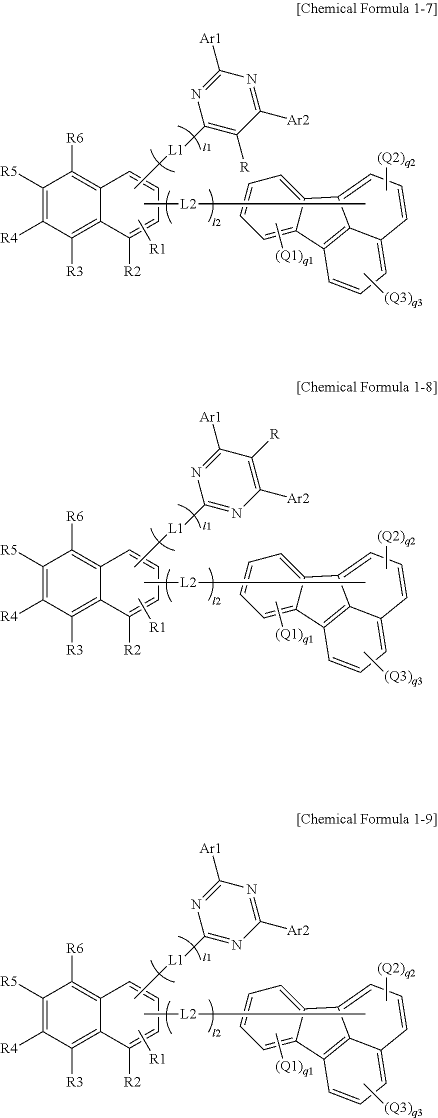
wherein in Chemical Formulae 1-7 to 1-9,
L1, L2, l1, l2, R1 to R6, Ar1, Ar2, Q1 to Q3 and q1 to q3 have the same definitions as in Chemical Formula 1, and
R is hydrogen or deuterium.

4. The compound of claim 1, wherein R1 to R6 and R are hydrogen,
L1 and L2 are the same as or different from each other, and each independently a direct bond; or a monocyclic or polycyclic arylene group having 6 to 30 carbon atoms,
Ar1 and Ar2 are the same as or different from each other, and each independently a linear or branched alkyl group having 1 to 30 carbon atoms; a monocyclic or polycyclic aryl group having 6 to 30 carbon atoms unsubstituted or substituted with a linear or branched alkyl group having 1 to 30 carbon atoms; or a monocyclic or polycyclic heteroaryl group having 2 to 30 carbon atoms, and
Q1 to Q3 are the same as or different from each other, and each independently hydrogen; a cyano group; or a monocyclic or polycyclic aryl group having 6 to 30 carbon atoms, or adjacent groups among Q1 to Q3 bond to each other to form a monocyclic or polycyclic aromatic hydrocarbon ring having 6 to 30 carbon atoms.

5. The compound of claim 1, wherein the compound of Chemical Formula 1 is any one selected from the following compounds:

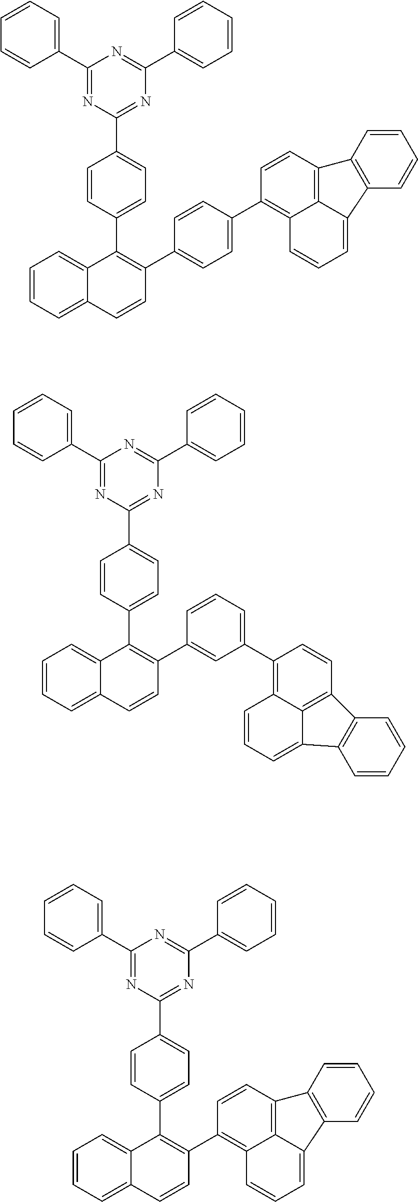 [SMILES: c1ccc(-c2nc(-c3ccccc3)nc(-c3ccc(-c4c(-c5ccc(-c6ccc7c8c(cccc68)-c6ccccc6-7)cc5)ccc5ccccc45)cc3)n2)cc1 ; c1ccc(-c2nc(-c3ccccc3)nc(-c3ccc(-c4c(-c5cccc(-c6ccc7c8c(cccc68)-c6ccccc6-7)c5)ccc5ccccc45)cc3)n2)cc1 ; c1ccc(-c2nc(-c3ccccc3)nc(-c3ccc(-c4c(-c5ccc6c7c(cccc57)-c5ccccc5-6)ccc5ccccc45)cc3)n2)cc1]

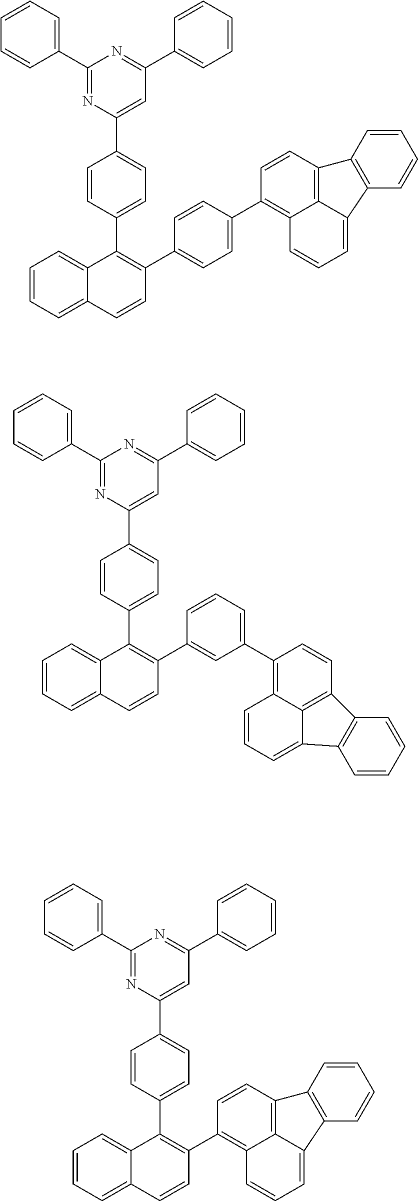 [SMILES: c1ccc(-c2cc(-c3ccc(-c4c(-c5ccc(-c6ccc7c8c(cccc68)-c6ccccc6-7)cc5)ccc5ccccc45)cc3)nc(-c3ccccc3)n2)cc1 ; c1ccc(-c2cc(-c3ccc(-c4c(-c5cccc(-c6ccc7c8c(cccc68)-c6ccccc6-7)c5)ccc5ccccc45)cc3)nc(-c3ccccc3)n2)cc1 ; c1ccc(-c2cc(-c3ccc(-c4c(-c5ccc6c7c(cccc57)-c5ccccc5-6)ccc5ccccc45)cc3)nc(-c3ccccc3)n2)cc1]

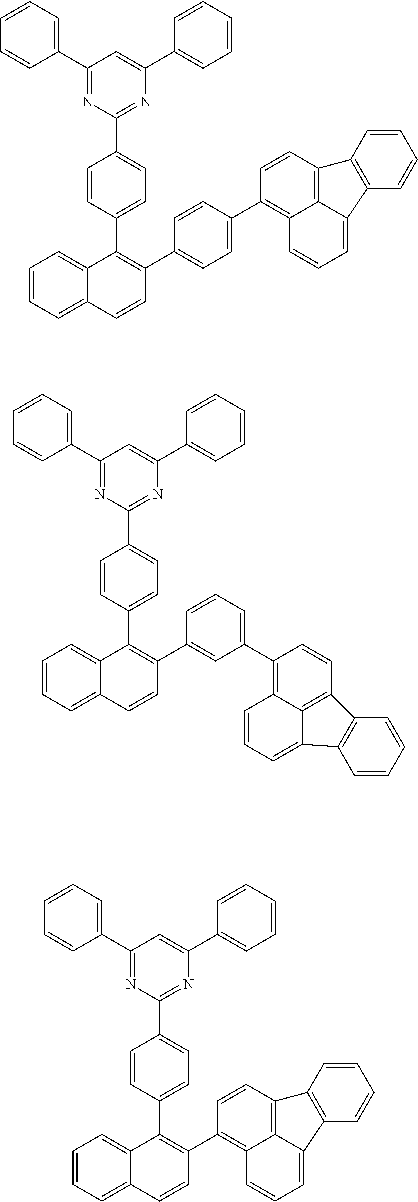 [SMILES: c1ccc(-c2cc(-c3ccccc3)nc(-c3ccc(-c4c(-c5ccc(-c6ccc7c8c(cccc68)-c6ccccc6-7)cc5)ccc5ccccc45)cc3)n2)cc1 ; c1ccc(-c2cc(-c3ccccc3)nc(-c3ccc(-c4c(-c5cccc(-c6ccc7c8c(cccc68)-c6ccccc6-7)c5)ccc5ccccc45)cc3)n2)cc1 ; c1ccc(-c2cc(-c3ccccc3)nc(-c3ccc(-c4c(-c5ccc6c7c(cccc57)-c5ccccc5-6)ccc5ccccc45)cc3)n2)cc1]

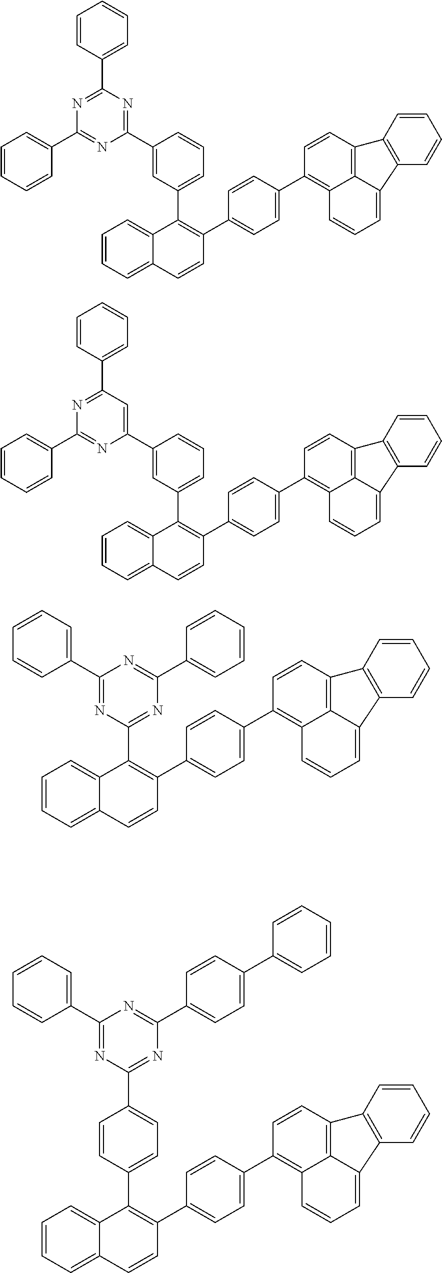

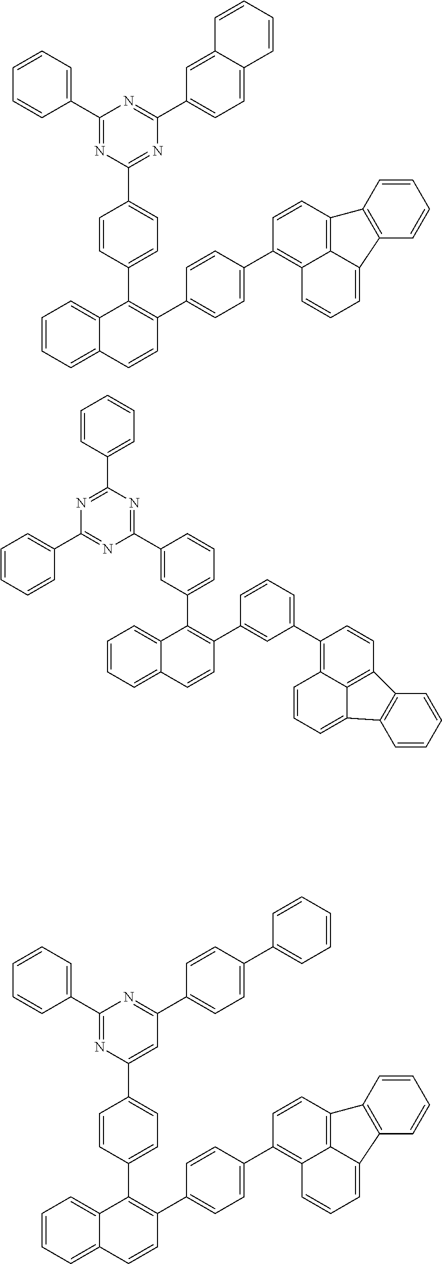

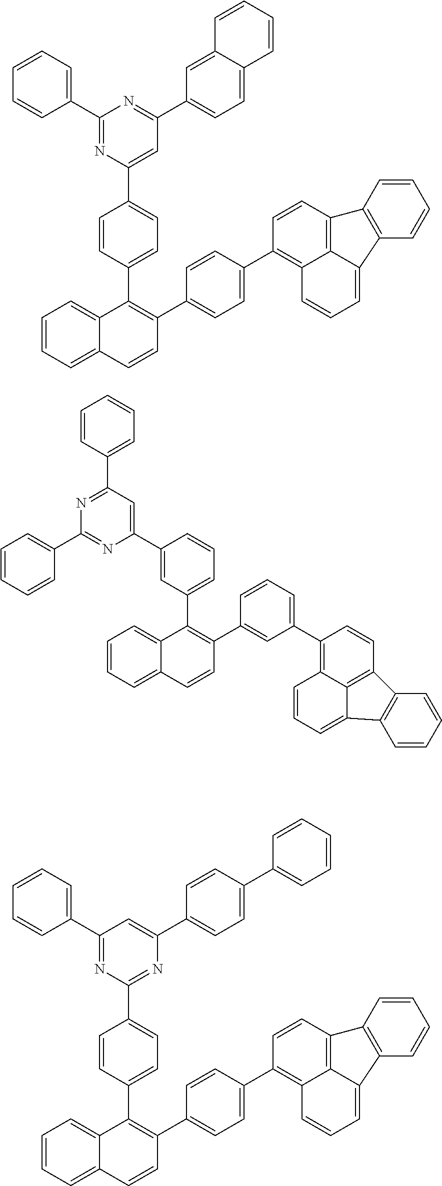

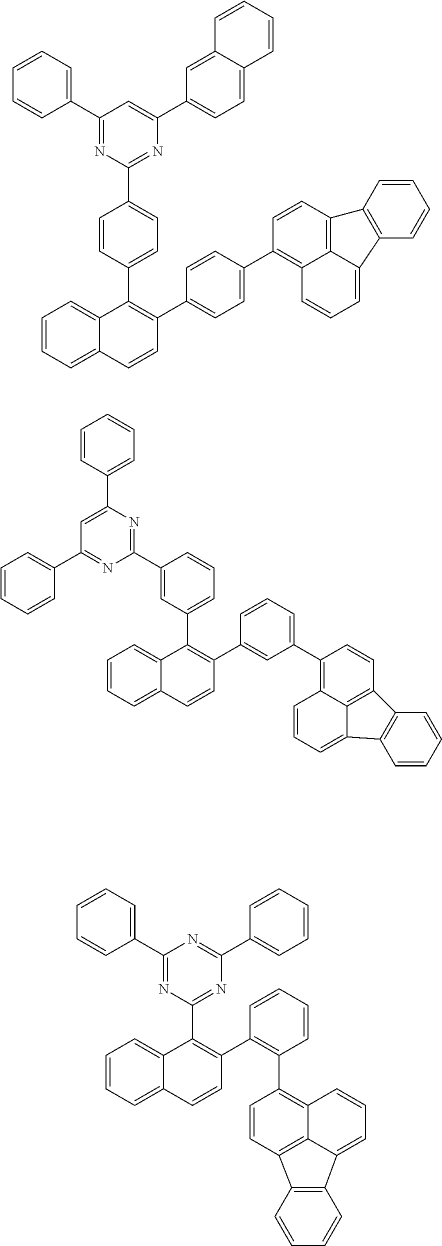 [SMILES: c1ccc(-c2cc(-c3ccc4ccccc4c3)nc(-c3ccc(-c4c(-c5ccc(-c6ccc7c8c(cccc68)-c6ccccc6-7)cc5)ccc5ccccc45)cc3)n2)cc1 ; c1ccc(-c2cc(-c3ccccc3)nc(-c3cccc(-c4c(-c5cccc(-c6ccc7c8c(cccc68)-c6ccccc6-7)c5)ccc5ccccc45)c3)n2)cc1 ; c1ccc(-c2nc(-c3ccccc3)nc(-c3c(-c4ccccc4-c4ccc5c6c(cccc46)-c4ccccc4-5)ccc4ccccc34)n2)cc1]

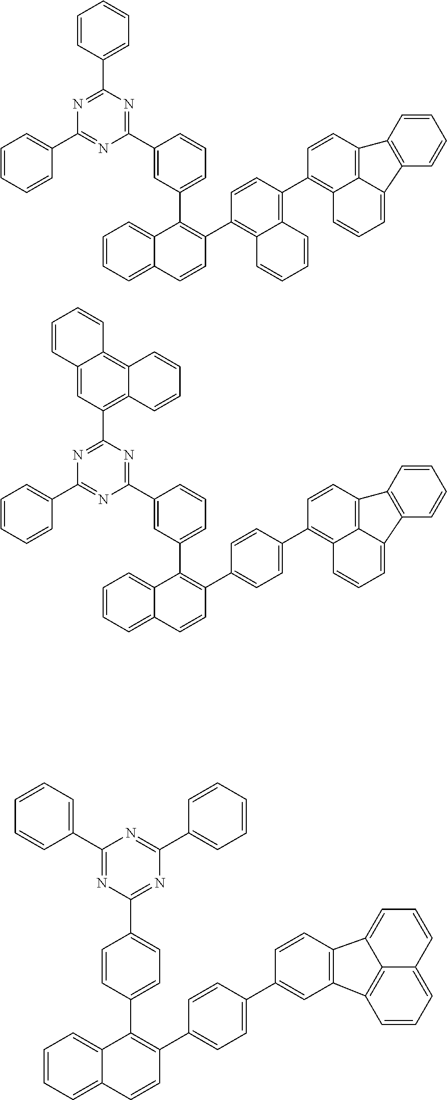 [SMILES: c1ccc(-c2nc(-c3ccccc3)nc(-c3cccc(-c4c(-c5ccc(-c6ccc7c8c(cccc68)-c6ccccc6-7)c6ccccc56)ccc5ccccc45)c3)n2)cc1 ; c1ccc(-c2nc(-c3cccc(-c4c(-c5ccc(-c6ccc7c8c(cccc68)-c6ccccc6-7)cc5)ccc5ccccc45)c3)nc(-c3cc4ccccc4c4ccccc34)n2)cc1 ; c1ccc(-c2nc(-c3ccccc3)nc(-c3ccc(-c4c(-c5ccc(-c6ccc7c(c6)-c6cccc8cccc-7c68)cc5)ccc5ccccc45)cc3)n2)cc1]

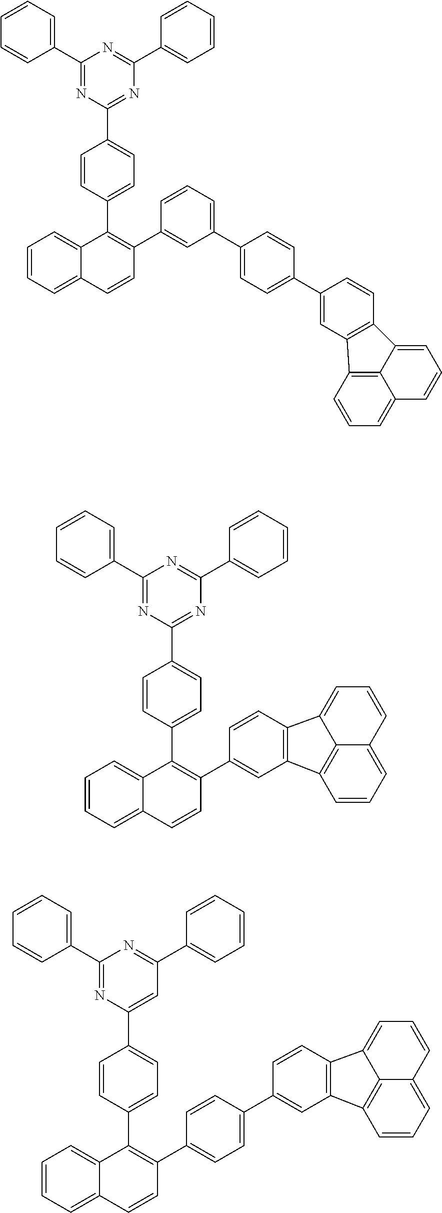 [SMILES: c1ccc(-c2nc(-c3ccccc3)nc(-c3ccc(-c4c(-c5cccc(-c6ccc(-c7ccc8c(c7)-c7cccc9cccc-8c79)cc6)c5)ccc5ccccc45)cc3)n2)cc1 ; c1ccc(-c2nc(-c3ccccc3)nc(-c3ccc(-c4c(-c5ccc6c(c5)-c5cccc7cccc-6c57)ccc5ccccc45)cc3)n2)cc1 ; c1ccc(-c2cc(-c3ccc(-c4c(-c5ccc(-c6ccc7c(c6)-c6cccc8cccc-7c68)cc5)ccc5ccccc45)cc3)nc(-c3ccccc3)n2)cc1]

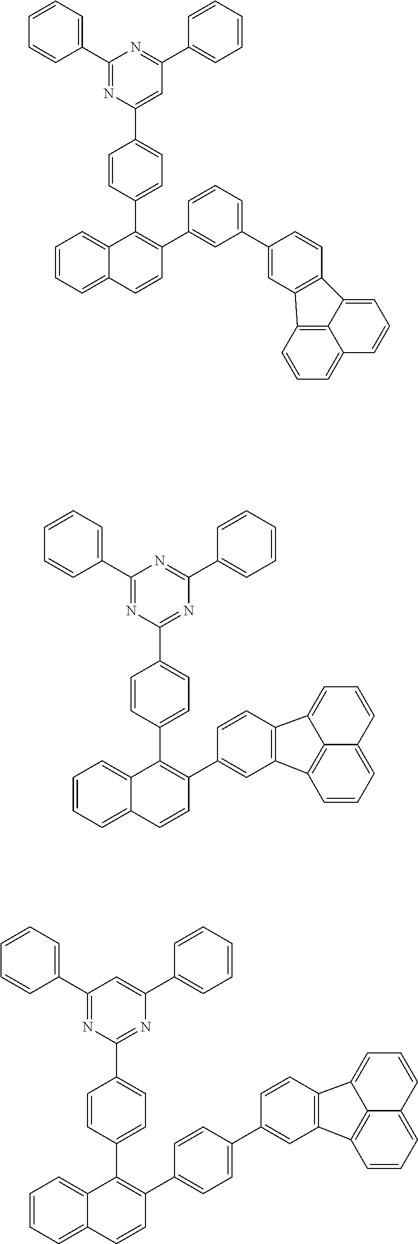 [SMILES: c1ccc(-c2cc(-c3ccc(-c4c(-c5cccc(-c6ccc7c(c6)-c6cccc8cccc-7c68)c5)ccc5ccccc45)cc3)nc(-c3ccccc3)n2)cc1 ; c1ccc(-c2nc(-c3ccccc3)nc(-c3ccc(-c4c(-c5ccc6c(c5)-c5cccc7cccc-6c57)ccc5ccccc45)cc3)n2)cc1 ; c1ccc(-c2cc(-c3ccccc3)nc(-c3ccc(-c4c(-c5ccc(-c6ccc7c(c6)-c6cccc8cccc-7c68)cc5)ccc5ccccc45)cc3)n2)cc1]

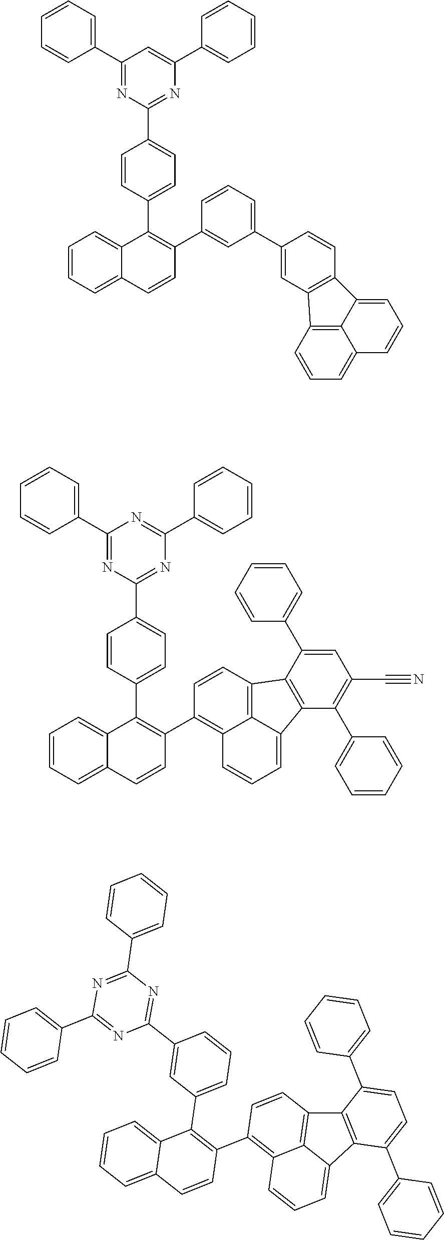 [SMILES: c1ccc(-c2cc(-c3ccccc3)nc(-c3ccc(-c4c(-c5cccc(-c6ccc7c(c6)-c6cccc8cccc-7c68)c5)ccc5ccccc45)cc3)n2)cc1 ; N#Cc1cc(-c2ccccc2)c2c(c1-c1ccccc1)-c1cccc3c(-c4ccc5ccccc5c4-c4ccc(-c5nc(-c6ccccc6)nc(-c6ccccc6)n5)cc4)ccc-2c13 ; c1ccc(-c2nc(-c3ccccc3)nc(-c3cccc(-c4c(-c5ccc6c7c(cccc57)-c5c(-c7ccccc7)ccc(-c7ccccc7)c5-6)ccc5ccccc45)c3)n2)cc1]

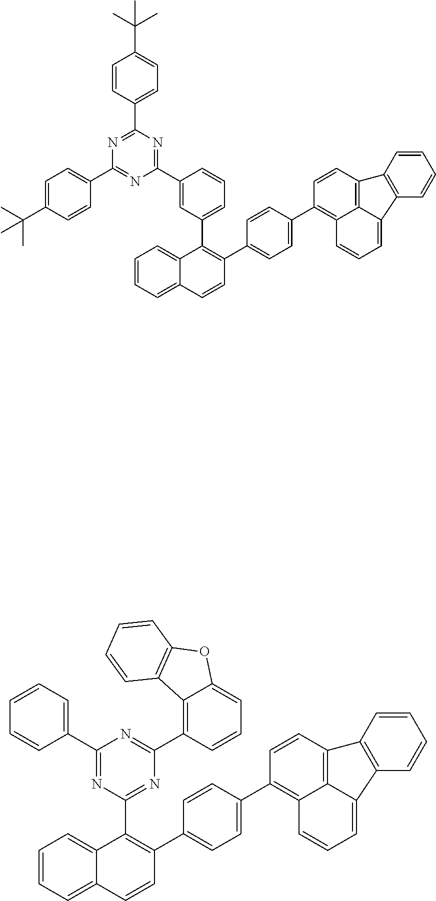 [SMILES: CC(C)(C)c1ccc(-c2nc(-c3ccc(C(C)(C)C)cc3)nc(-c3cccc(-c4c(-c5ccc(-c6ccc7c8c(cccc68)-c6ccccc6-7)cc5)ccc5ccccc45)c3)n2)cc1 ; c1ccc(-c2nc(-c3c(-c4ccc(-c5ccc6c7c(cccc57)-c5ccccc5-6)cc4)ccc4ccccc34)nc(-c3cccc4oc5ccccc5c34)n2)cc1]

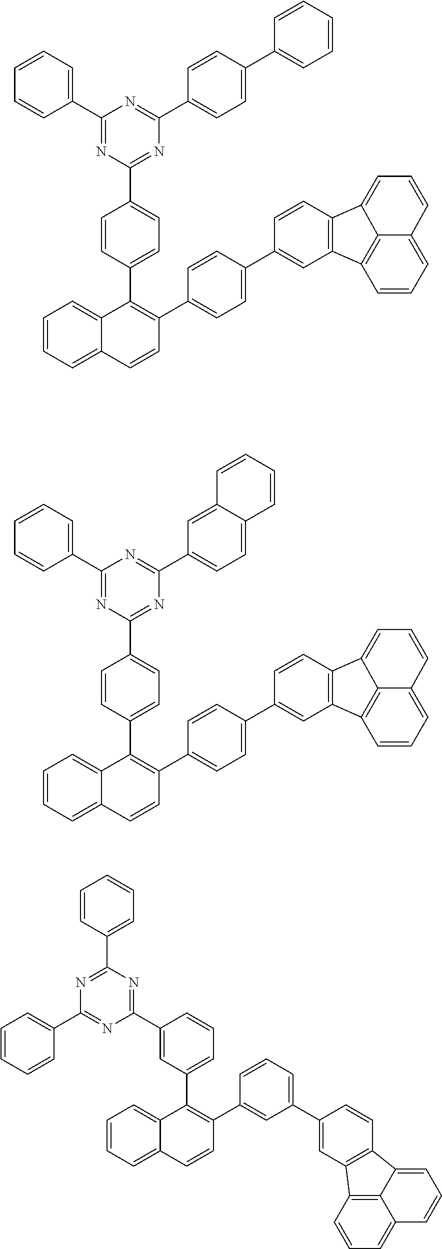 [SMILES: c1ccc(-c2ccc(-c3nc(-c4ccccc4)nc(-c4ccc(-c5c(-c6ccc(-c7ccc8c(c7)-c7cccc9cccc-8c79)cc6)ccc6ccccc56)cc4)n3)cc2)cc1 ; c1ccc(-c2nc(-c3ccc(-c4c(-c5ccc(-c6ccc7c(c6)-c6cccc8cccc-7c68)cc5)ccc5ccccc45)cc3)nc(-c3ccc4ccccc4c3)n2)cc1 ; c1ccc(-c2nc(-c3ccccc3)nc(-c3cccc(-c4c(-c5cccc(-c6ccc7c(c6)-c6cccc8cccc-7c68)c5)ccc5ccccc45)c3)n2)cc1]

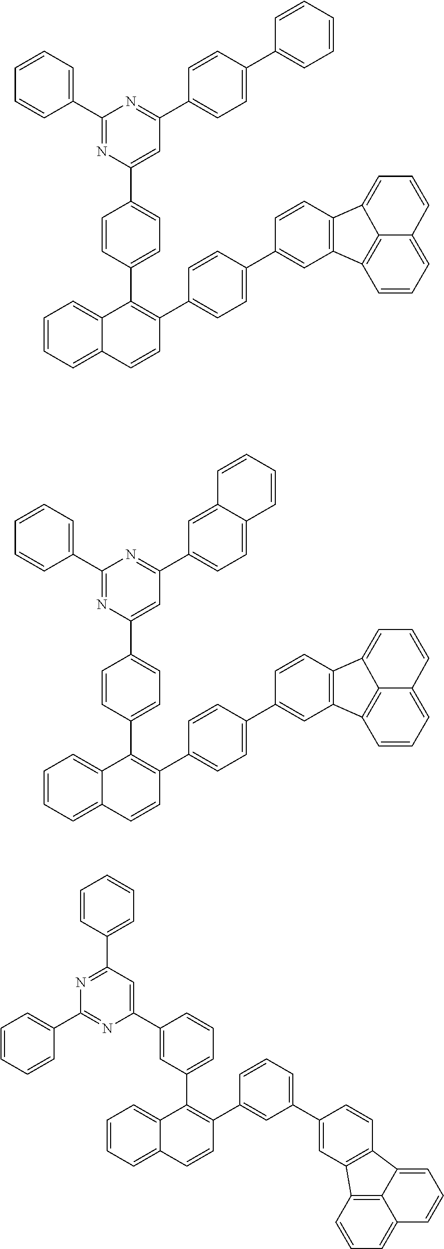 [SMILES: c1ccc(-c2ccc(-c3cc(-c4ccc(-c5c(-c6ccc(-c7ccc8c(c7)-c7cccc9cccc-8c79)cc6)ccc6ccccc56)cc4)nc(-c4ccccc4)n3)cc2)cc1 ; c1ccc(-c2nc(-c3ccc(-c4c(-c5ccc(-c6ccc7c(c6)-c6cccc8cccc-7c68)cc5)ccc5ccccc45)cc3)cc(-c3ccc4ccccc4c3)n2)cc1 ; c1ccc(-c2cc(-c3cccc(-c4c(-c5cccc(-c6ccc7c(c6)-c6cccc8cccc-7c68)c5)ccc5ccccc45)c3)nc(-c3ccccc3)n2)cc1]

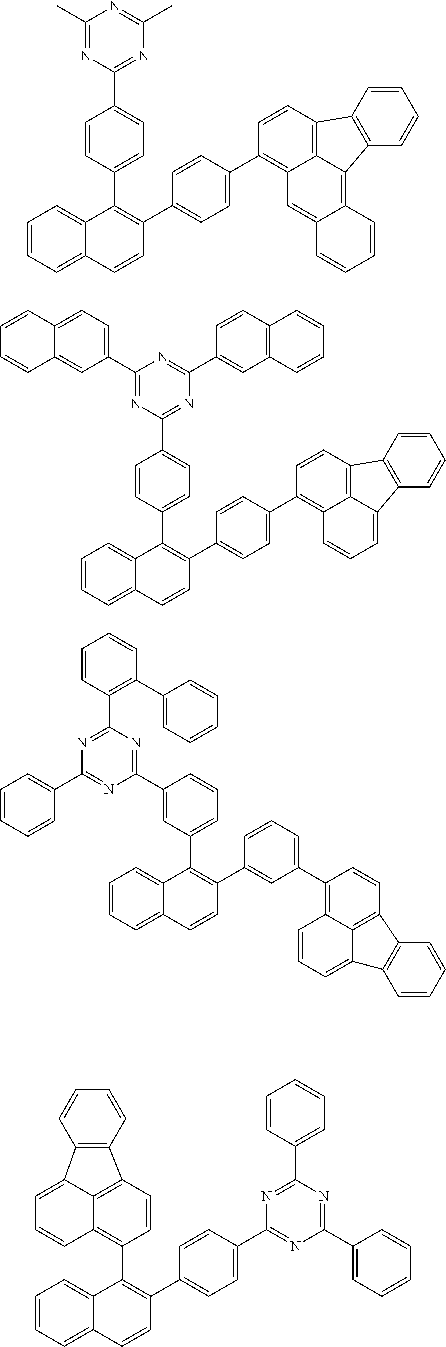

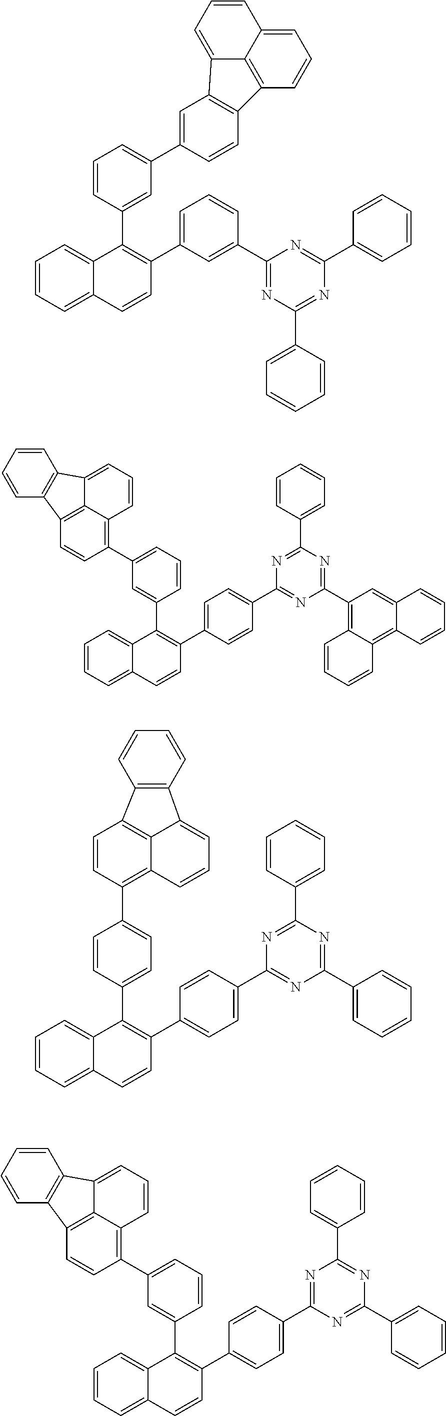 [SMILES: c1ccc(-c2nc(-c3ccccc3)nc(-c3cccc(-c4ccc5ccccc5c4-c4cccc(-c5ccc6c(c5)-c5cccc7cccc-6c57)c4)c3)n2)cc1 ; c1ccc(-c2nc(-c3ccc(-c4ccc5ccccc5c4-c4cccc(-c5ccc6c7c(cccc57)-c5ccccc5-6)c4)cc3)nc(-c3cc4ccccc4c4ccccc34)n2)cc1 ; c1ccc(-c2nc(-c3ccccc3)nc(-c3ccc(-c4ccc5ccccc5c4-c4ccc(-c5ccc6c7c(cccc57)-c5ccccc5-6)cc4)cc3)n2)cc1 ; c1ccc(-c2nc(-c3ccccc3)nc(-c3ccc(-c4ccc5ccccc5c4-c4cccc(-c5ccc6c7c(cccc57)-c5ccccc5-6)c4)cc3)n2)cc1]

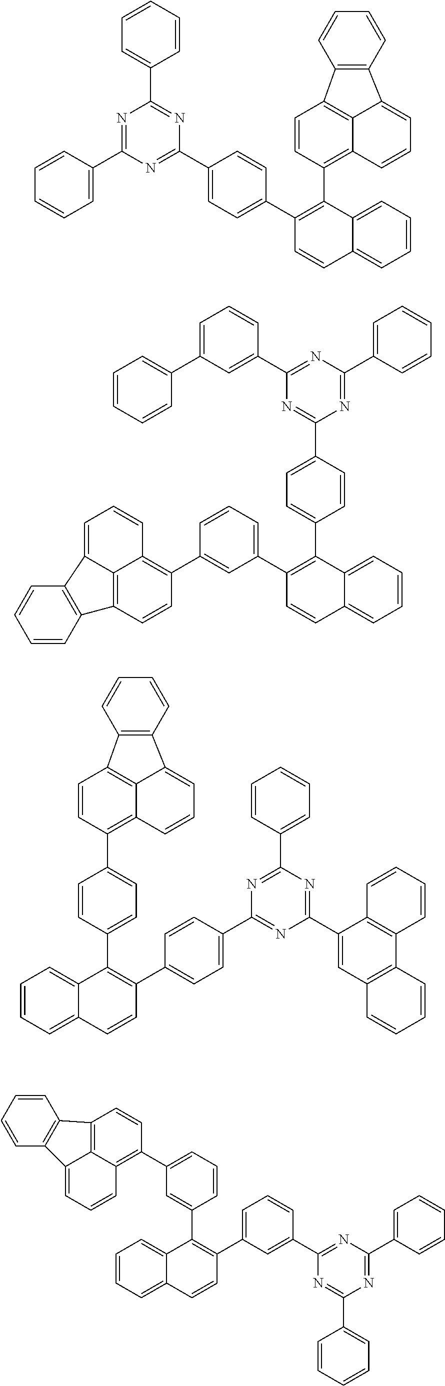

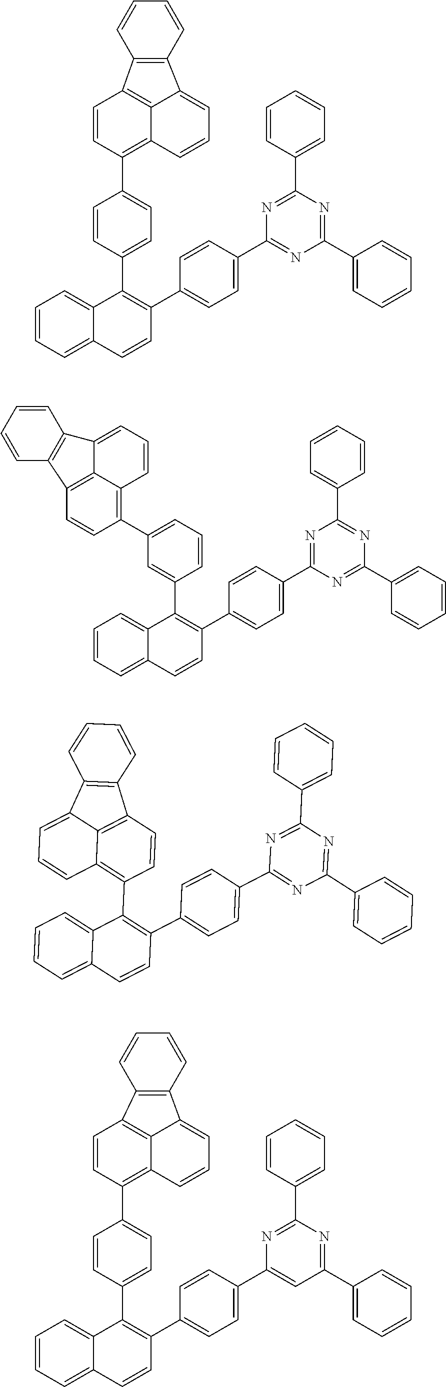 [SMILES: c1ccc(-c2nc(-c3ccccc3)nc(-c3ccc(-c4ccc5ccccc5c4-c4ccc(-c5ccc6c7c(cccc57)-c5ccccc5-6)cc4)cc3)n2)cc1 ; c1ccc(-c2nc(-c3ccccc3)nc(-c3ccc(-c4ccc5ccccc5c4-c4cccc(-c5ccc6c7c(cccc57)-c5ccccc5-6)c4)cc3)n2)cc1 ; c1ccc(-c2nc(-c3ccccc3)nc(-c3ccc(-c4ccc5ccccc5c4-c4ccc5c6c(cccc46)-c4ccccc4-5)cc3)n2)cc1 ; c1ccc(-c2cc(-c3ccc(-c4ccc5ccccc5c4-c4ccc(-c5ccc6c7c(cccc57)-c5ccccc5-6)cc4)cc3)nc(-c3ccccc3)n2)cc1]

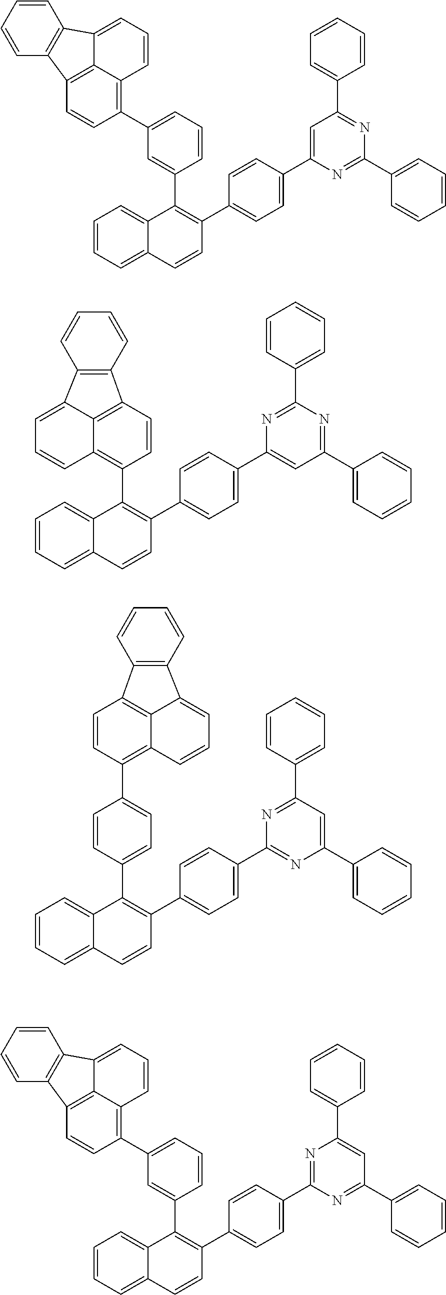

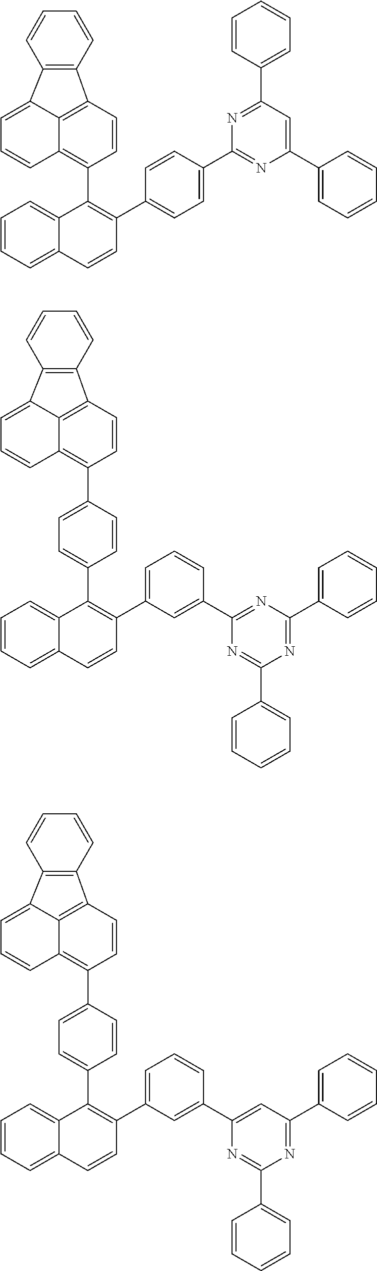

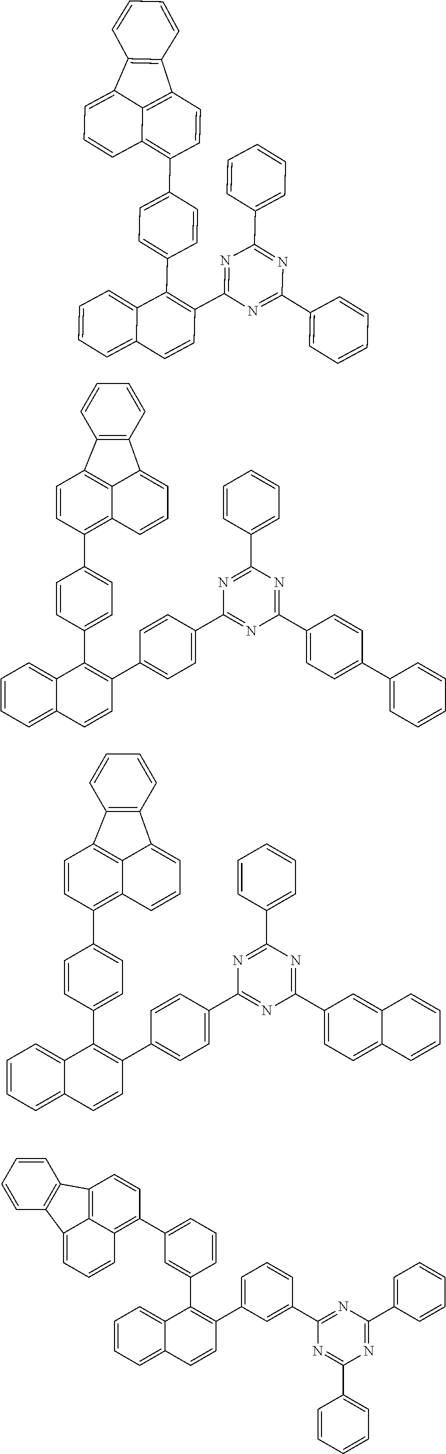

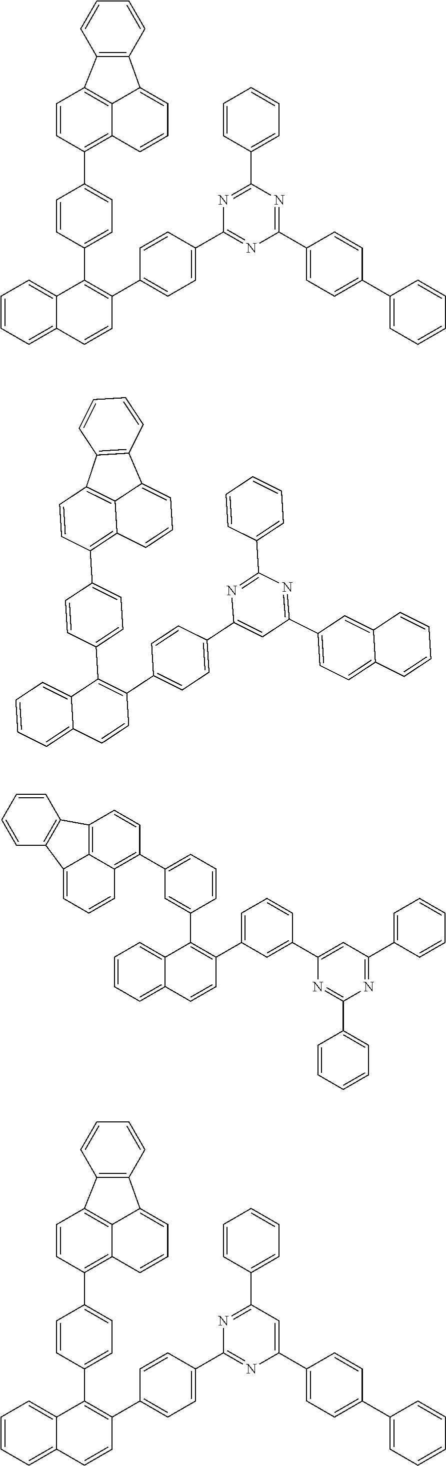 [SMILES: c1ccc(-c2ccc(-c3nc(-c4ccccc4)nc(-c4ccc(-c5ccc6ccccc6c5-c5ccc(-c6ccc7c8c(cccc68)-c6ccccc6-7)cc5)cc4)n3)cc2)cc1 ; c1ccc(-c2nc(-c3ccc(-c4ccc5ccccc5c4-c4ccc(-c5ccc6c7c(cccc57)-c5ccccc5-6)cc4)cc3)cc(-c3ccc4ccccc4c3)n2)cc1 ; c1ccc(-c2cc(-c3cccc(-c4ccc5ccccc5c4-c4cccc(-c5ccc6c7c(cccc57)-c5ccccc5-6)c4)c3)nc(-c3ccccc3)n2)cc1 ; c1ccc(-c2ccc(-c3cc(-c4ccccc4)nc(-c4ccc(-c5ccc6ccccc6c5-c5ccc(-c6ccc7c8c(cccc68)-c6ccccc6-7)cc5)cc4)n3)cc2)cc1]

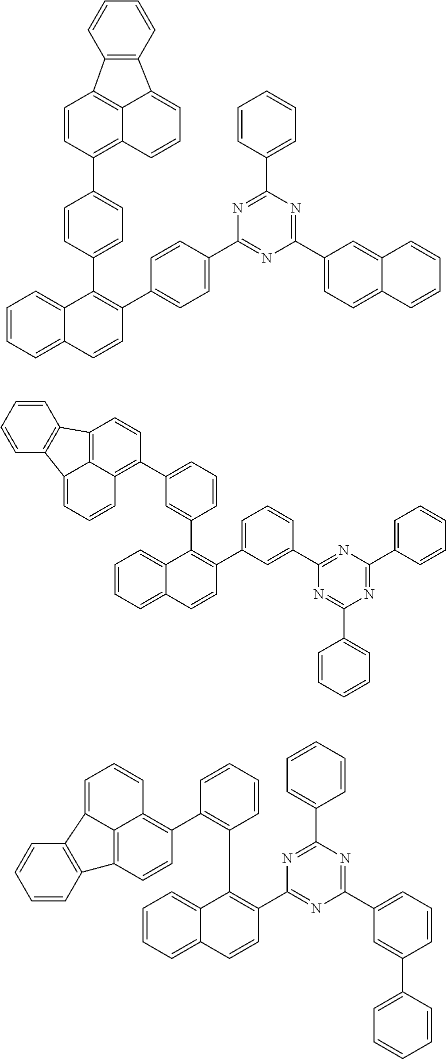

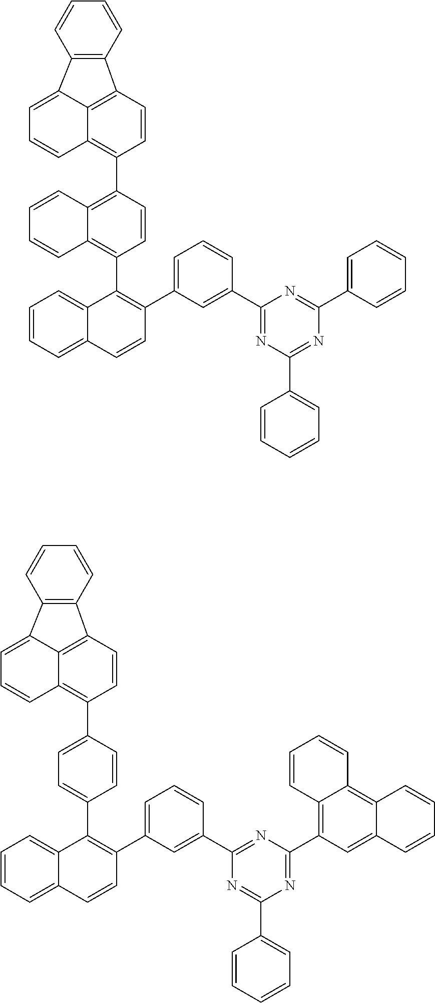 [SMILES: c1ccc(-c2nc(-c3ccccc3)nc(-c3cccc(-c4ccc5ccccc5c4-c4ccc(-c5ccc6c7c(cccc57)-c5ccccc5-6)c5ccccc45)c3)n2)cc1 ; c1ccc(-c2nc(-c3cccc(-c4ccc5ccccc5c4-c4ccc(-c5ccc6c7c(cccc57)-c5ccccc5-6)cc4)c3)nc(-c3cc4ccccc4c4ccccc34)n2)cc1]

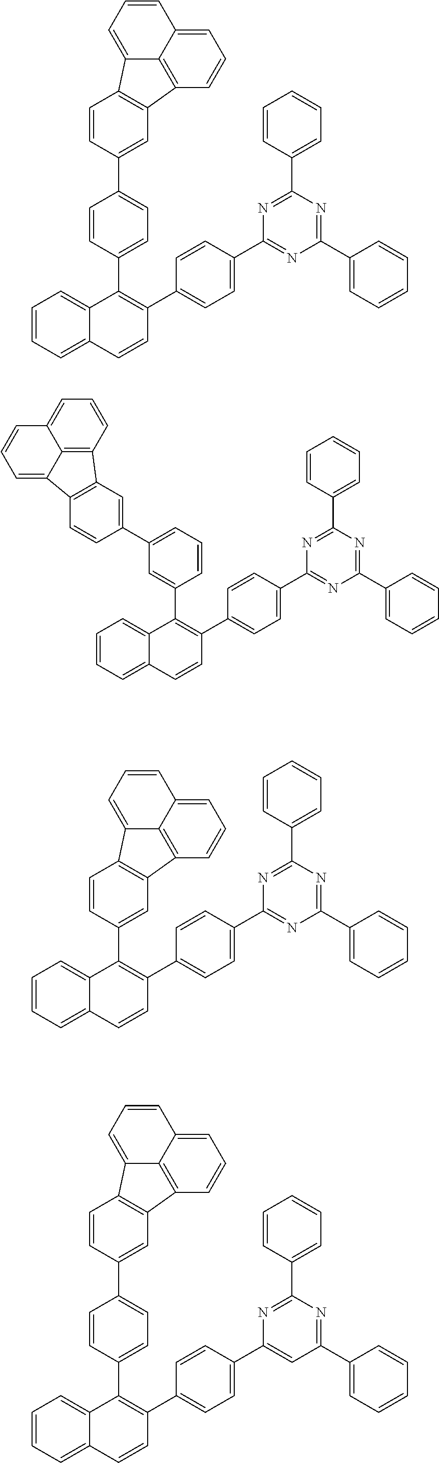

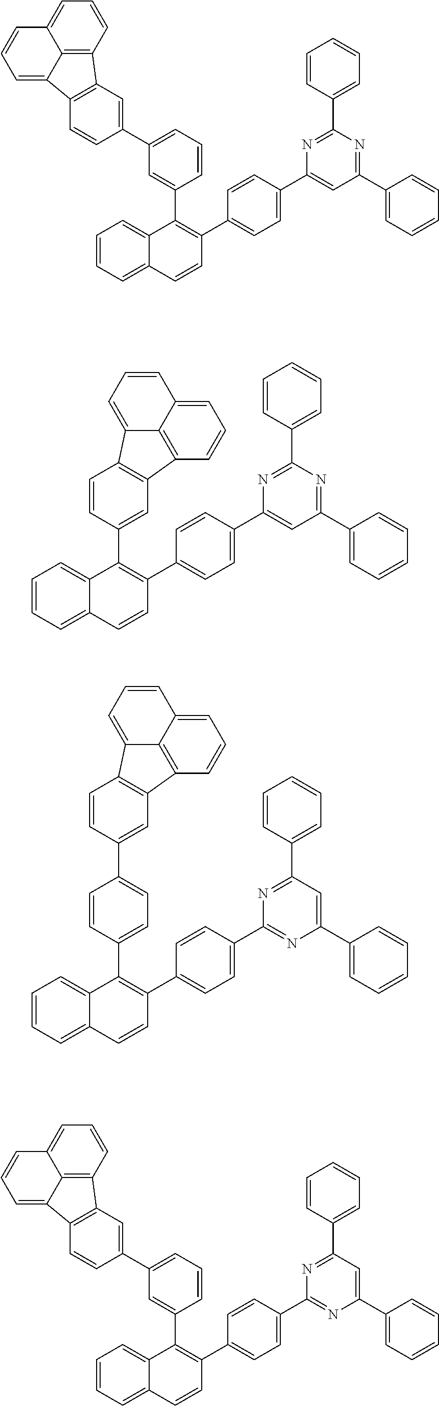 [SMILES: c1ccc(-c2cc(-c3ccc(-c4ccc5ccccc5c4-c4cccc(-c5ccc6c(c5)-c5cccc7cccc-6c57)c4)cc3)nc(-c3ccccc3)n2)cc1 ; c1ccc(-c2cc(-c3ccc(-c4ccc5ccccc5c4-c4ccc5c(c4)-c4cccc6cccc-5c46)cc3)nc(-c3ccccc3)n2)cc1 ; c1ccc(-c2cc(-c3ccccc3)nc(-c3ccc(-c4ccc5ccccc5c4-c4ccc(-c5ccc6c(c5)-c5cccc7cccc-6c57)cc4)cc3)n2)cc1 ; c1ccc(-c2cc(-c3ccccc3)nc(-c3ccc(-c4ccc5ccccc5c4-c4cccc(-c5ccc6c(c5)-c5cccc7cccc-6c57)c4)cc3)n2)cc1]

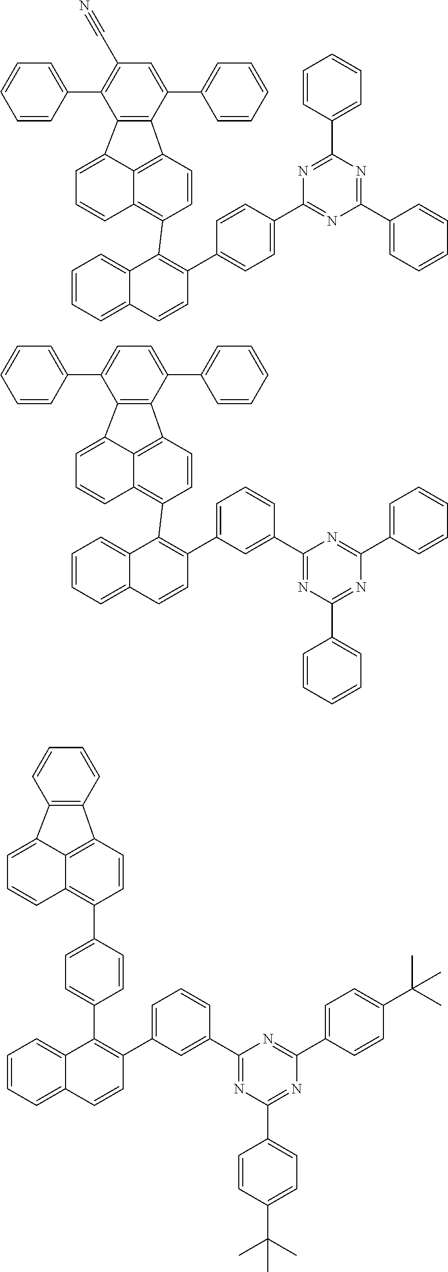

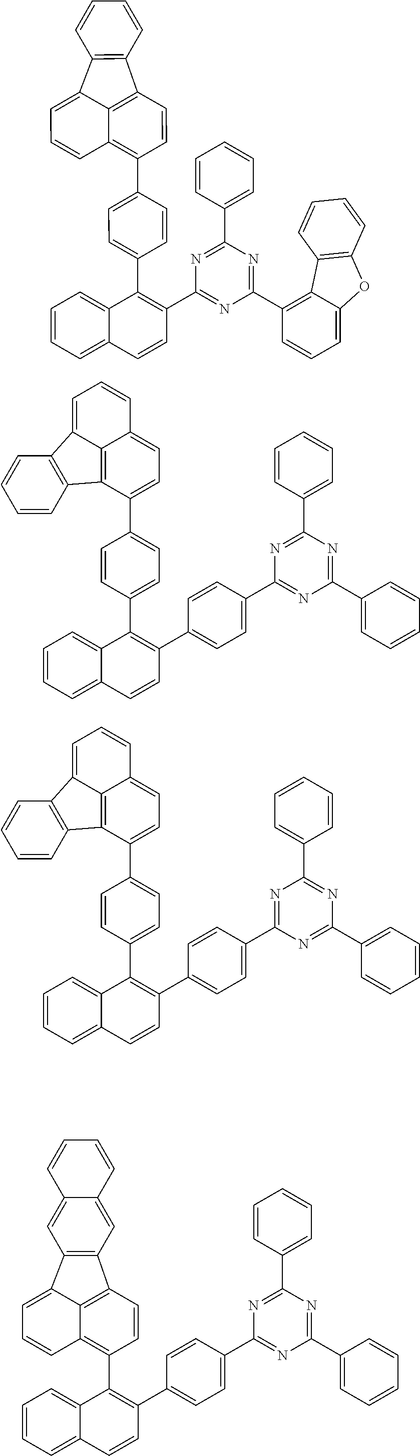

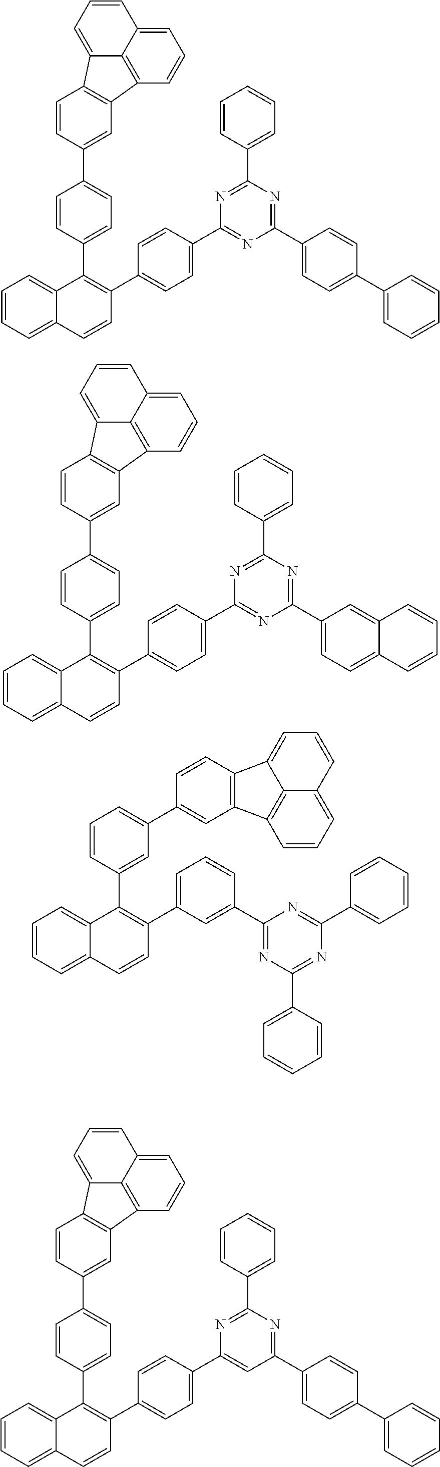

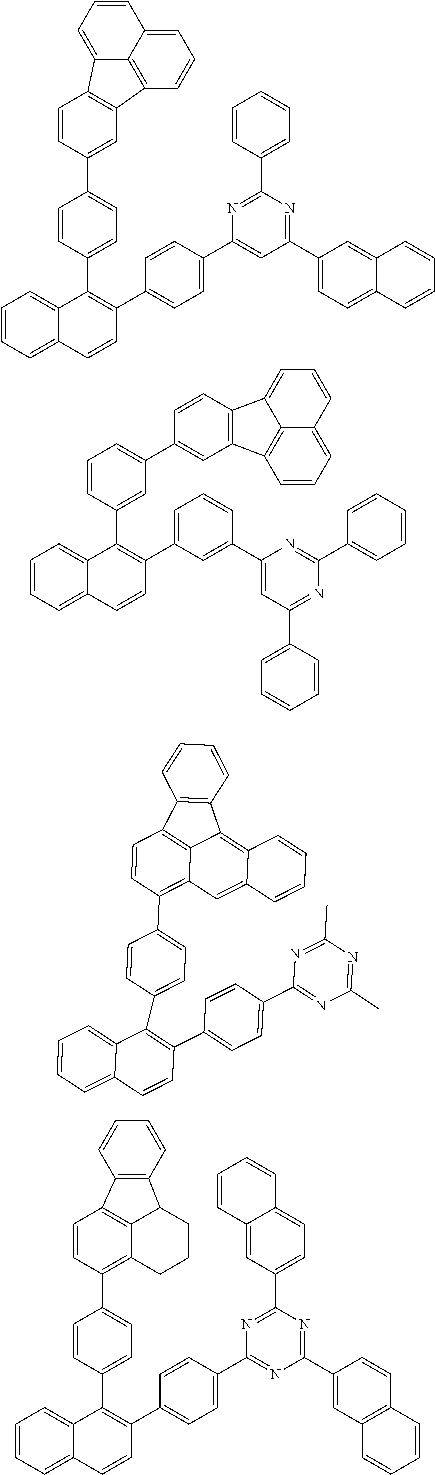

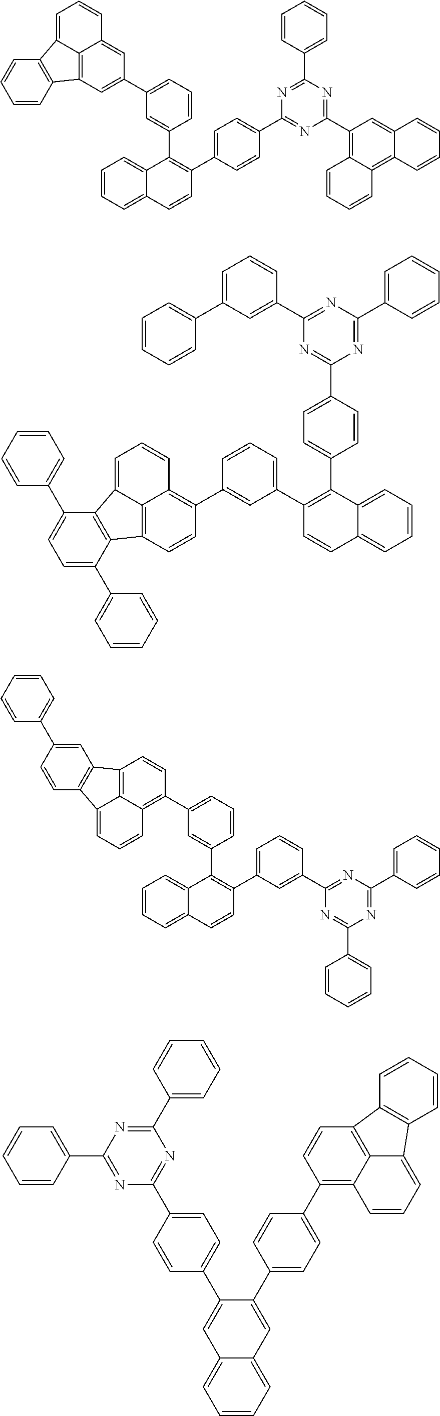 [SMILES: c1ccc(-c2nc(-c3ccc(-c4ccc5ccccc5c4-c4cccc(-c5cc6c7c(cccc7c5)-c5ccccc5-6)c4)cc3)nc(-c3cc4ccccc4c4ccccc34)n2)cc1 ; c1ccc(-c2cccc(-c3nc(-c4ccccc4)nc(-c4ccc(-c5c(-c6cccc(-c7ccc8c9c(cccc79)-c7c(-c9ccccc9)ccc(-c9ccccc9)c7-8)c6)ccc6ccccc56)cc4)n3)c2)cc1 ; c1ccc(-c2ccc3c(c2)-c2ccc(-c4cccc(-c5c(-c6cccc(-c7nc(-c8ccccc8)nc(-c8ccccc8)n7)c6)ccc6ccccc56)c4)c4cccc-3c24)cc1 ; c1ccc(-c2nc(-c3ccccc3)nc(-c3ccc(-c4cc5ccccc5cc4-c4ccc(-c5ccc6c7c(cccc57)-c5ccccc5-6)cc4)cc3)n2)cc1]

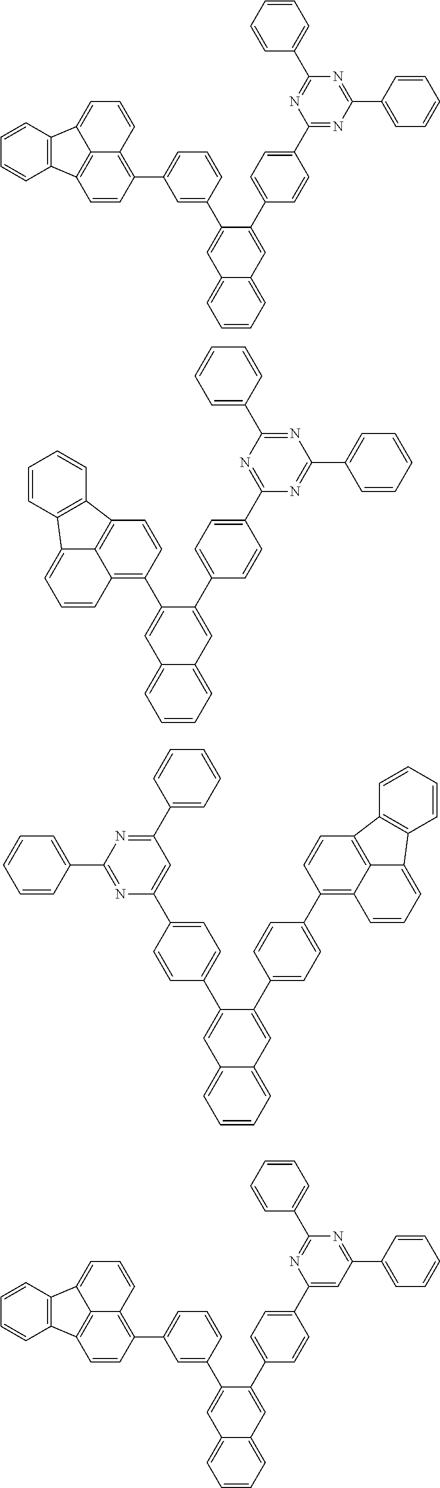

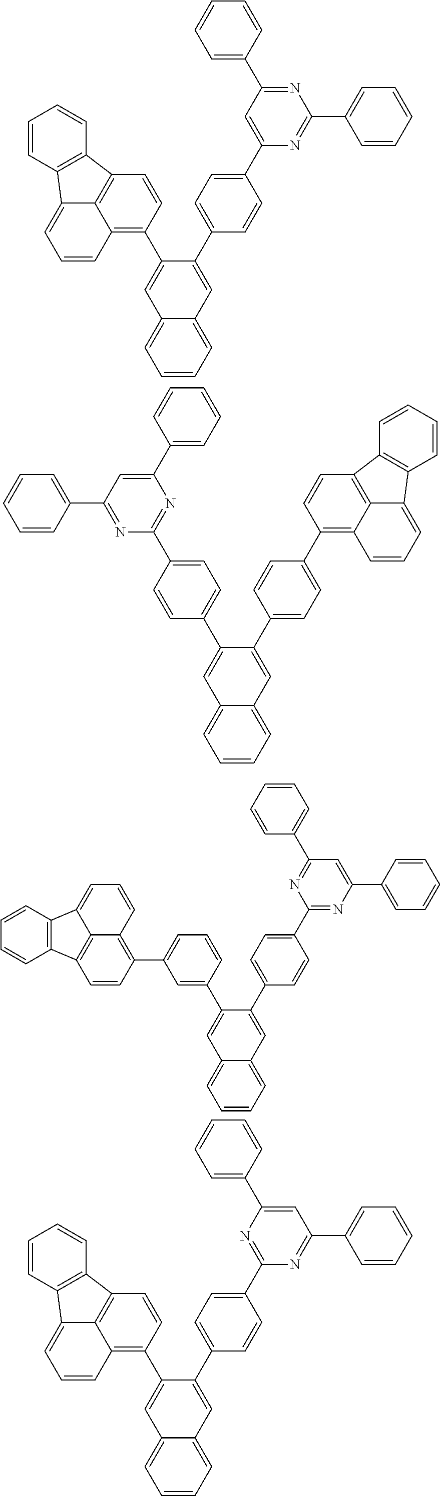

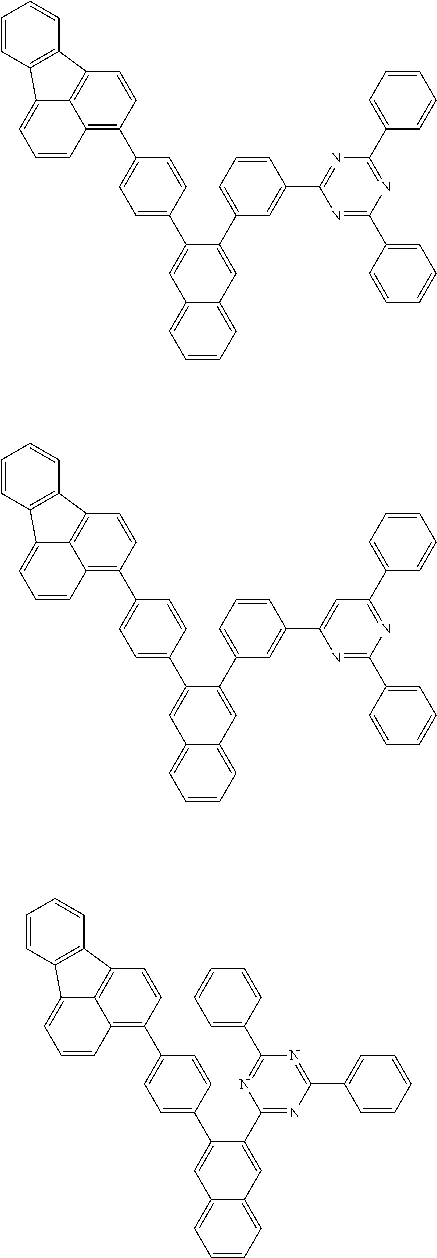 [SMILES: c1ccc(-c2nc(-c3ccccc3)nc(-c3cccc(-c4cc5ccccc5cc4-c4ccc(-c5ccc6c7c(cccc57)-c5ccccc5-6)cc4)c3)n2)cc1 ; c1ccc(-c2cc(-c3cccc(-c4cc5ccccc5cc4-c4ccc(-c5ccc6c7c(cccc57)-c5ccccc5-6)cc4)c3)nc(-c3ccccc3)n2)cc1 ; c1ccc(-c2nc(-c3ccccc3)nc(-c3cc4ccccc4cc3-c3ccc(-c4ccc5c6c(cccc46)-c4ccccc4-5)cc3)n2)cc1]

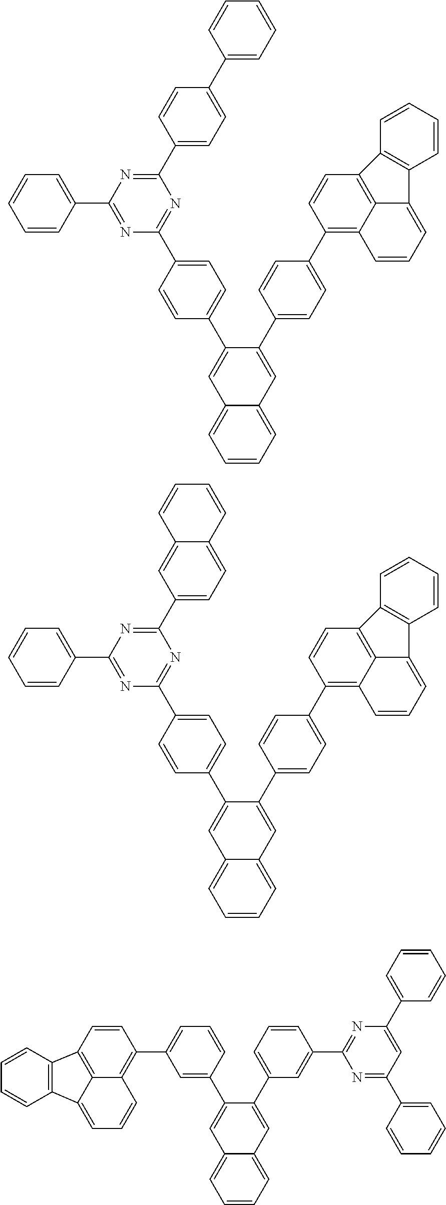

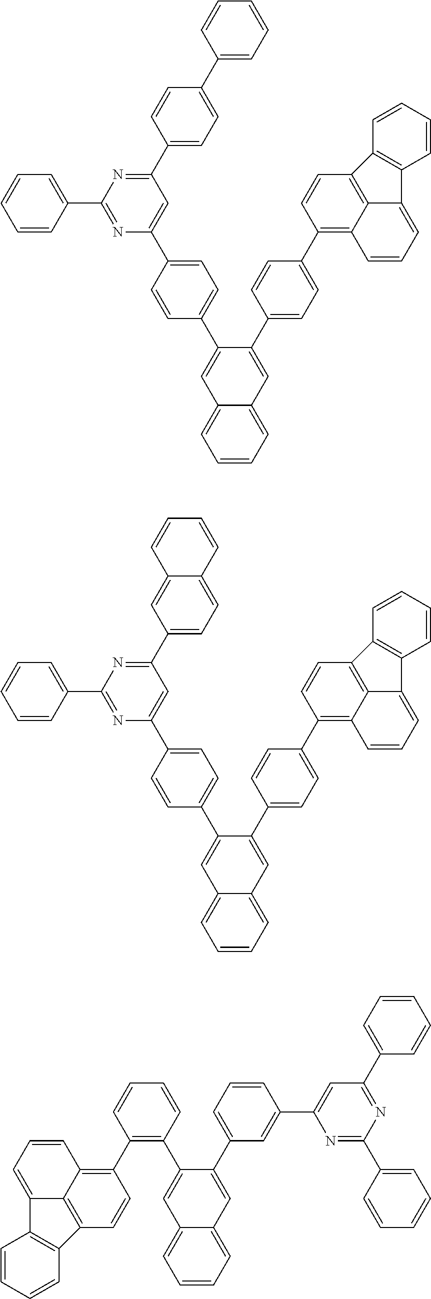 [SMILES: c1ccc(-c2ccc(-c3cc(-c4ccc(-c5cc6ccccc6cc5-c5ccc(-c6ccc7c8c(cccc68)-c6ccccc6-7)cc5)cc4)nc(-c4ccccc4)n3)cc2)cc1 ; c1ccc(-c2nc(-c3ccc(-c4cc5ccccc5cc4-c4ccc(-c5ccc6c7c(cccc57)-c5ccccc5-6)cc4)cc3)cc(-c3ccc4ccccc4c3)n2)cc1 ; c1ccc(-c2cc(-c3cccc(-c4cc5ccccc5cc4-c4ccccc4-c4ccc5c6c(cccc46)-c4ccccc4-5)c3)nc(-c3ccccc3)n2)cc1]

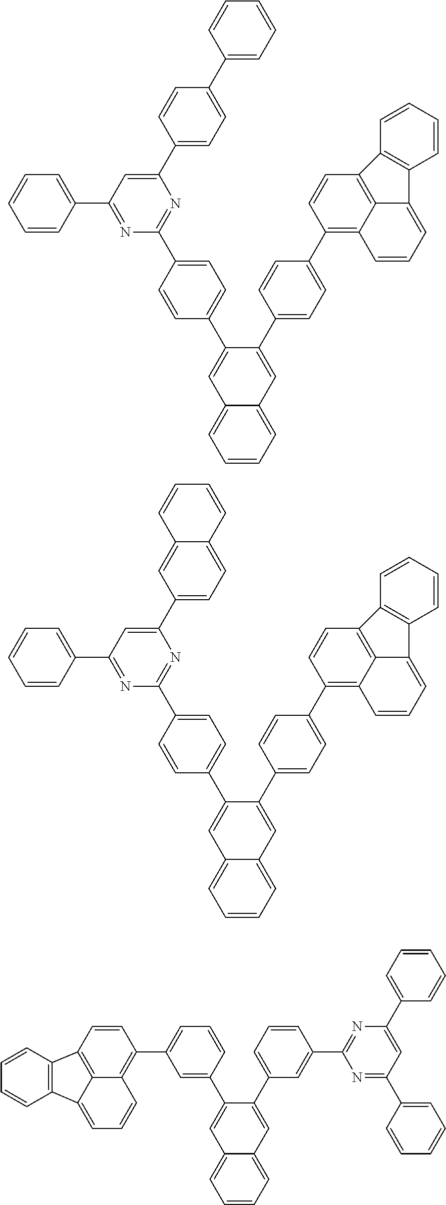

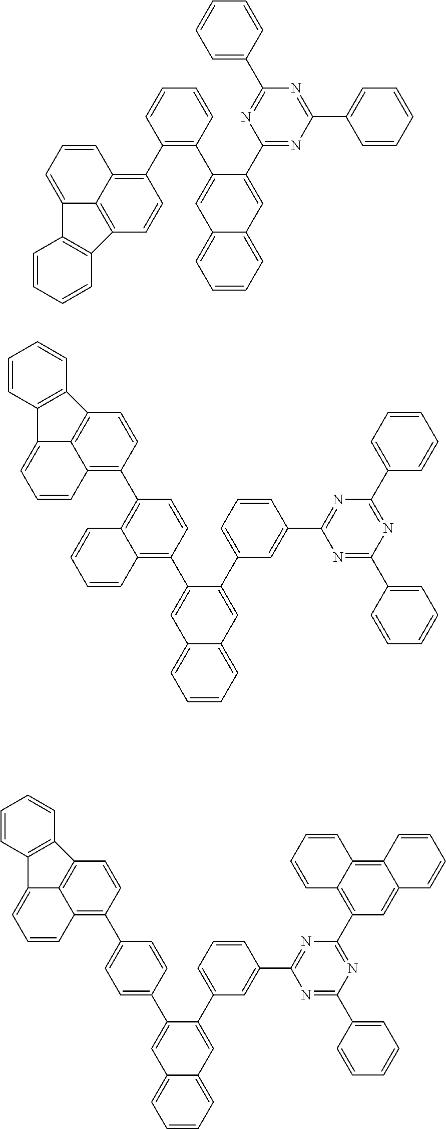

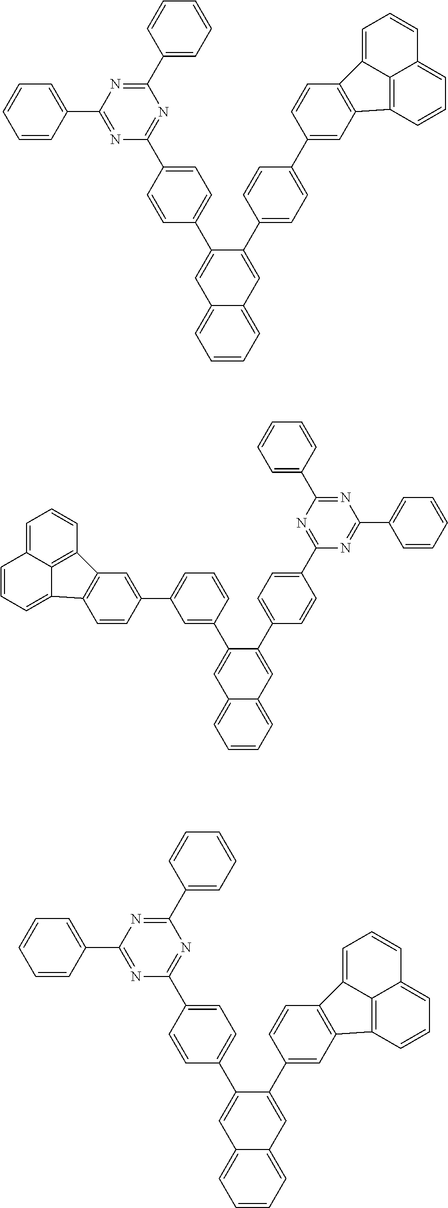 [SMILES: c1ccc(-c2nc(-c3ccccc3)nc(-c3ccc(-c4cc5ccccc5cc4-c4ccc(-c5ccc6c(c5)-c5cccc7cccc-6c57)cc4)cc3)n2)cc1 ; c1ccc(-c2nc(-c3ccccc3)nc(-c3ccc(-c4cc5ccccc5cc4-c4cccc(-c5ccc6c(c5)-c5cccc7cccc-6c57)c4)cc3)n2)cc1 ; c1ccc(-c2nc(-c3ccccc3)nc(-c3ccc(-c4cc5ccccc5cc4-c4ccc5c(c4)-c4cccc6cccc-5c46)cc3)n2)cc1]

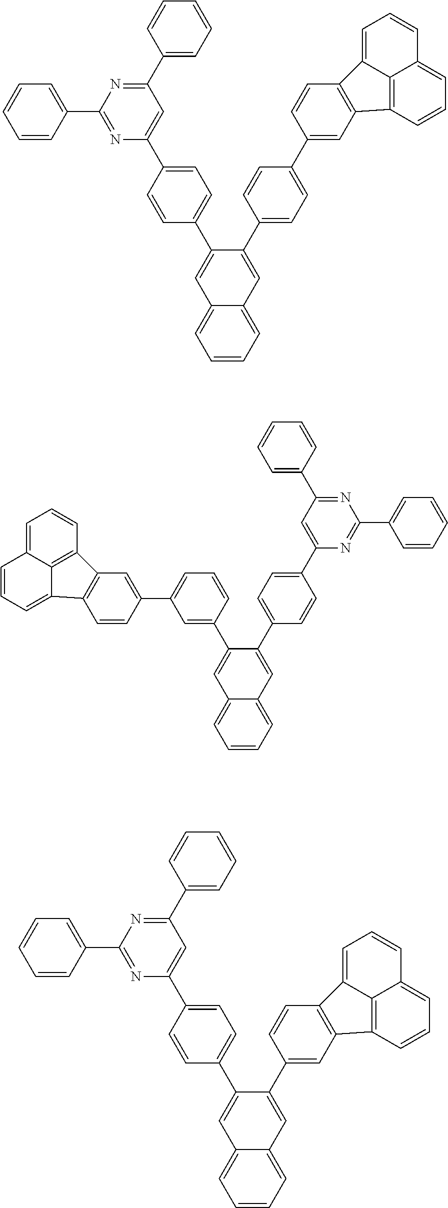 [SMILES: c1ccc(-c2cc(-c3ccc(-c4cc5ccccc5cc4-c4ccc(-c5ccc6c(c5)-c5cccc7cccc-6c57)cc4)cc3)nc(-c3ccccc3)n2)cc1 ; c1ccc(-c2cc(-c3ccc(-c4cc5ccccc5cc4-c4cccc(-c5ccc6c(c5)-c5cccc7cccc-6c57)c4)cc3)nc(-c3ccccc3)n2)cc1 ; c1ccc(-c2cc(-c3ccc(-c4cc5ccccc5cc4-c4ccc5c(c4)-c4cccc6cccc-5c46)cc3)nc(-c3ccccc3)n2)cc1]

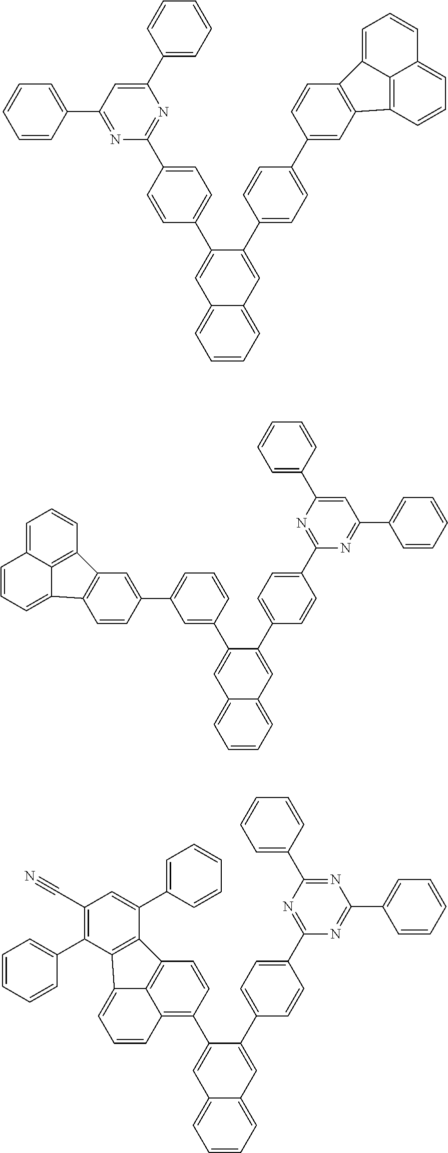 [SMILES: c1ccc(-c2cc(-c3ccccc3)nc(-c3ccc(-c4cc5ccccc5cc4-c4ccc(-c5ccc6c(c5)-c5cccc7cccc-6c57)cc4)cc3)n2)cc1 ; c1ccc(-c2cc(-c3ccccc3)nc(-c3ccc(-c4cc5ccccc5cc4-c4cccc(-c5ccc6c(c5)-c5cccc7cccc-6c57)c4)cc3)n2)cc1 ; N#Cc1cc(-c2ccccc2)c2c(c1-c1ccccc1)-c1cccc3c(-c4cc5ccccc5cc4-c4ccc(-c5nc(-c6ccccc6)nc(-c6ccccc6)n5)cc4)ccc-2c13]

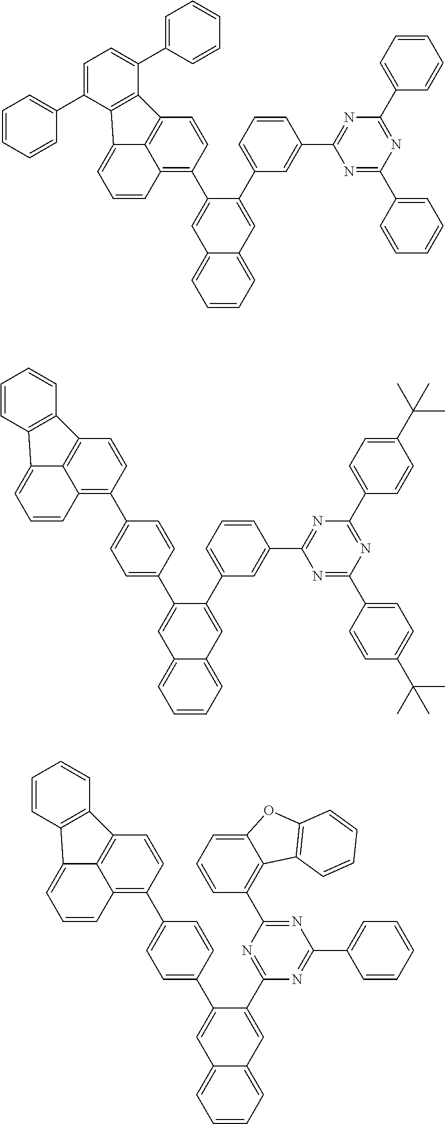 [SMILES: c1ccc(-c2nc(-c3ccccc3)nc(-c3cccc(-c4cc5ccccc5cc4-c4ccc5c6c(cccc46)-c4c(-c6ccccc6)ccc(-c6ccccc6)c4-5)c3)n2)cc1 ; CC(C)(C)c1ccc(-c2nc(-c3ccc(C(C)(C)C)cc3)nc(-c3cccc(-c4cc5ccccc5cc4-c4ccc(-c5ccc6c7c(cccc57)-c5ccccc5-6)cc4)c3)n2)cc1 ; c1ccc(-c2nc(-c3cc4ccccc4cc3-c3ccc(-c4ccc5c6c(cccc46)-c4ccccc4-5)cc3)nc(-c3cccc4oc5ccccc5c34)n2)cc1]

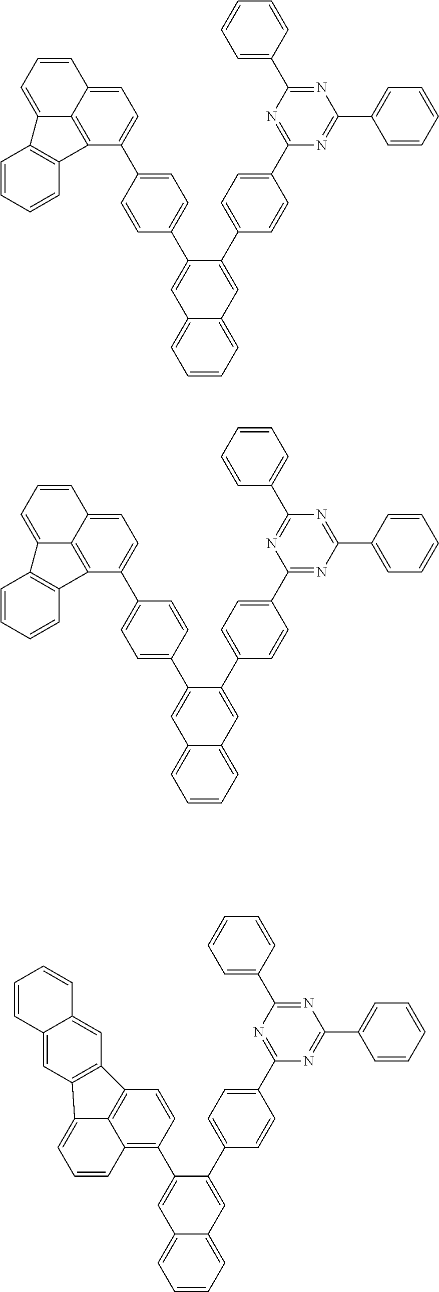 [SMILES: c1ccc(-c2nc(-c3ccccc3)nc(-c3ccc(-c4cc5ccccc5cc4-c4ccc(-c5ccc6cccc7c6c5-c5ccccc5-7)cc4)cc3)n2)cc1 ; c1ccc(-c2nc(-c3ccccc3)nc(-c3ccc(-c4cc5ccccc5cc4-c4ccc(-c5ccc6cccc7c6c5-c5ccccc5-7)cc4)cc3)n2)cc1 ; c1ccc(-c2nc(-c3ccccc3)nc(-c3ccc(-c4cc5ccccc5cc4-c4ccc5c6c(cccc46)-c4cc6ccccc6cc4-5)cc3)n2)cc1]

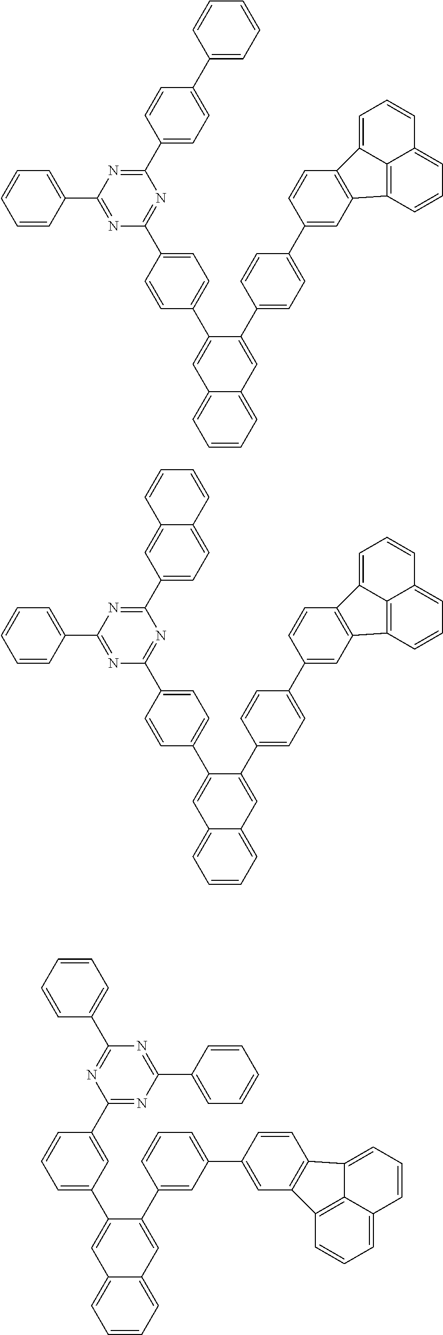

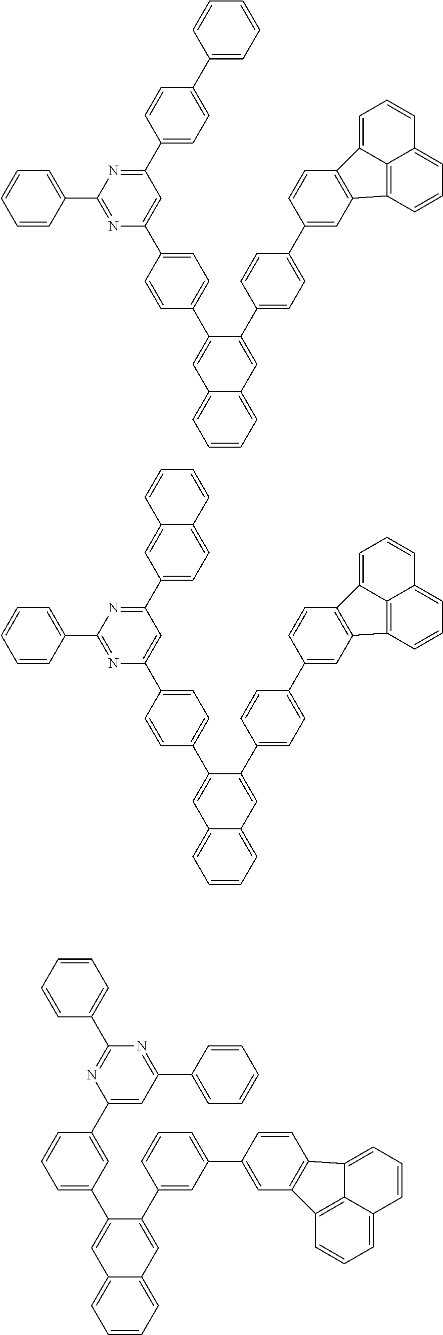

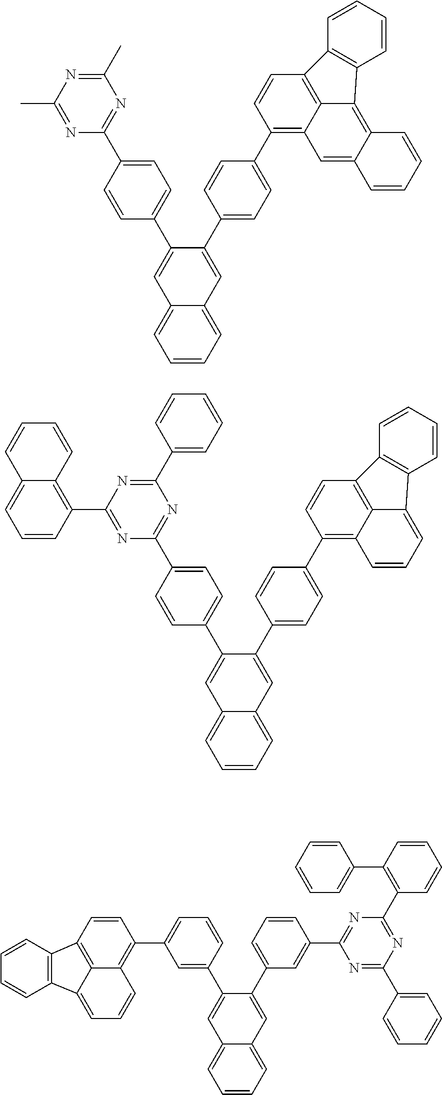

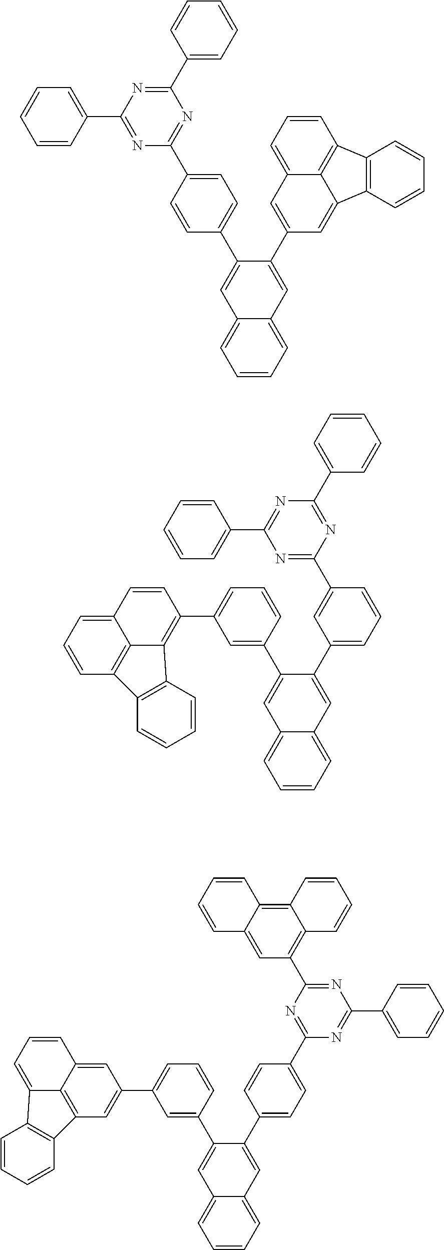 [SMILES: c1ccc(-c2nc(-c3ccccc3)nc(-c3ccc(-c4cc5ccccc5cc4-c4cc5c6c(cccc6c4)-c4ccccc4-5)cc3)n2)cc1 ; c1ccc(-c2nc(-c3ccccc3)nc(-c3cccc(-c4cc5ccccc5cc4-c4cccc(-c5ccc6cccc7c6c5-c5ccccc5-7)c4)c3)n2)cc1 ; c1ccc(-c2nc(-c3ccc(-c4cc5ccccc5cc4-c4cccc(-c5cc6c7c(cccc7c5)-c5ccccc5-6)c4)cc3)nc(-c3cc4ccccc4c4ccccc34)n2)cc1]

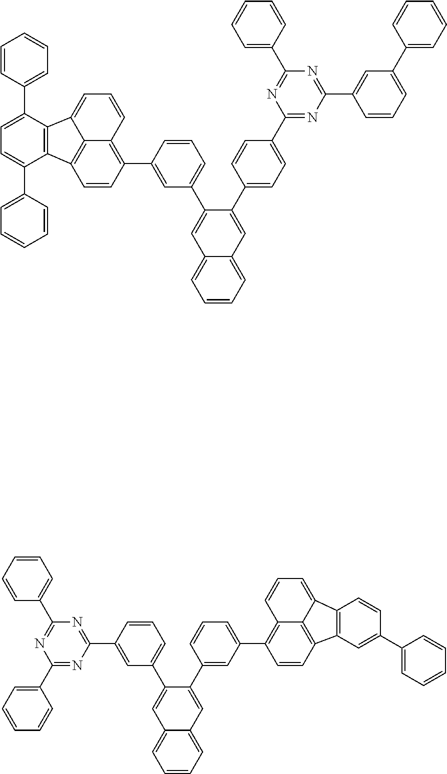 [SMILES: c1ccc(-c2cccc(-c3nc(-c4ccccc4)nc(-c4ccc(-c5cc6ccccc6cc5-c5cccc(-c6ccc7c8c(cccc68)-c6c(-c8ccccc8)ccc(-c8ccccc8)c6-7)c5)cc4)n3)c2)cc1 ; c1ccc(-c2ccc3c(c2)-c2ccc(-c4cccc(-c5cc6ccccc6cc5-c5cccc(-c6nc(-c7ccccc7)nc(-c7ccccc7)n6)c5)c4)c4cccc-3c24)cc1]

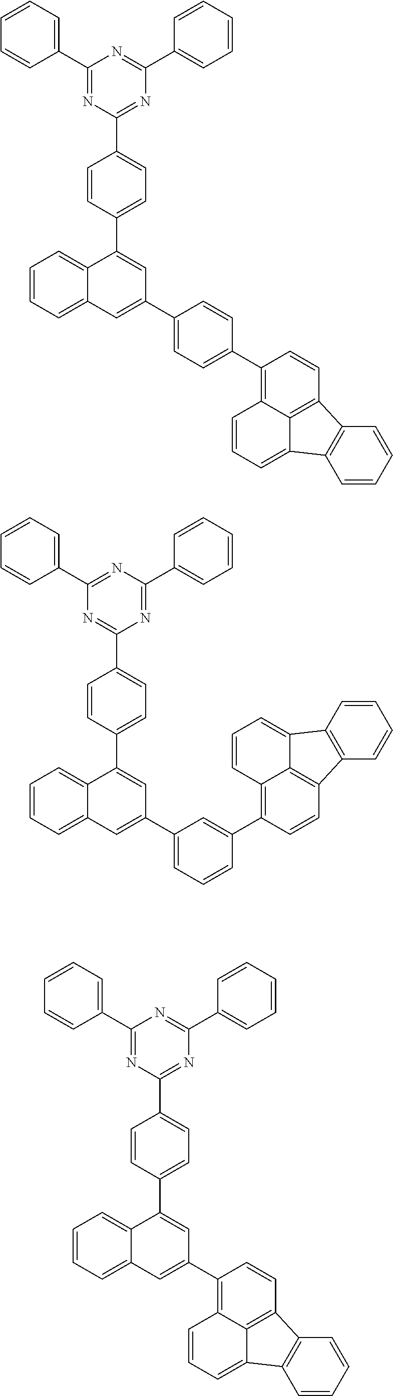 [SMILES: c1ccc(-c2nc(-c3ccccc3)nc(-c3ccc(-c4cc(-c5ccc(-c6ccc7c8c(cccc68)-c6ccccc6-7)cc5)cc5ccccc45)cc3)n2)cc1 ; c1ccc(-c2nc(-c3ccccc3)nc(-c3ccc(-c4cc(-c5cccc(-c6ccc7c8c(cccc68)-c6ccccc6-7)c5)cc5ccccc45)cc3)n2)cc1 ; c1ccc(-c2nc(-c3ccccc3)nc(-c3ccc(-c4cc(-c5ccc6c7c(cccc57)-c5ccccc5-6)cc5ccccc45)cc3)n2)cc1]

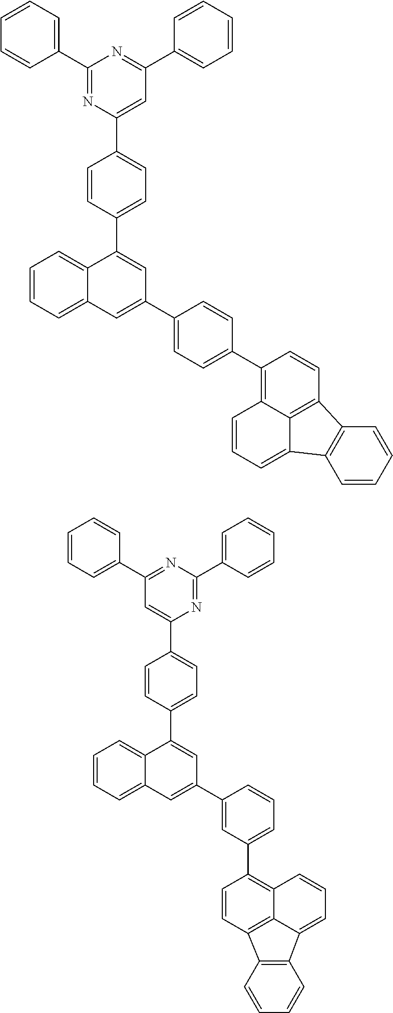 [SMILES: c1ccc(-c2cc(-c3ccc(-c4cc(-c5ccc(-c6ccc7c8c(cccc68)-c6ccccc6-7)cc5)cc5ccccc45)cc3)nc(-c3ccccc3)n2)cc1 ; c1ccc(-c2cc(-c3ccc(-c4cc(-c5cccc(-c6ccc7c8c(cccc68)-c6ccccc6-7)c5)cc5ccccc45)cc3)nc(-c3ccccc3)n2)cc1]

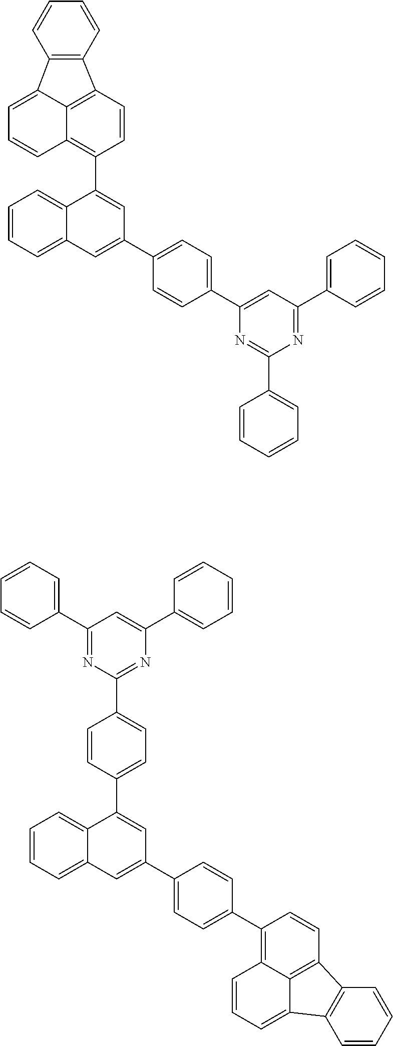 [SMILES: c1ccc(-c2cc(-c3ccc(-c4cc(-c5ccc6c7c(cccc57)-c5ccccc5-6)c5ccccc5c4)cc3)nc(-c3ccccc3)n2)cc1 ; c1ccc(-c2cc(-c3ccccc3)nc(-c3ccc(-c4cc(-c5ccc(-c6ccc7c8c(cccc68)-c6ccccc6-7)cc5)cc5ccccc45)cc3)n2)cc1]

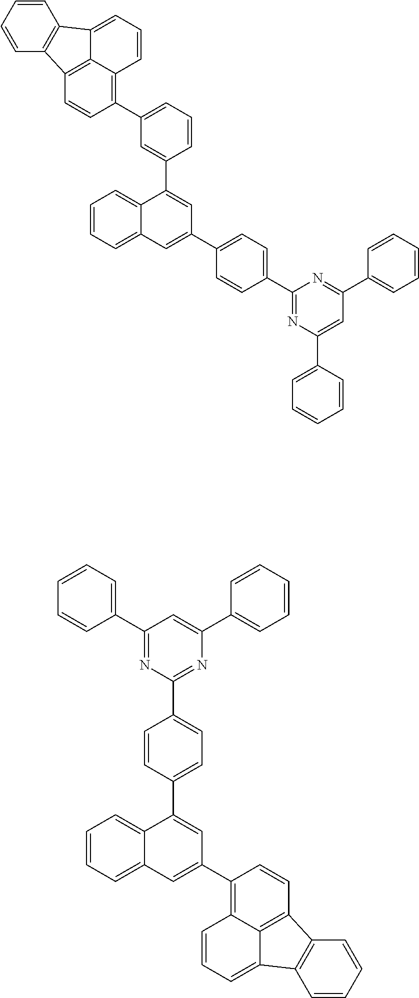 [SMILES: c1ccc(-c2cc(-c3ccccc3)nc(-c3ccc(-c4cc(-c5cccc(-c6ccc7c8c(cccc68)-c6ccccc6-7)c5)c5ccccc5c4)cc3)n2)cc1 ; c1ccc(-c2cc(-c3ccccc3)nc(-c3ccc(-c4cc(-c5ccc6c7c(cccc57)-c5ccccc5-6)cc5ccccc45)cc3)n2)cc1]

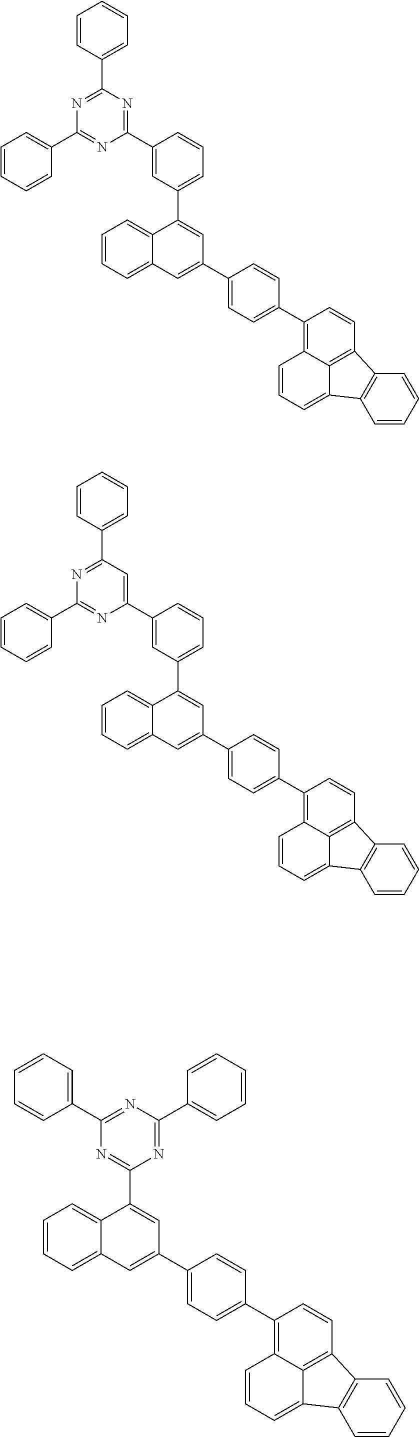 [SMILES: c1ccc(-c2nc(-c3ccccc3)nc(-c3cccc(-c4cc(-c5ccc(-c6ccc7c8c(cccc68)-c6ccccc6-7)cc5)cc5ccccc45)c3)n2)cc1 ; c1ccc(-c2cc(-c3cccc(-c4cc(-c5ccc(-c6ccc7c8c(cccc68)-c6ccccc6-7)cc5)cc5ccccc45)c3)nc(-c3ccccc3)n2)cc1 ; c1ccc(-c2nc(-c3ccccc3)nc(-c3cc(-c4ccc(-c5ccc6c7c(cccc57)-c5ccccc5-6)cc4)cc4ccccc34)n2)cc1]

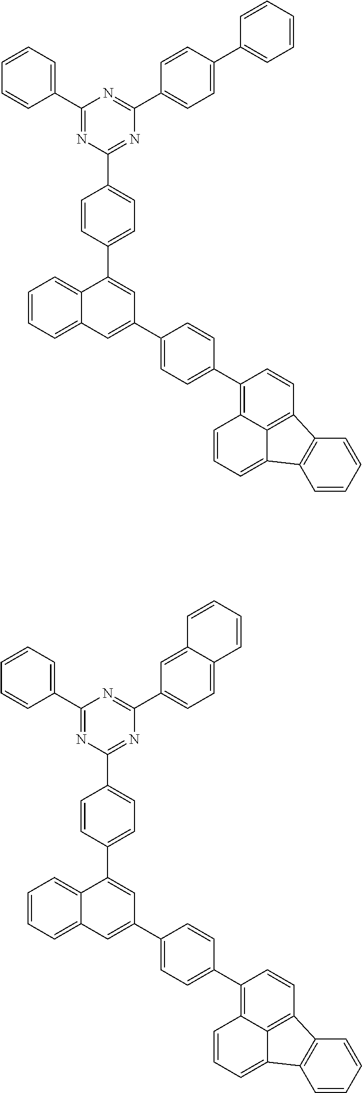 [SMILES: c1ccc(-c2ccc(-c3nc(-c4ccccc4)nc(-c4ccc(-c5cc(-c6ccc(-c7ccc8c9c(cccc79)-c7ccccc7-8)cc6)cc6ccccc56)cc4)n3)cc2)cc1 ; c1ccc(-c2nc(-c3ccc(-c4cc(-c5ccc(-c6ccc7c8c(cccc68)-c6ccccc6-7)cc5)cc5ccccc45)cc3)nc(-c3ccc4ccccc4c3)n2)cc1]

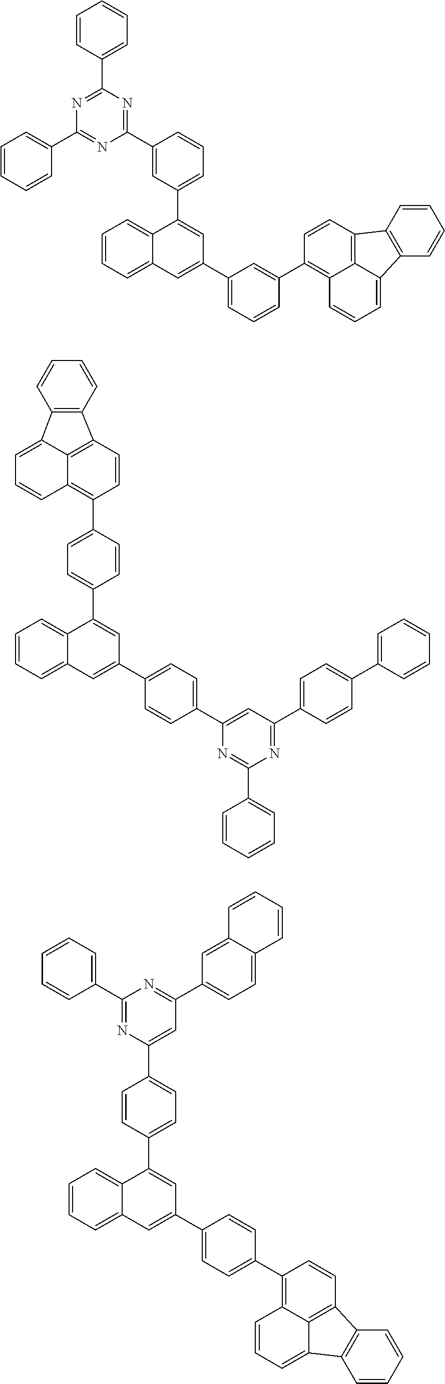

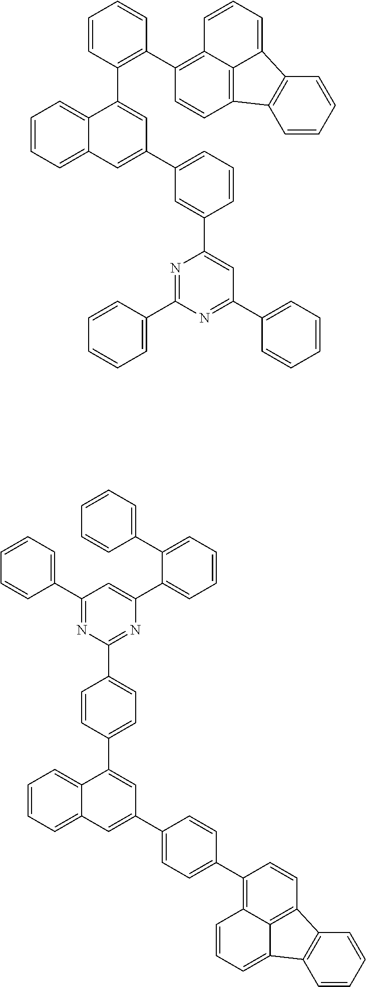 [SMILES: c1ccc(-c2cc(-c3cccc(-c4cc(-c5ccccc5-c5ccc6c7c(cccc57)-c5ccccc5-6)c5ccccc5c4)c3)nc(-c3ccccc3)n2)cc1 ; c1ccc(-c2cc(-c3ccccc3-c3ccccc3)nc(-c3ccc(-c4cc(-c5ccc(-c6ccc7c8c(cccc68)-c6ccccc6-7)cc5)cc5ccccc45)cc3)n2)cc1]

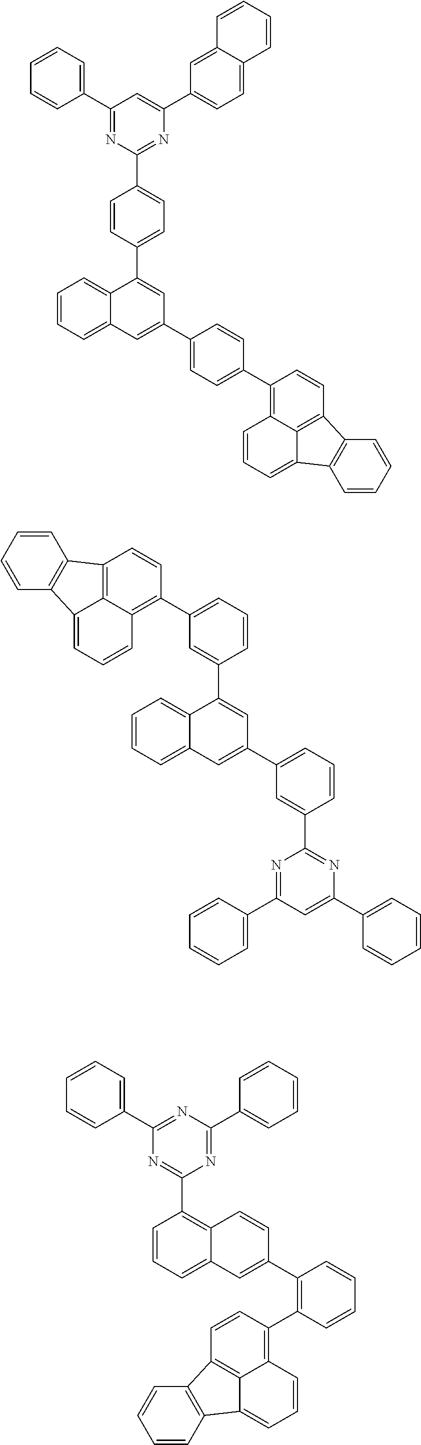 [SMILES: c1ccc(-c2cc(-c3ccc4ccccc4c3)nc(-c3ccc(-c4cc(-c5ccc(-c6ccc7c8c(cccc68)-c6ccccc6-7)cc5)cc5ccccc45)cc3)n2)cc1 ; c1ccc(-c2cc(-c3ccccc3)nc(-c3cccc(-c4cc(-c5cccc(-c6ccc7c8c(cccc68)-c6ccccc6-7)c5)c5ccccc5c4)c3)n2)cc1 ; c1ccc(-c2nc(-c3ccccc3)nc(-c3cccc4cc(-c5ccccc5-c5ccc6c7c(cccc57)-c5ccccc5-6)ccc34)n2)cc1]

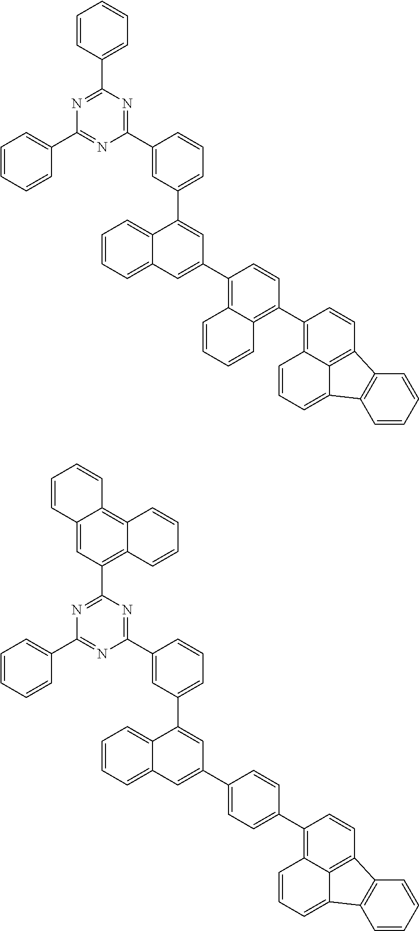 [SMILES: c1ccc(-c2nc(-c3ccccc3)nc(-c3cccc(-c4cc(-c5ccc(-c6ccc7c8c(cccc68)-c6ccccc6-7)c6ccccc56)cc5ccccc45)c3)n2)cc1 ; c1ccc(-c2nc(-c3cccc(-c4cc(-c5ccc(-c6ccc7c8c(cccc68)-c6ccccc6-7)cc5)cc5ccccc45)c3)nc(-c3cc4ccccc4c4ccccc34)n2)cc1]

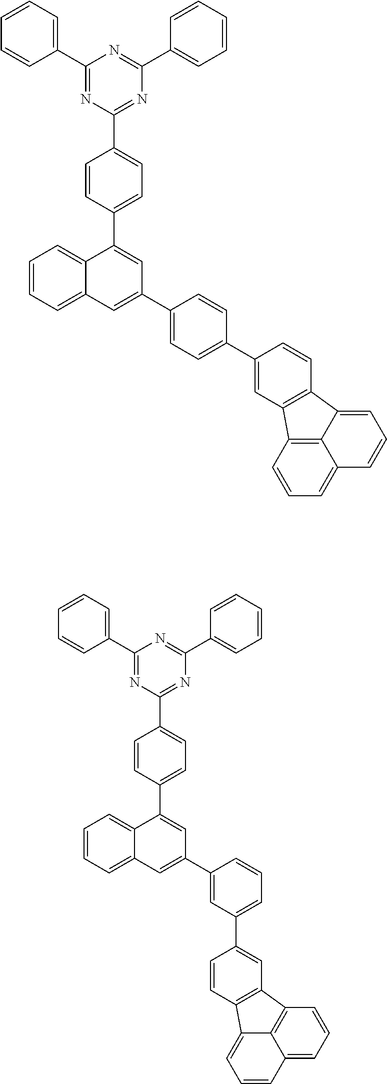 [SMILES: c1ccc(-c2nc(-c3ccccc3)nc(-c3ccc(-c4cc(-c5ccc(-c6ccc7c(c6)-c6cccc8cccc-7c68)cc5)cc5ccccc45)cc3)n2)cc1 ; c1ccc(-c2nc(-c3ccccc3)nc(-c3ccc(-c4cc(-c5cccc(-c6ccc7c(c6)-c6cccc8cccc-7c68)c5)cc5ccccc45)cc3)n2)cc1]

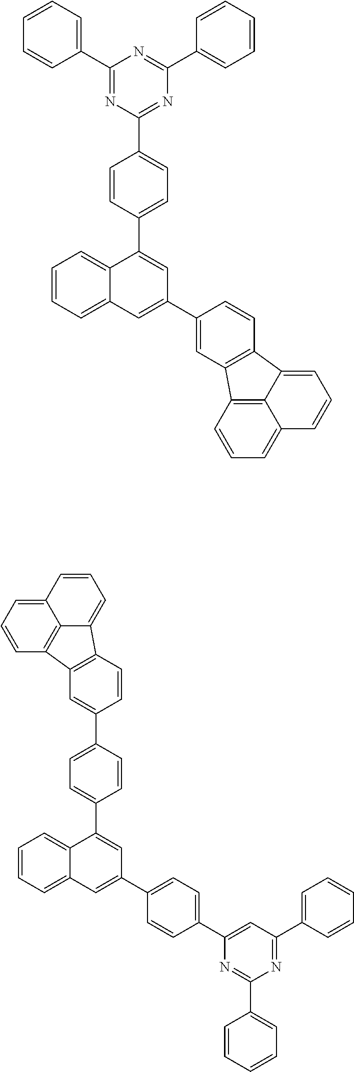 [SMILES: c1ccc(-c2nc(-c3ccccc3)nc(-c3ccc(-c4cc(-c5ccc6c(c5)-c5cccc7cccc-6c57)cc5ccccc45)cc3)n2)cc1 ; c1ccc(-c2cc(-c3ccc(-c4cc(-c5ccc(-c6ccc7c(c6)-c6cccc8cccc-7c68)cc5)c5ccccc5c4)cc3)nc(-c3ccccc3)n2)cc1]

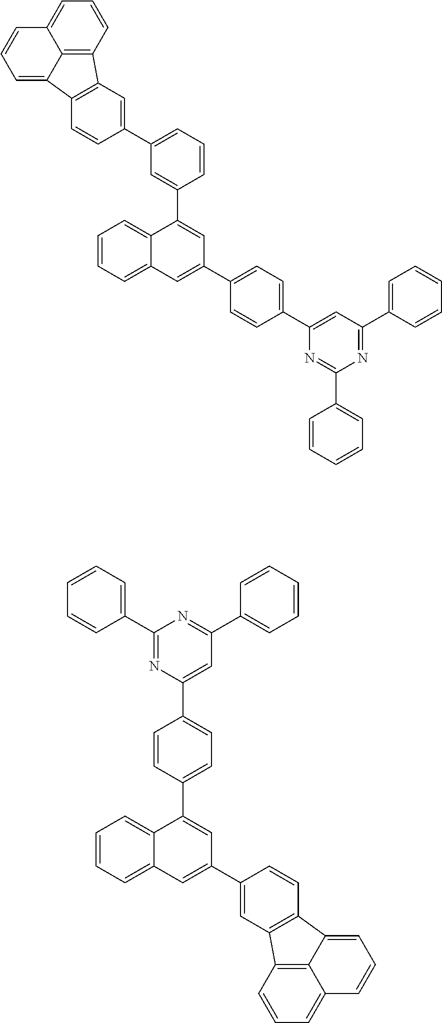 [SMILES: c1ccc(-c2cc(-c3ccc(-c4cc(-c5cccc(-c6ccc7c(c6)-c6cccc8cccc-7c68)c5)c5ccccc5c4)cc3)nc(-c3ccccc3)n2)cc1 ; c1ccc(-c2cc(-c3ccc(-c4cc(-c5ccc6c(c5)-c5cccc7cccc-6c57)cc5ccccc45)cc3)nc(-c3ccccc3)n2)cc1]

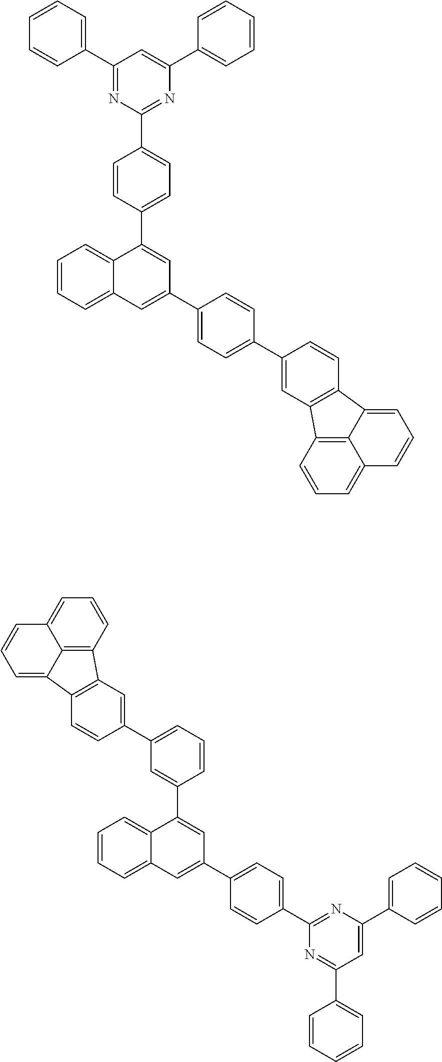 [SMILES: c1ccc(-c2cc(-c3ccccc3)nc(-c3ccc(-c4cc(-c5ccc(-c6ccc7c(c6)-c6cccc8cccc-7c68)cc5)cc5ccccc45)cc3)n2)cc1 ; c1ccc(-c2cc(-c3ccccc3)nc(-c3ccc(-c4cc(-c5cccc(-c6ccc7c(c6)-c6cccc8cccc-7c68)c5)c5ccccc5c4)cc3)n2)cc1]

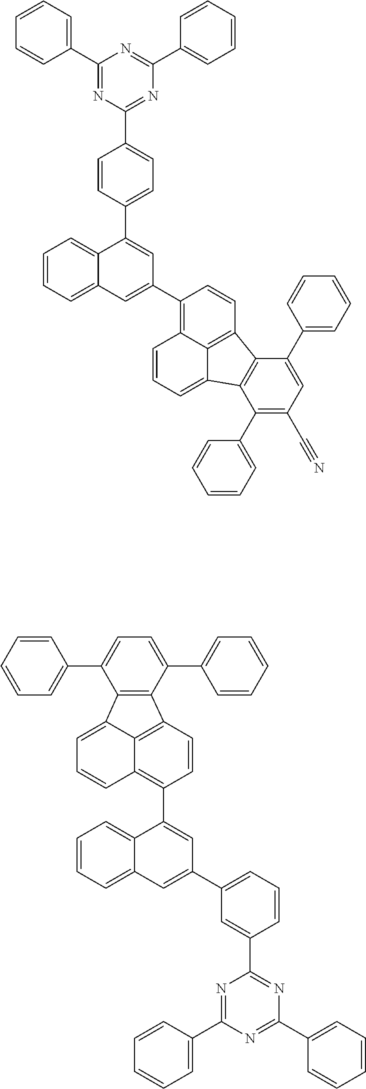 [SMILES: N#Cc1cc(-c2ccccc2)c2c(c1-c1ccccc1)-c1cccc3c(-c4cc(-c5ccc(-c6nc(-c7ccccc7)nc(-c7ccccc7)n6)cc5)c5ccccc5c4)ccc-2c13 ; c1ccc(-c2nc(-c3ccccc3)nc(-c3cccc(-c4cc(-c5ccc6c7c(cccc57)-c5c(-c7ccccc7)ccc(-c7ccccc7)c5-6)c5ccccc5c4)c3)n2)cc1]

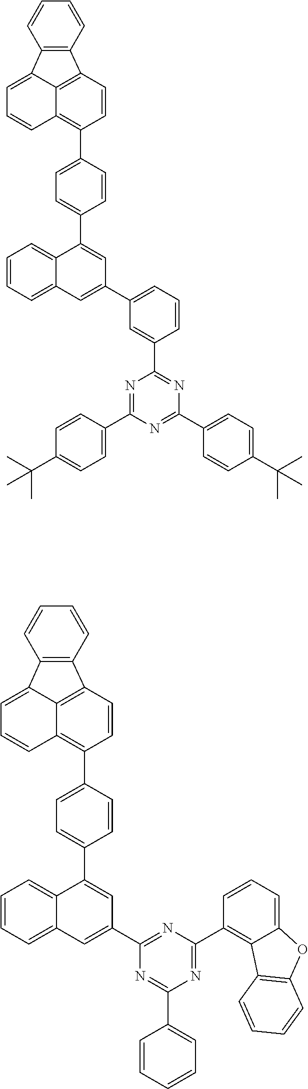 [SMILES: CC(C)(C)c1ccc(-c2nc(-c3ccc(C(C)(C)C)cc3)nc(-c3cccc(-c4cc(-c5ccc(-c6ccc7c8c(cccc68)-c6ccccc6-7)cc5)c5ccccc5c4)c3)n2)cc1 ; c1ccc(-c2nc(-c3cc(-c4ccc(-c5ccc6c7c(cccc57)-c5ccccc5-6)cc4)c4ccccc4c3)nc(-c3cccc4oc5ccccc5c34)n2)cc1]

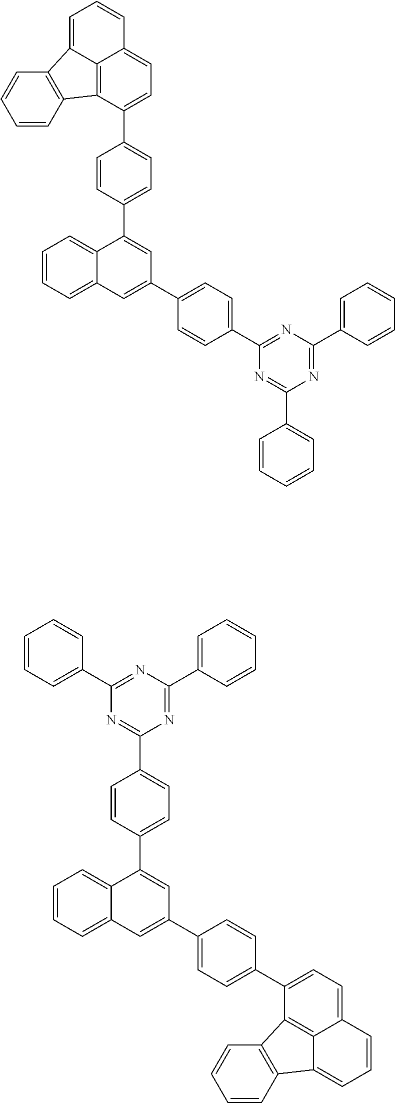 [SMILES: c1ccc(-c2nc(-c3ccccc3)nc(-c3ccc(-c4cc(-c5ccc(-c6ccc7cccc8c7c6-c6ccccc6-8)cc5)c5ccccc5c4)cc3)n2)cc1 ; c1ccc(-c2nc(-c3ccccc3)nc(-c3ccc(-c4cc(-c5ccc(-c6ccc7cccc8c7c6-c6ccccc6-8)cc5)cc5ccccc45)cc3)n2)cc1]

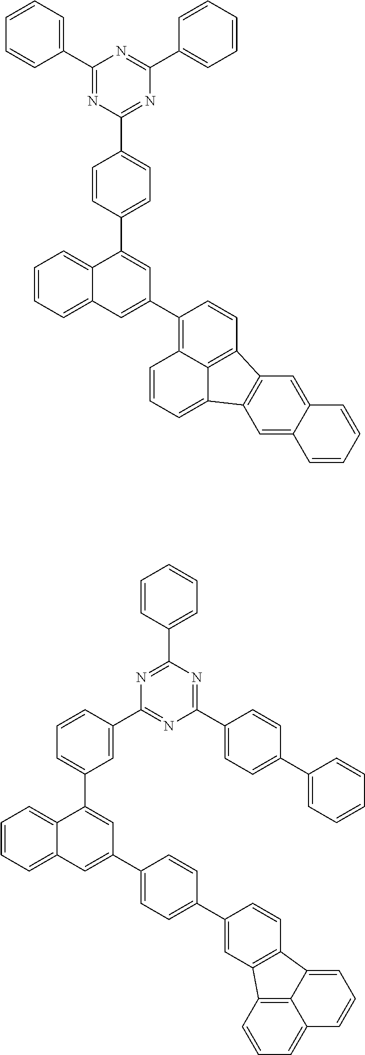 [SMILES: c1ccc(-c2nc(-c3ccccc3)nc(-c3ccc(-c4cc(-c5ccc6c7c(cccc57)-c5cc7ccccc7cc5-6)cc5ccccc45)cc3)n2)cc1 ; c1ccc(-c2ccc(-c3nc(-c4ccccc4)nc(-c4cccc(-c5cc(-c6ccc(-c7ccc8c(c7)-c7cccc9cccc-8c79)cc6)cc6ccccc56)c4)n3)cc2)cc1]

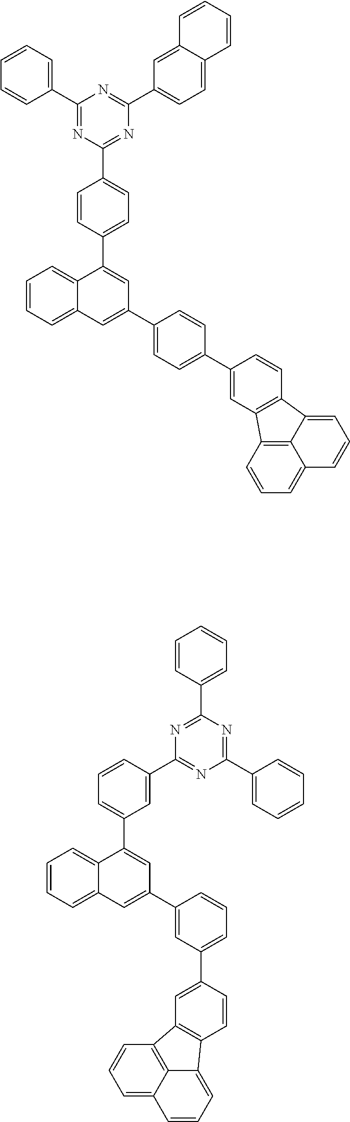 [SMILES: c1ccc(-c2nc(-c3ccc(-c4cc(-c5ccc(-c6ccc7c(c6)-c6cccc8cccc-7c68)cc5)cc5ccccc45)cc3)nc(-c3ccc4ccccc4c3)n2)cc1 ; c1ccc(-c2nc(-c3ccccc3)nc(-c3cccc(-c4cc(-c5cccc(-c6ccc7c(c6)-c6cccc8cccc-7c68)c5)cc5ccccc45)c3)n2)cc1]

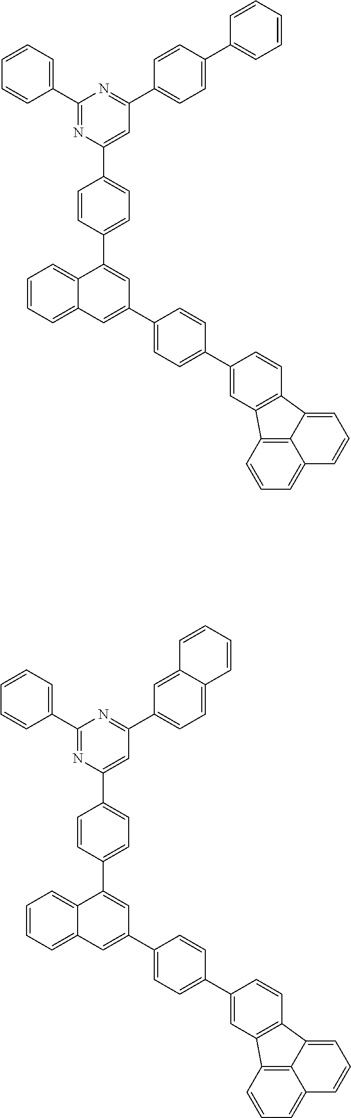 [SMILES: c1ccc(-c2ccc(-c3cc(-c4ccc(-c5cc(-c6ccc(-c7ccc8c(c7)-c7cccc9cccc-8c79)cc6)cc6ccccc56)cc4)nc(-c4ccccc4)n3)cc2)cc1 ; c1ccc(-c2nc(-c3ccc(-c4cc(-c5ccc(-c6ccc7c(c6)-c6cccc8cccc-7c68)cc5)cc5ccccc45)cc3)cc(-c3ccc4ccccc4c3)n2)cc1]

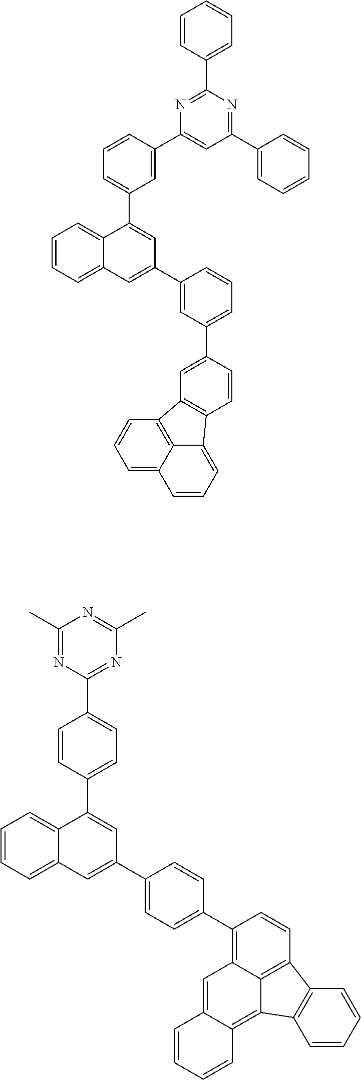 [SMILES: c1ccc(-c2cc(-c3cccc(-c4cc(-c5cccc(-c6ccc7c(c6)-c6cccc8cccc-7c68)c5)cc5ccccc45)c3)nc(-c3ccccc3)n2)cc1 ; Cc1nc(C)nc(-c2ccc(-c3cc(-c4ccc(-c5ccc6c7c(c8ccccc8cc57)-c5ccccc5-6)cc4)cc4ccccc34)cc2)n1]

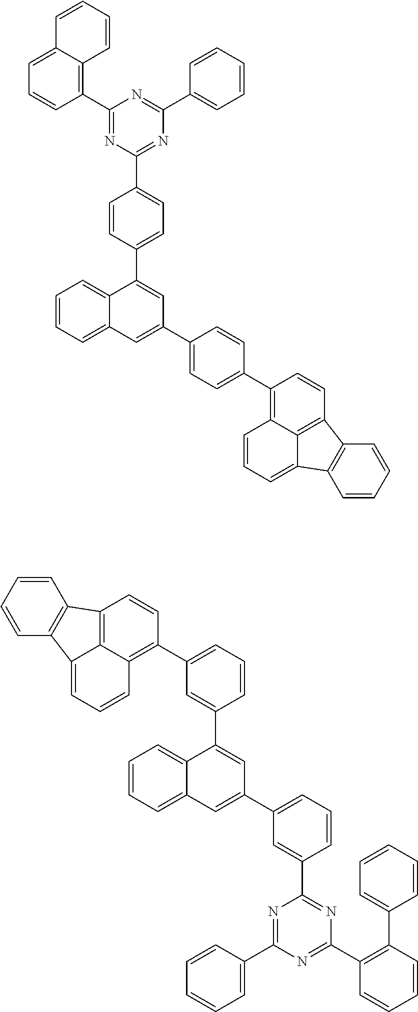 [SMILES: c1ccc(-c2nc(-c3ccc(-c4cc(-c5ccc(-c6ccc7c8c(cccc68)-c6ccccc6-7)cc5)cc5ccccc45)cc3)nc(-c3cccc4ccccc34)n2)cc1 ; c1ccc(-c2nc(-c3cccc(-c4cc(-c5cccc(-c6ccc7c8c(cccc68)-c6ccccc6-7)c5)c5ccccc5c4)c3)nc(-c3ccccc3-c3ccccc3)n2)cc1]

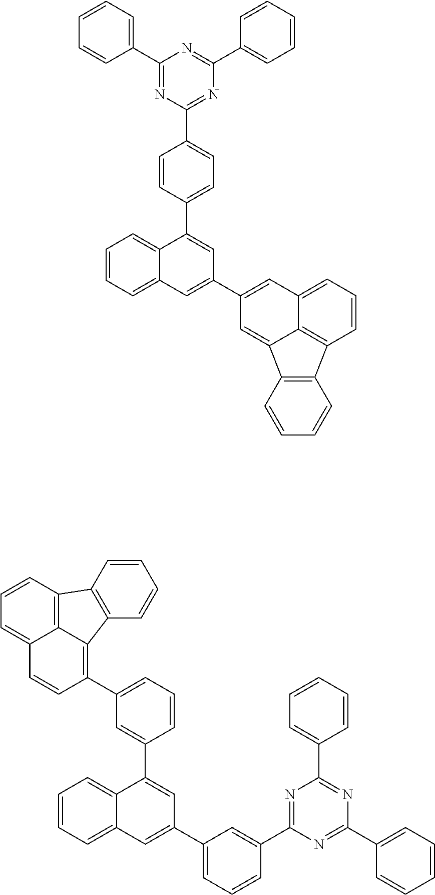 [SMILES: c1ccc(-c2nc(-c3ccccc3)nc(-c3ccc(-c4cc(-c5cc6c7c(cccc7c5)-c5ccccc5-6)cc5ccccc45)cc3)n2)cc1 ; c1ccc(-c2nc(-c3ccccc3)nc(-c3cccc(-c4cc(-c5cccc(-c6ccc7cccc8c7c6-c6ccccc6-8)c5)c5ccccc5c4)c3)n2)cc1]

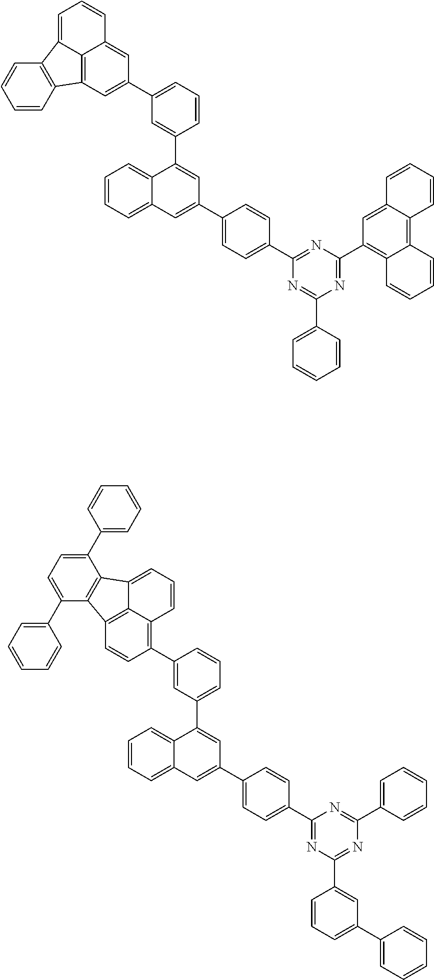 [SMILES: c1ccc(-c2nc(-c3ccc(-c4cc(-c5cccc(-c6cc7c8c(cccc8c6)-c6ccccc6-7)c5)c5ccccc5c4)cc3)nc(-c3cc4ccccc4c4ccccc34)n2)cc1 ; c1ccc(-c2cccc(-c3nc(-c4ccccc4)nc(-c4ccc(-c5cc(-c6cccc(-c7ccc8c9c(cccc79)-c7c(-c9ccccc9)ccc(-c9ccccc9)c7-8)c6)c6ccccc6c5)cc4)n3)c2)cc1]

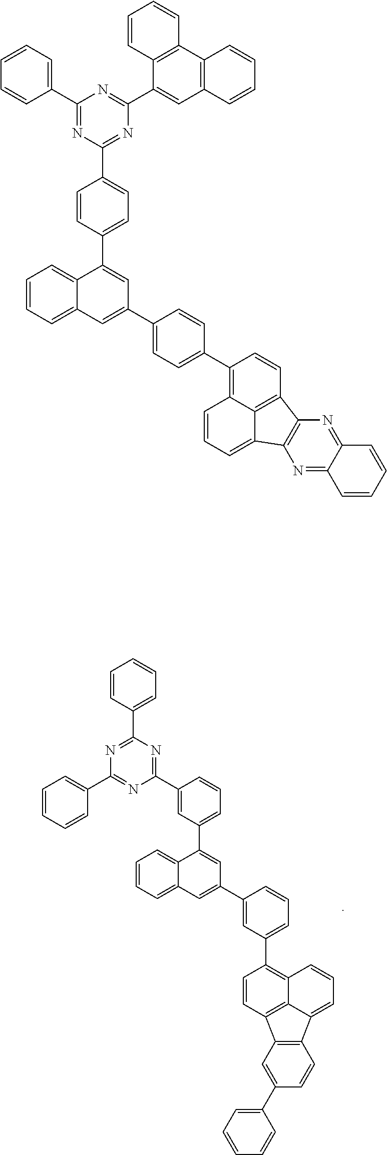 [SMILES: c1ccc(-c2nc(-c3ccc(-c4cc(-c5ccc(-c6ccc7c8c(cccc68)-c6nc8ccccc8nc6-7)cc5)cc5ccccc45)cc3)nc(-c3cc4ccccc4c4ccccc34)n2)cc1 ; c1ccc(-c2ccc3c(c2)-c2ccc(-c4cccc(-c5cc(-c6cccc(-c7nc(-c8ccccc8)nc(-c8ccccc8)n7)c6)c6ccccc6c5)c4)c4cccc-3c24)cc1]

6. An organic light emitting device comprising:
a first electrode;
a second electrode; and
one or more organic material layers provided between the first electrode and the second electrode,
wherein one or more layers of the organic material layers include the compound of claim 1.

7. The organic light emitting device of claim 6, wherein the one or more organic material layers include an electron injection layer, an electron transfer layer, or an electron injection and transfer layer, and the electron injection layer, the electron transfer layer, or the electron injection and transfer layer includes the compound.

8. The organic light emitting device of claim 6, wherein the one or more organic material layers include a hole blocking layer, and the hole blocking layer includes the compound.
